(19) United States
(12) Patent Application Publication
(10) Pub. No.: US 2024/0390491 A1
(43) Pub. Date: Nov. 28, 2024

(54) METHOD AND COMPOSITIONS FOR CELLULAR IMMUNOTHERAPY

(71) Applicants: Fred Hutchinson Cancer Center, Seattle, WA (US); Seattle Children's Hospital, dba Seattle Children's Research Institute, Seattle, WA (US)

(72) Inventors: Michael C. Jensen, Bainbridge Island, WA (US); Stanley R. Riddell, Sammamish, WA (US); Michael Hudecek, Leipzig (DE)

(21) Appl. No.: 18/433,277

(22) Filed: Feb. 5, 2024

Related U.S. Application Data
(63) Continuation of application No. 17/015,995, filed on Sep. 9, 2020, now abandoned.
Application No. 17/015,995 is a division of application No. 14/422,640, filed on Feb. 19, 2015, now Pat. No. 10,780,118.
(60) Provisional application No. 61/691,117, filed on Aug. 20, 2012.

(51) Int. Cl. A61K 39/00; C07K 14/705; C07K 14/715; C07K 14/725; C07K 16/28; C07K 16/32; C07K 16/40

(52) U.S. Cl. CPC A61K 39/4611; A61K 39/4631; A61K 39/464402; A61K 39/464406; A61K 39/464412; C07K 14/705; C07K 14/7051; C07K 14/70521; C07K 14/7151; C07K 16/2803; C07K 16/32; C07K 16/40; A61K 2239/17; A61K 2239/31; A61K 2239/48; C07K 2317/622; C07K 2317/64; C07K 2319/03; C07K 2319/30; C07K 2319/70

(57) ABSTRACT

The present invention provides nucleic acids, vectors, host cells, methods and compositions to confer and/or augment immune responses mediated by cellular immunotherapy, such as by adoptively transferring CD8+ central memory T cells or combinations of central memory T cells with CD4+ T cells that are genetically modified to express a chimeric receptor. In embodiments the genetically modified host cell comprises a nucleic acid comprising a polynucleotide coding for a ligand binding domain, a polynucleotide comprising a customized spacer region, a polynucleotide comprising a transmembrane domain, and a polynucleotide comprising an intracellular signaling domain. It has been surprisingly found that the length of the spacer region can affects the ability of chimeric receptor modified T cells to recognize target cells in vitro and affects in vivo efficacy of the chimeric receptor modified T cells. Pharmaceutical formulations produced by the method, and methods of using the same, are also described.

### STATEMENT REGARDING FEDERALLY SPONSORED RESEARCH

This invention was made with government support under grants CA136551 and CA114536 awarded by the National Institutes of Health. The United States government has certain rights in the invention.

### FIELD OF THE INVENTION

The present invention relates to the field of biomedicine and specifically methods useful for cancer therapy. In particular, embodiments of the invention relate to methods and compositions for carrying out cellular immunotherapy comprising T cells modified with tumor targeting receptors.

### REFERENCE TO AN ELECTRONIC SEQUENCE LISTING

The contents of the electronic sequence listing (425C2_SeqListing.xml; Size: 177,464 bytes; and Date of Creation: Feb. 5, 2024) is herein incorporated by reference in its entirety.

### BACKGROUND OF THE INVENTION

The adoptive transfer of human T lymphocytes that are engineered by gene transfer to express chimeric antigen receptors (chimeric receptors) specific for surface molecules expressed on tumor cells has the potential to effectively treat advanced malignancies.

Chimeric receptors are synthetic receptors that include an extracellular ligand binding domain, most commonly a single chain variable fragment of a monoclonal antibody (scFv) linked to intracellular signaling components, most commonly CD3 (alone or combined with one or more costimulatory domains. Much of the research in the design of chimeric receptors has focused on defining scFvs and other ligand binding elements that target malignant cells without causing serious toxicity to essential normal tissues, and on defining the optimal composition of intracellular signaling modules to activate T cell effector functions. However, it is uncertain whether the variations in chimeric receptor design that mediate superior in vitro function will translate reproducibly into improved in vivo therapeutic activity in clinical applications of chimeric receptor-modified T cells.

There is a need to identify methods for determining elements of chimeric receptor design that are important for therapeutic activity and cell populations to genetically modify and adoptively transfer that provide enhanced survival and efficacy in vivo.

### SUMMARY OF THE INVENTION

In one aspect, the present disclosure relates to methods and compositions to confer and/or augment immune responses mediated by cellular immunotherapy, such as by adoptively transferring tumor-specific, genetically modified subsets of CD8+ or CD4+ T cells alone, or in combination. The disclosure provides for chimeric receptor nucleic acids, and vectors and host cells including such nucleic acids. The nucleic acid sequence that encodes the chimeric receptor links together a number of modular components that can be excised and replaced with other components in order to customize the chimeric receptor for efficient T cell activation and recognition of a specific target molecule or an epitope on the target molecule.

In embodiments, a chimeric receptor nucleic acid comprises a polynucleotide coding for a ligand binding domain, wherein the ligand is a molecule expressed on malignant or infected cells, a polynucleotide coding for a polypeptide spacer wherein the polypeptide spacer is about 200 amino acids or less, a polynucleotide coding for a transmembrane domain; and a polynucleotide coding for intracellular signaling domains. In embodiments, the polypeptide spacer comprises a modified IgG4 hinge region containing an amino acid sequence X₁PPX₂P (SEQ ID NO:1) that may be linked to other amino acid sequences including but not limited to the CH2 and CH3 or CH3 only sequences of the Ig Fc. It has been surprisingly found that the length of the spacer region that is presumed not to have signaling capability affects the in vivo efficacy of the T cells modified to express the chimeric receptor and needs to be customized for individual target molecules for optimal tumor or target cell recognition.

Another aspect of the disclosure provides an isolated chimeric receptor nucleic acid comprising: a polynucleotide coding for a ligand binding domain, wherein the ligand is a tumor specific antigen, viral antigen, or any other molecule expressed on a target cell population that is suitable to mediate recognition and elimination by a lymphocyte; a polynucleotide coding for a polypeptide spacer wherein the polypeptide spacer is of a customized length that is specific for each targeted ligand, wherein the spacer provides for enhanced T cell proliferation and/or cytokine production as compared to a reference chimeric receptor; a polynucleotide coding for a transmembrane domain; and a polynucleotide coding for one or more intracellular signaling domains. In embodiments, a long spacer is employed if the epitope on the target ligand is in a membrane proximal position and a short spacer is employed if the epitope on the target ligand is in a membrane distal position. The disclosure includes expression vectors and host cells comprising the isolated chimeric receptor as described herein.

Another aspect of the disclosure provides a chimeric receptor polypeptide comprising a ligand binding domain, wherein the ligand is a tumor specific antigen, viral antigen or any other molecule that is expressed on a target cell population and can be targeted to mediate recognition and elimination by lymphocytes; a polypeptide spacer wherein the polypeptide spacer is about 10-229 amino acids; a transmembrane domain; and one or more intracellular signaling domains. In embodiments, the polypeptide spacer comprises a modified IgG hinge region containing the amino acid sequence X₁PPX₂P (SEQ ID NO:1).

In another aspect, the present disclosure provides compositions to confer and/or augment immune responses mediated by cellular immunotherapy, such as by adoptively transferring tumor-specific, subset specific genetically modified CD4+ T cells, wherein the CD4+ T cells confer and/or augment the ability of CD8+ T cells to sustain anti-tumor reactivity and increase and/or maximize tumor-specific proliferation. In embodiments, the CD4+ cells are genetically modified to express a chimeric receptor nucleic acid and/or chimeric receptor polypeptide as described herein.

In another aspect, the present disclosure provides compositions to confer and/or augment immune responses mediated by cellular immunotherapy, such as by adoptively transferring tumor-specific, subset specific genetically modified CD8+ T cells. In embodiments, the CD8+ cells express a chimeric receptor nucleic acid and/or chimeric receptor polypeptide as described herein.

In another embodiment, the present invention provides an adoptive cellular immunotherapy composition having a genetically modified CD8+ cytotoxic T lymphocyte cell preparation to confer and/or augment immune responses, wherein the cytotoxic T lymphocyte cell preparation comprises CD8+ T cells that express a chimeric receptor comprising a ligand binding domain for a ligand associated with the disease or disorder, a customized spacer region, a transmembrane domain; and an intracellular signaling domain of a T cell or other receptors, such as a costimulatory domain, and/or a genetically modified helper T lymphocyte cell preparation, wherein the helper T lymphocyte cell preparation has CD4+ T cells that express a chimeric receptor comprising an antibody variable domain specific for the ligand associated with the disease or disorder, a customized spacer region, a transmembrane domain; and one or more intracellular signaling domains.

In one embodiment, the present invention provides a method of performing cellular immunotherapy in a subject having a disease or disorder by administering to the subject a genetically modified cytotoxic T lymphocyte cell preparation that provides a cellular immune response, wherein the cytotoxic T lymphocyte cell preparation comprises CD8+ T cells that have a chimeric receptor comprising a polynucleotide coding for a ligand binding domain, wherein the ligand is a tumor specific antigen, viral antigen, or any other molecule expressed on a target cell population that is suitable to mediate recognition and elimination by a lymphocyte; a polynucleotide coding for a polypeptide spacer wherein the polypeptide spacer is of a customized length that is specific for each targeted ligand, wherein the spacer provides for enhanced T cell proliferation and/or cytokine production as compared to a reference chimeric receptor; a polynucleotide coding for a transmembrane domain; and a polynucleotide coding for one or more intracellular signaling domains. In embodiment, the ligand binding domain is an extracellular antibody variable domain specific for a ligand associated with the disease or disorder. An embodiment includes a genetically modified helper T lymphocyte cell preparation that wherein the helper T lymphocyte cell preparation comprises CD4+ T cells that have a chimeric receptor comprising an a polynucleotide coding for a ligand binding domain, wherein the ligand is a tumor specific antigen, viral antigen, or any other molecule expressed on a target cell population that is suitable to mediate recognition and elimination by a lymphocyte; a polynucleotide coding for a polypeptide spacer wherein the polypeptide spacer is of a customized length that is specific for each targeted ligand, wherein the spacer provides for enhanced T cell proliferation and/or cytokine production as compared to a reference chimeric receptor; a polynucleotide coding for a transmembrane domain; and a polynucleotide coding for one or more intracellular signaling domains. In embodiments, the genetically modified CD8+ and genetically modified CD4+ cell population are coadministered. In embodiments, the T cells are autologous or allogeneic T cells.

Various modifications of the above method are possible. For example, the chimeric receptor that is expressed by the CD4+ T cell and the CD8+ T cell can be the same or different.

In another aspect, the present invention provides a method of manufacturing an adoptive immunotherapy composition by obtaining a chimeric receptor modified tumor-specific CD8+ cytotoxic T lymphocyte cell preparation that elicits a cellular immune response and expresses an antigen-reactive chimeric receptor, wherein the modified cytotoxic T lymphocyte cell preparation comprises CD8+ T cells that have a chimeric receptor comprising a ligand binding domain, wherein the ligand is a tumor specific antigen, viral antigen, or any other molecule expressed on a target cell population that is suitable to mediate recognition and elimination by a lymphocyte; a polypeptide spacer wherein the polypeptide spacer is of a customized length that is specific for each targeted ligand, wherein the spacer provides for enhanced T cell proliferation and/or cytokine production as compared to a reference chimeric receptor; a transmembrane domain; and one or more intracellular signaling domains.; and/or obtaining a modified naïve or memory CD4+ T helper cell wherein the modified helper T lymphocyte cell preparation comprises CD4+ cells that have a chimeric receptor comprising a ligand binding domain, wherein the ligand is a tumor specific antigen, viral antigen, or any other molecule expressed on a target cell population that is suitable to mediate recognition and elimination by a lymphocyte; a polypeptide spacer wherein the polypeptide spacer is of a customized length that is specific for each targeted ligand, wherein the spacer provides for enhanced T cell proliferation and/or cytokine production as compared to a reference chimeric receptor; a transmembrane domain; and one or more intracellular signaling domains.

These and other embodiments of the invention are described further in the accompanying specification, drawings and claims.

### BRIEF DESCRIPTION OF THE DRAWINGS

FIG. 1 Library of spacer sequences. We constructed a plasmid library that contain codon optimized DNA sequences that encode extracellular components including of the IgG4 hinge alone, IgG4 hinge linked to CH2 and CH3 domains, or IgG4 hinge linked to the CH3 domain. Any scFV sequence (VH and VL) can be cloned 5′ to the sequences encoding this library of variable spacer domains. The spacer domains are in turn linked to CD28 transmembrane and intracellular signaling domains and to CD3 zeta. A T2A sequence in the vector separates the chimeric receptor from a selectable marker encoding a truncated human epidermal growth factor receptor (tEGFR).

FIG. 2: In vitro cytotoxicity, cytokine production, and proliferation of T-cells modified to express 2A2 ROR1 chimeric receptors with modified spacer length. (A) Phenotype of purified CD8⁺ T_{CM}-derived cell lines modified with each of the 2A2 ROR1 chimeric receptors with long, intermediate and short spacer domain. Staining with anti-F(ab) antibody that binds to an epitope in the 2A2 scFV shows surface expression of ROR1 chimeric receptors with full length or truncated spacer. (B) Cytolytic activity of T-cells expressing the various 2A2 ROR1 chimeric receptors with long (●), intermediate (▴) and short spacer (♦), or a tEGFR control lentiviral vector against ROR1⁺ (x) and control target cells. The bar diagram summarizes cytotoxicity data from 3 independent experiments (E:T=30:1) normalized to cytolytic activity by 2A2 ROR1 chimeric receptor ‘long’=1, and analyzed by Student's t-test. (C) CFSE dye dilution was used to measure proliferation of 2A2 ROR1 chimeric receptor and tEGFR control T-cells, 72 hours after stimulation with Raji/ROR1 (left panel) and primary CLL cells (right panel) without addition of exogenous cytokines. For analysis, triplicate wells were pooled and the proliferation of live (PI⁻), CD8⁺ T-cells analyzed. Numbers above each histogram indicate the number of cell divisions the proliferating subset underwent, and the fraction of T-cells in each gate that underwent ≥4/3/2/1 cell divisions is provided next to each plot. (D) Multiplex cytokine assay of supernatants obtained after 24 hours from triplicate co-cultures of 5×10⁴ T-cells expressing the various 2A2 ROR1 chimeric receptors with Raji/ROR1 and primary CLL cells. Multiplex cytokine data from 3 independent experiments were normalized (cytokine release by 2A2 ROR1 chimeric receptor ‘long’=1) and analyzed by Student's t-test (right bar diagram).

FIG. 3. R11 chimeric receptor requires a long spacer for recognition of ROR1⁺ tumor cells. The sequences encoding the scFV from the R11 monoclonal antibody that is specific for an epitope in the membrane proximal Kringle domain of the orphan tyrosine kinase receptor ROR1 were cloned upstream of IgG4 hinge only (short), IgG4 hinge/CH3 (intermediate), and IgG4 hinge/CH2/CH3 sequences in our chimeric receptor library containing the 4-1BB costimulatory domains and prepared as lentiviral vectors. A). Human CD8⁺ T cells were transduced and the transduction efficiency with each of the short, intermediate and long chimeric receptors was determined by staining for the tEGFR marker. B). Transduced T cells expressing the short (top), intermediate (middle), and long (bottom) were assayed for lysis of K562 leukemia cells alone or transfected to express ROR1. Only the T cells expressing the long spacer chimeric receptor efficiently killed ROR1+ K562 cells. C). Transduced T cells expressing the short (top), intermediate (middle), and long (bottom) were labeled with CFSE, stimulated with K562 cells expressing ROR1 or CD19 (control) and assayed for cell proliferation over 72 hours. The T cells expressing the long spacer chimeric receptor proliferated specifically to the ROR1+ K562 cells. D). Transduced T cells expressing the short (top), intermediate (middle), and long (bottom) were stimulated with Raji lymphoma cells and K562 cells that expressed ROR1 or CD19 (control) and assayed for the secretion of interferon gamma into the supernatant over 24 hours. The T cells expressing the long spacer chimeric receptor proliferated and produced the highest levels of interferon gamma in response to ROR1 positive target cells.

FIG. 4: Design of ROR1 chimeric receptors with modified spacer length and derived from the 2A2 and R12 scFV with different affinity. (A) Design of lentiviral transgene inserts encoding a panel of ROR1 chimeric receptors containing the 2A2 scFV, an IgG4-Fc derived spacer of ‘Hinge-CH2-CH3’ (long spacer, 229 AA), ‘Hinge-CH3’ (intermediate, 119 AA), or ‘Hinge’ only (short, 12 AA), and a signaling module with CD3 ζ and CD28. Each chimeric receptor cassette contains a truncated EGFR marker encoded downstream of a T2A element. (B) Lentiviral transgene inserts encoding ROR1-specific chimeric receptors derived from the R12 and 2A2 scFV with short IgG4-Fc ‘Hinge’ spacer (12 AA), and a signaling module containing CD28 or 4-1BB and CD3 (respectively (total: 4 constructs).

FIG. 5: Anti-tumor reactivity of T-cells modified with ROR1 chimeric receptors derived from mAb R12 with higher affinity than 2A2. (A) tEGFR expression on purified polyclonal CD8⁺ T_{CM}-derived T-cell lines modified with each of the R12 and 2A2 ROR1 chimeric receptors with short IgG4-Fc ‘Hinge’ spacer, and CD28 or 4-1BB costimulatory domain. (B) Cytotoxicity against ROR1⁺ and control target cells by T-cells expressing R12(28-▴; 4-1BB-Δ) and 2A2 ROR1 chimeric receptors (28-●; 4-1BB∘) or a tEGFR control vector (x). (C) Multiplex cytokine assay of supernatants obtained after 24 hours from co-cultures of 5×10⁴ T-cells expressing the various ROR1 chimeric receptors with Raji/ROR1 tumor cells. The middle/right bar diagrams show normalized multiplex data from 3 independent experiments (cytokine release by ROR1 chimeric receptor 2A2=1) analyzed by Student's t-test. (D) Proliferation of ROR1 chimeric receptor T-cells and tEGFR control T-cells 72 hours after stimulation with Raji/ROR1 cells and without addition of exogenous cytokines was assessed by CFSE dye dilution. Numbers above each histogram indicate the number of cell divisions the proliferating subset underwent, and the fraction of T-cells in each gate that underwent ≥4/3/2/1 cell divisions is provided above each plot.

FIG. 6: Analysis of cytokine production and proliferation of CD4⁺ T-cells lines modified with a ROR1 chimeric receptor derived from mAb R12 with higher affinity than 2A2. (A-B) The 2A2 and R12 ROR1 chimeric receptors had the short spacer and a CD28 costimulatory domain. (A) Multiplex cytokine analysis from supernatants obtained 24 hours after stimulation of 5×10⁴ CD4⁺ T-cells expressing the 2A2 and R12 ROR1 chimeric receptor with Raji/ROR1 tumor cells. (B) Proliferation of CD4⁺ R12 and 2A2 ROR1 chimeric receptor T-cells and tEGFR control T-cells 72 hours after stimulation with Raji/ROR1 cells and without addition of exogenous cytokines was assessed by CFSE dye dilution. Numbers above each histogram indicate the number of cell divisions the proliferating subset underwent, and the fraction of T-cells in each gate that underwent ≥5/4/3/2/1 cell divisions is provided above the histograms.

FIG. 7: Recognition of primary CLL by T-cells modified with 2A2 and R12 ROR1 chimeric receptors with optimal short spacer and 4-1BB costimulatory domain or with a CD19-specific chimeric receptor. (A) Expression of ROR1/CD19 on primary CLL, and CD80/86 on primary CLL and Raji/ROR1 tumor cells (black dot plots) that can engage CD28 on chimeric receptor T-cells (white histograms). Staining with matched isotype control mAbs is shown as grey dot plots/histograms. (B) Cytolytic activity of T-cells expressing the 2A2(●) and R12 ROR1 chimeric receptor (▪), a CD19-specific chimeric receptor (▴) and T-cells modified with a tEGFR control vector (x) against primary CLL (left diagram) and normal B cells (right diagram) analyzed by chromium release assay. Cytotoxicity data against primary CLL from 4 independent experiments (E:T=30:1) were normalized (cytolytic activity by ROR1 chimeric receptor 2A2=1) and analyzed by Student's t-test (bar diagram). (C) Multiplex cytokine analysis after a 24-hour stimulation of 5×10⁴ chimeric receptor T-cells with primary CLL cells. Cytokine release of unstimulated chimeric receptor T-cells was below 3.6 pg/ml (detection limit) (left bar diagram). ELISA for IFN-γ production by 5×10⁴ 2A2 and R12 ROR1 chimeric receptor T-cells after a 24-hour co-culture with primary CLL. O.D. of 1 corresponds to approximately 250 pg/ml (right bar diagram). (D) Proliferation of CD8⁺ T-cells modified with the 2A2 ROR1, R12 ROR1 and a CD19 chimeric receptor, 72 hours after stimulation with primary CLL cells. Numbers above each histogram indicate the number of cell divisions, and the fraction of T-cells in each gate that underwent ≥3/2/1 cell divisions is provided next to each plot.

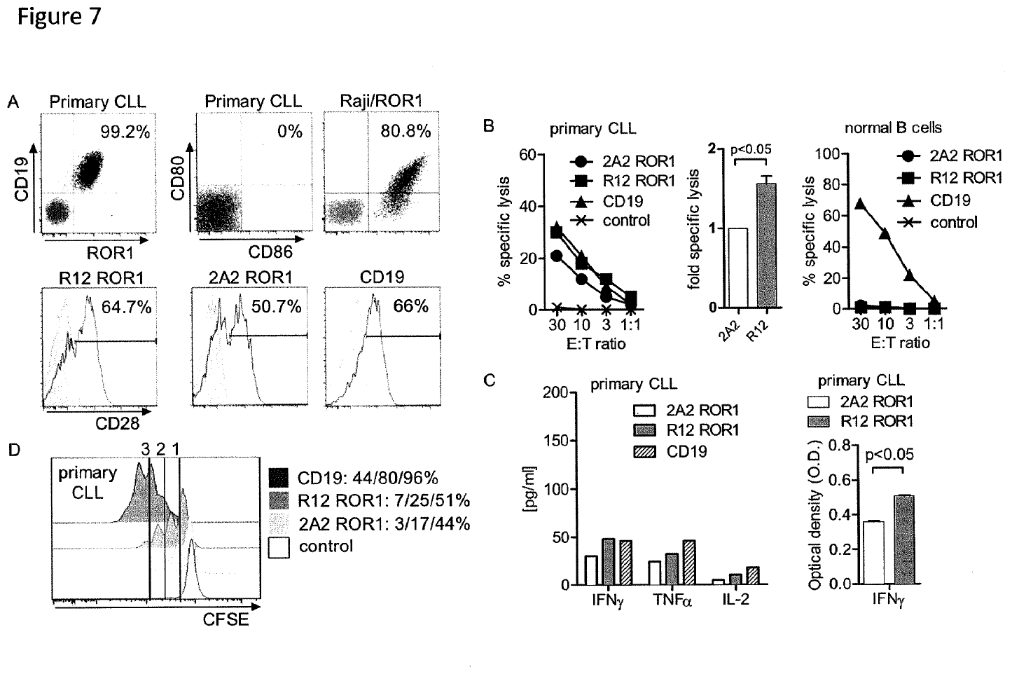

FIG. 8: The function of ROR1-chimeric receptor and CD19-chimeric receptor modified CD8⁺ T-cells against primary CLL is augmented by chimeric receptor-modified CD4⁺ helper T-cells. (A) ELISA for IL-2 production from triplicate co-cultures of 5×10⁴ CD8⁺ and CD4⁺ T-cells expressing the R12 ROR1 and CD19-chimeric receptor respectively, incubated with primary CLL for 24-hours. O.D. of 1 corresponds to approx. 800 pg/ml. (B) Proliferation of chimeric receptor-modified CD8⁺ T-cells in response to primary CLL is enhanced by addition of chimeric receptor-modified CD4⁺ T-cells. CFSE-labeled CD8⁺ T-cells expressing the 2A2 ROR1, R12 ROR1 and CD19-chimeric receptor respectively, were co-cultured with tumor cells and with 2A2 ROR1, R12 ROR1 and CD19-chimeric receptor transduced or control untransduced CD4⁺ T-cells (CD8⁺:CD4⁺=1:1). Proliferation of the CD8⁺ subset was analyzed 72 hours after stimulation. Numbers above each histogram indicate the number of cell divisions, and the fraction of T-cells in each gate that underwent ≥3/2/1 cell divisions is provided above each plot.

FIG. 9: In vivo anti-tumor efficacy of 2A2 ROR1, R12 ROR1 and CD19 chimeric receptor T-cells. Cohorts of mice were inoculated with 0.5×10⁶ JeKo-1/ffluc MCL via tail vein injection, and 5×10⁶ 2A2 ROR1, R12 ROR1 or CD19 chimeric receptor T-cells, or T-cells expressing a tEGFR control vector were administered 7 days after tumor inoculation. All chimeric receptor constructs had the short IgG4 ‘Hinge-only’ spacer and a 4-1BB costimulatory domain. (A, B) Serial bioluminescence imaging of tumor in cohorts of mice treated with T-cells expressing the 2A2 ROR1 chimeric receptor (▾), the high affinity R12 ROR1 chimeric receptor (▪), a CD19-specific chimeric receptor (▴), with T-cells transduced with tEGFR alone (●), and untreated mice. Bioluminescence imaging showed tumor manifestations in the bone marrow and thorax and thus, signal intensity was measured in regions of interest that encompassed the entire body and thorax of each individual mouse. (C) Kaplan-Meier analysis of survival in individual treatment and control groups. Statistical analyses were performed using the log-rank test. The data shown in A-C are representative of results obtained in 2 independent experiments. (D) Proliferation of 2A2 ROR1, R12 ROR1 and CD19 chimeric receptor T-cells in vivo. Tumor bearing NSG/JeKo-1 mice received a single dose of 5×10⁶ CFSE-labeled 2A2 ROR1, R12 ROR1 or CD19 chimeric receptor T-cells on day 7 after tumor inoculation, and 72 h later peripheral blood, bone marrow and spleen were collected from each individual mouse. The frequency and proliferation of live (PI⁻), CD45⁺ CD8⁺ tEGFR⁺ T-cells was analyzed. The frequency of 2A2 ROR1, R12 ROR1 and CD19 chimeric receptor T-cells respectively is provided on the left of each histogram as percentage of live cells, and the fraction of T-cells that underwent ≥4/3/2/1 cell divisions is provided above each plot.

FIG. 10 Expression of ROR1 and NKG2D ligands on epithelial cancer cell lines. (A) Expression of ROR1 on the triple negative breast cancer cell lines MDA-MB-231 and 468, and the renal cell cancer lines FARP, TREP and RWL (black histograms). Staining with matched isotype control antibody is shown as grey histograms. (B) Expression of CD80/86 and the NKG2D ligands MICA/B on MDA-MB-231 and Raji/ROR1 tumor cells, and NKG2D (CD314) on 2A2 and R12 ROR1-chimeric receptor T-cells. Staining with matched isotype control mAbs is shown as grey dot plots/histograms.

FIG. 11: ROR1-chimeric receptor modified T-cells recognize ROR1⁺ epithelial tumor cells in vitro. (A) Chromium release assay to evaluate the cytolytic activity of R12 ROR1-chimeric receptor modified T-cells (short spacer/4-1BB costimulatory domain, closed symbols) and tEGFR control T-cells (open symbols) against ROR1⁺ breast cancer and renal cell cancer lines. (A-D) The 2A2 and R12 ROR1-chimeric receptors had the optimal short spacer and a 4-1BB costimulatory domain. (B) Multiplex cytokine analysis after stimulation of T-cells expressing the 2A2 and R12 ROR1-chimeric receptor with MDA-MB-231 and Raji/ROR1 tumor cells. (C) Proliferation of CD8⁺ T-cells modified with the 2A2 and R12 ROR1-chimeric receptor 72 hours after stimulation with MDA-MB-231 tumor cells. For analysis, triplicate wells were pooled and the proliferation of live (PI⁻), CD8⁺ T-cells analyzed. Numbers above each histogram indicate the number of cell divisions the proliferating subset underwent, and the fraction of T-cells in each gate that underwent ≥4/3/2/1 cell divisions is provided next to each histogram. (D) ELISA for IL-2 production by R12 ROR1-chimeric receptor T-cells after a 24-hour co-culture with MDA-MB-231 in plain medium, and after addition of an antibody cocktail blocking of the NKG2D pathway [anti-NKG2D (clone 1D11), anti-MICA/B (clone 6D4) and anti-ULBP] or matched isotype control mAbs. O.D. of 0.6 corresponds to approximately 1900 μg/ml.

FIG. 12. Effect of extracellular spacer length on recognition and triggering of tumor cell lysis by CD8+ human T cells that express a HER2-specific chimeric receptor. A.) Depiction of Herceptin Fab epitope location on tumor cell membrane proximal epitope on human HER2, B.) Structural formats of Herceptin scFv CAR spacer length variants as—T2A-linked polypeptides with the carboxyl EGFRt marker transmembrane protein, C.) Western blot detection of short, medium, and long spacer Herceptin-CAR variant expression in human CD8+ CTL's, D.) Flow cytometric detection of EGFRt by transduced human CD8+ CTL's transduced with Herceptin CAR variants then immunomagnetically purified by Herceptin-biotin, anti-biotin microbeads, E.) Distinct cytolytic function by T cells transduced to express the Herceptin CAR variants (short—S; medium—M; and long—L) against HER2⁺ Med411FH and D283 human medulloblastoma cell lines (D341 is a HER2⁻ control medulloblastoma cell line, inset flow plots are tumor target lines stained with anti-HER2 specific mAb). Green=full IgG4 (Long Spacer, ▾), Blue=IgG4hinge:CH3 (Medium Spacer; ▴), Red=IgG4hinge only (Short Spacer; ▪).

FIG. 13: CD19-chimeric receptor vectors and generation of CD19-chimeric receptor T cells. (A) Design of lentiviral transgene inserts encoding a panel of CD19-specific chimeric receptors that differ in extracellular spacer length and intracellular co-stimulation. Each chimeric receptor encoded the CD19-specific single chain variable fragment derived from the FMC63 mAb in a VL-VH orientation, an IgG4-derived spacer domain of Hinge-CH2-CH3 (long spacer, 229 AA) or Hinge only (short spacer, 12 AA), and a signaling module containing CD3 (with CD28 or 4-1BB alone or in tandem. Each chimeric receptor cassette contains a truncated EGFR marker encoded downstream of a cleavable 2A element. (B, C) Polyclonal T cell lines modified with each of the CD19-chimeric receptor constructs were prepared from purified CD8⁺ CD45RO⁺ CD62L⁺ central memory T cells (T_{CM}) of normal donors. Following lentiviral transduction, transgene-positive T cells in each cell line were purified using the tEGFR marker and expanded for in vitro and in vivo experiments. (D) MFI after staining for the tEGFR marker shows equivalent transgene expression in T cells modified with each of the CD19-chimeric receptors.

FIG. 14: In vitro cytotoxicity, cytokine production, and proliferation of T cells modified with distinct CD19-chimeric receptors. (A) Cytolytic activity of T cells expressing the various CD19-chimeric receptors against CD19⁺ and control target cells. (B) Multiplex cytokine assay of supernatants obtained after 24 hours from triplicate co-cultures of T cells expressing the various CD19-chimeric receptors and K562 cells transfected with CD19, and CD19⁺ Raji cells. (C) Comparison of cytokine production by T cells expressing the various CD19-chimeric receptors. Multiplex cytokine data from 6 independent experiments were normalized (cytokine release by CD19-chimeric receptor ‘short/CD28’ CTL=1) and analyzed by Student's t-test. (D) CFSE dye dilution was used to measure proliferation of CD19-chimeric receptor T cells 72 hours after stimulation with K562/CD19 (upper panel) and CD19⁺ Raji tumor cells (lower panel) without addition of exogenous cytokines. For analysis, triplicate wells were pooled and the proliferation of live (PI⁻), CD8⁺ T cells analyzed. Numbers above each histogram indicate the number of cell divisions the proliferating subset underwent, and the fraction of T cells in each gate that underwent ≥4/3/2/1 cell divisions is provided in the upper left of each plot. (E) PI staining was performed at the end of a 72-hour co-culture of T cells expressing the various CD19-chimeric receptors with Raji tumor cells. The percentage of PI⁺ cells within in chimeric receptor T cell line (CD3⁺) is provided in each histogram.

FIG. 15: CD19-chimeric receptor T cells with a short extracellular spacer domain eradicate Raji tumors in NOD/SCID mice. (A) Cohorts of mice were inoculated with Raji-ffluc via tail vein injection, and T cells transduced with CD19-chimeric receptors containing long and short spacer domains or with tEGFR alone were administered 2 and 9 days after tumor inoculation by tail vein injection. Tumor progression and distribution was evaluated by serial bioluminescence imaging after injection of luciferin substrate. (B) Serial bioluminescence imaging of tumor in cohorts of mice either treated with T cells expressing CD19-chimeric receptors with short spacer (‘short/CD28’ and ‘short/4-1BB’) and long spacer (‘long/CD28’ and ‘long/4-1BB’) domains, with T cells transduced with the tEGFR control vector, or untreated. Each diagram representing cohorts of mice treated with CD19-chimeric receptor or tEGFR transduced T cells also shows the mean of tumor progression in untreated mice for comparison (red triangles). (C) Kaplan-Meier analyses of survival of untreated mice and mice treated with T cells expressing CD19-chimeric receptors with short spacer (‘short/CD28’ and ‘short/4-1BB’), long spacer (‘long/CD28’ and ‘long/4-1BB’) domains, and with control tEGFR. Statistical analyses were performed using the log-rank test. The data shown in B and C are representative of results obtained in 3 independent experiments.

FIG. 16: CD19-chimeric receptor T cells with a short spacer (short/4-1BB) eradicate established Raji tumors in NSG mice in a dose-dependent manner. (A) Mice were inoculated with Raji-ffluc via tail vein injection and tumor engraftment confirmed by bioluminescence imaging on day 6. On day 7, mice received a single i.v. injection of various doses of T cells transduced with the CD19-chimeric receptor ‘short/4-1BB’ or with the tEGFR-control lentivirus. (B, C) Dose dependent anti-tumor efficacy of T cells expressing the CD19-chimeric receptor ‘short/4-1BB’. A control cohort of mice received a single high dose of T cells modified with tEGFR alone. (D) Persistence of CD19-chimeric receptor T cells following adoptive transfer into NSG/Raji mice. Flow cytometric analysis of peripheral blood (eye bleeds) in the cohort of mice treated with 2.5×10⁶ CD19-chimeric receptor ‘short/4-1BB’ T cells. The frequency of CD8⁺ tEGFR⁺ T cells is shown as percentage of live peripheral blood cells.

FIG. 17: T cells expressing CD19-chimeric receptors with a short spacer and either CD28 or 4-1BB are more effective against established lymphoma than those expressing CD19-chimeric receptors with a long spacer. (A) NSG mice were inoculated with Raji-ffluc on day 0, and treated on day 7 with one dose of 2.5×10⁶ CD19 chimeric receptor T cells expressing short or long spacer and either CD28 or 4-1BB costimulatory domain. (B) Kaplan-Meier analyses of survival of mice in each of the treatment groups. Statistical analyses were performed using the log-rank test. (C) Bioluminescence imaging of cohorts of mice treated with T cells expressing CD19-chimeric receptors with short spacers (‘short/CD28’ and ‘short/4-1BB’), and long spacers (‘long/CD28 and ‘long/4-1BB’). The mean tumor burden observed in untreated mice at each time point is shown in each diagram for comparison (triangles). (D) In vivo persistence of T cells expressing CD19-chimeric receptor with short spacer domain is enhanced compared to T cells expressing CD19-chimeric receptors with long spacer domain. The frequency of CD8⁺ tEGFR⁺ T cells in the peripheral blood obtained at day 3 and 10 after transfer was determined by flow cytometry and is shown as percentage of live (PI⁻) peripheral blood cells. Statistical analyses were performed by Student's t-test. The data shown in B-D are representative for results obtained in 3 independent experiments.

FIG. 18: Increasing chimeric receptor T cell dose or augmenting costimulatory signaling does not improve the anti-tumor efficacy of CD19-chimeric receptors with a long spacer domain against established lymphoma. (A) Cytolytic activity of T cells expressing ‘long/CD28’, ‘long/4-1BB’ and ‘long/CD28_4-1BB’ CD19 chimeric receptors against CD19⁺ and control target cells. (B) Multiplex cytokine assay of supernatant obtained after 24 hours from triplicate co-cultures of K562/CD19 and Raji tumor cells with T cells expressing the various CD19-chimeric receptors. (C) Evaluation of proliferation of CD19-chimeric receptor T cells 72 hours after stimulation with CD19⁺ tumor cells (K562/CD19—left panel; Raji—right panel) by CFSE dye dilution. For analysis, triplicate wells were pooled and the proliferation of live (PI⁻) CD8⁺ T cells analyzed. Numbers above each histogram indicate the number of cell divisions the proliferating subset underwent, and the fraction of T cells in each gate that underwent ≥4/3/2/1 cell divisions is provided in the upper left of each plot. (D) Kaplan-Meier analyses of survival of mice treated with T cells expressing CD19-chimeric receptors with short (‘short/CD28’) and long spacer domain (‘long/CD28’ and ‘long/CD28_4-1BB’), or T cells modified with a tEGFR-encoding control lentiviral vector. Statistical analyses were performed using the log-rank test. (E) Bioluminescence imaging of cohorts of mice treated with T cells expressing CD19-chimeric receptors with short spacer (‘short/CD28’), and long spacers (‘long/CD28 and ‘long/CD28_4-1BB’). Diagrams show mean tumor progression in untreated mice for comparison (red triangles). (F) In vivo persistence of T cells expressing the various CD19-chimeric receptors. The frequency of CD8⁺ tEGFR⁺ T cells in the peripheral blood obtained at day 3 and 10 after transfer was determined by flow cytometry and is shown as percentage of live (PI⁻) peripheral blood cells. Statistical analyses were performed by Student's t-test.

FIG. 19: CD19-chimeric receptor T cells with a long spacer domain are activated by tumor in vivo but fail to increase in cell number. (A) Expression of CD69 and CD25 on T cells modified with each CD19-chimeric receptor prior to transfer into NSG/Raji mice. (B) Cohorts of mice were inoculated with Raji-ffluc tumor cells and 7 days later received CFSE-labeled CD19-chimeric receptor transduced or control T cells. Bone marrow and spleens were harvested from subgroups of mice 24 and 72 hours after T cell administration. (C, D) Multiparameter flow cytometric analysis of bone marrow mononuclear cells obtained 24 hours (C) and 72 hours (D) after T cell transfer. Dot plots show anti CD3 and anti CD45 staining after gating on PI⁻ cells to detect viable human T cells. The CD3⁻ CD45⁺ gate contains Raji tumor cells. Expression of CD25 and CD69 on live (PI⁻) CD3⁺ CD45⁺ T cells is shown in the histograms. (E) Frequency of CD3⁺ CD45⁺ T cells in spleens obtained 24 and 72 hours after T cell transfer. Dot plots are gated on live PI-splenocytes and the percentage of CD3⁺ CD45⁺ T cells is shown in each plot. (F) PI staining of bone marrow and splenocytes hours after T cell transfer into NSG/Raji mice. The numbers in the histograms indicate the percentage of PI⁺ cells within the CD3⁺ population. (G) Bioluminescence imaging of cohorts of mice treated with T cells expressing CD19-chimeric receptors with short spacer (‘short/CD28’ and ‘short/4-1BB’), long spacers (‘long/CD28 and ‘long/4-1BB’), or control T cells.

FIG. 20: T cells expressing CD19 chimeric receptors with 4-1BB and CD3zeta and a modified IgG4-Fc hinge exhibit superior in vitro and in vivo function compared to T cells expressing CD19 chimeric receptors with 4-1BB and CD3zeta and a CD8 alpha hinge. A. Cytolytic activity of CD19 chimeric receptor modified T-cells with IgG4 Fe hinge, CD8 alpha hinge and control T cells against Cr⁵¹-labeled K562 cells transfected with CD19, Raji lymphoma cells that express CD19, and K562 control T cells. Lysis is shown at different E/T ratios in a 4 hour Cr⁵¹ release assay. B. Interferon gamma production by 5×10⁴ T cells expressing a CD19 chimeric receptor with an IgG4 Fe hinge or CD8 alpha hinge after a 24-hour coculture with Raji tumor cells. O.D. of 1 corresponds to ˜500 pg/ml of interferon gamma. C. CFSE dye dilution assay to measure proliferation of T cells expressing a CD19 chimeric receptor with an IgG4 Fe hinge or CD8 alpha hinge and T cells that express tEGFR alone (control) after 72 hours coculture with CD19 positive Raji lymphoma cells. Numbers above each histogram indicate the number of cell divisions the proliferating cell subset underwent. The fraction of T cells in each gate that underwent ≥3/2/1 cell divisions is provided next to the plot. D. In vivo antitumor activity of T cells expressing a CD19 chimeric receptor with an IgG4 Fe hinge (group 1) or CD8 alpha hinge (group 2) and T cells that express tEGFR alone (group 3) in NSG mice inoculated with Raji tumor cells expressing firefly luciferase (ffluc). Mice were imaged 17 days after tumor inoculation and 10 days after T cell inoculation. The data shows greater tumor burden in mice treated with control tEGFR T cells (group 3) or with CD19 chimeric receptor CD8 alpha hinge T cells (group 2) compared with mice treated with CD19 chimeric receptor IgG4 Fe hinge T cells (group 1).

### DETAILED DESCRIPTION

Unless defined otherwise, all technical and scientific terms used herein have the same meaning as commonly understood by one of ordinary skill in the art to which the invention pertains.

“About” as used herein when referring to a measurable value is meant to encompass variations of ±20% or ±10%, more preferably ±5%, even more preferably ±1%, and still more preferably ±0.1% from the specified value.

“Activation”, as used herein, refers to the state of a T cell that has been sufficiently stimulated to induce detectable cellular proliferation, cytokine production or expression of cell surface markers such as CD69 and CD25, or detectable effector functions.

“Activation Induced cell death” as used herein refers to a state of a T cell that is activated but is not able to proliferate for more than 2 generations and exhibits markers of apoptosis.

“Antigen” or “Ag” as used herein refers to a molecule that provokes an immune response. This immune response may involve either antibody production, or the activation of specific immunologically-competent cells, or both. It is readily apparent that an antigen can be generated synthesized, produced recombinantly or can be derived from a biological sample. Such a biological sample can include, but is not limited to a tissue sample, a tumor sample, a cell or a biological fluid.

“Anti-tumor effect” as used herein, refers to a biological effect, which can be manifested by a decrease in tumor volume, a decrease in the number of tumor cells, a decrease in the number of metastases, an increase in life expectancy, or a decrease of various physiological symptoms associated with the cancerous condition. An “anti-tumor effect” can also be manifested by a decrease in recurrence or an increase in the time before recurrence.

“Chimeric receptor” as used herein refers to a synthetically designed receptor comprising a ligand binding domain of an antibody or other protein sequence that binds to a molecule associated with the disease or disorder and is linked via a spacer domain to one or more intracellular signaling domains of a T cell or other receptors, such as a costimulatory domain.

“Co-stimulatory domain,” as the term is used herein refers to a signaling moiety that provides to T cells a signal which, in addition to the primary signal provided by for instance the CD3 zeta chain of the TCR/CD3 complex, mediates a T cell response, including, but not limited to, activation, proliferation, differentiation, cytokine secretion, and the like. A co-stimulatory domain can include all or a portion of, but is not limited to, CD27, CD28, 4-1BB, OX40, CD30, CD40, ICOS, lymphocyte function-associated antigen-1 (LFA-1), CD2, CD7, LIGHT, NKG2C, B7-H3, and a ligand that specifically binds with CD83. In embodiments, the co-stimulatory domain is an intracellular signaling domain that interacts with other intracellular mediators to mediate a cell response including activation, proliferation, differentiation and cytokine secretion, and the like.

“Coding for” are used herein refers to the property of specific sequences of nucleotides in a polynucleotide, such as a gene, a cDNA, or an mRNA, to serve as templates for synthesis of other macromolecules such as a defined sequence of amino acids. Thus, a gene codes for a protein if transcription and translation of mRNA corresponding to that gene produces the protein in a cell or other biological system. A “nucleic acid sequence coding for a polypeptide” includes all nucleotide sequences that are degenerate versions of each other and that code for the same amino acid sequence.

“Cytotoxic T lymphocyte” (CTL) as used herein refers to a T lymphocyte that expresses CD8 on the surface thereof (i.e., a CD8⁺ T cell). In some embodiments such cells are preferably “memory” T cells (T_{M} cells) that are antigen-experienced.

“Central memory” T cell (or “T_{CM}”) as used herein refers to an antigen experienced CTL that expresses CD62L or CCR-7 and CD45RO on the surface thereof, and does not express or has decreased expression of CD45RA as compared to naive cells. In embodiments, central memory cells are positive for expression of CD62L, CCR7, CD28, CD127, CD45RO, and CD95, and have decreased expression of CD54RA as compared to naïve cells.

“Effector memory” T cell (or “T_{EM}”) as used herein refers to an antigen experienced T cell that does not express or has decreased expression of CD62L on the surface thereof as compared to central memory cells, and does not express or has decreased expression of CD45RA as compared to naïve cell. In embodiments, effector memory cells are negative for expression of CD62L and CCR7, compared to naïve cells or central memory cells, and have variable expression of CD28 and CD45RA.

“Naïve” T cells as used herein refers to a non antigen experienced T lymphocyte that expresses CD62L and CD45RA, and does not express CD45RO− as compared to central or effector memory cells. In some embodiments, naïve CD8+ T lymphocytes are characterized by the expression of phenotypic markers of naïve T cells including CD62L, CCR7, CD28, CD127, and CD45RA.

“Effector” “T_{E}” T cells as used herein refers to a antigen experienced cytotoxic T lymphocyte cells that do not express or have decreased expression of CD62L, CCR7, CD28, and are positive for granzyme B and perforin as compared to central memory or naïve T cells.

“Enriched” and “depleted” as used herein to describe amounts of cell types in a mixture refers to the subjecting of the mixture of the cells to a process or step which results in an increase in the number of the “enriched” type and a decrease in the number of the “depleted” cells. Thus, depending upon the source of the original population of cells subjected to the enriching process, a mixture or composition may contain about 60, 70, 80, 90, 95, or 99 percent or more (in number or count) of the “enriched” cells and about 40, 30, 20, 10, 5 or 1 percent or less (in number or count) of the “depleted” cells.

“Epitope” as used herein refers to a part of an antigen or molecule that is recognized by the immune system including antibodies, T cells, and/or B cells. Epitopes usually have at least 7 amino acids and can be linear or conformational.

“Isolated,” when used to describe the various polypeptides disclosed herein, means polypeptide or nucleic acid that has been identified and separated and/or recovered from a component of its natural environment. Preferably, the isolated polypeptide or nucleic acid is free of association with all components with which it is naturally associated. Contaminant components of its natural environment are materials that would typically interfere with diagnostic or therapeutic uses for the polypeptide or nucleic acid, and may include enzymes, hormones, and other proteinaceous or non-proteinaceous solutes.

“Intracellular signaling domain” as used herein refers to all or a portion of one or more domains of a molecule (here the chimeric receptor molecule) that provides for activation of a lymphocyte. Intracellular domains of such molecules mediate a signal by interacting with cellular mediators to result in proliferation, differentiation, activation and other effector functions. In embodiments, such molecules include all or portions of CD28, CD3, 4-1BB, and combinations thereof.

“Ligand” as used herein refers to a substance that binds specifically to another substance to form a complex. Example of ligands include epitopes on antigens, molecules that bind to receptors, substrates, inhibitors, hormones, and activators. “Ligand binding domain” as used herein refers to substance or portion of a substance that binds to a ligand. Examples of ligand binding domains include antigen binding portions of antibodies, extracellular domains of receptors, and active sites of enzymes.

“Operably linked” as used herein refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

“Percent (%) amino acid sequence identity” with respect to the chimeric receptor polypeptide sequences identified herein is defined as the percentage of amino acid residues in a candidate sequence that are identical with the amino acid residues in the reference sequence for each of the ligand binding domain, spacer, transmembrane domain, and/or the lymphocyte activating domain, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, including any algorithms needed to achieve maximal alignment over the full-length of the sequences being compared. For example, % amino acid sequence identity values generated using the WU-BLAST-2 computer program [Altschul et al., Methods in Enzymology, 266:460-480 (1996)] uses several search parameters, most of which are set to the default values. Those that are not set to default values (i.e., the adjustable parameters) are set with the following values: overlap span=1, overlap fraction=0.125, word threshold (T)=11 and scoring matrix=BLOSUM62. A % amino acid sequence identity value is determined by dividing (a) the number of matching identical amino acid residues between the each or all of the polypeptide amino acid sequence of the reference chimeric receptor sequence provided in Table 2 and the comparison amino acid sequence of interest as determined by WU-BLAST-2 by (b) the total number of amino acid residues of the polypeptide of interest.

“Chimeric receptor variant polynucleotide” or “chimeric receptor variant nucleic acid sequence” as used herein refers to a polypeptide-encoding nucleic acid molecule as defined below having at least about 80% nucleic acid sequence identity with the polynucleotide acid sequence shown in Table 1 or a specifically derived fragment thereof, such as polynucleotide coding for an antigen binding domain, a polynucleotide encoding a spacer domain, a polynucleotide coding for a transmembrane domain and/or a polynucleotide coding for a lymphocyte stimulatory domain. Ordinarily, a chimeric receptor variant of polynucleotide or fragment thereof will have at least about 80% nucleic acid sequence identity, more preferably at least about 81% nucleic acid sequence identity, more preferably at least about 82% nucleic acid sequence identity, more preferably at least about 83% nucleic acid sequence identity, more preferably at least about 84% nucleic acid sequence identity, more preferably at least about 85% nucleic acid sequence identity, more preferably at least about 86% nucleic acid sequence identity, more preferably at least about 87% nucleic acid sequence identity, more preferably at least about 88% nucleic acid sequence identity, more preferably at least about 89% nucleic acid sequence identity, more preferably at least about 90% nucleic acid sequence identity, more preferably at least about 91% nucleic acid sequence identity, more preferably at least about 92% nucleic acid sequence identity, more preferably at least about 93% nucleic acid sequence identity, more preferably at least about 94% nucleic acid sequence identity, more preferably at least about 95% nucleic acid sequence identity, more preferably at least about 96% nucleic acid sequence identity, more preferably at least about 97% nucleic acid sequence identity, more preferably at least about 98% nucleic acid sequence identity and yet more preferably at least about 99% nucleic acid sequence identity with the nucleic acid sequence as shown in Table or a derived fragment thereof. Variants do not encompass the native nucleotide sequence. In this regard, due to the degeneracy of the genetic code, one of ordinary skill in the art will immediately recognize that a large number of chimeric receptor variant polynucleotides having at least about 80% nucleic acid sequence identity to the nucleotide sequence of Table 1 will encode a polypeptide having an amino acid sequence which is identical to the amino acid sequence of Table 2.

“Substantially purified” refers to a molecule that is essentially free of other molecule types or a cell that is essentially free of other cell types. A substantially purified cell also refers to a cell, which has been separated from other cell types with which it is normally associated in its naturally occurring state. In some instances, a population of substantially purified cells refers to a homogenous population of cells.

“Not substantially found” when used in reference the presence of a tumor antigen or other molecules on normal cells refers to the percentage of a normal cell type that has the antigen or molecule, and/or the density of the antigen on the cells. In embodiments, not substantially found means that the antigen or molecule is found on less than 50% of normal cell type and/or at a 50% less density as compared to the amount of cells or antigen found on a tumor cell or other diseased cell.

“T cells” or “T lymphocytes” as used herein may be from any mammalian, preferably primate, species, including monkeys, dogs, and humans. In some embodiments the T cells are allogeneic (from the same species but different donor) as the recipient subject; in some embodiments the T cells are autologous (the donor and the recipient are the same); in some embodiments the T cells arc syngeneic (the donor and the recipients are different but are identical twins).

### Modes of the Disclosure

The disclosure provides for chimeric receptor nucleic acids, and vectors and host cells including such nucleic acids. The chimeric receptor nucleic acid comprises a number of modular components that can be excised and replaced with other components in order to customize the chimeric receptor for a specific target molecule. The disclosure provides that one of the modular components is the spacer component. It has been surprisingly found that the length of the spacer region that is presumed not to have signaling capability affects the in vivo efficacy of the T cells modified to express the chimeric receptor and needs to be customized for individual target molecules for enhanced therapeutic activity.

In one aspect, methods and nucleic acid constructs are provided to design a chimeric receptor that has improved tumor recognition, increased T cell proliferation and/or cytokine production in response to the ligand as compared to a reference chimeric receptor. In embodiments, a library of nucleic acids is provided, wherein each nucleic acid codes for a spacer region that differs from the others in sequence and length. Each of the nucleic acids can then be used to form a chimeric receptor nucleic acid construct that can be tested in vivo (in an animal model) and/or in vitro so that a spacer can be selected that provides for improved tumor recognition, increased T cell proliferation and/or cytokine production in response to the ligand.

In embodiments, a chimeric receptor nucleic acid comprises a polynucleotide coding for a ligand binding domain, wherein the ligand is a tumor or viral specific antigen or molecule, a polynucleotide coding for a customized polypeptide spacer, wherein the spacer provides for enhanced T cell proliferation; a polynucleotide coding for a transmembrane domain; and a polynucleotide coding for one or more intracellular signaling domains. In embodiments, a long spacer is employed if the epitope of the target molecule is membrane proximal on the target cell and a short spacer is employed if the epitope of the target molecule is membrane distal on the target cell.

The design of a chimeric receptor can be customized depending on the type of tumor or virus, the target antigen or molecule present on the tumor, the affinity of the antibody for the target molecule, the flexibility needed for the antigen binding domain, and/or the intracellular signaling domain. In embodiments, a number of chimeric receptor constructs are tested in vitro and in in vivo models to determine the ability of T cells modified with the receptor to kill tumor cells in immunodeficient mice and to proliferate and persist after adoptive transfer. In embodiments, a chimeric receptor is selected that provides for capability of at least 30% of the cells to proliferate through at least two generations in vitro and/or within 72 hours after introduction in vivo. In embodiments, a chimeric receptor is not selected that results in greater than 50% of the cells undergoing activation induced cell death (AICD) within 72 hours in vivo in immunodeficient mice, and fails to eradicate tumor cells.

Depending on whether the target molecule is present on a subject's tumor cells, the chimeric receptor includes a ligand binding domain that specifically binds to that target molecule. In embodiments, a subject's tumor cells are characterized for cell surface tumor molecules. The target molecule may be selected based on a determination of its presence on a particular subject's tumor cells. In embodiments, a target molecule is selected that is a cell surface molecule found predominantly on tumor cells and not found on normal tissues to any substantial degree. In embodiments, an antibody is selected to bind to an epitope on the targeted cell surface molecule. In some cases, the epitope is characterized with respect to its proximity to the cell membrane. An epitope is characterized as proximal to the membrane when it is predicted or known by structural analysis to reside closer to the target cell membrane than alternative epitopes that are predicted or known by structural analysis to reside a greater distance from the target cell membrane. In embodiments, the affinity of the antibody from which the scFV is constructed is compared by binding assays, and antibodies with different affinities are examined in chimeric receptor formats expressed in T cells to determine which affinity confers optimal tumor recognition, based on superior cytotoxicity of target cells, and/or T cell cytokine production and proliferation.

In addition, the spacer region of the chimeric receptor may be varied to optimize T cell recognition of the ligand on the target cell. In embodiments, when an antibody binds to an epitope on the target cell that is very proximal to the membrane, a spacer is selected that is longer than about 15 amino acids. For example, in embodiments, if the epitope or portion thereof on the target antigen is in the first 100 amino acids of the linear sequence of the extracellular domain adjacent to the transmembrane domain, a long spacer region may be selected. In embodiments, when an antibody binds to an epitope on the target cell that is distal to the membrane, a spacer is selected that is about 119 or 15 amino acids or less. For example, in embodiments, when the epitope or portion thereof is found in the 150 amino acids of the linear sequence of the extracellular domain from the terminus, a short or intermediate spacer may be utilized. In embodiments, a spacer comprises an amino acid sequence X₁PPX₂P (SEQ ID NO:1).

A variety of combinations of primary and costimulatory intracellular signaling domain may be employed to enhance the in vivo efficacy of the chimeric receptor. In embodiments, different constructs of the chimeric receptor can be tested in an in vivo animal model to determine efficacy for tumor killing. In embodiments, a costimulatory intracellular signaling domain is selected from the group consisting of CD28 and modified versions thereof, 4-1BB and modified versions thereof and combinations thereof. Other costimulatory domains, such as OX40 may be incorporated.

CD8+ central memory T cells have an intrinsic programming that allows them to persist for extended periods after administration, which makes them a preferred subset of CD8+ T cells for immunotherapy. In embodiments, CD19 specific chimeric receptor modified cytotoxic T cells prepared from sort purified CD8+ central memory T cells are administered in the presence or absence of CD4+CD19 specific chimeric receptor-modified T cells. In embodiments, tumor-specific CD4+ T cells exert anti-tumor reactivity and provide help to tumor-specific CD8+ T cells in vitro and in vivo. In a specific embodiment, tumor-specific CD4+ T cells or CD4⁺ T cells selected from the naïve or the central memory subsets are utilized alone or in combination with CD8⁺ T_{CM}.

### Nucleic Acids, Vectors, and Polypeptides

The disclosure provides a chimeric receptor nucleic acid useful for transforming or transducing lymphocytes for use in adoptive immunotherapy. In embodiments, the nucleic acid contains a number of modular components that provide for easy substitution of elements of the nucleic acid. While not meant to limit the scope of the disclosure, it is believed that the chimeric receptor for each tumor antigen is desirably customized in terms of components in order to provide for in vivo efficacy and efficient expression in mammalian cells. For example, in a specific embodiment, for efficacy of a chimeric receptor comprising a scFV that binds to a ROR1 epitope located in the membrane distal Ig/Frizzled domain, a spacer that is about 15 amino acids or less is employed. In another specific embodiment, for efficacy of a chimeric receptor comprising a scFV that binds to a ROR1 epitope located in the membrane proximal Kringle domain, a spacer that is longer than 15 amino acids is employed. In another embodiment, for efficacy of a chimeric receptor comprising a scFV that binds to CD19, a spacer that is 15 amino acids or less is employed.

In embodiments, an isolated chimeric receptor nucleic acid comprises a polynucleotide coding for a ligand binding domain, wherein the target molecule is a tumor specific antigen, a polynucleotide coding for a polypeptide spacer wherein the polypeptide spacer is about 229 amino acids or less; a polynucleotide coding for a transmembrane domain; and a polynucleotide coding for an intracellular signaling domain. In embodiments, an expression vector comprises a chimeric nucleic acid as described herein. Polypeptides encoded by all of or a portion of the chimeric receptor nucleic acids are also included herein.

### Ligand Binding Domain

In embodiments, the chimeric receptor nucleic acid comprises a polynucleotide coding for a ligand binding domain. In embodiments, the ligand binding domain specifically binds to a tumor or viral specific antigen. In embodiments, the ligand binding domain is an antibody or fragment thereof. A nucleic acid sequence coding for an antibody or antibody fragment can readily be determined. In a specific embodiment, the polynucleotide codes for a single chain Fv that specifically binds CD19. In other specific embodiments, the polynucleotide codes for a single chain Fv that specifically binds ROR1. The sequences of these antibodies are known to or can readily be determined by those of skill in the art.

Tumor antigens are proteins that are produced by tumor cells that elicit an immune response. The selection of the ligand binding domain of the invention will depend on the type of cancer to be treated, and may target tumor antigens or other tumor cell surface molecules. A tumor sample from a subject may be characterized for the presence of certain biomarkers or cell surface markers. For example, breast cancer cells from a subject may be positive or negative for each of Her2Neu, Estrogen receptor, and/or the Progesterone receptor. A tumor antigen or cell surface molecule is selected that is found on the individual subject's tumor cells. Tumor antigens and cell surface molecules are well known in the art and include, for example, carcinoembryonic antigen (CEA), prostate specific antigen, PSMA, Her2/neu, estrogen receptor, progesterone receptor, ephrinB2, CD19, CD20, CD22, CD23, CD123, CS-1, ROR1, mesothelin, c-Met, GD-2, and MAGE A3 TCR. In embodiments a target molecule is a cell surface molecule that is found on tumor cells and is not substantially found on normal tissues, or restricted in its expression to non-vital normal tissues.

Other target molecules include but are not limited to antigens derived from infectious pathogens such as HIV (human immunodeficiency virus), HBV (hepatitis B virus), HPV (human papilloma virus) and Hepatitis C virus.

In one embodiment, the target molecule on the tumor comprises one or more epitopes associated with a malignant tumor. Malignant tumors express a number of proteins that can serve as target antigens for T cell receptor or chimeric receptor mediated recognition. Other target molecules belong to the group of cell transformation-related molecules such as the oncogene HER-2/Neu/ErbB2. In embodiments, the tumor antigen is selectively expressed or overexpressed on the tumor cells as compared to control cells of the same tissue type. In other embodiments, the tumor antigen is a cell surface polypeptide.

Once a tumor cell surface molecule that might be targeted with a chimeric receptor is identified, an epitope of the target molecule is selected and characterized. In embodiments, an epitope is selected that is proximal to the tumor cell membrane. In other embodiments, an epitope is selected that is distal to the tumor cell membrane. An epitope is characterized as proximal to the membrane when it is predicted or known by structural analysis to reside closer to the target cell membrane than alternative epitopes that are predicted or known by structural analysis to reside a greater distance from the target cell membrane.

Antibodies that specifically bind a tumor cell surface molecule can be prepared using methods of obtaining monoclonal antibodies, methods of phage display, methods to generate human or humanized antibodies, or methods using a transgenic animal or plant engineered to produce human antibodies. Phage display libraries of partially or fully synthetic antibodies are available and can be screened for an antibody or fragment thereof that can bind to the target molecule. Phage display libraries of human antibodies are also available. In embodiments, antibodies specifically bind to a tumor cell surface molecule and do not cross react with nonspecific components such as bovine serum albumin or other unrelated antigens. Once identified, the amino acid sequence or polynucleotide sequence coding for the antibody can be isolated and/or determined.

Antibodies or antigen binding fragments include all or a portion of polyclonal antibodies, a monoclonal antibody, a human antibody, a humanized antibody, a synthetic antibody, a chimeric antibody, a bispecific antibody, a minibody, and a linear antibody. Antibody fragments” comprise a portion of an intact antibody, preferably the antigen binding or variable region of the intact antibody and can readily be prepared. Examples of antibody fragments include Fab, Fab′, F(ab′)₂, and Fv fragments; diabodies; linear antibodies; single-chain antibody molecules; and multispecific antibodies formed from antibody fragments.

In embodiments, a number of different antibodies that bind to a particular tumor cell surface molecule can be isolated and characterized. In embodiments, the antibodies are characterized based on epitope specificity of the targeted molecule. In addition, in some cases, antibodies that bind to the same epitope can be selected based on the affinity of the antibody for that epitope. In embodiments, an antibody has an affinity of at least 1 mM, and preferably <50 nM. In embodiments, an antibody is selected that has a higher affinity for the epitope as compared to other antibodies. For example, an antibody is selected that has at least a 2 fold, at least a 5 fold, at least a 10 fold, at least a 20 fold, at least a 30 fold, at least a 40 fold, or at least a 50 fold greater affinity than a reference antibody that binds to the same epitope.

In embodiments, target molecules are selected from the group consisting of CD19, CD20, CD22, CD23, CD123, CS-1, ROR1, mesothelin, Her2, c-Met, PSMA, GD-2, MAGE A3 TCR and combinations thereof.

In specific embodiments, the target antigen is CD19. A number of antibodies specific for CD19 are known to those of skill in the art and can be readily characterized for sequence, epitope binding, and affinity. In a specific embodiment, the chimeric receptor construct includes a scFV sequence from FMC63 antibody. In other embodiments, the scFV is a human or humanized scFv comprising a variable light chain comprising a CDRL1 sequence of RASQDISKYLN (SEQ ID NO:57), CDRL2 sequence of SRLHSGV (SEQ ID NO:58), and a CDRL3 sequence of GNTLPYTFG (SEQ ID NO:59). In other embodiments, the scFV is a human or humanized scFv comprising a variable heavy chain comprising CDRH1 sequence of DYGVS (SEQ ID NO:74), CDRH2 sequence of VIWGSETTYYNSALKS (SEQ ID NO:60), and a CDRH3 sequence of YAMDYWG (SEQ ID NO:61). The disclosure also contemplates variable regions that have at least 90% amino acid sequence identity to that of the scFv for FMC63 and that have at least the same affinity for CD19. In embodiments, the chimeric receptor has a short or intermediate spacer of 119 amino acids or less, or 12 amino acids or less. In a specific embodiment, the spacer is 12 amino acid or less and has a sequence of SEQ ID NO:4.

In embodiments, CDR regions are found within antibody regions as numbered by Kabat as follows: for the light chain; CDRL1 amino acids 24-34; CDRL2 amino acids 50-56; CDRL3 at amino acids 89-97; for the heavy chain at CDRH1 at amino acids 31-35; CDRH2 at amino acids 50-65; and for CDRH3 at amino acids 95-102. CDR regions in antibodies can be readily determined.

In specific embodiments, the target antigen is ROR1. A number of antibodies specific for ROR1 are known to those of skill in the art and can be readily characterized for sequence, epitope binding, and affinity. In a specific embodiment, the chimeric receptor construct includes a scFV sequence from R12 antibody. In other embodiments, the scFV is a human or humanized ScFv comprising a variable light chain comprising a CDRL1 sequence of ASGFDFSAYYM (SEQ ID NO:62), CDRL2 sequence of TIYPSSG (SEQ ID NO:63), and a CDRL3 sequence of ADRATYFCA (SEQ ID NO:64). In other embodiments, the scFV is a human or humanized ScFv comprising a variable heavy chain comprising CDRH1 sequence of DTIDWY (SEQ ID NO:65), CDRH2 sequence of VQSDGSYTKRPGVPDR (SEQ ID NO:66), and a CDRH3 sequence of YIGGYVFG (SEQ ID NO:67). The disclosure also contemplates variable regions that have at least 90% amino acid sequence identity to that of the scFv for R12 and that have at least the same affinity for ROR1. In embodiments, the chimeric receptor has a short or intermediate spacer of 119 amino acids or less, or 12 amino acids or less. In a specific embodiment, the spacer is 12 amino acid or less and has a sequence of SEQ ID NO:4.

In specific embodiments, the target antigen is ROR1. A number of antibodies specific for ROR1 are known to those of skill in the art and can be readily characterized for sequence, epitope binding, and affinity. In a specific embodiment, the chimeric receptor construct includes a scFV sequence from R11 antibody. In other embodiments, the scFV is a human or humanized ScFv comprising a variable light chain comprising a CDRL1 sequence of SGSDINDYPIS (SEQ ID NO:68), CDRL2 sequence of INSGGST (SEQ ID NO:69), and a CDRL3 sequence of YFCARGYS (SEQ ID NO:70). In other embodiments, the scFV is a human or humanized scFv comprising a variable heavy chain comprising CDRH1 sequence of SNLAW (SEQ ID NO:71), CDRH2 sequence of RASNLASGVPSRFSGS (SEQ ID NO:72), and a CDRH3 sequence of NVSYRTSF (SEQ ID NO:73). The disclosure also contemplates variable regions that have at least 90% amino acid sequence identity to that of the scFv for R11 and that have at least the same affinity for ROR1. In embodiments, the chimeric receptor has a long spacer of 229 amino acids or less. In a specific embodiment, the spacer is 229 amino acids and has a sequence of SEQ ID NO:50.

In specific embodiments, the target antigen is Her2. A number of antibodies specific for Her2 are known to those of skill in the art and can be readily characterized for sequence, epitope binding, and affinity. In a specific embodiment, the chimeric receptor construct includes a scFV sequence from Herceptin antibody. In other embodiments, the scFV is a human or humanized ScFv comprising a variable light chain comprising a CDRL1 sequence, CDRL2 sequence and a CDRL3 sequence of the Herceptin antibody. In other embodiments, the scFV is a human or humanized ScFv comprising a variable heavy chain comprising CDRH1 sequence, CDRH2, and a CDRH3 sequence of Herceptin. The CDR sequences can readily be determined from the amino acid sequence of Herceptin. The disclosure also contemplates variable regions that have at least 90% amino acid sequence identity to that of the scFv for Herceptin and that have at least the same affinity for Her2. In embodiments, the chimeric receptor has a long spacer of 229 amino acids or less. In a specific embodiment, the spacer is 229 amino acids and has a sequence of SEQ ID NO:50.

In embodiments, a polynucleotide coding for a ligand binding domain is operably linked to a polynucleotide coding for a spacer region. In embodiments, the polynucleotide coding for a ligand binding domain may also have one or more restriction enzyme sites at the 5′ and/or 3′ ends of the coding sequence in order to provide for easy excision and replacement of the polynucleotide with another polynucleotide coding for a ligand binding domain coding for a different antigen or that has different binding characteristics. For example, a restriction site, NheI, is encoded upstream of the leader sequence; and a 3′ RsrII located within the hinge region allows subcloning of any desirable scFv into a chimeric receptor vector. In embodiments, the polynucleotide is codon optimized for expression in mammalian cells.

In embodiments, the polynucleotide coding for a ligand binding domain is operably linked to a signal peptide. In embodiments the signal peptide is a signal peptide for granulocyte colony stimulating factor. Polynucleotides coding for other signal peptides such as CD8 alpha can be utilized.

In embodiments, the polynucleotide coding for a ligand binding domain is operably linked to a promoter. A promoter is selected that provides for expression of the chimeric antigen receptor in a mammalian cell. In a specific embodiment the promoter is the elongation growth factor promoter (EF-1). Another example of a suitable promoter is the immediate early cytomegalovirus (CMV) promoter sequence. However, other constitutive promoter sequences may also be used, including, but not limited to the simian virus 40 (SV 40) early promoter, mouse mammary tumor virus (MMTV), human immunodeficiency virus (H1V) long terminal repeat (LTR) promoter, MuMoLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Inducible promoters are also contemplated. Examples of inducible promoters include, but are not limited to a metallothionine promoter, a glucocorticoid promoter, a progesterone promoter, and a tetracycline promoter.

A specific embodiment of a polynucleotide coding for a ligand binding domain is shown in Table 1 as the scFv from an antibody that specifically binds CD19, such as FMC63. A polynucleotide encoding for a flexible linker including the amino acids GSTSGSGKPGSGEGSTKG (SEQ ID NO:36) separates the VH and VL chains in the scFV.

The amino acid sequence of the scFv including the linker is shown in Table 2. (SEQ ID NO:11) Other CD19-targeting antibodies such as SJ25C1 and HD37 are known. (SJ25C1: Bejcek et al. Cancer Res 2005, PMID 7538901; HD37: Pezutto et al. JI 1987, PMID 2437199).

### Spacer

In embodiments, the chimeric receptor nucleic acid comprises a polynucleotide coding for a spacer region. It has been surprisingly found that the length of the spacer region that is presumed not to have signaling capability affects the in vivo efficacy of the T cells modified to express the chimeric receptor and needs to be customized for individual target molecules for optimal tumor or target cell recognition. In embodiments, the chimeric receptor nucleic acid comprises a polynucleotide coding for a customizable spacer region selected from a library of polynucleotides coding for spacer regions. In embodiments, a spacer length is selected based upon the location of the epitope, affinity of the antibody for the epitope, and/or the ability of the T cells expressing the chimeric receptor to proliferate in vitro and/or in vivo in response to antigen recognition.

Typically a spacer region is found between the ligand binding domain and the transmembrane domain of the chimeric receptor. In embodiments, a spacer region provides for flexibility of the ligand binding domain, allows for high expression levels in lymphocytes. A CD19-specific chimeric receptor having a spacer domain of about 229 amino acids had less antitumor activity than a CD19-specific chimeric receptor with a short spacer region comprised of the modified IgG4 hinge only. Other chimeric receptors, such as those constructed from the R12 or 2A2 scFvs also require a short spacer for optimal triggering of T cell effector functions, while a chimeric receptor constructed with the R11 ROR1 scFv requires a long spacer domain of about 229 amino acids for tumor recognition.

In embodiments, a spacer region has at least about 10 to 229 amino acids, about 10 to 200 amino acids, about 10 to 175 amino acids, about 10 to 150 amino acids, about 10 to 125 amino acids, about 10 to 100 amino acids, about 10 to 75 amino acids, about 10 to 50 amino acids, about 10 to 40 amino acids, about 10 to 30 amino acids, about 10 to 20 amino acids, or about 10 to 15 amino acids, and including any integer between the endpoints of any of the listed ranges. In embodiments, a spacer region has about 12 amino acids or less, about 119 amino acids or less, or about 229 amino acids or less.

In some embodiments, the spacer region is derived from a hinge region of an immunoglobulin like molecule. In embodiments, a spacer region comprises all or a portion of the hinge region from a human IgG1, human IgG2, a human IgG3, or a human IgG4, and may contain one or more amino acid substitutions. Exemplary sequences of the hinge regions are provided in Table 8. In embodiments, a portion of the hinge region includes the upper hinge amino acids found between the variable heavy chain and the core, and the core hinge amino acids including a polyproline region. Typically, the upper hinge region has about 3 to 10 amino acids. In some cases, the spacer region comprises an amino acid sequence of X₁PPX₂P (SEQ ID NO:1). In embodiments, X₁ is a cysteine, glycine, or arginine and X₂ is a cysteine or a threonine.

In embodiments, hinge region sequences can be modified in one or more amino acids in order to avoid undesirable structural interactions such as dimerization. In a specific embodiment, the spacer region comprises a portion of a modified human hinge region from IgG4, for example, as shown in Table 2 or Table 8 (SEQ ID NO:21). A representative of a polynucleotide coding for a portion of a modified IgG4 hinge region is provided in Table 1. (SEQ ID NO:4) In embodiments, a hinge region can have at least about 90%, 92%, 95%, or 100% sequence identity with a hinge region amino acid sequence identified in Table 2 or Table 8. In a specific embodiment, a portion of a human hinge region from IgG4 has an amino acid substitution in the core amino acids from CPSP to CPPC.

In some embodiments, all or a portion of the hinge region is combined with one or more domains of a constant region of an immunoglobulin. For example, a portion of a hinge region can be combined with all or a portion of a CH2 or CH3 domain or variant thereof. In embodiments, the spacer region does not include the 47-48 amino acid hinge region sequence from CD8apha or the spacer region consisting of an extracellular portion of the CD28 molecule.

In embodiments, a short spacer region has about 12 amino acids or less and comprises all or a portion of a IgG4 hinge region sequence or variant thereof, an intermediate spacer region has about 119 amino acids or less and comprises all or a portion of a IgG4 hinge region sequence and a CH3 region or variant thereof, and a long spacer has about 229 amino acids or less and comprises all or a portion of a IgG4 hinge region sequence, a CH2 region, and a CH3 region or variant thereof.

A polynucleotide coding for a spacer region can be readily prepared by synthetic or recombinant methods from the amino acid sequence. In embodiments, a polynucleotide coding for a spacer region is operably linked to a polynucleotide coding for a transmembrane region. In embodiments, the polynucleotide coding for the spacer region may also have one or more restriction enzyme sites at the 5′ and/or 3′ ends of the coding sequence in order to provide for easy excision and replacement of the polynucleotide with another polynucleotide coding for a different spacer region. In embodiments, the polynucleotide coding for the spacer region is codon optimized for expression in mammalian cells.

In embodiments, a library of polynucleotides, each coding for different spacer region is provided. In an embodiment, the spacer region is selected from the group consisting of a hinge region sequence from IgG1, IgG2, IgG3, or IgG4 or portion thereof, a hinge region sequence from IgG1, IgG2, IgG3, or IgG4 in combination with all or a portion of a CH2 region or variant thereof, a hinge region sequence from IgG1, IgG2, IgG3, or IgG4 in combination with all or a portion of a CH3 region or variant thereof, and a hinge region sequence from IgG1, IgG2, IgG3, or IgG4 in combination with all or a portion of a CH2 region or variant thereof, and a CH3 region or variant thereof. In embodiments, a short spacer region is a modified IgG4 hinge sequence (SEQ ID NO:4) having 12 amino acids or less, an intermediate sequence is a IgG4 hinge sequence with a CH3 sequence having 119 amino acids or less (SEQ ID NO:49); or a IgG4 hinge sequence with a CH2 and CH3 region having 229 amino acids or less (SEQ ID NO:50)

In embodiments, a method of selecting a spacer region for a chimeric receptor is provided herein. Surprisingly some chimeric receptor constructs, although effective to activate T cells and direct their killing of tumor cells in vitro, were not effective in vivo. In addition, the side effect profile of the chimeric receptor modified T cells can be such as to result in more cells undergoing activation induced cell death or causing an increase in in vivo cytokines. In embodiments, a method comprises providing a plurality of chimeric receptor nucleic acids, wherein the chimeric receptor nucleic acids differ only in the spacer region; introducing each of the chimeric receptor nucleic acids into a separate T lymphocyte population; expanding each separate lymphocyte population in vitro, and introducing each lymphocyte population into an animal bearing a tumor to determine the anti-tumor efficacy of each of the chimeric receptors when expressed in T cells, and selecting a chimeric receptor that provides anti-tumor efficacy as compared to each of the other separate lymphocyte populations modified with each of the other chimeric receptors.

Animal models of different tumors are known. Anti-tumor efficacy can be measured by identifying a decrease in tumor volume, by determining animal death, persistence of the genetically modified T cells in vivo, activation of genetically modified T cells (for example, by detecting an increase in expression of CD25 and/CD69), and/or proliferation of genetically modified T cells in vivo. In an embodiment, a chimeric receptor is selected that provides for the best anti-tumor efficacy in vivo as determined by one or more of these parameters. Lack of anti-tumor efficacy can be determined by lack of persistence of the genetically modified lymphocytes in vivo, animal death, an increase in apoptosis as measured by an increase in induction of caspase-3, and/or a decrease in proliferation of genetically modified lymphocytes.

In other embodiments, a method for selecting a spacer comprises selecting an epitope of a target molecule and characterizing the location of the epitope with respect to the cell membrane, selecting a spacer region that is long or short depending on the location of the epitope with respect to the cell membrane, selecting an antibody or fragment thereof that has an affinity for the epitope that is higher or lower as compared to a reference antibody, and determining whether the chimeric receptor construct provides for enhanced T cell proliferation or cytokine production in vitro and/or in vivo.

In some embodiments, if the target epitope or portion thereof is located proximal to the membrane it is located in the first 100 amino acids of the linear sequence of the extracellular domain adjacent to the transmembrane domain. If the epitope is located proximal to the membrane, a long spacer (e.g., 229 amino acids or less and greater than 119 amino acids) is selected. In some embodiments, if the target epitope is located distal to the membrane, it is located in the first 150 amino acids of the linear sequence of the extracellular domain terminus. If the epitope is located distal to the membrane, an intermediate or short spacer is selected (e.g. 119 amino acids or less or 12-15 amino acids or less). Alternatively, whether the epitope is proximal or distal to the membrane can be determined by modeling of the three dimensional structure or based on analysis of the crystal structure,

In some embodiments, a chimeric receptor is selected that provides for at least 30% of the cells proliferating through two generations in vitro and/or in vivo. In other embodiments a chimeric receptor is not selected if it results in at least 50% of the cells undergoing activation induced cell death in 72 hours. In embodiments, a short spacer (e.g. 15 amino acids or less) is selected if the epitope is distal to the membrane. In embodiments, a long spacer (e.g. 229 amino acid or less and greater than 119 amino acids) is selected if the epitope is proximal to the membrane.

In embodiments, providing a plurality of chimeric receptor nucleic acids, wherein the chimeric receptor nucleic acids differ only in the spacer region comprises providing a chimeric receptor construct comprising a polynucleotide coding for a ligand binding domain, wherein the ligand is a tumor specific antigen, viral antigen, or any other molecule expressed on a target cell population that is suitable to mediate recognition and elimination by a lymphocyte; a polynucleotide coding for a first polypeptide spacer having a defined restriction site at the 5′ and 3′ end of the coding sequence for the first polypeptide spacer; a polynucleotide coding for a transmembrane domain; and a polynucleotide coding for one or more intracellular signaling domains.

In embodiments, a method further comprises providing one or more polynucleotides, each encoding a different spacer region. In embodiments, the different spacer regions are selected from the group consisting of a hinge region sequence from IgG1, IgG2, IgG3, or IgG4 or variant thereof or portion thereof, a hinge region sequence from IgG1, IgG2, IgG3, or IgG4 in combination with all or a portion of a CH2 region or variant thereof, a hinge region sequence from IgG1, IgG2, IgG3, or IgG4 in combination with all or a portion of a CH3 region or variant thereof, and a hinge region sequence from IgG1, IgG2, IgG3, or IgG4 in combination with all or a portion of a CH2 region or variant thereof and a CH3 region or variant thereof. In embodiments, CH2 or CH3 regions may be modified by one or more deletions or amino acid substitutions in order to provide for expression in lymphocytes and/or in order to minimize interactions with other molecules. In embodiments, a portion of a hinge region comprises at least the upper amino acids and the core sequence. In embodiments, a hinge region comprises the sequence X₁PPX₂P (SEQ ID NO:1). In embodiments, a method further comprises replacing the polynucleotide coding for the spacer region with a polynucleotide encoding a different spacer region to form a chimeric receptor nucleic acid with a different spacer region. The method can be repeated to form any number of chimeric receptor nucleic acids, each differing in the spacer region. In embodiments, the chimeric receptor nucleic acids differ from one another only in the spacer region.

### Transmembrane Domain

In embodiments, the chimeric receptor nucleic acid comprises a polynucleotide coding for a transmembrane domain. The transmembrane domain provides for anchoring of the chimeric receptor in the membrane.

In an embodiment, the transmembrane domain that naturally is associated with one of the domains in the chimeric receptor is used. In some cases, the transmembrane domain can be selected or modified by amino acid substitution to avoid binding of such domains to the transmembrane domains of the same or different surface membrane proteins to minimize interactions with other members of the receptor complex.

The transmembrane domain may be derived either from a natural or a synthetic source. When the source is natural, the domain may be derived from any membrane-bound or transmembrane protein. Transmembrane regions comprise at least the transmembrane region(s) of) the alpha, beta or zeta chain of the T-cell receptor, CD28, CD3, CD45, CD4, CD8, CD9, CD16, CD22; CD33, CD37, CD64, CD80, CD86, CD134, CD137 and CD154. In a specific embodiment, the transmembrane domain comprises the amino acid sequence of the CD28 transmembrane domain as shown in Table 2. A representative polynucleotide sequence coding for the CD28 transmembrane domain is shown in Table 1 (SEQ ID NO:5).

A transmembrane domain may be synthetic or a variant of a naturally occurring transmembrane domain. In embodiments, synthetic or variant transmembrane domains comprise predominantly hydrophobic residues such as leucine and valine. In embodiments, a transmembrane domain can have at least about 80%, 85%, 90%, 95%, or 100% amino acid sequence identity with a transmembrane domain as shown in Table 2 or Table 6. Variant transmembrane domains preferably have a hydrophobic score of at least 50 as calculated by Kyte Doolittle.

A polynucleotide coding for a transmembrane domain can be readily prepared by synthetic or recombinant methods. In embodiments, a polynucleotide coding for a transmembrane domain is operably linked to a polynucleotide coding for a intracellular signaling region. In embodiments, the polynucleotide coding for a transmembrane domain may also have one or more restriction enzyme sites at the 5′ and/or 3′ ends of the coding sequence in order to provide for easy excision and replacement of the polynucleotide coding for a transmembrane domain with another polynucleotide coding for a different transmembrane domain. In embodiments, the polynucleotide coding for a transmembrane domain is codon optimized for expression in mammalian cells.

### Intracellular Signaling Domain

In embodiments, the chimeric receptor nucleic acid comprises a polynucleotide coding for an intracellular signaling domain. The intracellular signaling domain provides for activation of one function of the transduced cell expressing the chimeric receptor upon binding to the ligand expressed on tumor cells. In embodiments, the intracellular signaling domain contains one or more intracellular signaling domains. In embodiments, the intracellular signaling domain is a portion of and/or a variant of an intracellular signaling domain that provides for activation of at least one function of the transduced cell.

Examples of intracellular signaling domains for use in a chimeric receptor of the disclosure include the cytoplasmic sequences of the CD3 zeta chain, and/or co-receptors that act in concert to initiate signal transduction following chimeric receptor engagement, as well as any derivative or variant of these sequences and any synthetic sequence that has the same functional capability. T cell activation can be said to be mediated by two distinct classes of cytoplasmic signaling sequence: those that initiate antigen-dependent primary activation and provide a T cell receptor like signal (primary cytoplasmic signaling sequences) and those that act in an antigen-independent manner to provide a secondary or co-stimulatory signal (secondary cytoplasmic signaling sequences). Primary cytoplasmic signaling sequences that act in a stimulatory manner may contain signaling motifs which are known as receptor tyrosine-based activation motifs or ITAMs. Examples of ITAM containing primary cytoplasmic signaling sequences include those derived from CD3 zeta, FcR gamma, CD3 gamma, CD3 delta, CD3 epsilon, CD5, CD22, CD79a, CD79b, and CD66d. In embodiments, the primary signaling intracellular domain can have at least about 80%, 85%, 90%, or 95% sequence identity to CD3zeta having a sequence provided in Table 2. In embodiments variants, of CD3 zeta retain at least one, two, three or all ITAM regions as shown in Table 7.

In a preferred embodiment, the intracellular signaling domain of the chimeric receptor can be designed to comprise the CD3-zeta signaling domain by itself or combined with any other desired cytoplasmic domain(s). For example, the intracellular signaling domain of the chimeric receptor can comprise a CD3zeta chain and a costimulatory signaling region.

The costimulatory signaling region refers to a portion of the chimeric receptor comprising the intracellular domain of a costimulatory molecule. A costimulatory molecule is a cell surface molecule other than an antigen receptor or their ligands that is required for a response of lymphocytes to an antigen. Examples of such molecules include CD27, CD28, 4-1BB (CD 137), OX40, CD30, CD40, lymphocyte function-associated antigen-1 (LFA-1), CD2, CD7, LIGHT, NKG2C, B7-H3, and a ligand that specifically binds with CD83. In embodiments, the costimulatory signaling domain can have at least about 80%, 85%, 90%, or 95% amino acid sequence identity to the intracellular domain of CD28 as shown in Table 5 or to 4-1BB having a sequence provided in Table 2. In an embodiment, a variant of the CD28 intracellular domain comprises an amino acid substitution at positions 186-187, wherein LL is substituted with GG.

The intracellular signaling sequences of the chimeric receptor may be linked to each other in a random or specified order. Optionally, a short oligo- or polypeptide linker, preferably between 2 and 10 amino acids in length may form the linkage. In one embodiment, the intracellular signaling domains comprises all or a portion of the signaling domain of CD3-zeta or variant thereof and all or a portion of the signaling domain of CD28 or a variant thereof. In another embodiment, the intracellular signaling domain comprises all or a portion of the signaling domain of CD3-zeta or variant thereof and all or a portion of the signaling domain of 4-1BB or variant thereof. In yet another embodiment, the intracellular signaling domain comprises all or a portion of the signaling domain of CD3-zeta or variant thereof, all or a portion of the signaling domain of CD28 or variant thereof, and all or a portion of the signaling domain of 4-1BB or variant thereof. In a specific embodiment, the amino acid sequence of the intracellular signaling domain comprising a variant of CD3zeta and a portion of the 4-1BB intracellular signaling domain is provided in Table 2. A representative nucleic acid sequence is provided in Table 1 (SEQ ID NO:6; SEQ ID NO:7).

In an embodiment, a polynucleotide coding for an intracellular signaling domain comprises a 4-1BB intracellular domain linked to a portion of a CD3zeta domain. In other embodiments, a 4-1BB intracellular domain and a CD28 intracellular domain are linked to a portion of a CD3 zeta domain.

A polynucleotide coding for an intracellular signaling domain can be readily prepared by synthetic or recombinant methods from the amino acid sequence. In embodiments, the polynucleotide coding for an intracellular signaling domain may also have one or more restriction enzyme sites at the 5′ and/or 3′ ends of the coding sequence in order to provide for easy excision and replacement of the polynucleotide coding for an intracellular signaling domain with another polynucleotide coding for a different intracellular signaling domain. In embodiments, the polynucleotide coding for an intracellular signaling domain is codon optimized for expression in mammalian cells.

### Marker Sequences

In embodiments, the chimeric receptor nucleic acid optionally further comprises a polynucleotide sequence coding for a marker sequence. A marker sequence can provide for selection of transduced cells, and identification of transduced cells. In embodiments, the marker sequence is operably linked to a polynucleotide sequence coding for a linker sequence. In embodiments, the linker sequence is a cleavable linker sequence.

A number of different marker sequences can be employed. Typically a marker sequence has a functional characteristic that allows for selection of transduced cells and/or detection of transduced cells. In embodiments, the marker sequence is compatible with transduction of human lymphocytes.

The positive selectable marker may be a gene, which upon being introduced into the host cell, expresses a dominant phenotype permitting positive selection of cells carrying the gene. Genes of this type are known in the art, and include, inter alia, hygromycin-B phosphotransferase gene (hph) which confers resistance to hygromycin B, the amino glycoside phosphotransferase gene (neo or aph) from Tn5 which codes for resistance to the antibiotic G418, the dihydrofolate reductase (DHFR) gene, the adenosine deaminase gene (ADA), and the multi-drug resistance (MDR) gene.

In an embodiment, a chimeric receptor nucleic acid further comprises a polynucleotide coding for a marker sequence. In an embodiment, the marker sequence is a truncated epidermal growth factor receptor as shown in Table 2. An exemplary polynucleotide for the truncated epidermal growth factor receptor is shown in Table 1. (SEQ ID NO:9) In embodiments, the polynucleotide coding for the marker sequence is operably linked to a polynucleotide coding for a linker sequence. In a specific embodiment, the linker sequence is a cleavable linker sequence T2A as shown in Table 2. An exemplary polynucleotide sequence coding for the T2A linker is provided in Table 1. (SEQ ID NO:8)

A polynucleotide coding for marker sequence can be readily prepared by synthetic or recombinant methods from the amino acid sequence. In embodiments a polynucleotide coding for a marker sequence is operably linked to a polynucleotide coding for an intracellular signaling domain. In embodiments, the polynucleotide coding for a marker sequence may also have one or more restriction enzyme sites at the 5′ and/or 3′ ends of the coding sequence in order to provide for easy excision and replacement of the polynucleotide coding for a marker sequence with another polynucleotide coding for a different marker sequence. In embodiments, the polynucleotide coding for a marker sequence is codon optimized for expression in mammalian cells.

### Vectors, Cells and Methods of Transducing Cells

### (a) Selection and Sorting of T Lymphocyte Populations

The compositions described herein provide for CD4+ and/or CD8+ T lymphocytes. T lymphocytes can be collected in accordance with known techniques and enriched or depleted by known techniques such as affinity binding to antibodies such as flow cytometry and/or immunomagnetic selection. After enrichment and/or depletion steps, in vitro expansion of the desired T lymphocytes can be carried out in accordance with known techniques (including but not limited to those described in U.S. Pat. No. 6,040,177 to Riddell et al.), or variations thereof that will be apparent to those skilled in the art. In embodiments, the T cells are autologous T cells obtained from the patient.

For example, the desired T cell population or subpopulation may be expanded by adding an initial T lymphocyte population to a culture medium in vitro, and then adding to the culture medium feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). The non-dividing feeder cells can comprise gamma-irradiated PBMC feeder cells. In some embodiments, the PBMC are irradiated with gamma rays in the range of about 3000 to 3600 rads to prevent cell division. The order of addition of the T cells and feeder cells to the culture media can be reversed if desired. The culture can typically be incubated under conditions of temperature and the like that are suitable for the growth of T lymphocytes. For the growth of human T lymphocytes, for example, the temperature will generally be at least about 25 degrees Celsius, preferably at least about 30 degrees, more preferably about 37 degrees.

The T lymphocytes expanded include CD8⁺ cytotoxic T lymphocytes (CTL) and CD4⁺ helper T lymphocytes that may be specific for an antigen present on a human tumor or a pathogen.

Optionally, the expansion method may further comprise the step of adding non-dividing EBV-transformed lymphoblastoid cells (LCL) as feeder cells. LCL can be irradiated with gamma rays in the range of about 6000 to 10,000 rads. The LCL feeder cells may be provided in any suitable amount, such as a ratio of LCL feeder cells to initial T lymphocytes of at least about 10:1.

Optionally, the expansion method may further comprise the step of adding anti-CD3 and/or anti CD28 antibody to the culture medium (e.g., at a concentration of at least about 0.5 ng/ml). Optionally, the expansion method may further comprise the step of adding IL-2 and/or IL-15 to the culture medium (e.g., wherein the concentration of IL-2 is at least about 10 units/ml).

After isolation of T lymphocytes both cytotoxic and helper T lymphocytes can be sorted into naïve, memory, and effector T cell subpopulations either before or after expansion.

CD8+ cells can be obtained by using standard methods. In some embodiments, CD8+ cells are further sorted into naïve, central memory, and effector memory cells by identifying cell surface antigens that are associated with each of those types of CD8+ cells. In embodiments, memory T cells are present in both CD62L+ and CD62L− subsets of CD8+ peripheral blood lymphocytes. PBMC are sorted into CD62L−CD8+ and CD62L+CD8+ fractions after staining with anti-CD8 and anti-CD62L antibodies. In some embodiments, the expression of phenotypic markers of central memory T_{CM} include CD45RO, CD62L, CCR7, CD28, CD3, and CD127 and are negative or low for granzyme B. In some embodiments, central memory T cells are CD45RO+, CD62L+, CD8+ T cells. In some embodiments, effector T_{E} are negative for CD62L, CCR7, CD28, and CD127, and positive for granzyme B and perforin. In some embodiments, naïve CD8+ T lymphocytes are characterized by the expression of phenotypic markers of naïve T cells including CD62L, CCR7, CD28, CD3, CD127, and CD45RA.

Whether a cell or cell population is positive for a particular cell surface marker can be determined by flow cytometry using staining with a specific antibody for the surface marker and an isotype matched control antibody. A cell population negative for a marker refers to the absence of significant staining of the cell population with the specific antibody above the isotype control, positive refers to uniform staining of the cell population above the isotype control. In some embodiments, a decrease in expression of one or markers refers to loss of 1 log 10 in the mean fluorescence intensity and/or decrease of percentage of cells that exhibit the marker of at least about 20% of the cells, 25% of-the cells, 30% of the cells, 35% of the cells, 40% of the cells, 45% of the cells, 50% of the cells, 55% of the cells, 60% of the cells, 65% of the cells, 70% of the cells, 75% of the cells, 80% of the cells, 85% of the cells, 90% of the cell, 95% of the cells, and 100% of the cells and any % between 20 and 100% when compared to a reference cell population. In some embodiments, a cell population positive for one or markers refers to a percentage of cells that exhibit the marker of at least about 50% of the cells, 55% of the cells, 60% of the cells, 65% of the cells, 70% of the cells, 75% of the cells, 80% of the cells, 85% of the cells, 90% of the cell, 95% of the cells, and 100% of the cells and any % between 50 and 100% when compared to a reference cell population.

CD4+ T helper cells are sorted into naïve, central memory, and effector cells by identifying cell populations that have cell surface antigens. CD4+ lymphocytes can be obtained by standard methods. In some embodiments, naïve CD4+ T lymphocytes are CD45RO−, CD45RA+, CD62L+, CD4+ T cells. In some embodiments, central memory CD4+ cells are CD62L+ and CD45RO+. In some embodiments, effector CD4+ cells are CD62L− and CD45RO−.

In embodiments, populations of CD4+ and CD8+ that are antigen specific can be obtained by stimulating naïve or antigen specific T lymphocytes with antigen. For example, antigen-specific T cell lines or clones can be generated to Cytomegalovirus antigens by isolating T cells from infected subjects and stimulating the cells in vitro with the same antigen. Naïve T cells may also be used. Any number of antigens from tumor cells may be utilized as targets to elicit T cell responses. In some embodiments, the adoptive cellular immunotherapy compositions are useful in the treatment of a disease or disorder including a solid tumor, hematologic malignancy, breast cancer or melanoma.

### (b) Modification of T Lymphocyte Populations

In some embodiments it may be desired to introduce functional genes into the T cells to be used in immunotherapy in accordance with the present disclosure. For example, the introduced gene or genes may improve the efficacy of therapy by promoting the viability and/or function of transferred T cells; or they may provide a genetic marker to permit selection and/or evaluation of in vivo survival or migration; or they may incorporate functions that improve the safety of immunotherapy, for example, by making the cell susceptible to negative selection in vivo as described by Lupton S. D. et al., Mol. and Cell Biol., 11:6 (1991); and Riddell et al., Human Gene Therapy 3:319-338 (1992); see also the publications of PCT/US91/08442 and PCT/US94/05601 by Lupton et al. describing the use of bifunctional selectable fusion genes derived from fusing a dominant positive selectable marker with a negative selectable marker. This can be carried out in accordance with known techniques (see, e.g., U.S. Pat. No. 6,040,177 to Riddell et al. at columns 14-17) or variations thereof that will be apparent to those skilled in the art based upon the present disclosure.

In embodiments, T cells are modified with chimeric receptors as described herein. In some embodiments, the T cells are obtained from the subject to be treated. In other embodiments, the lymphocytes are obtained from allogeneic human donors, preferably healthy human donors.

In some embodiments, chimeric receptors comprise a ligand binding domain that specifically binds to a tumor cell surface molecule, a polypeptide spacer region, a transmembrane domain and an intracellular signaling domain as described herein. In embodiments, the ligand binding domain is a single-chain antibody fragment (scFv) that is derived from the variable heavy (VH) and variable light (VL) chains of a monoclonal antibody (mAb). Costimulatory signals can also be provided through the chimeric receptor by fusing the costimulatory domain of CD28 and/or 4-1BB to the CD3ζ chain. Chimeric receptors are specific for cell surface molecules independent from HLA, thus overcoming the limitations of TCR-recognition including HLA-restriction and low levels of HLA-expression on tumor cells.

Chimeric receptors can be constructed with a specificity for any cell surface marker by utilizing antigen binding fragments or antibody variable domains of, for example, antibody molecules. The antigen binding molecules can be linked to one or more cell signaling modules. In embodiments, cell signaling modules include CD3 transmembrane domain, CD3 intracellular signaling domains, and CD28 transmembrane domains. In embodiments, the intracellular signaling domain comprises a CD28 transmembrane and signaling domain linked to a CD3 zeta intracellular domain. In some embodiments, a chimeric receptor can also include a transduction marker such as tEGFR.

In embodiments, the same or a different chimeric receptor can be introduced into each of population of CD4+ and CD8+ T lymphocytes. In embodiments, the chimeric receptor in each of these populations has a ligand binding domain that specifically binds to the same ligand on the tumor or infected cell. The cellular signaling modules can differ. In embodiments, the intracellular signaling domain of the CD8+ cytotoxic T cells is the same as the intracellular signaling domain of the CD4+ helper T cells. In other embodiments, the intracellular signaling domain of the CD8+ cytotoxic T cells is different than the intracellular signaling domain of the CD4+ helper T cells.

In embodiments each of the CD4 or CD8 T lymphocytes can be sorted in to naïve, central memory, effector memory or effector cells prior to transduction as described herein. In alternative embodiments, each of the CD4 or CD8 T lymphocytes can be sorted in to naïve, central memory, effector memory, or effector cells after transduction.

Various transduction techniques have been developed which utilize recombinant infectious virus particles for gene delivery. This represents a currently preferred approach to the transduction of T lymphocytes of the present invention. The viral vectors which have been used in this way include virus vectors derived from simian virus 40, adenoviruses, adeno-associated virus (AAV), lentiviral vectors, and retroviruses. Thus, gene transfer and expression methods are numerous but essentially function to introduce and express genetic material in mammalian cells. Several of the above techniques have been used to transduce hematopoietic or lymphoid cells, including calcium phosphate transfection, protoplast fusion, electroporation, and infection with recombinant adenovirus, adeno-associated virus and retrovirus vectors. Primary T lymphocytes have been successfully transduced by electroporation and by retroviral or lentiviral infection.

Retroviral and lentiviral vectors provide a highly efficient method for gene transfer into eukaryotic cells. Moreover, retroviral or lentiviral integration takes place in a controlled fashion and results in the stable integration of one or a few copies of the new genetic information per cell.

It is contemplated that overexpression of a stimulatory factor (for example, a lymphokine or a cytokine) may be toxic to the treated individual. Therefore, it is within the scope of the invention to include gene segments that cause the T cells of the invention to be susceptible to negative selection in vivo. By “negative selection” is meant that the infused cell can be eliminated as a result of a change in the in vivo condition of the individual. The negative selectable phenotype may result from the insertion of a gene that confers sensitivity to an administered agent, for example, a compound. Negative selectable genes are known in the art, and include, inter alia the following: the Herpes simplex virus type I thymidine kinase (HSV-I TK) gene, which confers ganciclovir sensitivity; the cellular hypoxanthine phosphribosyltransferase (HPRT) gene, the cellular adenine phosphoribosyltransferase (APRT) gene, and bacterial cytosine deaminase.

In some embodiments it may be useful to include in the T cells a positive marker that enables the selection of cells of the negative selectable phenotype in vitro. The positive selectable marker may be a gene that upon being introduced into the host cell expresses a dominant phenotype permitting positive selection of cells carrying the gene. Genes of this type are known in the art, and include, inter alia, hygromycin-B phosphotransferase gene (hph) which confers resistance to hygromycin B, the amino glycoside phosphotransferase gene (neo or aph) from Tn5 which codes for resistance to the antibiotic G418, the dihydrofolate reductase (DHFR) gene, the adenosine deaminase gene (ADA), and the multi-drug resistance (MDR) gene.

A variety of methods can be employed for transducing T lymphocytes, as is well known in the art. In embodiments, transduction is carried out using lentiviral vectors.

In embodiments, CD4+ and CD8+ cells each can separately be modified with an expression vector encoding a chimeric receptor to form defined populations. In embodiments, these cells are then further sorted into subpopulations of naïve, central memory and effector cells as described above by sorting for cell surface antigens unique to each of those cell populations. In addition, CD4+ or CD8+ cell populations may be selected by their cytokine profile or proliferative activities. For example, CD4+ T lymphocytes that have enhanced production of cytokines such as IL-2, IL-4, IL-10, TNFa, and IFNγ as compared to sham transduced cells or transduced CD8+ cells when stimulated with antigen can be selected. In other embodiments, naïve or central memory CD4+ T cells that have enhanced production of IL-2 and/or TNFa are selected. Likewise, CD8+ cells that have enhanced IFNγ production are selected as compared to sham transduced CD8+ cells.

In embodiments, CD4+ and CD8+ cells that proliferate in response to antigen or tumor targets are selected. For example, CD4+ cells that proliferate vigorously when stimulated with antigen or tumor targets as compared to sham transduced cells, or CD8+ transduced cells are selected. In some embodiments, CD4+ and CD8+ cells are selected that are cytotoxic for antigen bearing cells. In embodiments, CD4+ are expected to be weakly cytotoxic as compared to CD8+ cells.

In a preferred embodiment, transduced lymphocytes, such as CD8+ central memory cells, are selected that provide for tumor cell killing in vivo using an animal model established for the particular type of cancer. Such animal models are known to those of skill in the art and exclude human beings. As described herein, not all chimeric receptor constructs transduced into lymphocytes confer the ability to kill tumor cells in vivo despite the ability to become activated and kill tumor cells in vitro. In particular, for some target molecules T cells having chimeric receptor constructs with a long spacer region were less effective at killing tumor cells in vivo as compared to T cells having a chimeric receptor with short spacer region. For other target molecules, T cells having chimeric receptor constructs with a short spacer region were less effective at killing tumor cells in vivo as compared to T cells having chimeric receptors with a long spacer region.

In yet other embodiments, transduced chimeric receptor expressing T cells are selected that can persist in vivo using an animal model established for the particular type of cancer. In embodiments, transduced chimeric receptor CD8+ central memory cells with a short spacer region have been shown to persist in vivo after introduction into the animal for about 3 day or more, 10 days or more, 20 days or more, 30 days or more, 40 days or more, or 50 days or more.

The disclosure contemplates that combinations of CD4+ and CD8+ T cells will be utilized in the compositions. In one embodiment, combinations of chimeric receptor transduced CD4+ cells can be combined with chimeric receptor transduced CD8+ cells of the same ligand specificity or combined with CD8+ T cells that are specific for a distinct tumor ligand. In other embodiments, chimeric receptor transduced CD8+ cells are combined with chimeric receptor transduced CD4+ cells specific for a different ligand expressed on the tumor. In yet another embodiment, chimeric receptor modified CD4+ and CD8+ cells are combined. In embodiments CD8+ and CD4+ cells can be combined in different ratios for example, a 1:1 ratio of CD8+ and CD4+, a ratio of 10:1 of CD8+ to CD4+, or a ratio of 100:1 of CD8+ to CD4+. In embodiments, the combined population is tested for cell proliferation in vitro and/or in vivo, and the ratio of cells that provides for proliferation of cells is selected.

As described herein, the disclosure contemplates that CD4+ and CD8+ cells can be further separated into subpopulations, such as naïve, central memory, and effector memory cell populations. As described herein, in some embodiments, naïve CD4+ cells are CD45RO−, CD45RA+, CD62L+, CD4+ positive T cells. In some embodiments, central memory CD4+ cells are CD62L positive and CD45RO positive. In some embodiments, effector CD4+ cells are CD62L negative and CD45RO positive. Each of these populations may be independently modified with a chimeric receptor.

As described herein, in embodiments, memory T cells are present in both CD62L+ and CD62L− subsets of CD8+ peripheral blood lymphocytes. PBMC are sorted into CD62L− CD8+ and CD62L+CD8+ fractions after staining with anti-CD8 and anti-CD62L antibodies.

In some embodiments, the expression of phenotypic markers of central memory T cells (TCM) include CD62L, CCR7, CD28, CD3, and CD127 and are negative or low for granzyme B. In some embodiments, central memory T cells are CD45RO+, CD62L+, CD8+ T cells. In some embodiments, effector T cells (T_{E}) are negative for CD62L, CCR7, CD28, and CD127, and positive for granzyme B and perforin. In some embodiments, naïve CD8+T lymphocytes are characterized by CD8+, CD62L+, CD45RO+, CCR7+, CD28+CD127+, and CD45RO+. Each of these populations may be independently modified with a chimeric receptor.

After transduction and/or selection for chimeric receptor bearing cells, the cell populations are preferably expanded in vitro until a sufficient number of cells are obtained to provide for at least one infusion into a human subject, typically around 10⁴ cells/kg to 10¹ cells/kg In embodiments, the transduced cells are cultured in the presence of antigen bearing cells, anti CD3, anti CD28, and IL 2, IL-7, IL 15, IL-21 and combinations thereof.

Each of the subpopulations of CD4+ and CD8+ cells can be combined with one another. In a specific embodiment, modified naïve or central memory CD4+ cells are combined with modified central memory CD8+ T cells to provide a synergistic cytotoxic effect on antigen bearing cells, such as tumor cells.

### Compositions

The disclosure provides for an adoptive cellular immunotherapy composition comprising a genetically modified T lymphocyte cell preparation as described herein.

In embodiments, the T lymphocyte cell preparation comprises CD4+ T cells that have a chimeric receptor comprising an extracellular antibody variable domain specific for a ligand associated with the disease or disorder, a customizable spacer region, a transmembrane domain, and an intracellular signaling domain of a T cell receptor or other receptors as described herein. In other embodiments, an adoptive cellular immunotherapy composition further comprises a chimeric receptor modified tumor-specific CD8+ cytotoxic T lymphocyte cell preparation that provides a cellular immune response, wherein the cytotoxic T lymphocyte cell preparation comprises CD8+ T cells that have a chimeric receptor comprising an extracellular single chain antibody specific for a ligand associated with the disease or disorder, a customizable spacer region, a transmembrane domain, and an intracellular signaling domain of a T cell receptor as described herein. In embodiments, the chimeric receptor modified T cell population of the disclosure can persist in vivo for at least about 3 days or longer.

In some embodiments, an adoptive cellular immunotherapy composition comprises a chimeric receptor modified tumor-specific CD8+ cytotoxic T lymphocyte cell preparation that provides a cellular immune response, wherein the cytotoxic T lymphocyte cell preparation comprises CD8+ T cells that have a chimeric receptor comprising an extracellular single chain antibody specific for a ligand associated with the disease or disorder, a customizable spacer region, a transmembrane domain, and an intracellular signaling domain of a T cell receptor, in combination with an antigen-reactive chimeric receptor modified naïve CD4+ T helper cell derived from CD45RO−CD62L+CD4+ T cells, and a pharmaceutically acceptable carrier.

In other embodiments, an adoptive cellular immunotherapy composition comprises an antigen specific CD8+ cytotoxic T lymphocyte cell preparation that provides a cellular immune response derived from the patient combined with an antigen-reactive chimeric receptor modified naïve CD4+ T helper cell that augments the CD8+ immune response, wherein the helper T lymphocyte cell preparation comprises CD4+ T cells that have a chimeric receptor comprising an extracellular antibody variable domain specific for the antigen associated with the disease or disorder, a customizable spacer region, a transmembrane domain, and an intracellular signaling domain of a T cell receptor.

In a further embodiment, an adoptive cellular immunotherapy composition comprises an antigen-reactive chimeric receptor modified naïve CD4+ T helper cell that augments the CD8+ immune response, wherein the helper T lymphocyte cell preparation comprises CD4+ T cells that have a chimeric receptor comprising an extracellular antibody variable domain specific for a ligand associated with a disease or disorder, a customizable spacer region, a transmembrane domain, and an intracellular signaling domain of a T cell receptor.

In embodiments, the CD4+ T helper lymphocyte cell is selected from the group consisting of naïve CD4+ T cells, central memory CD4+ T cells, effector memory CD4+ T cells, or bulk CD4+ T cells. In some embodiments, CD4+ helper lymphocyte cell is a naïve CD4+ T cell, wherein the naïve CD4+ T cell comprises a CD45RO−, CD45RA+, CD62L+CD4+ T cell. In embodiments, the CD8+ T cytotoxic lymphocyte cell is selected from the group consisting of naïve CD8+ T cells, central memory CD8+ T cells, effector memory CD8+ T cells or bulk CD8+ T cells. In some embodiments, the CD8+ cytotoxic T lymphocyte cell is a central memory T cell wherein the central memory T cell comprises a CD45RO+, CD62L+, CD8+ T cell. In yet other embodiments, the CD8+ cytotoxic T lymphocyte cell is a central memory T cell and the CD4+ helper T lymphocyte cell is a naïve or central memory CD4+ T cell.

Methods The disclosure provides methods of making adoptive immunotherapy compositions and uses or methods of using these compositions for performing cellular immunotherapy in a subject having a disease or disorder. In embodiments, the chimeric receptor modified T cells as described herein are able to persist in vivo for at least 3 days, or at least 10 days. In embodiments, the chimeric receptor modified T cells as described herein can proliferate in vivo through at least 2, or at least 3 generations as determined by CFSE dye dilution. Proliferation and persistence of the chimeric receptor modified T cells can be determined by using an animal model of the disease or disorder and administering the cells and determining persistence and/or proliferative capacity of the transferred cells. In other embodiments, proliferation and activation can be tested in vitro by going through multiple cycles of activation with antigen bearing cells.

In embodiments, a method of manufacturing the compositions comprises obtaining a modified naïve CD4+ T helper cell, wherein the modified helper T lymphocyte cell preparation comprises CD4+ T cells that have a chimeric receptor comprising a ligand binding domain specific for a tumor cell surface molecule, a customized spacer domain, a transmembrane domain, and an intracellular signaling domain as described herein.

In another embodiment, a method further comprises obtaining a modified CD8+ cytotoxic T cell, wherein the modified cytotoxic T lymphocyte cell preparation comprises CD8+ cells that have a chimeric receptor comprising a ligand binding domain specific for a tumor cell surface molecule, a customized spacer domain, a transmembrane domain, and an intracellular signaling domain as described herein.

In another embodiment, a method comprises obtaining a modified CD8+ cytotoxic T cell, wherein the modified cytotoxic T lymphocyte cell preparation comprises CD8+ T cells that have a chimeric receptor comprising a ligand binding domain specific for a tumor cell surface molecule, a customized spacer domain, a transmembrane domain, and an intracellular signaling domain as described herein, and further comprising combining the modified CD8+ cytotoxic T cells with a CD4+ helper cell lymphocyte cell preparation.

The preparation of the CD4+ and CD8+ cells that are modified with a chimeric receptor has been described above as well as in the examples. Antigen specific T lymphocytes can be obtained from a patient having the disease or disorder or can be prepared by in vitro stimulation of T lymphocytes in the presence of antigen. Subpopulations of CD4+ and CD8+ T lymphocytes that are not selected for antigen specificity can also be isolated as described herein and combined in the methods of manufacturing. In embodiments, the combination of cell populations can be evaluated for uniformity of cell surface makers, the ability to proliferate through at least two generations, to have a uniform cell differentiation status. Quality control can be performed by co-culturing a cell line expressing the target ligand with chimeric receptor modified T cells to determine if the chimeric receptor modified T cells recognize the cell line using cytotoxicity, proliferation, or cytokine production assays that are known in the field. Cell differentiation status and cell surface markers on the chimeric receptor modified T cells can be determined by flow cytometry. In embodiments, the markers and cell differentiation status on the CD8+ cells include CD3, CD8, CD62L, CD28, CD27, CD69, CD25, PD-1, CTLA-4, CD45RO, and CD45RA. In embodiments, the markers and the cell differentiation status on the CD4+ cells include CD3, CD4, CD62L, CD28, CD27, CD69, CD25, PD-1, CTLA-4 CD45RO, and CD45RA.

In embodiments, a method of selecting a spacer region for a chimeric receptor is provided herein. Surprisingly some chimeric receptor constructs, although effective to activate T cells in vitro, were not effective in vivo. In embodiments, a method comprises providing a plurality of chimeric receptor nucleic acids, wherein the chimeric receptor nucleic acids differ only in the spacer region; introducing each of the chimeric receptor nucleic acids into a separate T lymphocyte population; expanding each separate lymphocyte population in vitro, and introducing each lymphocyte population into an animal bearing a tumor to determine the anti-tumor efficacy of each of the chimeric receptor modified T cells, and selecting a chimeric receptor that provides anti-tumor efficacy as compared to each of the other separate lymphocyte populations modified with each of the other chimeric receptor modified T cells.

Animal models of different tumors are known. Anti-tumor efficacy can be measured by identifying a decrease in tumor volume, by determining animal death, persistence of the genetically modified T cells in vivo, activation of genetically modified T cells (for example, by detecting an increase in expression of CD25 and/CD69), and/or proliferation of genetically modified T cells in vivo. In an embodiment, a chimeric receptor is selected that provides for the best anti-tumor efficacy in vivo as determined by one or more of these parameters. Lack of anti-tumor efficacy can be determined by lack of persistence of the genetically modified lymphocytes in vivo, animal death, an increase in apoptosis as measured by an increase in induction of caspase-3, and/or a decrease in proliferation of genetically modified lymphocytes.

In embodiments, providing a plurality of chimeric receptor nucleic acids, wherein the chimeric receptor nucleic acids differ only in the spacer region comprises providing a chimeric receptor construct comprising a polynucleotide coding for a ligand binding domain, wherein the ligand is a tumor specific antigen, viral antigen, or any other molecule expressed on a target cell population that is suitable to mediate recognition and elimination by a lymphocyte; a polynucleotide coding for a first polypeptide spacer having a defined restriction site at the 5′ and 3′ end of the coding sequence for the first polypeptide spacer; a polynucleotide coding for a transmembrane domain; and a polynucleotide coding for an intracellular signaling domain.

The disclosure also provides methods of performing cellular immunotherapy in a subject having a disease or disorder comprising: administering a composition of lymphocytes expressing a chimeric receptor as described herein. In other embodiments, a method comprises administering to the subject a genetically modified cytotoxic T lymphocyte cell preparation that provides a cellular immune response, wherein the cytotoxic T lymphocyte cell preparation comprises CD8+ T cells that have a chimeric receptor comprising a ligand binding domain specific for a tumor cell surface molecule, a customized spacer domain, a transmembrane domain, and an intracellular signaling domain as described herein, and a genetically modified helper T lymphocyte cell preparation that elicits direct tumor recognition and augments the genetically modified cytotoxic T lymphocyte cell preparations ability to mediate a cellular immune response, wherein the helper T lymphocyte cell preparation comprises CD4+ T cells that have a chimeric receptor comprising a ligand binding domain specific for a tumor cell surface molecule, a customized spacer domain, a transmembrane domain, and an intracellular signaling domain as described herein.

While not limiting the scope of the disclosure, it is believed by selecting the chimeric receptor modified T cell population that can persist and proliferate in vivo prior to administration may result in the ability to use a lower dose of T cells and provide more uniform therapeutic activity. In embodiments, the dose of T cells can be reduced at least 10%, 20%, or 30% or greater. Reduction in the dose of T cells may be beneficial to reduce the risk or tumor lysis syndrome and cytokine storm.

In another embodiment, a method of performing cellular immunotherapy in subject having a disease or disorder comprises: administering to the subject a genetically modified helper T lymphocyte cell preparation, wherein the modified helper T lymphocyte cell preparation comprises CD4+ T cells that have a chimeric receptor comprising a ligand binding domain specific for a tumor cell surface molecule, a customized spacer domain, a transmembrane domain, and an intracellular signaling domain as described herein. In an embodiments, the method further comprises administering to the subject a genetically modified cytotoxic T lymphocyte cell preparation, wherein the modified cytotoxic T lymphocyte cell preparation comprises CD8+ cells that have a chimeric receptor comprising a ligand binding domain specific for a tumor cell surface molecule, a customized spacer domain, a transmembrane domain, and an intracellular signaling domain as described herein.

Another embodiment describes a method of performing cellular immunotherapy in a subject having a disease or disorder comprising: analyzing a biological sample of the subject for the presence of a target molecule associated with the disease or disorder and administering the adoptive immunotherapy compositions described herein, wherein the chimeric receptor specifically binds to the target molecule.

In some embodiments, the CD4+ T helper lymphocyte cell is selected prior to introduction of the chimeric receptor from the group consisting of naïve CD4+ T cells, central memory CD4+ T cells, effector memory CD4+ T cells or bulk CD4+ T cells. In a specific embodiment, CD4+ helper lymphocyte cell is a naïve CD4+ T cell, wherein the naïve CD4+ T cell comprises a CD45RO−, CD45RA+, CD62L+CD4+ T cell. In yet other embodiments, the CD8+ T cytotoxic lymphocyte cell is selected prior to introduction of the chimeric receptor from the group consisting of naïve CD8+ T cells, central memory CD8+ T cells, effector memory CD8+ T cells or bulk CD8+ T cells. In a specific embodiment, the CD8+ cytotoxic T lymphocyte cell is a central memory T cell wherein the central memory T cell comprises a CD45RO+, CD62L+, CD8+ T cell. In a specific embodiment, the CD8+ cytotoxic T lymphocyte cell is a central memory T cell and the CD4+ helper T lymphocyte cell is a naïve CD4+ T cell.

In embodiments, the CD8+ T cell and the CD4+ T cell are both genetically modified with a chimeric receptor comprising an antibody heavy chain domain that specifically binds a tumor-specific cell surface molecule. In other embodiments, the intracellular signaling domain of the CD8 cytotoxic T cells is the same as the intracellular signaling domain of the CD4 helper T cells. In yet other embodiments, the intracellular signaling domain of the CD8 cytotoxic T cells is different than the intracellular signaling domain of the CD4 helper T cells.

Subjects that can be treated by the present invention are, in general, human and other primate subjects, such as monkeys and apes for veterinary medicine purposes. The subjects can be male or female and can be any suitable age, including infant, juvenile, adolescent, adult, and geriatric subjects.

The methods are useful in the treatment of, for example, hematologic malignancy, melanoma, breast cancer, and other epithelial malignancies or solid tumors. In some embodiments, the molecule associated with the disease or disorder is selected from the group consisting of orphan tyrosine kinase receptor ROR1, Her2, CD19, CD20, CD22, mesothelin, CEA, and hepatitis B surface antigen.

Subjects that can be treated include subjects afflicted with cancer, including but not limited to colon, lung, liver, breast, renal, prostate, ovarian, skin (including melanoma), bone, and brain cancer, etc. In some embodiments the tumor associated antigens or molecules are known, such as melanoma, breast cancer, squamous cell carcinoma, colon cancer, leukemia, myeloma, and prostate cancer. In other embodiments the tumor associated molecules can be targeted with genetically modified T cells expressing an engineered chimeric receptor.

Examples include but are not limited to B cell lymphoma, breast cancer, prostate cancer, and leukemia.

Cells prepared as described above can be utilized in methods and compositions for adoptive immunotherapy in accordance with known techniques, or variations thereof that will be apparent to those skilled in the art based on the instant disclosure.

In some embodiments, the cells are formulated by first harvesting them from their culture medium, and then washing and concentrating the cells in a medium and container system suitable for administration (a “pharmaceutically acceptable” carrier) in a treatment-effective amount. Suitable infusion medium can be any isotonic medium formulation, typically normal saline, Normosol R (Abbott) or Plasma-Lyte A (Baxter), but also 5% dextrose in water or Ringer's lactate can be utilized. The infusion medium can be supplemented with human serum albumin, fetal bovine serum or other human serum components.

A treatment effective amount of cells in the composition is at least 2 cell subsets (for example, 1 CD8+ central memory T cell subset and 1 CD4+ helper T cell subset) or is more typically greater than 10² cells, and up to 10⁶, up to and including 10⁸ or 10⁹ cells and can be more than 10¹⁰ cells. The number of cells will depend upon the ultimate use for which the composition is intended as will the type of cells included therein. For example, if cells that are specific for a particular antigen are desired, then the population will contain greater than 70%, generally greater than 80%, 85% and 90-95% of such cells. For uses provided herein, the cells are generally in a volume of a liter or less, can be 500 mis or less, even 250 mls or 100 mls or less. Hence the density of the desired cells is typically greater than 10⁴ cells/ml and generally is greater than 10⁷ cells/ml, generally 10⁸ cells/ml or greater. The clinically relevant number of immune cells can be apportioned into multiple infusions that cumulatively equal or exceed 10⁶, 10⁷, 10⁸, 10⁸, 10⁹, 10¹⁰ or 10¹¹ cells.

In some embodiments, the lymphocytes of the invention may be used to confer immunity to individuals. By “immunity” is meant a lessening of one or more physical symptoms associated with a response to infection by a pathogen, or to a tumor, to which the lymphocyte response is directed. The amount of cells administered is usually in the range present in normal individuals with immunity to the pathogen. Thus, the cells are usually administered by infusion, with each infusion in a range of from 2 cells, up to at least 10⁶ to 3×10¹⁰ cells, preferably in the range of at least 10⁷ to 10⁹ cells. The T cells may be administered by a single infusion, or by multiple infusions over a range of time. However, since different individuals are expected to vary in responsiveness, the type and amount of cells infused, as well as the number of infusions and the time range over which multiple infusions are given are determined by the attending physician, and can be determined by routine examination. The generation of sufficient levels of T lymphocytes (including cytotoxic T lymphocytes and/or helper T lymphocytes) is readily achievable using the rapid expansion method of the present invention, as exemplified herein. See, e.g., U.S. Pat. No. 6,040,177 to Riddell et al. at column 17.

In embodiments, the composition as described herein are administered intravenously, intraperitoneally, intratumorly, into the bone marrow, into the lymph node, and/or into cerebrospinal fluid. In embodiments, the chimeric receptor engineered compositions are delivered to the site of the tumor. Alternatively, the compositions as described herein can be combined with a compound that targets the cells to the tumor or the immune system compartments and avoid sites such as the lung.

In embodiments, the compositions as described herein are administered with chemotherapeutic agents and/or immunosuppressants. In an embodiment, a patient is first treated with a chemotherapeutic agent that inhibits or destroys other immune cells followed by the compositions described herein. In some cases, chemotherapy may be avoided entirely.

The present invention is illustrated further in the examples set forth below.

### EXPERIMENTAL

### Example I. Customizing Spacer Domain Length and scFv Affinity for Optimal Recognition of ROR1 with Chimeric Receptor Modified T Cells

We constructed chimeric receptors specific for the ROR1 molecule that is expressed on a large number of human malignancies including chronic lymphocytic leukemia, mantle cell lymphoma, acute lymphoblastic leukemia, and breast, lung prostate, pancreas and ovarian cancer. The ROR1 chimeric receptors were designed from ROR1 specific scFVs with different affinities and containing extracellular IgG4-Fc spacer domains of different lengths. The ability of T-cells expressing each ROR-1 specific chimeric receptor to recognize ROR1⁺ hematopoietic and epithelial tumors in vitro, and to eliminate human mantle cell lymphoma engrafted into immunodeficient mice was analyzed.

### Materials and Methods

### Human Subjects

Peripheral blood mononuclear cells (PBMC) were obtained from healthy donors and patients after written informed consent on research protocols approved by the Institutional Review Board of the Fred Hutchinson Cancer Research Center (FHCRC).

### Cell Lines

The K562, Raji, JeKo-1, MDA-MB-231, MDA-MB-468, and 293T cell lines were obtained from the American Type Culture Collection. Dr. Edus H. Warren (FHCRC) kindly provided the renal cell cancer lines FARP, TREP and RWL. K562/ROR1 and Raji/ROR1 were generated by lentiviral transduction with the full-length ROR1-gene. To derive JeKo-1/ffluc, native JeKo-1 cells were transduced with a lentiviral vector encoding the firefly luciferase (ffluc)-gene upstream of a T2A sequence and eGFP. The transduced JeKo-1 cells were sorted for eGFP expression, and expanded for in vivo experiments.

### Immunophenotyping

PBMC and cell lines were stained with the following conjugated mAbs: CD3, CD4, CD5, CD8, CD19, CD28, CD45RO, CD62L, CD314 (NKG2D), MICA/B and matched isotype controls (BD Biosciences). Propidium iodide (PI) staining was performed for live/dead cell discrimination. Cell surface expression of ROR1 was analyzed using a polyclonal goat anti-human-ROR1 antibody (R&D Systems).

Surface expression of 2A2 ROR1chimeric receptor was analyzed using a polyclonal goat anti-mouse-IgG antibody (Fab-specific) (Jackson ImmunoResearch). Flow analyses were done on a FACSCanto®, sort-purifications on a FACSAriaII® (Becton Dickinson) and data analyzed using FlowJo® software (Treestar).

### Vector Construction and Preparation of Chimeric Receptor Encoding Lentivirus

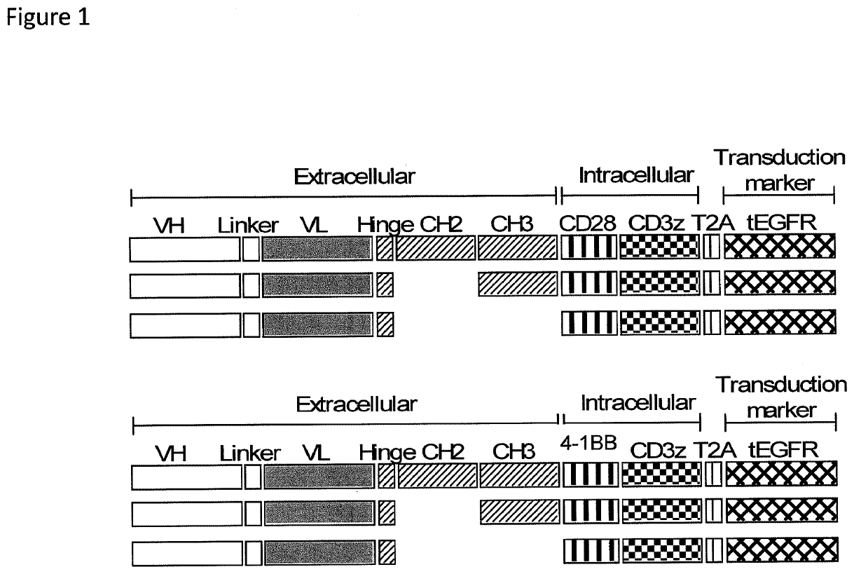

ROR1-specific and CD19-specific chimeric receptors were constructed using VL and VH chain segments of the 2A2, R12, and R11 mAbs (ROR1) and FMC63 mAb (CD19). (Variable region sequences for R11 and R12 are provided in Yang et al, Plos One 6(6):e21018, Jun. 15, 2011) Each scFV was linked by a (G₄S)₃ (SEQ ID NO:12) peptide to a spacer domain derived from IgG4-Fc (Uniprot Database: P01861, SEQ ID NO:13) comprising either ‘Hinge-CH2-CH3’ (229 AA, SEQ ID NO:), ‘Hinge-CH3’ (119 AA, SEQ ID NO:) or ‘Hinge’ only (12 AA, SEQ. ID NO:4) sequences (FIG. 1). All spacers contained a S→P substitution within the ‘Hinge’ domain located at position 108 of the native IgG4-Fc protein, and were linked to the 27 AA transmembrane domain of human CD28 (Uniprot: P10747, SEQ ID NO:14) and to a signaling module comprising either (i) the 41 AA cytoplasmic domain of human CD28 with an LL→GG substitution located at positions 186-187 of the native CD28 protein (SEQ ID NO:14) or (ii) the 42 AA cytoplasmic domain of human 4-1BB (Uniprot: Q07011, SEQ ID NO:15), each of which was linked to the 112 AA cytoplasmic domain of isoform 3 of human CD3ζ (Uniprot: P20963, SEQ ID NO;16). The construct encoded a T2A ribosomal skip element (SEQ ID NO:8)) and a tEGFR sequence (SEQ ID NO:9) downstream of the chimeric receptor. Codon-optimized nucleotide sequences encoding each transgene were synthesized (Life Technologies) and cloned into the epHIV7 lentiviral vector

ROR1-chimeric receptor, CD19-chimeric receptor or tEGFR-encoding lentiviruses were produced in 293T cells using the packaging vectors pCHGP-2, pCMV-Rev2 and pCMV-G, and Calphos® transfection reagent (Clontech).

### Generation of T-Cell Lines Expressing ROR1 and CD19-Chimeric Receptors

CD8⁺CD45RO⁺ CD62L⁺ central memory T-cells (T_{CM}) or bulk CD4⁺ T-cells were sorted from PBMC of normal donors, activated with anti-CD3/CD28 beads (Life Technologies), and transduced on day 3 after activation by centrifugation at 800 g for 45 min at 32° C. with lentiviral supernatant (MOI=3) supplemented with 1 μg/mL polybrene (Millipore). T-cells were expanded in RPMI with 10% human serum, 2 mM L-glutamine and 1% penicillin-streptomycin (CTL medium), supplemented with recombinant human IL-2 to a final concentration of 50 U/mL. The tEGFR⁺ subset of each T-cell line was enriched by immunomagnetic selection with biotin-conjugated anti-EGFR mAb (ImClone Systems) and streptavidin-beads (Miltenyi). ROR1-chimeric receptor and tEGFR control T-cells were expanded using a rapid expansion protocol (Riddell S R, Greenberg P D, The use of anti-CD3 and anti-CD28 monoclonal antibodies to clone and expand human antigen-specific T cells J Immunol Methods. 1990; 128(2):189-201. Epub 1990/04/17.), and CD19-chimeric receptor modified T-cells were expanded by stimulation with irradiated (8,000 rad) B-LCL at a T-cell:LCL ratio of 1:7. T-cells were cultured in CTL medium with 50 U/mL IL-2.

### Cytotoxicity, Cytokine Secretion and Proliferation Assays

Target cells were labeled with ⁵¹Cr (PerkinElmer), washed and incubated in triplicate at 1-2×10³ cells/well with effector chimeric receptor modified T-cells at various effector to target (E:T) ratios. Supernatants were harvested for y-counting after a 4-hour incubation and specific lysis calculated using the standard formula. For analysis of cytokine secretion, 5×10⁴ T-cells were plated in triplicate with target cells at an E:T ratio of 1:1 (primary CLL), 2:1 (Raji/ROR1; JeKo-1), 4:1 (K562/ROR1, K562/CD19 and K562) or 10:1 (MDA-MB-231), and IFN-γ, TNF-α and IL-2 measured by ELISA or multiplex cytokine immunoassay (Luminex) in supernatant removed after 24-h incubation. In experiments blocking NKG2D signaling, anti-NKG2D (clone 1D11), anti-MICA/B (clone 6D4, all from BD) and anti-ULBP (kindly provided by Dr. Veronika Groh, FHCRC) were used at saturating concentrations. For analysis of proliferation, T-cells were labeled with 0.2 μM carboxyfluorescein succinimidyl ester (CFSE, Invitrogen), washed and plated in triplicate with stimulator cells in medium without exogenous cytokines. After 72-h incubation, cells were labeled with anti-CD8 mAb and PI, and analyzed by flow cytometry to assess cell division of live CD8+ T-cells.

### Experiments in NOD/SCID/γc^{-/-} (NSG) Mice

The Institutional Animal Chimeric receptor and Use Committee approved all mouse experiments. Six- to 8-week old female NOD.Cg-Prkdc^{scid} Il2rg^{tm1Wjl}/SzJ (NSG) mice were obtained from the Jackson Laboratory or bred in-house. Mice were injected with 0.5×10⁶ JeKo−1/ffluc tumor cells via tail vein and received a subsequent tail vein injection of chimeric receptor-modified or control T-cells.

For bioluminescence imaging of tumor growth, mice received intraperitoneal injections of luciferin substrate (Caliper Life Sciences) resuspended in PBS (15 μg/g body weight). Mice were anesthetized with isoflurane and imaged using an Xenogen IVIS Imaging System (Caliper) 10, 12 and 14 minutes after the injection of luciferin in small binning mode at an acquisition time of 1 s to 1 min to obtain unsaturated images. Luciferase activity was analyzed using Living Image Software (Caliper) and the photon flux analyzed within regions of interest that encompassed the entire body or the thorax of each individual mouse.

### Statistical Analyses

Statistical analyses were performed using Prism Software (GraphPad®). Student's t-test was performed as a two-sided paired test with a confidence interval of 95% and results with a p-value of p<0.05 were considered significant. Statistical analysis of survival were done by log-rank testing and results with a p-value of p<0.05 considered significant.

### Results

### Truncating the Spacer Domain of the 2A2 ROR1-Chimeric Receptor Confers Superior Recognition of ROR1⁺ Tumors

We previously reported the design of a ROR1-specific chimeric receptor using the 2A2 scFV, which binds to an epitope in the NH2-terminal, membrane distal Ig-like/Frizzled portion of ROR1-1. The initial 2A2 ROR1-chimeric receptor had a long 229 AA spacer that included the ‘Hinge-CH2-CH3’ region of IgG4-Fc, and incorporated CD28 costimulatory and CD3ζ signaling domains (Hudecek M et al. Blood, 2010). This chimeric receptor conferred specific recognition of ROR1⁺ tumors, but we hypothesized that because of the membrane distal location of the ROR1 epitope, truncating the spacer domain might enhance tumor recognition and T-cell signaling. Therefore, we constructed 2 additional chimeric receptors in which the IgG4-Fc spacer domain was sequentially deleted to derive ‘Hinge-CH3’ (119 AA, intermediate), and ‘Hinge-only’ (12 AA, short) variants. Each of the new receptors contained the identical 2A2 scFV, and CD28 and CD3ζ signaling modules. The transgene cassette included a truncated EGFR (tEGFR) to serve as a transduction, selection and in vivo tracking marker for chimeric receptor-modified T-cells.

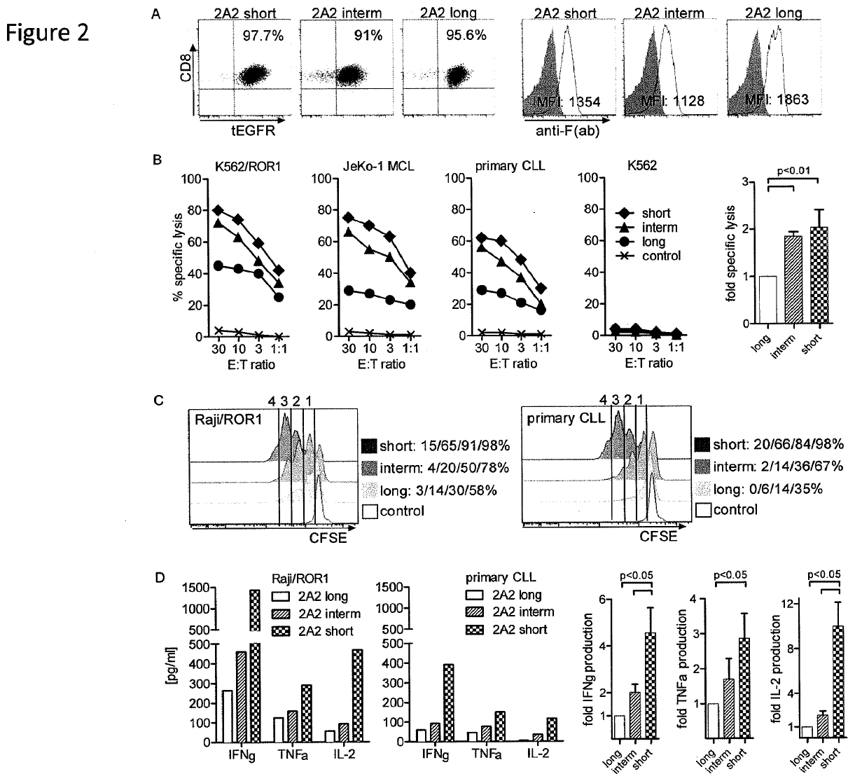

We transduced purified CD8+ T_{CM} with the 2A2 ROR1-chimeric receptors containing full length or truncated IgG4-Fc spacers, and with a tEGFR control vector. The mean transduction efficiency was 15% (range 9-22%), and transgene-positive T-cells were enriched to uniform purity (>90%) on day 10 by selection for tEGFR expression, and expanded (FIG. 2A). Surface expression of each of the chimeric receptors was confirmed by staining with F(ab)-specific antibodies (FIG. 2A).

Analysis of the in vitro function of CD8+ T-cells modified to express each of the 2A2 ROR1-chimeric receptors demonstrated that each receptor conferred specific lysis of JeKo-1 MCL and primary CLL cells that naturally express ROR1, and of K562 cells that had been transduced with ROR1, but did not confer recognition of control ROR1⁻ targets (FIG. 2B). T-cells expressing the short ‘Hinge-only’ 2A2 ROR1-chimeric receptor had maximum cytolytic activity, and a hierarchy (short>intermediate>>long) of tumor lysis was clearly evident against all ROR1⁺ tumor targets (FIG. 2B), illustrating the importance of spacer domain length on the recognition of ROR1⁺ tumor cells.

Anti-tumor efficacy of adoptive T-cell therapy correlates with proliferation and survival of transferred T-cells, which could be altered by signaling through the chimeric receptor. We used CFSE dilution assays to analyze proliferation of T-cells modified with each of the 2A2 ROR1-chimeric receptors after engagement of Raji/ROR1 or CLL, and found that the short spacer construct promoted the greatest T-cell proliferation following stimulation (FIG. 2C). To ensure that the enhanced proliferation was not associated with greater activation induced cell death (AICD), we also analyzed the proportion of 2A2 ROR 1 chimeric receptor modified T-cells that stained with propidium iodide (PI) after stimulation with Raji/ROR1 and JeKo-1 tumor cells. We detected a much lower frequency of PI⁺ CD8⁺ T-cells in the T-cell line modified with the short (Raji/ROR1: 17.2%/JeKo-1: 20.2%) compared to the intermediate (41.6%/42.4%) and long (44.5%/48.5%) spacers.

Quantitative analysis of cytokine production in response to stimulation with Raji/ROR1 and primary CLL cells showed production of IFN-γ, TNF-α and IL-2 by T-cells expressing each of the 2A2 ROR1 chimeric receptors. As observed in cytotoxicity assays, the short spacer construct was superior in mediating cytokine secretion after tumor recognition (FIG. 2D). Thus, this analysis shows that truncating the extracellular IgG4-Fc spacer domain of the 2A2 ROR1-chimeric receptor leads to a significant increase in cytotoxicity, proliferation and in vitro effector functions after tumor recognition.

The RII scFv that is Specific for a Membrane Proximal Epitope in the ROR1 Kringle Domain Requires a Long Extracellular Spacer Domain.

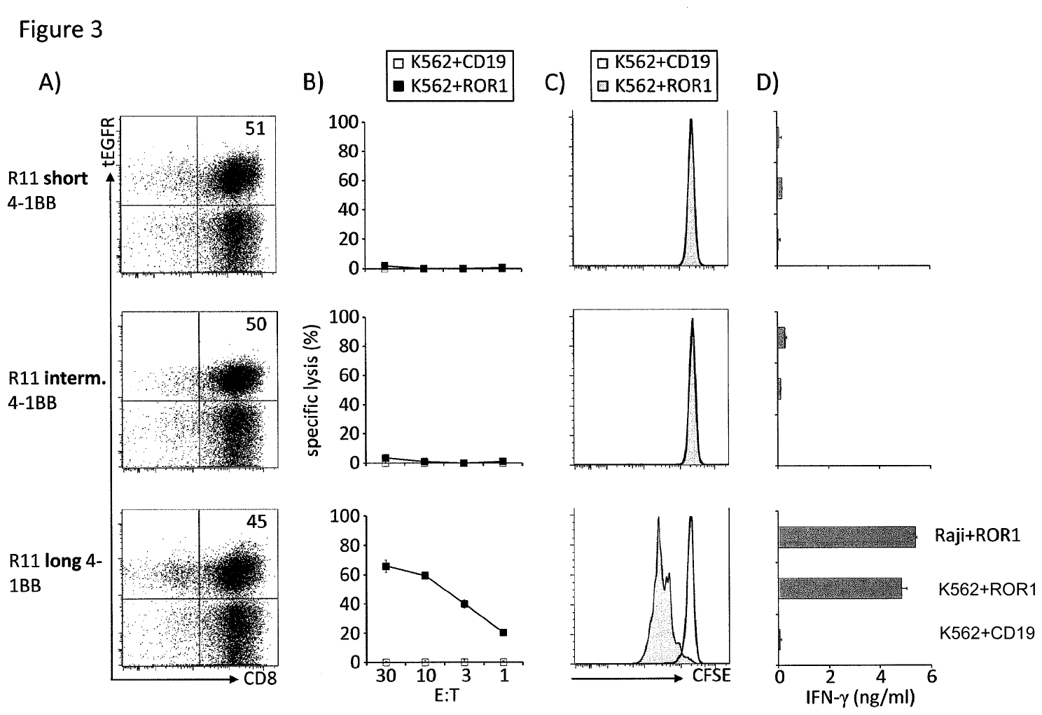

We transduced purified CD8⁺ T cells with ROR1-chimeric receptors containing the R11 scFv that is specific for the Kringle domain of ROR1 and containing full length or truncated IgG4-Fc spacers (CH3 and hinge only). The transduction efficiency with each of the short (IgG4 hinge only), intermediate (IgG4 hinge/CH3), and long (IgG4 hinge/CH2/CH3) vectors was comparable (45-51%) as measured by EGFR expression. (FIG. 3A). T cells transduced with each of the vectors were assayed for cytolysis (FIG. 3 B), proliferation (FIG. 3C), and cytokine production (FIG. 3D) in response to leukemia or lymphoma cells that did or did not express ROR1. As shown, only T cells transduced with the R11 chimeric receptor containing a long spacer sequence were able to efficiently recognize ROR1+ tumors and mediate effector functions.

ROR1 Chimeric Receptors Derived from a mAb R12 with Higher Affinity than 2A2 Mediate Superior Anti-Tumor Reactivity

We next examined whether increasing the affinity of the scFV used to construct the ROR1 chimeric receptor might influence tumor recognition and T-cell function. We generated ROR1-specific chimeric receptors from the mAb R12 that like 2A2, binds to an epitope in the NH2-terminal Ig/Frizzled domain of ROR1 but with >50-fold higher monovalent binding affinity.

R12 ROR1 chimeric receptors were constructed with both long and short IgG4-Fc spacers to determine whether the optimal spacer design for this higher affinity scFV differed from that for a lower affinity scFV. We found that similar to 2A2, the short spacer R12 ROR1 chimeric receptor conferred improved cytolytic activity, cytokine secretion and proliferation (data not shown), suggesting that the shorter spacer length provides superior spatial engagement of the T-cell and ROR1⁺ target cell for T-cell activation.

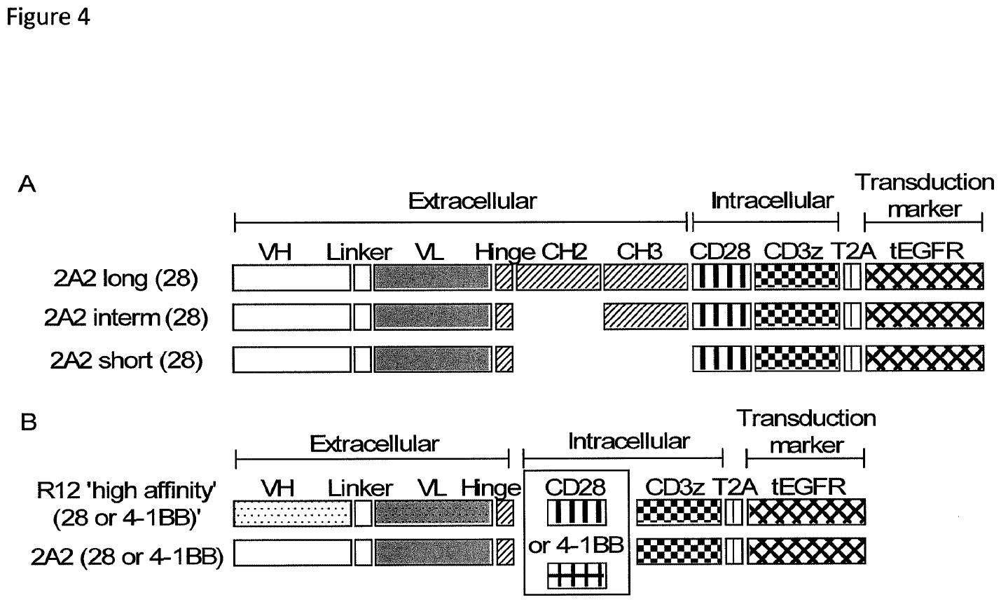

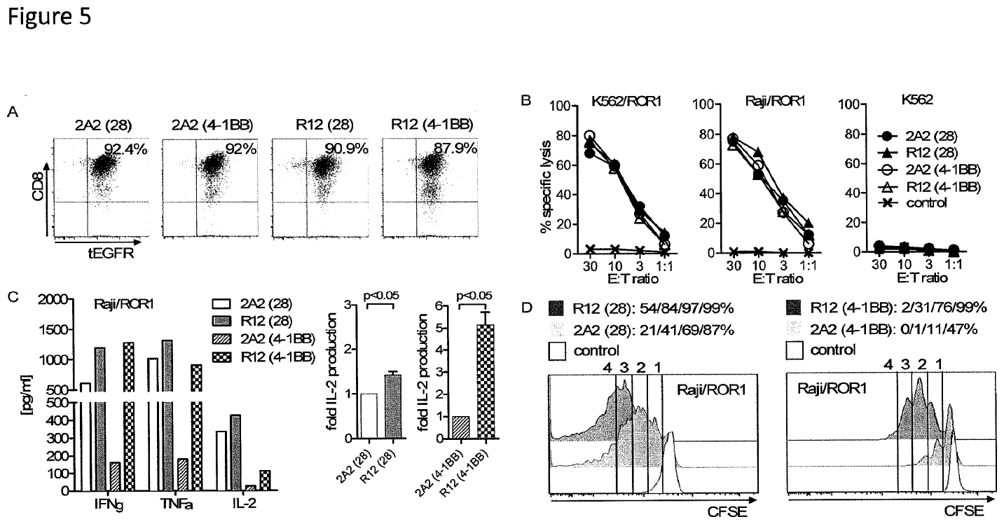

We then designed R12 and 2A2 ROR1 chimeric receptors that contained an optimal (short) extracellular spacer, and either a CD28 or 4-1BB costimulatory domain in tandem with CD3ζ (4 constructs) for comparison (FIG. 4A.B). These ROR1-chimeric receptor constructs were expressed in purified CD8⁺ T_{CM} of healthy donors, and we confirmed equivalent transgene expression by tEGFR staining (FIG. 5A). T-cells modified with each of the 2A2 and R12 ROR1-chimeric receptors specifically lysed K562/ROR1 and Raji/ROR1 tumor cells with approximately equivalent efficiency (FIG. 5B). However, analysis of cytokine production showed that the high affinity R12 ROR1 chimeric receptors that contained CD28 or 4-1BB conferred significantly higher IFN-γ, TNF-α and IL-2 production compared to the corresponding 2A2 constructs (FIG. 5C). We found that T-cells expressing chimeric receptors with a CD28 costimulatory domain produced more IFN-γ, TNF-α and IL-2 compared to those with 4-1BB.

Experiments to analyze the proliferation of ROR1 chimeric receptor T-cells showed a higher percentage of proliferating T-cells and a higher number of cell divisions in T-cells expressing the high affinity R12 ROR1 chimeric receptors with CD28 and 4-1BB domain compared to T-cells expressing the respective 2A2 counterparts (FIG. 4D). There was more vigorous proliferation in T-cells that expressed chimeric receptors with a CD28 domain, consistent with higher IL-2 production induced by these receptors. There was a lower frequency of AICD as measured by PI staining in T-cell lines modified with R12 compared to 2A2 ROR1-chimeric receptors after stimulation with Raji/ROR1 and JeKo-1 tumor cells respectively (R12: 5.6%/6.9% vs. 2A2: 10%/9.65%). T-cell lines that expressed chimeric receptors with a CD28 domain also had lower AICD compared to 4-1BB in response to Raji/ROR1 and JeKo-1 tumor cells respectively (R12: 16.4%/18.4% vs. 2A2 38.1%/39.6%).

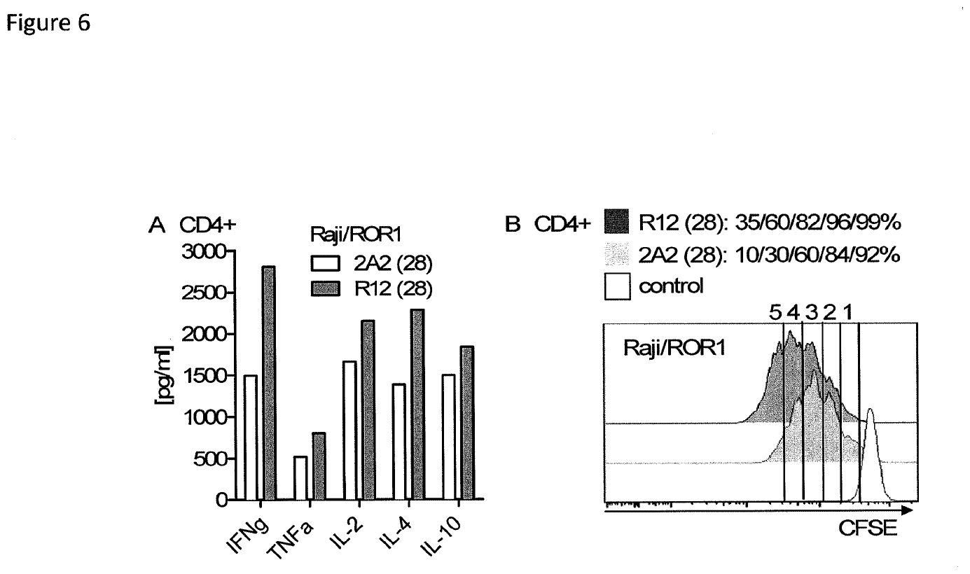

To determine if the enhanced function observed with R12 ROR1 chimeric receptors in CD8⁺ T-cells extended to CD4⁺ T-cells, we transduced bulk CD4⁺ T-cells with the 2A2 and R12 ROR1 chimeric receptors containing the short spacer and CD28 costimulatory domain. In response to Raji/ROR1⁺ tumor cells, CD4⁺ T-cells that expressed the high affinity R12 scFV produced higher levels of IFN-γ, TNF-α, IL-2, IL-4, and IL-10, and underwent greater proliferation than CD4⁺ T-cells that expressed 2A2 (FIG. 5A,B). Both cytokine production and proliferation was superior in CD4⁺ compared to CD8⁺ T-cells modified with the same ROR1 chimeric receptors. In summary, our data demonstrate that tailoring both the length of the non-signaling extracellular chimeric receptor spacer domain and scFV affinity are independent parameters that affect the function of ROR1-chimeric receptor T-cells. D8⁺ T-cells modified with a high affinity ROR1 chimeric receptor have comparable activity to a CD19 chimeric receptor against primary CLL in vitro ROR1 and CD19 are both uniformly expressed on all primary CLL (FIG. 6A), however the absolute number of ROR1-molecules per tumor cell is estimated to be 10-fold lower than that of CD19, which has been successfully targeted in clinical trials with CD19 chimeric receptor T-cells. We compared recognition of primary CLL by CD8⁺ T-cells expressing the optimized R12 and 2A2 ROR1 chimeric receptors, and a CD19 chimeric receptor derived from the FMC63 scFV.

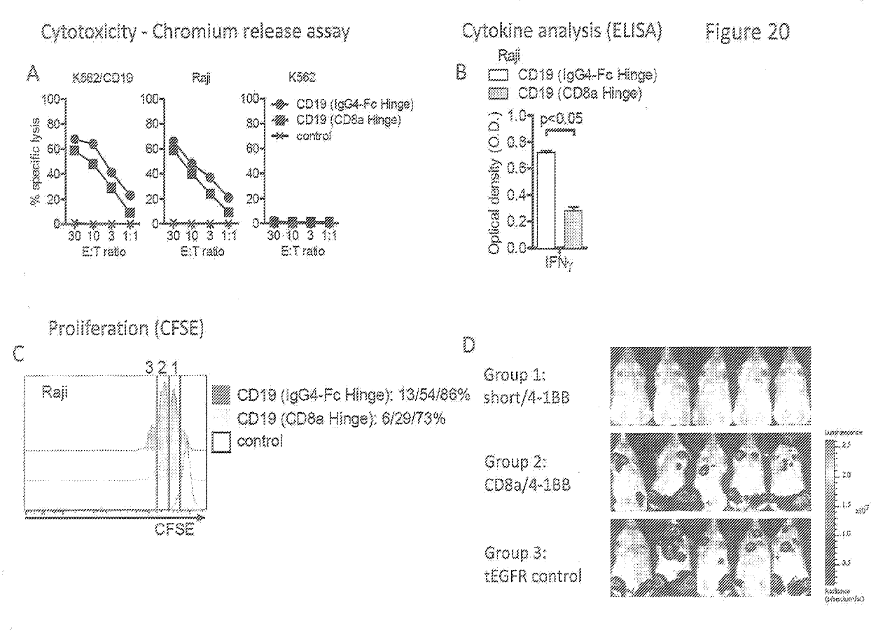

We used purified CD8⁺ T_{CM} for chimeric receptor-modification to provide a uniform cell product and each chimeric receptor contained a short IgG4-Fc ‘Hinge-only’ spacer and 4-1BB costimulatory domain. We confirmed our CD19 chimeric receptor (IgG4 Hinge) was at least as and more effective in recognizing CD19+ tumors as a CD19 chimeric receptor with CD8a Hinge spacer and 4-1BB costimulatory domain that is being used in ongoing clinical trials. (FIG. 20). T cells expressing CD19 chimeric receptors with 4-1BB and CD3zeta and a modified IgG4-Fc hinge exhibit superior in vitro and in vivo function compared to T cells expressing CD19 chimeric receptors with 4-1BB and CD3zeta and a CD8 alpha hinge. In FIG. 20D, in vivo antitumor activity of T cells expressing a CD19 chimeric receptor with an IgG4 Fc hinge (group 1) or CD8 alpha hinge (group 2) and T cells that express tEGFR alone (group 3) in NSG mice inoculated with Raji tumor cells expressing firefly luciferase (ffluc) were compared. Mice were imaged 17 days after tumor inoculation and 10 days after T cell inoculation. The data shows greater tumor burden in mice treated with control tEGFR T cells (group 3) or with CD19 chimeric receptor CD8 alpha hinge T cells (group 2) compared with mice treated with CD19 chimeric receptor IgG4 Fc hinge T cells (group 1).

The cytolytic activity of R12 ROR1 chimeric receptor T-cells against primary tumor cells from multiple CLL patients (n=4) was higher compared to T-cells modified with the lower affinity 2A2 ROR1 chimeric receptor, and equivalent to the lysis observed with CD19 chimeric receptor T-cells (FIG. 6B). Multiplex cytokine analysis showed nearly equivalent production of IFN-γ and TNF-α, but less IL-2 production by CD8⁺ T-cells expressing the R12 ROR1 compared with those expressing the CD19-chimeric receptor after co-culture with primary CLL (FIG. 6C). 2A2 ROR1 chimeric receptor T-cells produced lower amounts of all cytokines than R12 ROR1 chimeric receptor T-cells as noted previously. Cytokine production by all of the chimeric receptor-transduced T-cells after stimulation with CLL was substantially less than with Raji/ROR1, which unlike CLL expresses both CD80 and CD86 that can engage CD28 expressed on chimeric receptor T-cells (FIG. 6A, C).

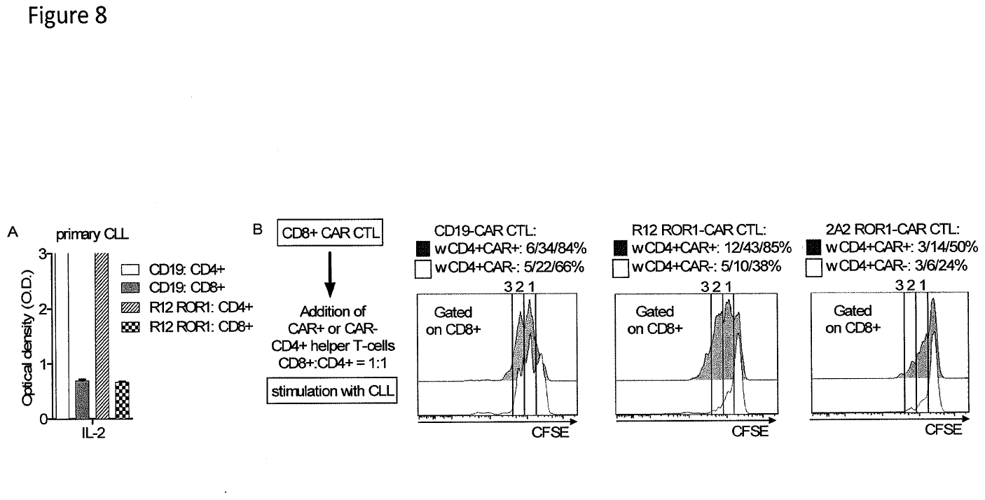

We observed less proliferation of T-cells expressing the R12 and 2A2 ROR1 chimeric receptor compared to the CD19 chimeric receptor after stimulation with CLL (CD19>R12>2A2) (FIG. 6D). We hypothesized that proliferation of CD8⁺ ROR1 chimeric receptor T-cells in response to CLL may be augmented in the presence of chimeric receptor-modified CD4⁺ T-cells because of their higher secretion of IL-2 compared to CD8⁺ T_{CM}(FIG. 4A; FIG. 8A). To test this possibility, we performed in vitro co-culture experiments where CD4⁺ and CD8 T_{CM} were separately modified with the R12 ROR1, 2A2 ROR1 and CD19 chimeric receptors respectively, enriched for chimeric receptor expression, and combined at a 1:1 ratio to ensure equivalent proportions of CD8⁺ and CD4⁺ T-cells modified with each of the vectors. These cells were CFSE-labeled and stimulated with primary CLL. We observed a dramatic increase in proliferation of CD8⁺ R12 ROR1 chimeric receptor T-cells after addition of chimeric receptor-transduced, but not untransduced CD4⁺ T-cells (FIG. 8B). Notably, when provided with CD4-help, we observed equivalent proliferation of R12 ROR1 and CD19 chimeric receptor CD8⁺ T-cells in response to CLL, whereas proliferation of CD8⁺ T-cells expressing the lower affinity 2A2 ROR1 chimeric receptor remained less. Collectively, our data show that the high affinity R12 ROR1 chimeric receptor confers superior reactivity compared to 2A2 against primary CLL cells in vitro.

### ROR1-Chimeric Receptor T-Cells Mediate In Vivo Anti-Tumor Activity in a Mouse Model of Systemic Mantle Cell Lymphoma

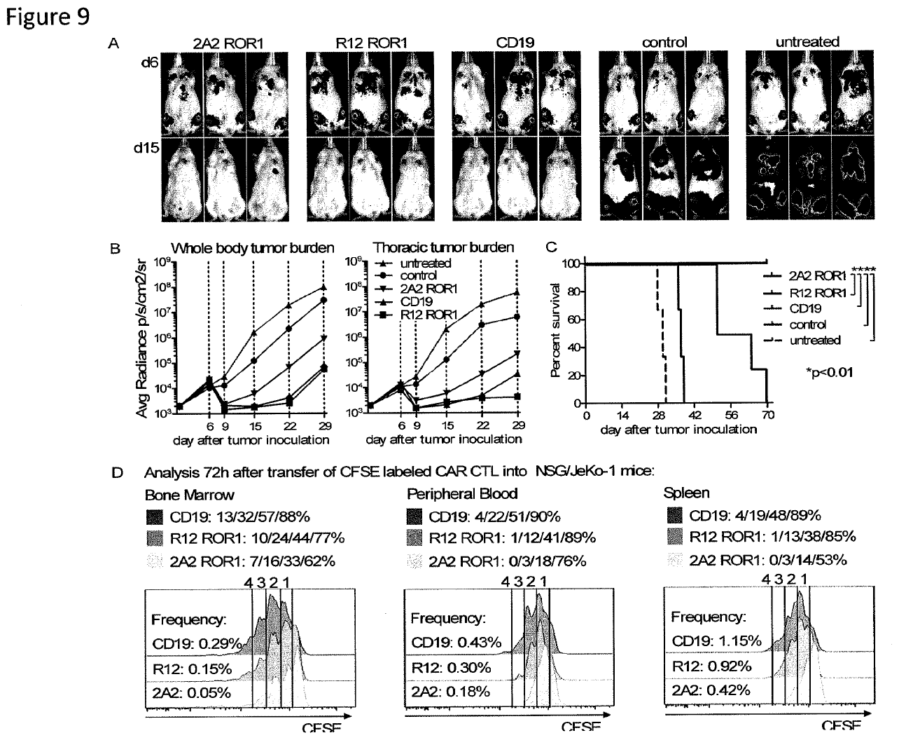

It remained uncertain whether the superior in vitro activity of T-cells modified with the higher affinity R12 chimeric receptor would translate into improved anti-tumor activity in vivo, and how targeting ROR1 would compare to targeting CD19. To address these questions, we inoculated cohorts of immunodeficient NSG mice with the human MCL line JeKo-1/ffluc by tail vein injection, and seven days later when tumor was disseminated, treated the mice with a single intravenous dose of R12 ROR1, 2A2 ROR1 or CD19 chimeric receptor CD8⁺ T-cells. Control mice were treated with tEGFR T-cells or untreated. All chimeric receptors had the optimal short spacer and the 4-1BB costimulatory domain. Untreated NSG/JeKo-1 mice developed a rapidly progressive systemic lymphoma necessitating euthanasia approximately 4 weeks after tumor inoculation (FIG. 9A-C).

We observed tumor regression and improved survival in all mice treated with R12 ROR1, 2A2 ROR1 and CD19 chimeric receptor T-cells. Mice treated with R12 ROR1 chimeric receptor T-cells had a superior anti-tumor response and survival compared to mice treated with 2A2 ROR1 chimeric receptor T-cells (p<0.01), and comparable anti-tumor activity to mice treated with CD19 chimeric receptor T-cells (FIG. 9A-C).

We analyzed the frequency of chimeric receptor T-cells in the peripheral blood following adoptive transfer and detected higher numbers of tEGFR⁺ T-cells in mice treated with the R12 ROR1 chimeric receptor compared to the 2A2 ROR1 chimeric receptor, suggesting more vigorous proliferation in vivo improved tumor control. To confirm this, we administered CFSE-labeled CD19 chimeric receptor, R12 and 2A2 ROR1 chimeric receptor T-cells to cohorts of NSG mice bearing JeKo-1/ffluc, and analyzed T-cell proliferation in the peripheral blood, bone marrow and spleen 72 hours after transfer. A higher percentage of the R12 and CD19 chimeric receptor T-cells proliferated and underwent a greater number of cell divisions compared to 2A2 ROR1 chimeric receptor T-cells (FIG. 9D). The JeKo-1 tumor eventually recurred in all mice treated with ROR1 or CD19 chimeric receptor T-cells (FIG. 9A-C). Tumor recurrence was not a result of the selection of ROR1 or CD19 loss variants, as recurrent tumors were positive for both molecules.

For comparison, we analyzed anti-tumor efficacy of CD19 chimeric receptor T-cells in NSG mice engrafted with Raji tumors and observed complete tumor eradication, indicating the recurrence of JeKo-1 reflects difficulty eradicating this tumor (data not shown). In summary, this data is the first to show that ROR1 chimeric receptor T-cells have anti-tumor efficacy in vivo, and suggest that for B-cell malignancies, an optimized ROR1 chimeric receptor such as R12 may be effective and spare normal CD19⁺ B-cells that lack ROR1 expression.

T-Cells Expressing the R12 ROR1 Chimeric Receptor have Superior Reactivity Compared to 2A2 Against ROR1⁺ Epithelial Tumor Cells

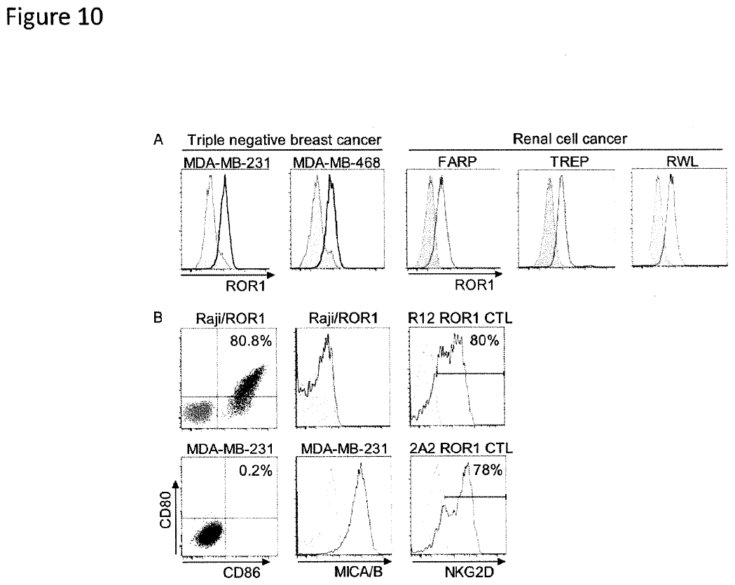

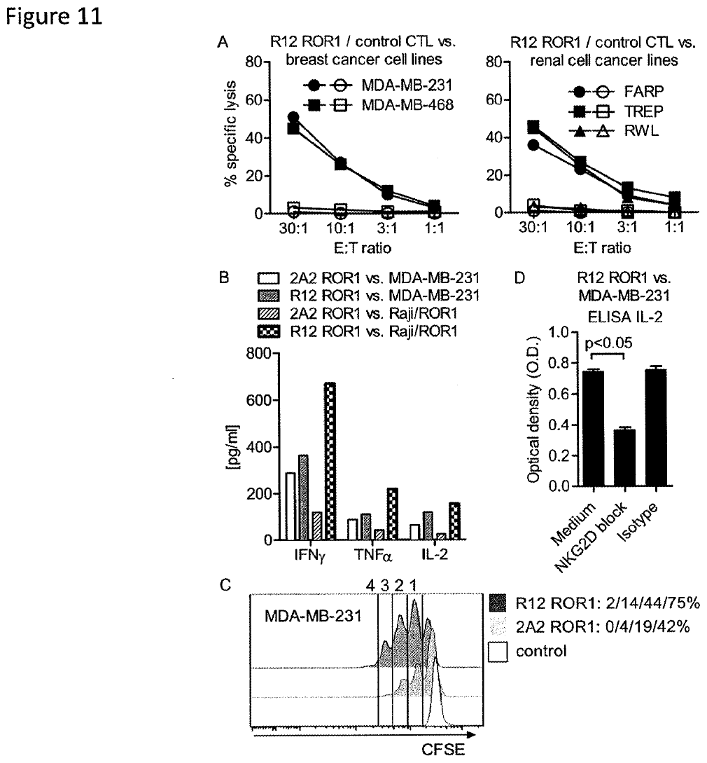

ROR1 has been detected on many epithelial tumors, although it is unknown whether ROR1 expression is sufficient for recognition by ROR1 chimeric receptor T-cells. Using flow cytometry, we confirmed ROR1 expression on breast cancer lines MDA-MB-231 and 468, and on the renal cell carcinoma lines FARP, TREP, and RWL (FIG. 10A). We then analyzed tumor recognition by CD8⁺ T-cells transduced with the R12 ROR1 chimeric receptors with the optimal short spacer and 4-1BB domain, and observed efficient recognition of MDA-MB-231, MDA-MB-468, FARP, TREP and RWL (FIG. 11A). We analyzed cytokine secretion and proliferation of T-cells modified with the R12 and 2A2 ROR1-chimeric receptors after co-culture with MDA-MB-231, and observed greater cytokine production and proliferation with the R12 ROR1 chimeric receptor (FIG. 11 B, C). Similar to what we observed with ROR1⁺ B cell malignancies, the superior activation of R12 ROR1 chimeric receptor T cells after stimulation with MDA-MB-231 was not associated with increased AICD (R12: 9.8% vs. 2A2: 10.9%).

### Discussion

ROR1 has attracted interest as a potential target for cancer immunotherapy due to its expression on the surface of many B-lymphoid and epithelial cancers, including subsets of lung, colorectal and renal cell cancer. We previously showed that CLL and MCL were specifically recognized by T-cells modified to express a ROR1-specific chimeric receptor (Hudecek M, et al. Blood. 2010; 116(22):4532-41. Epub 2010/08/13). The design and function of ROR1-chimeric receptors has been improved through modification of the extracellular spacer domain and deriving the chimeric receptor from a scFV of higher affinity, and demonstrate that T-cells modified with designed ROR1 chimeric receptors have in vivo activity against ROR1⁺ B-cell lymphoma and in vitro activity against a wide range of epithelial tumors.

We compared the function of T-cells modified with ROR1 chimeric receptors derived from the 2A2 mAb that contained either the original long IgG4-Fc ‘Hinge-CH2-CH3’ spacer that we have shown enables high level cell surface expression, or truncated intermediate ‘Hinge-CH3’ and short ‘Hinge-only’ spacer variants. We preserved the 12 AA Hinge domain in our short spacer construct based on prior data that a flexible spacer was required for separating the scFV from the T-cell membrane and allowing antigen recognition on tumor cells (Fitzer-Attas C J, et al., Harnessing Syk family tyrosine kinases as signaling domains for chimeric single chain of the variable domain receptors: optimal design for T cell activation. J Immunol. 1998; 160(1):145-54. Epub 1998/04/29.) Our studies with the 2A2 ROR1 chimeric receptor show that T-cell cytokine secretion and proliferation after tumor cell recognition are superior with the intermediate and short spacer constructs compared to the long spacer construct. Staining with anti-F(ab) Abs showed equivalent chimeric receptor expression of all three receptors, demonstrating the improved T-cell function with the short spacer chimeric receptor was not due to differences in chimeric receptor density. This data supports the principle that the design of extracellular spacers should be tailored for each target molecule and epitope.

The affinity of the scFV selected for designing a chimeric receptor is an additional parameter that could affect T-cell recognition. We generated and characterized a panel of ROR1-specific mAbs of different affinities and selected the R12 mAb, which recognizes an epitope in the Ig-like/Frizzled region as 2A2. R12 has a higher affinity for ROR1-protein due to a much slower dissociation. The R12 chimeric receptor, like the 2A2 chimeric receptor conferred optimal T-cell recognition and function when designed with a short extracellular spacer. A direct comparison of proliferation and cytokine production after tumor engagement by T-cells modified with the 2A2 and R12 chimeric receptors demonstrated that the R12 chimeric receptor derived from the higher affinity mAb was superior. We were concerned that the slower dissociation of R12 from ROR1 could prolong T-cell activation and confer an increased susceptibility to AICD. However, we detected a lower rate of AICD in T-cells modified with the R12 ROR1-chimeric receptor compared to 2A2, demonstrating that the increased affinity of R12 had no detrimental effect on T-cell survival in our preclinical models.

ROR1 has a potential advantage over CD19 as a target for CLL and MCL since it is not expressed on normal mature naïve and memory B-cells. However, there is a lower number of ROR1 molecules on B-cell tumors compared with CD19 and it is uncertain if an optimized ROR1 chimeric receptor would be as effective as a CD19 chimeric receptor similar in design to those being used in the clinic. Unfortunately, B-cell tumor xenograft models used previously in NSG mice to evaluate the function of CD19 chimeric receptor T-cells including Raji, Daudi and Nalm-6, are not derived from CLL or MCL and do not constitutively express ROR1. Thus, to compare targeting CD19 and ROR1 in vivo, we used the JeKo-1 MCL cell line, which naturally expresses both CD19 and ROR1 and engrafts in NSG mice. To make our model clinically relevant, we inoculated JeKo-1 lymphoma cells intravenously to generate systemic tumors, and treated mice with T-cell products of uniform consistency once tumors were established. We found that T-cells expressing the high affinity R12 chimeric receptor conferred equivalent anti-tumor activity in vivo as CD19 chimeric receptor T-cells. Consistent with our in vitro analysis, the R12 ROR1 chimeric receptor also mediated superior activity in vivo compared to the optimal 2A2 ROR1-chimeric receptor. These results should be interpreted cautiously since murine tumor models may not predict the efficacy of adoptive therapy in clinical settings. However, the results suggest that ROR1 warrants consideration as an alternative to CD19, or to provide an additional target to minimize the potential for CD19 loss variants to emerge.

ROR1 appears to play a decisive role in survival of some epithelial tumors. Thus, an advantage of targeting ROR1 is that a single chimeric receptor may be useful to treat patients with a large number of hematopoietic and non-hematopoietic tumors.

Our data shows for the first time that T-cells that express a designed ROR1 chimeric receptor efficiently recognize epithelial cancers in vitro. Cytokine secretion and T-cell proliferation induced by ROR1⁺ breast cancer cells were higher than that induced by leukemia cells, despite the absence of the CD80/86 costimulatory ligand. The studies reported here demonstrate that the design of the extracellular spacer domain and chimeric receptor affinity are parameters that can be modulated to enhance the recognition of ROR1+ hematologic and epithelial tumors in vitro and in vivo by ROR1-chimeric receptor modified T-cells. The development of ROR1-chimeric receptors with enhanced tumor reactivity provides the opportunity for clinical applications in a variety of human cancers.

### Example 2. Effect of Extracellular Spacer Domain Length on Triggering of Tumor Cell Lysis with a Her2-Specific Chimeric Receptor that Recognizes an Epitope Located Proximal to the Tumor Cell Membrane

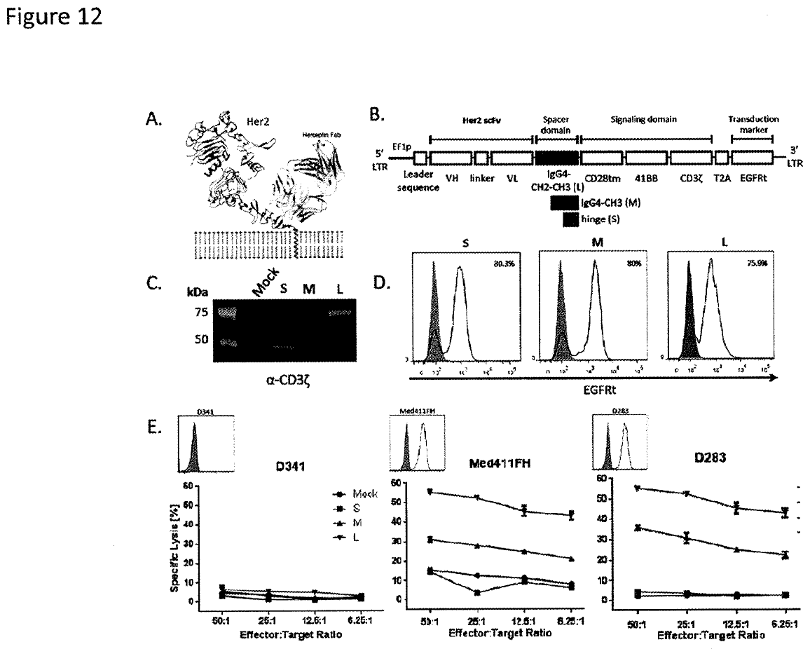

The effect of CAR spacer length on recognition and triggering of tumor cell recognition by CD8+ human T lymphocytes that expressed a HER2-specific chimeric receptor was examined using similar methods to those described above for ROR1. HER2-specific chimeric receptors were constructed using VL and VH chain segments of a HER2-specific mAb that recognized a membrane proximal epitope on HER2 (FIG. 12A), and the scFVs were linked to IgG4 hinge/CH2/CH3, IgG4 hinge/CH3, and IgG4 hinge only extracellular spacer domains and to the CD28 transmembrane domain, 4-1BB and CD3 zeta signaling domains (FIG. 12B). Primary CD8+ T cells were transduced with each of the HER2 chimeric receptors and selected for expression of the EGFR transducton marker (FIG. 12D). Expression of the HER 2 chimeric receptors and the size of each receptor was confirmed by Western Blot (FIG. 12C). The T cells were then expanded with anti CD3 mAb and feeder cells and examined for their ability to recognize HER2+ tumor cells. As observed with the R11 ROR 1 specific chimeric receptor, the HER2 chimeric receptor that contained a long extracellular spacer domain conferred superior T cell recognition of HER2+ tumor cells (FIG. 12E).

### Discussion

This example of the effect of extracellular spacer length on chimeric receptor modified T cell recognition of tumor cells used a chimeric receptor comprising a scFv built from the V_{H+L} sequences of the Herceptin chimeric mAb. Studies by Cho et al (Nature 421:756, 2003) localized to epitope location of Herceptin to a membrane proximal location on the HER2 (ERRB2) extracellular domain (FIG. 12A). Based on our understanding of the structure of human IgG4 hinge:Fc variants (FIG. 12B), we hypothesize that a membrane proximal location of the targeting epitope on an extracellular tumor cell antigen would best recognized by effector T cells that express a chimeric receptor encoding a long spacer. Our data demonstrating a gradient of cytolytic activity from near back ground activity by T cells expressing a short spacer Herceptin chimeric receptor, to intermediate activity by T cells expressing a medium length spacer chimeric receptor, and maximal lysis by T cells that expressed the long spacer chimeric receptor. Thus, the extracellular spacer has definitive effects on tumor recognition by T cells, and this data provides further support for the need to tailor chimeric receptor design based on epitope location of tumor expressed target molecules.

### Example 3. Customizing Spacer Length and Sequence for Optimal Recognition and In Vivo Efficacy of CD19 with Chimeric Receptor Modified T Cells

### Materials and Methods

### Human Subjects

Blood samples were obtained from healthy donors who provided written informed consent to participate in research protocols approved by the Institutional Review Board of the Fred Hutchinson Cancer Research Center (FHCRC). Peripheral blood mononuclear cells (PBMC) were isolated by centrifugation over Ficoll-Hypaque (Sigma, St. Louis, MO), and cryopreserved in RPMI, 20% human serum and 10% dimethyl sulfoxide.

### Cell Lines

The K562, Raji, JeKo-1, and 293T cell lines were obtained from the American Type Culture Collection (Manassas, VA) and cultured as directed. A lentivirus encoding the ffluc-gene upstream of a T2A sequence and eGFP was produced in 293T cells and used to transduce Raji and JeKo-1 tumor cells. Raji, and JeKo-1 cells were expanded after lentiviral transduction and the eGFP positive subset sort-purified,

### Immunophenotyping

PBMC and T-cell lines were stained with one or more of the following conjugated monoclonal antibodies: (CD3, (CD4, CD8, CD25, CD45RA, CD45RO, CD62L, CD69 and matched isotype controls (BD Biosciences), Staining with propidium iodide (PI, BD Biosciences) was performed for live/dead cell discrimination as directed by the manufacturer. Flow analyses were done on a FACSCanto, sort-purifications on a FACSAriaII (Becton Dickinson) and data analyzed using FlowJo software (Treestar).

### Vector Construction and Preparation of CD119 Chimeric Receptor Encoding Lentivirus

CD19 specific chimeric receptors were constructed using: (1) the VL and VH chain segments of the CD19-specific mAb FMC63 (SEQ ID NO:3), linked by a (G₄S)₃ linker (SEQ ID NO:12) peptide (VL-linker-VH); (2) a spacer domain derived from IgG4-Fc (Uniprot Database: P01861, (SEQ ID NO: 13)) comprising either the Hinge-CH2-CH3 portion (229 AA, (SEQ ID NO:)) or Hinge only (12 AA; (SEQ MD NO:4)). Both spacers contained a S→P substitution within the Hinge domain located at position 108 of the native IgG4-Fec protein; the 27 AA transmembrane domain of human CD28 (Uniprot Database: P10747, (SEQ ID NO:14)); (4) a signaling module comprising either (i) the 41 AA cytoplasmic domain of human CD28 with an LL→GG substitution located at position 186-187 of the native CD28 protein (SEQ 11) NO:14); and/or (ii) the 42 AA cytoplasmic domain of human 4-1BB (Uniprot Database: Q07011, (SEQ ID NO:15)); linked to (iii) the 112 AA cytoplasmic domain of isoform 3 of human CD3 (Uniprot Database: P20963, (SEQ ID NO:16)); the self cleaving T2A sequence (SEQ ID NO:8); and (6) a truncated epidermal growth factor receptor (EGFR) sequence (SEQ ID NO:9).

Codon-optimized nucleotide sequences encoding each trans gene were synthesized (LifeTechnologies, Carlsbad. CA) and cloned into the epHIV7 lentiviral vector using NheI and Not1 restriction sites. The epHIV7 lentiviral vector had been derived from the pHIV7 vector by replacing the cytomegalovirus promoter of pHIV7 with an EF-1 promoter.

CD19 chimeric receptor or tEGFR-encoding lentivirus was produced in 293T cells co-transfected with the lentiviral vector and the packaging vectors pCHGP-2, pCMV-Rev2 and pCMV-G using Calphos transfection reagent (Clontech). Medium was changed 16 h after transfection, and lentivirus collected after 24, 48 and 72 h.

### Generation of T-Cell Lines Expressing the CD19 Chimeric Receptors

Sort-purified CD8⁺ CD45RA⁻ CD45RO⁺ CD62L⁺ central memory T-cells (T_{CM}) of normal donors were activated with anti-CD3/CD28 beads (Life Technologies) according to the manufacturer's instructions, and transduced with lentiviral supernatant (MOI=3) supplemented with 1 μg/mL polybrene (Millipore) on day 3 after activation by centrifugation at 2,100 rpm for 45 min at 32° C. T cells were expanded in RPM1, 10% human serum, 2 mM L-glutamine and 1% penicillin-streptomycin (CTL medium), supplemented with recombinant human (rh) IL-2 to a final concentration of 50 U/mL every 48 h. After expansion, an aliquot of each transduced T cell line was stained with biotin-conjugated anti-EGFR (epithelial growth factor receptor) antibody and streptavidin-beads (Miltenyi), and tEGFR+ T cells isolated by immunomagnetic selection.

The tEGFR+ T-cell subset was then stimulated with irradiated (8,000 rad) TM EBV-LCL at a T cell: LCL ratio of 1:7, and expanded for 8 days in CTL medium with addition of 50 U/mL rh IL-2 every 48 h.

### Chromium Release, Cytokine Secretion and CFSE Proliferation Assays

Target cells were labeled with ⁵¹Cr (PerkinElmer) overnight, washed and incubated in triplicate at 1·2×10³ cells/well with effector T cells at various effector to target (E:T) ratios. Supernatants were harvested for y counting after a 4-hour incubation and specific lysis calculated using the standard formula. For analyses of cytokine secretion, target and effector cells were plated in triplicate wells at an E:T ratio of 2:1 (Raji) or 4:1 (K562/CDI9 and K562), and INF-γ, TNF-α, IL-2, IL-4, IL-6 and IL-10 measured by multiplex cytokine immunoassay (Luminex) in supernatant removed after a 24-hour incubation.

For analysis of proliferation, T cells were labeled with 0.2 μM carboxyfluorescein succinimidyl ester (CFSE, Invitrogen), washed and plated in triplicate wells with stimulator cells at a ratio of 2:1 (Raji) or 4:1 (K562/CD19 and K562) in CTL medium without exogenous cytokines. After 72 h of incubation, cells were labeled with anti-CD3 mAb and propidium iodide (PI) to exclude dead cells from analysis. Samples were analyzed by flow cytometry and cell division of live CD3+ T-cells assessed by CFSE dilution.

### Experiments in NOD/SCID and NOD/SCID/γc^{-/-} (NSG) Mice

All mouse experiments were approved by the FRCRC Institutional Animal Chimeric receptore and Use Committee. Six- to 8-week old female NOD.CB17-Prkdc^{scid}/J (NOD/SCID) and NOD.Cg-Prkdc^{scid} Il2rg^{tmlWj1}/SzJ (NSG) mice were obtained from the Jackson Laboratory or bred in-house (FRCRC. Mice were injected intravenously (i. v.) with 0.5×10⁶ Raji-ffluc tumor cells via tail vein injection, and received injections of chimeric receptor-modified T cells, control T cells, or PBS via tail vein injection as indicated.

For bioluminescence imaging, mice received intraperitoneal (i.p.) injections of freshly prepared luciferin substrate (Caliper Life Sciences, MA) resuspended in PBS (15 μg/g body weight) and were then anesthetized with isoflurane in an induction chamber. After induction of deep anesthesia., mice were imaged using an Xenogen IVIS In Vivo Imaging System (Caliper Life Sciences, MA) at 10, 12 and 14 minutes post i.p. injection of luciferin at an acquisition time of 1 second to 1 minute in small binning mode to obtain unsaturated images. Luciferase activity was analyzed using Living Image Software (Caliper Life Sciences, MA) and the photon flux analyzed within regions of interest that encompassed the entire body of each individual mouse.

### Statistical Analyses

Statistical analyses were performed using Prism Software (GraphPad, CA). Student's t-test was performed as a two-sided test with a confidence interval of 95% and results considered significant with a p-value of p<0.05. Statistical analysis of survival were done by Log-rank testing and results considered significant with a p-value of p<0.05.

### Results

Preparation of Polyclonal CD8⁺ T_{CM}-Derived Cell Lines that Express CD19 Chimeric Receptors with Long and Short Extracellular Spacers

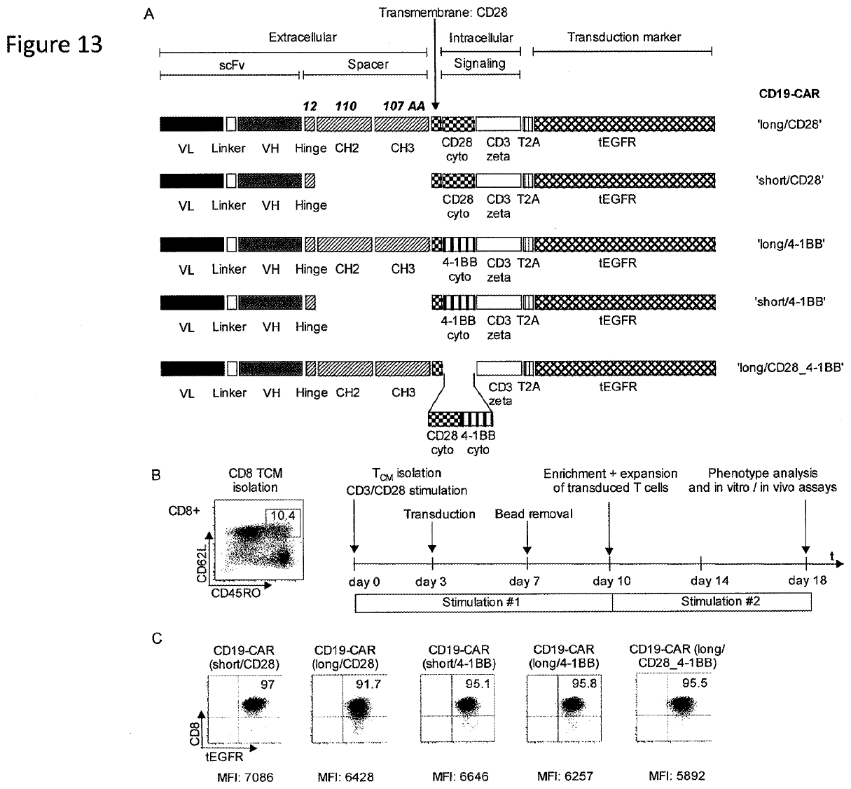

We constructed individual lentiviral vectors encoding a panel of codon-optimized CD 19 chimeric receptor genes to examine the influence of extracellular spacer length on the in vitro function and in vivo antitumor activity of CD19 chimeric receptor-modified T cells. Each chimeric receptor was comprised of a single chain variable fragment corresponding to the sequence of the CD19-specific mAb FMC63 (scFv: VL-VH), a spacer derived from IgG4-Fc including either the ‘Hinge-CH2-CH3’ domain (229 AA, long spacer) or the ‘Hinge’ domain only (12 AA, short spacer), and a signaling module of CD3ζ with membrane proximal CD28 or 4-1 BB costimulatory domains, either alone or in tandem (FIG. 13A). The transgene cassette included a truncated EGFR (tEGFR) downstream from the chimeric receptor gene and separated by a cleavable T2A element, to serve as a transduction, selection and in vivo tracking marker for chimeric receptor-modified T cells,

We isolated a CD8+ CD45RO+ CD62L+ central memory T cell (T_{CM}) cell population by cell sorting from the blood of normal donors for transduction and expansion, because of the superior ability of T_{CM} to persist in vivo after adoptive transfer. CD8⁺ T cells were stimulated with anti CD3/28 beads, transduced with each of the lentiviral vectors, and expanded in culture for 18 days before being used for in vitro and in vivo experiments. (FIG. 13B) Similar transduction efficiencies were achieved with each of the lentiviral vectors (mean 25%) and transgene-positive T cells were enriched to uniform purity by immunomagnetic selection using a biotinylated anti-EGFR mAb and streptavidin beads.

Following tEGFR-enrichment, each of the CD19 chimeric receptor T cell lines were expanded by a single stimulation with CD19+13− LCL, without apparent differences in in vitro growth kinetics between T cell lines expressing the various CD 19 chimeric receptor constructs. After expansion, the tEGFR marker was expressed at equivalent levels on >90% of the T cells transduced with each of the vectors (FIG. 13C).

CD19 Chimeric Receptors with Long and Short Extracellular Spacer Domain Confer Specific Anti-Tumor Reactivity In Vitro

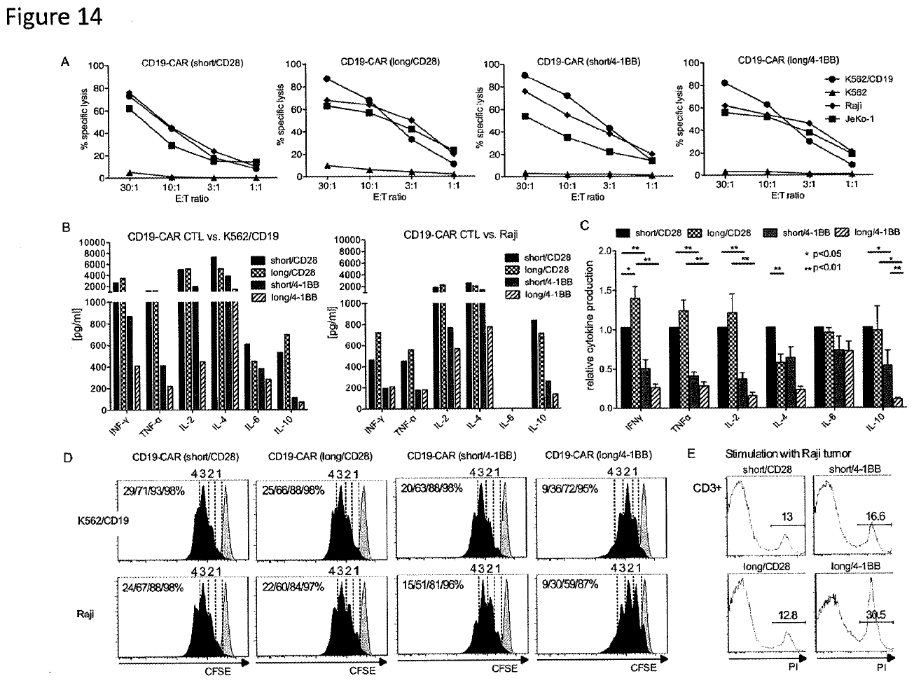

We compared the effector function of Tc-derived T cell lines modified to express CD19 chimeric receptors with CD28 and 4-11313 costimulatory signaling moieties, and either a short (‘short/CD28’; ‘short/4-1BB’) or long (‘long/CD28’; ‘long/4-1BB’) extracellular spacer domain respectively. T cells expressing each of the 4 CD19 chimeric receptor constructs conferred specific cytolytic activity against CD19⁺ Raji and JeKo-1 lymphoma cells, and against K562 cells that had been stably transfected with CD19, but not native CD19⁻ K562 cells (FIG. 14A). Quantitative analyses of cytokine production in response to stimulation with K562/CD19 or Raji tumor cells by multiplex cytokine assay (Luminex) showed production of IFN-γ, TNF-α, IL-2, IL-4, IL-6, and IL-10 by T cells expressing each of the CD19 chimeric receptors (FIG. 14B). T cells expressing CD19 chimeric receptors with a CD28 costimulatory domain produced significantly higher levels of IFN-γ, TNF-α, IL-2 and IL-10 compared to the corresponding constructs with a 4-113B costimulatory domain (FIG. 14B, C). There was significantly higher IFN-γ production and significantly less IL-4 production by T cells expressing the CD19 ‘long/CD28’ chimeric receptor compared with those expressing the ‘short/CD28’ chimeric receptor. Amongst the CD19 chimeric receptors with 4-1BB costimulatory signaling module, we detected significantly higher levels of IFN-γ, TNF-α, IL-2, IL-4, and IL-10 secretion in T cells expressing the construct with the short spacer domain (FIG. 14B, C).

We used CFSE dye dilution to analyze proliferation of T cells modified with each of the CD 19 chimeric receptors after engagement of CD 19+ tumor cells. Specific and vigorous proliferation of each of the CD19 chimeric receptor T cell lines was observed 72 hours following stimulation with either K562/CD19 or Raji. The average number of cell divisions was higher for CD19 chimeric receptor T cells with a CD28 costimulatory domain compared to those with 4-1BB, consistent with greater IL-2 production by T cells expressing a CD28 containing chimeric receptor (FIG. 14B-D). We also analyzed the proportion of chimeric receptor T cells that underwent activation induced cell death after stimulation with K562/CD19 and Raji tumor cells at the end of the 72-hours by containing the culture with CD3+ and PI. We detected a higher frequency of CD3+CD8⁺ PI⁺ T cells in the CD 19 chimeric receptor cell line ‘long/4-1 BB’, but few PI+ cells were observed with the other CD19 chimeric receptors. (FIG. 14E).

This analysis of in vitro effector functions was consistent with prior studies that have compared CD28 and 4-1BB costimulatory domains, and did not reveal differences in T cell function that would suggest that a particular CD19 chimeric receptor construct from this panel would lack anti-tumor efficacy in vivo.

T Cells Expressing CDI9 Chimeric Receptors with Short Extracellular Spacer Domains but not Long Extracellular Spacer Domains Eradicate Raji Tumors in Immunodeficient Mouse Models

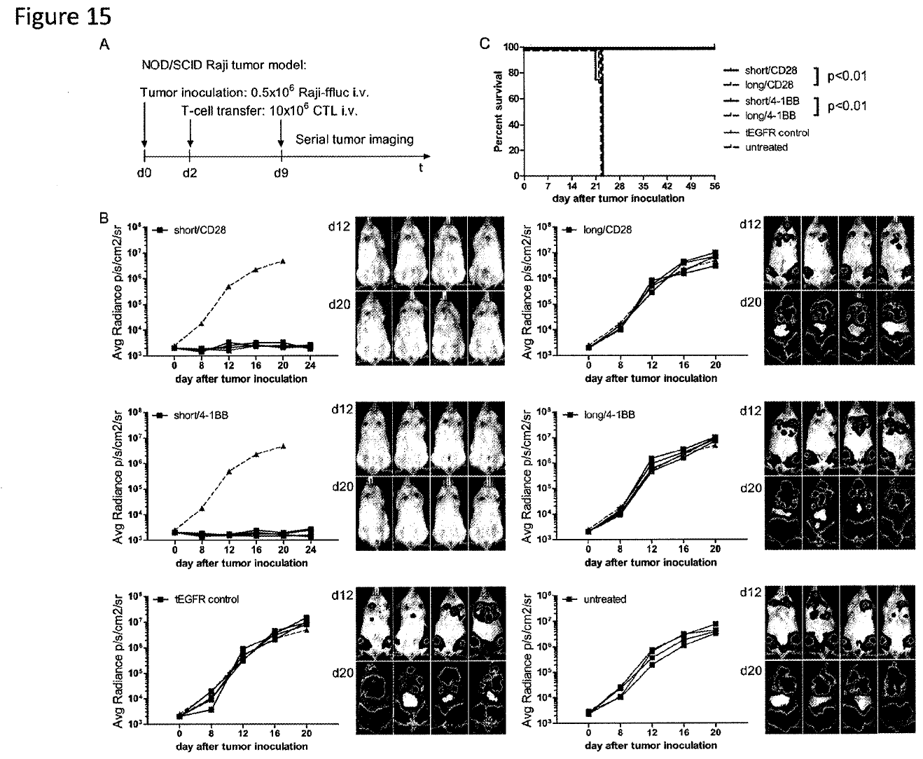

We next evaluated the in vivo antitumor efficacy of T cells modified with each of the CD19 chimeric receptors in immunodeficient (NOD/SCID) mice engrafted with firefly luciferase transfected Raji cells (Raji-ffluc), which enables sequential quantitative analyses of tumor burden and distribution using bioluminescence imaging. NOD/SCID mice inoculated with 0.5×10⁶ Raji-ffluc cells via tail vein injection developed disseminated lymphoma, which if untreated led to hind limb paralysis after approximately 3.5 weeks, necessitating euthanasia. Tumor bearing mice were treated with 2 doses of CD8+ T_{CM}-derived T cells modified with each of the CD19 chimeric receptors or with a tEGFR control vector administered on day 2 and day 9 after tumor inoculation (FIG. 15A).

Surprisingly, only T cells modified to express CD19 chimeric receptors with short extracellular spacer domain (‘short/CD28’ and ‘short/4-1BB’) eradicated Raji tumors in this model, whereas mice treated with T cells expressing CD19 chimeric receptors with long spacer (‘long/CD28’ and ‘long/4-1BB’) developed systemic lymphoma and hind limb paralysis with nearly identical kinetics as untreated mice or mice treated with control tEGFR+ T cells (FIG. 1513, C). The striking difference in antitumor activity between CD19 chimeric receptors with short and long spacer domains was highly significant and reproducible in multiple experiments with chimeric receptor T cell lines generated from 3 different normal donors.

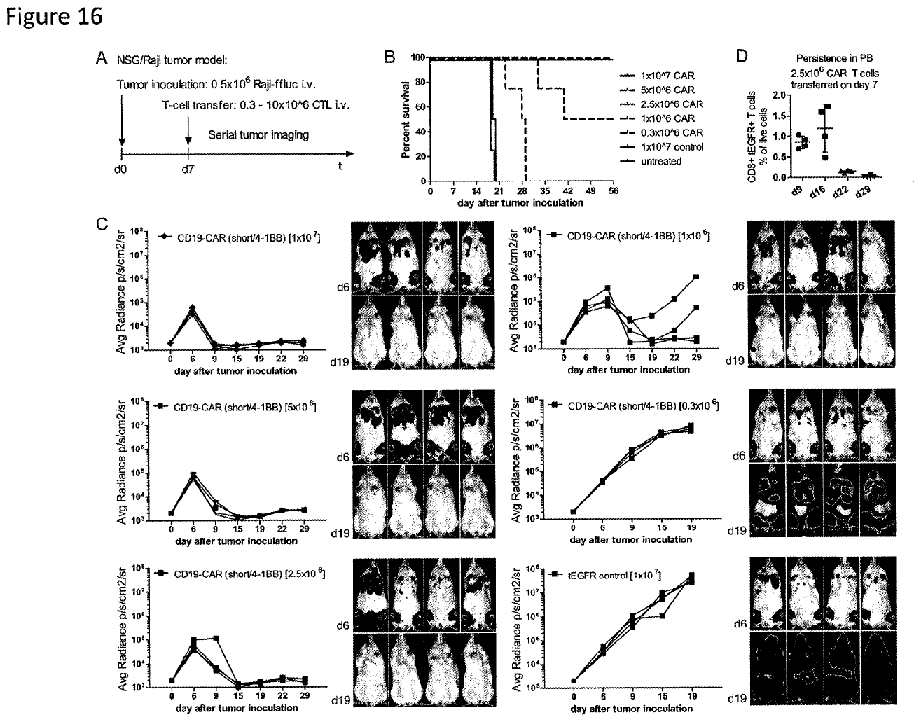

The NOD/SCID lymphoma model may be suboptimal for predicting anti-tumor activity in a clinical setting because of the short interval between tumor inoculation and T cell administration and the greater resistance to engraftment of human cells compared to more immunodeficient mouse strains such as NOD/SCID/γc^{-/-} (NSG). Thus, we evaluated antitumor activity of adoptive therapy in a more clinically relevant model in which Raji-ffluc lymphoma was established in NSG mice, and the CD19 chimeric receptor T cells were administered after 7 days when the tumor was readily detectable in the bone marrow by bioluminescence imaging (FIG. 16A). We performed initial dose titration experiments to determine the minimal dose of T cells transduced with the CD19 ‘short/4-1BB’ chimeric receptor that was required for eradication of established Raji tumors. A single dose of 2.5×10⁶T cells expressing CD19-chimeric receptor ‘short/4-1BB’ promoted complete regression of established Raji tumors and resulted in long-term tumor-free survival in 100% of mice (FIG. 16B,C). At the 2.5×10⁶ dose level, the T-cells were easily detected in the peripheral blood of NSG mice for at least 3 weeks following adoptive transfer and tumor eradication. Thus, this model enabled comparative studies both of antitumor activity and persistence of T cells modified with each of the CD19-chimeric receptors in our panel (FIG. 16D).

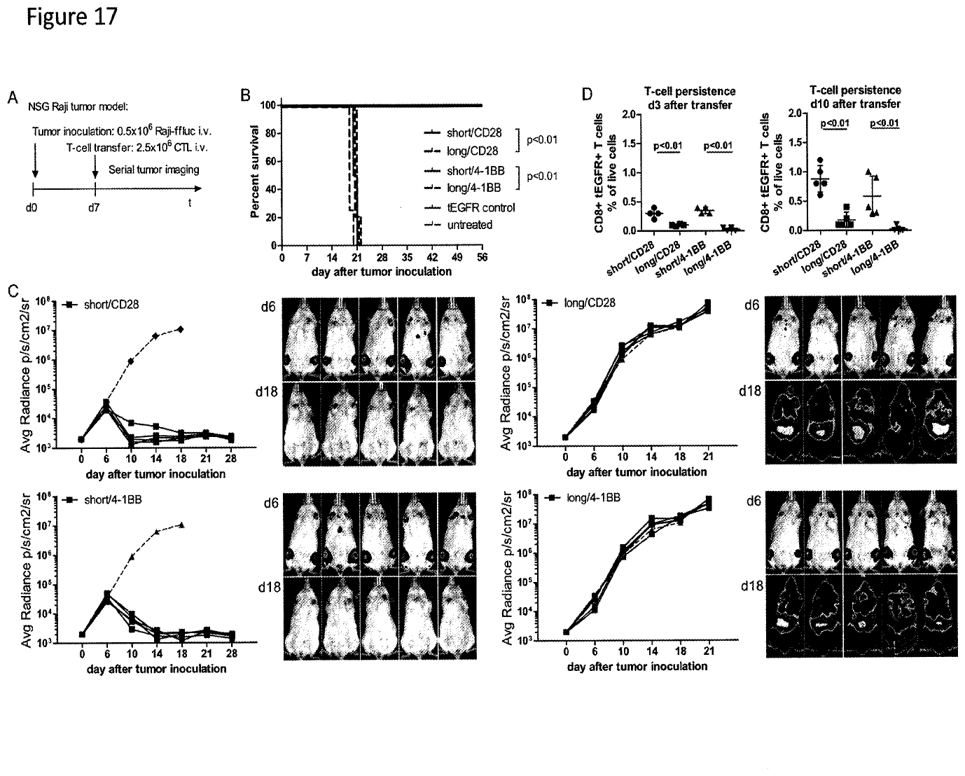

We then treated cohorts of NSG mice that were engrafted with Raji lymphoma with PBS alone, with a single dose of 2.5×10⁶ T cells expressing each of the CD19 chimeric receptors or with T cells modified with a tEGFR encoding control vector (FIG. 17A). In this model of established lymphoma, T cells expressing CD19 chimeric receptors with a short extracellular spacer domain and either 4-1BB or CD28 costimulatory domains (‘short/CD28‘and’ short/4-1BB’) mediated complete tumor regression over 7-10 days and all mice survived tumor free for >56 days. By contrast, mice treated with T cells modified to express CD19 chimeric receptors with a long spacer domain (‘long/CD28’ and ‘long/4-1BB’) exhibited tumor progression and had to be sacrificed at a similar time as mice that had received control tEGFR T cells (FIG. 17B, C). The lack of in vivo antitumor activity of the chimeric receptor constructs with long spacers was unexpected given the ability of T cells expressing these constructs to lyse tumor cells in vitro, and the enhanced IL-2 production and proliferation after engagement of T cells expressing the ‘long/CD28’CD19 chimeric receptor compared to the 4-1BB constructs.

To provide insight into the basis for the lack of efficacy, we performed sequential flow cytometry on peripheral blood samples of mice at intervals after the T cell infusion. All mice treated with T cells expressing the ‘short/CD28’ and ‘short/4-1BB’ CD19 chimeric receptors had significantly higher levels of transferred T cells in the blood at all time points after adoptive transfer, compared to mice treated with T cells that expressed corresponding CD19 chimeric receptors with long extracellular spacer (p<0.01) (FIG. 17D). We did not observe significant differences in T-cell persistence in the peripheral blood of mice that had received T cells expressing CD19 chimeric receptors with CD28 or 4-1BB co-stimulatory domains and short spacer domains (FIG. 17D).

The In Vivo Anti-Tumor Efficacy of CD19 Chimeric Receptors with Long Spacers is not Improved by Increasing T Cell Dose or Providing an Additional Costimulatory Domain

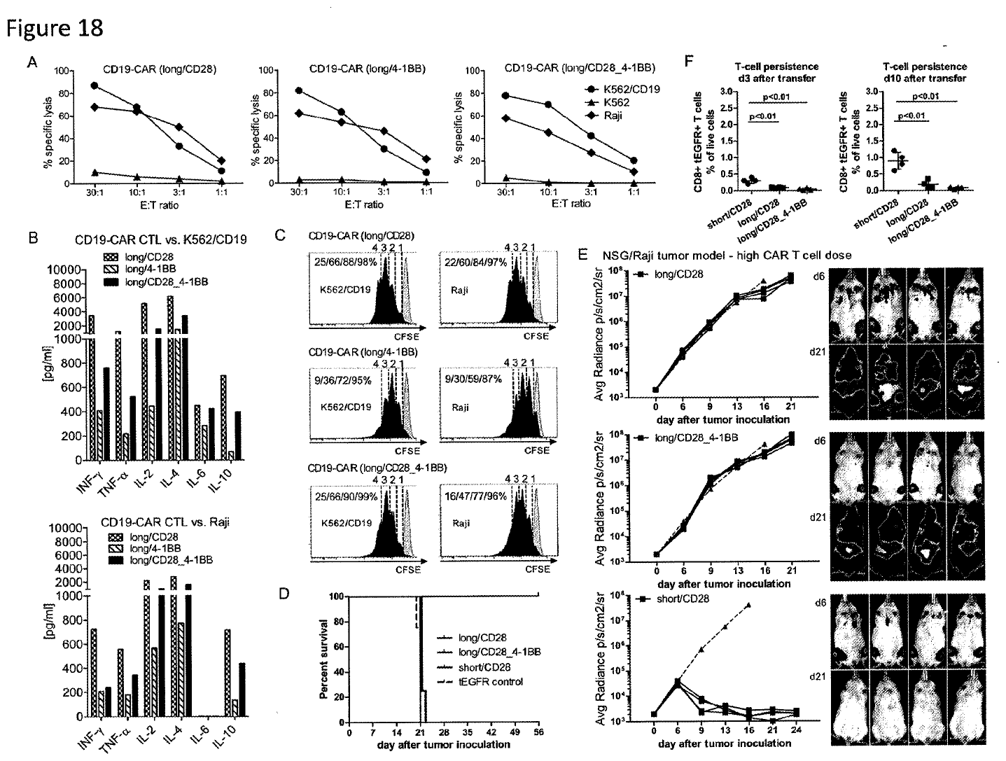

The lack of in vivo anti-tumor efficacy and the lower level of persisting chimeric receptor T cells in mice treated with T cells modified with CD19 chimeric receptors with long spacer domains suggested that efficacy might be improved by increasing the chimeric receptor T cell dose or by including both CD28 and 4-IBB domains into the chimeric receptor to augment costimulatory signaling, To evaluate this possibility we modified CD8-+T_{CM} with ‘long/CD28’, ‘short CD28’, and ‘long/CD28_4-1BB’ CD19 chimeric receptor vectors and confirmed that the long/CD28_4-1BB’ CD19 chimeric receptor conferred specific lysis and cytokine production in vitro after recognition of CD19+ target cells (FIG. 18A-C).

Consistent with previous studies of CD19 chimeric receptors, the level of cytokine production and proliferation in vitro in T cells expressing the CD28_4-IBB′ CDI9 chimeric receptor was inferior compared to the identical construct with CD28 alone, and superior to T cells expressing the ‘long 4-IBB CD19 chimeric receptor (FIG. 18B, C).

Groups of NSG mice with established Raji tumors were then treated with a high dose of T cells (10×10⁶) T cells expressing the ‘long/CD28’ CD9 chimeric receptor, the ‘long/CD28_4-IBB’ CDI9 chimeric receptor, the ‘short/CD28’ CD19-chimeric receptor, and tEGFR alone. Tumor burden was measured by bioluminescence imaging and serial flow cytometric analyses of peripheral blood samples performed to determine the frequency of transferred T cells. Consistent with the results of our prior experiments using much lower doses of T cells, Raji tumors were completely eradicated in mice treated with T cells expressing the ‘short/CD28’ CD19-chimeric receptor. However, even with a 4-fold higher T cell dose, treatment with T cells expressing the ‘long/CD28’ CD19 chimeric receptor or the ‘long/CD28_4-1BB’ CDI9 chimeric receptor did not provide a discernible antitumor effect (FIG. 18D,E).

Thus, increasing the chimeric receptor T cell dose and adding a 4-11B13 costimulatory domain to CD19 chimeric receptors failed to overcome the negative impact of the longer spacer domain on antitumor activity in vivo. Thus, in this model, anti-tumor reactivity of CD19 chimeric receptors is dictated to a great extent by the length of the extracellular spacer domain, and not by the intracellular costimulatory signaling modules.

T Cells Modified with CD19 Chimeric Receptors that Possess Long Extracellular Spacers Undergo Activation Induced Cell Death In Vivo

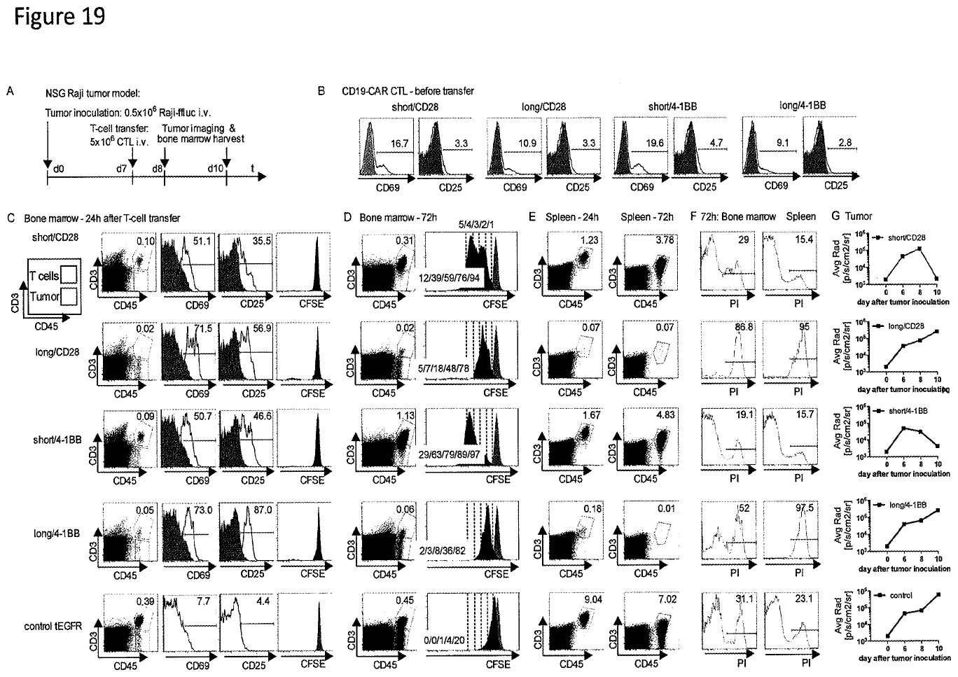

We sought to determine potential mechanisms underlying the inferior in vivo antitumor activity of T cells that express CD19 chimeric receptors with long spacer domains. Because lower numbers of transferred T cells modified to express CD19 chimeric receptors with long spacer domains were present in the blood, we considered the possibility that the T cells were not efficiently activated by tumor cells in vivo or conversely, that they underwent activation induced T cell death in vivo. Therefore, we labeled CD19 chimeric receptor modified and corresponding control T cells with CFSE and administered these T cells to tumor bearing NSG/Raji mice to examine activation, proliferation and survival of T cells modified with each of the CD19 chimeric receptor constructs at tumor sites in vivo (FIG. 19A). At the end of their in vitro expansion and immediately prior to CFSE labeling and infusion into NSG mice bearing established Raji tumors, T cells transduced with each of the CD19 chimeric receptors expressed low levels of the activation markers CD69 and CD25 (FIG. 19B).

Bone marrow was obtained from subgroups of mice 24 and 72 hours after the T cell infusion to examine the frequency, activation and proliferation of transferred T cells. At 24 hours, tumor cells (CD45+CD3-) were present in the bone marrow in all treatment groups and a large fraction of chimeric receptor T cells, but not control T cells, had upregulated CD69 and CD25. There was no measurable dilution of CFSE in the transferred chimeric receptor T cells. (FIG. 19C) Both CD69 and CD25 were expressed in a higher proportion of T cells modified with ‘long spacer’ CD19 chimeric receptors, suggesting these cells may have received a stronger stimulus compared to T cells with ‘short spacer’ CD19 chimeric receptors (FIG. _C). Despite evidence of T cell activation at 24 hours there were significantly lower numbers of chimeric receptor T cells in the bone marrow of mice treated with T cells modified with the CD28 and 4-IBB ‘long spacer’ constructs compared to those modified with the CD28 and 4-IBB ‘short spacer’ constructs, or with the control tEGFR vector (FIG. 19C, E).

At 72 hours after T cell transfer, T cells expressing the ‘short/CD28’ and ‘short/4-1BB’ CD19 chimeric receptors had increased 3 to >10 fold in frequency in the bone marrow and spleen, and had undergone several cell divisions (FIG. 19D,E). Control tEGFR+ T cells remained present in the bone marrow and spleen at 72 hours at a level similar to that observed at 24 hours, and had not divided as measured by CFSE dilution. By contrast, the numbers of T cells expressing the ‘long/CD28’ and ‘long/4-IBB’ CD19 chimeric receptors had not increased in the bone marrow and spleen. (FIG. 19D, E) Consistent with lower cell numbers, analysis of CFSE staining in viable PI− ‘long/CD28’ and ‘long/4-IBB’ CD19 chimeric receptor T cells demonstrated these cells had undergone a much lower number of cell divisions compared with ‘short/CD28’ and ‘short/4-IBB’ CD19 chimeric receptor T cells. (FIG. 19D) When the flow data was analyzed to include PI+ T cells, we detected a much higher frequency of PI+ CD3+ T cells in bone marrow and spleen of mice that received CD19 chimeric receptor T cells with ‘long spacer’ domains, demonstrating that a significant proportion of T cells, despite being activated by tumor in vivo had undergone cell death (FIG. 19F). Consistent with the bioluminescence imaging, CD45+ CD3− Raji tumor cells were present in greater numbers in the bone marrow of mice treated with T cells expressing CD19 chimeric receptors with long spacer domains or expressing tEGFR only compared to mice treated with CD19 chimeric receptors with short spacer domains (FIG. 19D,E, G).

Collectively, the data provides evidence that CD19 chimeric receptors with long extracellular spacer domain, despite mediating equivalent or superior effector function in vitro and recognizing tumor in vivo, induce a high level of activation induced cell death in vivo and fail to eradicate established lymphoma.

### Discussion

Chimeric receptors are artificial receptors that include an extracellular antigen-binding scFv, a spacer domain that provides separation of the scFv from the cell membrane and an intracellular signaling module that mediates T cell activation. Chimeric receptors that contain a scFv derived from the CD19-specific FMC63 mAb studied here, have advanced to testing in clinical trials in patients with B-cell malignancies. Antitumor activity and T cell persistence have varied substantially in different trials. Each of these clinical trials differed in potentially critical variables, including different gene transfer vectors, cell culture methodologies, and conditioning regimens prior to CD19 chimeric receptor T cell transfer.

We examined the possibility that the extracellular spacer domain of CD19 chimeric receptors may be an important determinant of anti-tumor activity in vivo, independent of the costimulatory signaling provided by the chimeric receptor. We derived spacer domains from IgG4-Fc, which enables high levels of chimeric receptor cell surface expression and is less likely to provoke recognition by innate immune cells compared to other IgG isotypes. We used the IgG4 ‘Hinge-CH2-CH3’ in the design of the long (229 AA) spacer constructs and the IgG4 ‘Hinge’ domain in our short (12 AA) spacer chimeric receptors. To compare the individual chimeric receptor constructs, we used purified (>90%) chimeric receptor positive CD8⁺ T_{CM}-derived T cells to remove differences in the cellular composition and transduction frequency as a potential source of bias in the analysis of in vitro and in vivo function. CD8⁺ T_{CM} have been shown to have superior traits for adoptive immunotherapy, compared with other more prevalent T cell subsets in blood that persist poorly and are ineffective in tumor therapy. The CD19 chimeric receptor T cells were generated using a standardized culture protocol that is similar to that used to derive chimeric receptor T cells for clinical trials. Our data show that CD19 chimeric receptors with a short IgG4 ‘Hinge’ spacer conferred potent anti-tumor reactivity in vitro and in vivo, whereas corresponding CD19 chimeric receptors with a long spacer of IgG4 ‘Hinge-CH2-CH3’, despite equivalent or superior reactivity in vitro, failed to confer significant anti-tumor effects in murine lymphoma models. Surprisingly, the length of the spacer domain proved to be a decisive element for in vivo antitumor activity, and the lack of efficacy of the ‘long spacer’ chimeric receptor could not be overcome by increasing the T cell dose.

We also observed major differences in cytokine secretion and proliferation in vitro between T cells expressing CD19 chimeric receptors containing CD28 and 4-1BB costimulatory domains, with CD28 augmenting secretion of IFN-γ, IL-2, and TNF-α compared with 4-1BB. CD19 chimeric receptors that possessed a tandem CD28_4-1BB also produced higher levels of these cytokines compared to chimeric receptors encoding 4-1BB only. However, our data shows that these differences in in vitro function were not predictive of in vivo anti-tumor efficacy, since CD19 chimeric receptors with either CD28 or 4-1BB costimulatory domain and a short spacer were similarly effective at eradicating advanced established Raji tumors in NSG mice. In contrast, CD19 chimeric receptors with suboptimal spacer length and CD28, 4-1BB, or both costimulatory domains, despite conferring similar in vitro function as the identical chimeric receptor construct with a short spacer domain, lacked significant anti-tumor activity in vivo, demonstrating the contribution of spacer length to in vivo function of chimeric receptor T cells.

Our studies provide insight into the mechanism responsible for the lack of in vivo efficacy of CD19 chimeric receptors with long spacer domains. T cells expressing CD19 chimeric receptors with both long and short spacer domains could be detected in the bone marrow and spleen after adoptive transfer into NSG mice bearing established Raji lymphoma, and the majority were activated as demonstrated by upregulation of CD25 and CD69. However, T cells modified to express a CD19 chimeric receptor with a long spacer domain exhibited a steep decline in cell number, in contrast to the marked in vivo expansion of T cells expressing CD19 chimeric receptors with a short spacer domain. The decline in T cell number was a consequence of much higher levels of cell death in the first 72 hours after adoptive transfer compared with T cells with short spacer domains, and control T cells that did not express a CD19 chimeric receptor. Collectively, these data indicate that recognition of tumor cells in vivo resulted in death of T cells expressing CD19-chimeric receptors with long spacer domains. A similar mechanism may explain the short duration and low levels of T cell persistence in the clinical trials that employed long spacer CD19-chimeric receptors (14).

The studies reported here are the first to show that the spacer domains of CD19 chimeric receptors that lack intrinsic signaling properties have dramatic effects on in vivo antitumor activity independent of costimulatory signaling, and identify the importance of analyzing the optimal composition of this region in the design of chimeric receptors for clinical applications.

The foregoing is illustrative of the present invention, and is not to be construed as limiting thereof. The invention is defined by the following claims, with equivalents of the claims to be included therein. All references and documents referred to herein are hereby incorporated by reference.

### Table 1.

TABLE 1
Sequence of anti-CD19 short spacer chimeric
receptor
GMCSFRss-CD19scFv-IgG4hinge-CD28tm-41BB-Zeta-
T2A-EGFRt

Atgctgctgctggtgaccagcctgctgctgtgcgagctgccccaccccgcc
tttctgctgatcccc (GMCSFRss) (SEQ ID NO: 2)
Gacatccagatgacccagaccacctccagcctgagcgccagcctgggcgac
cgggtgaccatcagctgccgggccagccaggacatcagcaagtacctgaac
tggtatcagcagaagcccgacggcaccgtcaagctgctgatctaccacacc
agccggctgcacagcggcgtgcccagccggtttagcggcagcggctccggc
accgactacagcctgaccatctccaacctggaacaggaagatatcgccacc
tacttttgccagcagggcaacacactgccctacaccifiggcggcggaaca
aagctggaaatcaccggcagcacctccggcagcggcaagcctggcagcggc
gagggcagcaccaagggcgaggtgaagctgcaggaaagcggccctggcctg
gtggcccccagccagagcctgagcgtgacctgcaccgtgagcggcgtgagc
ctgcccgactacggcgtgagctggatccggcagccccccaggaagggcctg
gaatggctgggcgtgatctggggcagcgagaccacctactacaacagcgcc
ctgaagagccggctgaccatcatcaaggacaacagcaagagccaggtgttc
ctgaagatgaacagcctgcagaccgacgacaccgccatctactactgcgcc
aagcactactactacggcggcagctacgccatggactactggggccagggc
accagcgtgaccgtgagcagc (CD19scFv) (SEQ ID NO: 3)
Gaatctaagtacggaccgccctgccccccttgccct (IgG4hinge)
(SEQ ID NO: 4)
Atgttctgggtgctggtggtggtcggaggcgtgctggcctgctacagcctg
ctggtcaccgtggccttcatcatcttttgggtg (CD28tm-)(SEQ ID
NO: 5)
Aaacggggcagaaagaaactcctgtatatattcaaacaaccatttatgaga
ccagtacaaactactcaagaggaagatggctgtagctgccgatttccagaa
gaagaagaaggaggatgtgaactg (41BB) (SEQ ID NO: 6)
Cgggtgaagttcagcagaagcgccgacgcccctgcctaccagcagggccag
aatcagctgtacaacgagctgaacctgggcagaagggaagagtacgacgtc
ctggataagcggagaggccgggaccctgagatgggcggcaagcctcggcgg
aagaacccccaggaaggcctgtataacgaactgcagaaagacaagatggcc
gaggcctacagcgagatcggcatgaagggcgagcggaggcggggcaagggc
cacgacggcctgtatcagggcctgtccaccgccaccaaggatacctacgac
gccctgcacatgcaggccctgcccccaagg (CD3Zeta)- (SEQ ID
NO: 7)
Ctcgagggcggcggagagggcagaggaagtcttctaacatgcggtgacgtg
gaggagaatcccggccctagg (T2A) (SEQ ID NO: 9)
Atgcttctcctggtgacaagccttctgctctgtgagttaccacacccagca
ttcctcctgatcccacgcaaagtgtgtaacggaataggtattggtgaattt
aaagactcactctccataaatgctacgaatattaaacacttcaaaaactgc
acctccatcagtggcgatctccacatcctgccggtggcatttaggggtgac
tccttcacacatactcctcctctggatccacaggaactggatattctgaaa
accgtaaaggaaatcacagggtttttgctgattcaggcttggcctgaaaac
aggacggacctccatgcctttgagaacctagaaatcatacgcggcaggacc
aagcaacatggtcagttttctcttgcagtcgtcagcctgaacataacatcc
ttgggattacgctccctcaaggagataagtgatggagatgtgataatttca
ggaaacaaaaatttgtgctatgcaaatacaataaactggaaaaaactgttt
gggacctccggtcagaaaaccaaaattataagcaacagaggtgaaaacagc
tgcaaggccacaggccaggtctgccatgccttgtgctcccccgagggctgc
tggggcccggagcccagggactgcgtctcttgccggaatgtcagccgaggc
agggaatgcgtggacaagtgcaaccttctggagggtgagccaagggagttt
gtggagaactctgagtgcatacagtgccacccagagtgcctgcctcaggcc
atgaacatcacctgcacaggacggggaccagacaactgtatccagtgtgcc
cactacattgacggcccccactgcgtcaagacctgcccggcaggagtcatg
ggagaaaacaacaccctggtctggaagtacgcagacgccggccatgtgtgc
cacctgtgccatccaaactgcacctacggatgcactgggccaggtcttgaa
ggctgtccaacgaatgggcctaagatcccgtccatcgccactgggatggtg
ggggccctcctcttgctgctggtggtggccctggggatcggcctcttcatg
tga (EGFRt) (SEQ ID NO: 9)

TABLE 2

| | |
|---|---|
| | GMCSFRss |
| DNA: | ATGCTGCTGCTGGTGACCAGCCTGCTGCTGTGCGAGCTGCCCCACCCCGCC |
| AA: | M L L L V T S L L L C E L P H P A |
| | CD19scFv |
| DNA: | TTTCTGCTGATCCCC:GACATCCAGATGACCCAGACCACCTCCAGCCTGAGC |
| AA: | F L L I P D I Q M T Q T T S S L S |
| DNA: | GCCAGCCTGGGCGACCGGGTGACCATCAGCTGCCGGGCCAGCCAGGACATC |
| AA: | A S L G D R V T I S C R A S Q D I |
| DNA: | AGCAAGTACCTGAACTGGTATCAGCAGAAGCCCGACGGCACCGTCAAGCTG |
| AA: | S K Y L N W Y Q Q K P D G T V K L |
| DNA: | CTGATCTACCACACCAGCCGGCTGCACAGCGGCGTGCCCAGCCGGTTTAGC |
| AA: | L I Y H T S R L H S G V P S R F S |
| DNA: | GGCAGCGGCTCCGGCACCGACTACAGCCTGACCATCTCCAACCTGGAACAG |
| AA: | G S G S G T D Y S L T I S N L E Q |
| DNA: | GAAGATATCGCCACCTACTTTTGCCAGCAGGGCAACACACTGCCCTACACC |
| AA: | E D I A T Y F C Q Q G N T L P Y T |
| DNA: | TTTGGCGGCGGAACAAAGCTGGAAATCACCGGCAGCACCTCCGGCAGCGGC |
| AA: | F G G G T K L E I T G S T S G S G |
| DNA: | AAGCCTGGCAGCGGCGAGGGCAGCACCAAGGGCGAGGTGAAGCTGCAGGAA |
| AA: | K P G S G E G S T K G E V K L Q E |
| DNA: | AGCGGCCCTGGCCTGGTGGCCCCCAGCCAGAGCCTGAGCGTGACCTGCACC |
| AA: | S G P G L V A P S Q S L S V T C T |
| DNA: | GTGAGCGGCGTGAGCCTGCCCGACTACGGCGTGAGCTGGATCCGGCAGCCC |
| AA: | V S G V S L P D Y G V S W I R Q P |
| DNA: | CCCAGGAAGGGCCTGGAATGGCTGGGCGTGATCTGGGGCAGCGAGACCACC |
| AA: | P R K G L E W L G V I W G S E T T |
| DNA: | TACTACAACAGCGCCCTGAAGAGCCGGCTGACCATCATCAAGGACAACAGC |
| AA: | Y Y N S A L K S R L T I I K D N S |
| DNA: | AAGAGCCAGGTGTTCCTGAAGATGAACAGCCTGCAGACCGACGACACCGCC |
| AA: | K S Q V F L K M N S L Q T D D T A |
| DNA: | ATCTACTACTGCGCCAAGCACTACTACTACGGCGGCAGCTACGCCATGGAC |
| AA: | I Y Y C A K H Y Y Y G G S Y A M D |
| | IgG4hinge |
| DNA: | TACTGGGGCCAGGGCACCAGCGTGACCGTGAGCAGC:GAGAGCAAGTACGGA |
| AA: | Y W G Q G T S V T V S S E S K Y G |
| | CD28tm |
| DNA: | CCGCCCTGCCCCCCTTGCCCT:ATGTTCTGGGTGCTGGTGGTGGTCGGAGGC |
| AA: | P P C P P C P M F W V L V V V G G |
| DNA: | GTGCTGGCCTGCTACAGCCTGCTGGTCACCGTGGCCTTCATCATCTTTTGG |
| AA: | V L A C Y S L L V T V A F I I F W |
| | 41BB |
| DNA: | GTG:AAACGGGGCAGAAAGAAACTCCTGTATATATTCAAACAACCATTTATG |
| AA: | V K R G R K K L L Y I F K Q P F M |
| DNA: | AGACCAGTACAAACTACTCAAGAGGAAGATGGCTGTAGCTGCCGATTTCCA |
| AA: | R P V Q T T Q E E D G C S C R F P |
| | CD3Zeta |
| DNA: | GAAGAAGAAGAAGGAGGATGTGAACTGCGGGTGAAG:TTCAGCAGAAGCGCC |
| AA: | E E E E G G C E L R V K F S R S A |
| DNA: | GACGCCCCTGCCTACCAGCAGGGCCAGAATCAGCTGTACAACGAGCTGAAC |
| AA: | D A P A Y Q Q G Q N Q L Y N E L N |
| DNA: | CTGGGCAGAAGGGAAGAGTACGACGTCCTGGATAAGCGGAGAGGCCGGGAC |
| AA: | L G R R E E Y D V L D K R R G R D |
| DNA: | CCTGAGATGGGCGGCAAGCCTCGGCGGAAGAACCCCCAGGAAGGCCTGTAT |
| AA: | P E M G G K P R R K N P Q E G L Y |
| DNA: | AACGAACTGCAGAAAGACAAGATGGCCGAGGCCTACAGCGAGATCGGCATG |
| AA: | N E L Q K D K M A E A Y S E I G M |
| DNA: | AAGGGCGAGCGGAGGCGGGGCAAGGGCCACGACGGCCTGTATCAGGGCCTG |
| AA: | K G E R R R G K G H D G L Y Q G L |
| DNA: | TCCACCGCCACCAAGGATACCTACGACGCCCTGCACATGCAGGCCCTGCCC |
| AA: | S T A T K D T Y D A L H M Q A L P |
| | T2A |
| DNA: | CCAAGG:CTCGAGGGCGGCGGAGAGGGCAGAGGAAGTCTTCTAACATGCGGT |
| AA: | P R L E G G G E G R G S L L T C G |
| | EGFRt |
| DNA: | GACGTGGAGGAGAATCCCGGCCCTAGG:ATGCTTCTCCTGGTGACAAGCCTT |
| AA: | D V E E N P G P R M L L L V T S L |
| DNA: | CTGCTCTGTGAGTTACCACACCCAGCATTCCTCCTGATCCCACGCAAAGTG |
| AA: | L L C E L P H P A F L L I P R K V |
| DNA: | TGTAACGGAATAGGTATTGGTGAATTTAAAGACTCACTCTCCATAAATGCT |
| AA: | C N G I G I G E F K D S L S I N A |
| DNA: | ACGAATATTAAACACTTCAAAAACTGCACCTCCATCAGTGGCGATCTCCAC |
| AA: | T N I K H F K N C T S I S G D L H |
| DNA: | ATCCTGCCGGTGGCATTTAGGGGTGACTCCTTCACACATACTCCTCCTCTG |
| AA: | I L P V A F R G D S F T H T P P L |
| DNA: | GATCCACAGGAACTGGATATTCTGAAAACCGTAAAGGAAATCACAGGGTTT |
| AA: | D P Q E L D I L K T V K E I T G F |
| DNA: | TTGCTGATTCAGGCTTGGCCTGAAAACAGGACGGACCTCCATGCCTTTGAG |
| AA: | L L I Q A W P E N R T D L H A F E |
| DNA: | AACCTAGAAATCATACGCGGCAGGACCAAGCAACATGGTCAGTTTTCTCTT |
| AA: | N L E I I R G R T K Q H G Q F S L |
| DNA: | GCAGTCGTCAGCCTGAACATAACATCCTTGGGATTACGCTCCCTCAAGGAG |
| AA: | A V V S L N I T S L G L R S L K E |
| DNA: | ATAAGTGATGGAGATGTGATAATTTCAGGAAACAAAAATTTGTGCTATGCA |
| AA: | I S D G D V I I S G N K N L C Y A |
| DNA: | AATACAATAAACTGGAAAAAACTGTTTGGGACCTCCGGTCAGAAAACCAAA |
| AA: | N T I N W K K L F G T S G Q K T K |
| DNA: | ATTATAAGCAACAGAGGTGAAAACAGCTGCAAGGCCACAGGCCAGGTCTGC |
| AA: | I I S N R G E N S C K A T G Q V C |
| DNA: | CATGCCTTGTGCTCCCCCGAGGGCTGCTGGGGCCCGGAGCCCAGGGACTGC |
| AA: | H A L C S P E G C W G P E P R D C |
| DNA: | GTCTCTTGCCGGAATGTCAGCCGAGGCAGGGAATGCGTGGACAAGTGCAAC |
| AA: | V S C R N V S R G R E C V D K C N |
| DNA: | CTTCTGGAGGGTGAGCCAAGGGAGTTTGTGGAGAACTCTGAGTGCATACAG |
| AA: | L L E G E P R E F V E N S E C I Q |
| DNA: | TGCCACCCAGAGTGCCTGCCTCAGGCCATGAACATCACCTGCACAGGACGG |
| AA: | C H P E C L P Q A M N I T C T G R |
| DNA: | GGACCAGACAACTGTATCCAGTGTGCCCACTACATTGACGGCCCCCACTGC |
| AA: | G P D N C I Q C A H Y I D G P H C |
| DNA: | GTCAAGACCTGCCCGGCAGGAGTCATGGGAGAAAACAACACCCTGGTCTGG |
| AA: | V K T C P A G V M G E N N T L V W |
| DNA: | AAGTACGCAGACGCCGGCCATGTGTGCCACCTGTGCCATCCAAACTGCACC |
| AA: | K Y A D A G H V C H L C H P N C T |
| DNA: | TACGGATGCACTGGGCCAGGTCTTGAAGGCTGTCCAACGAATGGGCCTAAG |
| AA: | Y G C T G P G L E G C P T N G P K |
| DNA: | ATCCCGTCCATCGCCACTGGGATGGTGGGGGCCCTCCTCTTGCTGCTGGTG |
| AA: | I P S I A T G M V G A L L L L L V |
| DNA: | GTGGCCCTGGGGATCGGCCTCTTCATGTGA (SEQ ID NO: 10) |
| AA: | V A L G I G L F M * (SEQ ID NO: 11) |

TABLE 3
ZXR-014 Nucleotide and amino acid sequences (map of sections)
GMCSFRss: nt2084-2149
CD19scFv: nt2150-2884
Igg4Hinge: nt2885-2920
CD28tm: nt2921-3004
41BB: nt3005-3130
Zeta: nt3131-3466
T2A: nt3467-3538
EGFRt: nt3539-4612
Primers for sequencing:

| Oligo name | Sequence | Region |
|---|---|---|
| oJ02649 | ATCAAAAGAATAGACCGAGATAGGGT | pre-U5(SEQ ID NO: 22) |
| oJ02648 | CCGTACCTTTAAGACCAATGACTTAC | delU3(SEQ ID NO: 23) |
| oJ02650 | TTGAGAGTTTTCGCCCCG | mid-Ampr(SEQ ID NO: 24) |
| oJ02651 | AATAGACAGATCGCTGAGATAGGT | post-Ampr(SEQ ID NO: 25) |
| oJ02652 | CAGGTATCCGGTAAGCGG | CoE1 ori(SEQ ID NO: 26) |
| oJ02653 | CGACCAGCAACCATAGTCC | SV40(SEQ ID NO: 27) |
| oJ02654 | TAGCGGTTTGACTCACGG | CMV(SEQ ID NO: 28) |
| oJ02655 | GCAGGGAGCTAGAACGATTC | psi(SEQ ID NO: 29) |
| oJ02656 | ATTGTCTGGTATAGTGCAGCAG | RRE(SEQ ID NO: 30) |
| oJ02657 | TCGCAACGGGTTTGCC | EF1p(SEQ ID NO: 31) |
| oJ02658 | AGGAAGATATCGCCACCTACT | CD19Rop(SEQ ID NO: 32) |
| oJ02601 | CGGGTGAAGTTCAGCAGAAG | Zeta(SEQ ID NO: 33) |
| oJ02735 | ACTGTGTTTGCTGACGCAAC | WPRE(SEQ ID NO: 34) |
| oJ02715 | ATGCTTCTCCTGGTGACAAG | EGFRt(SEQ ID NO: 35) |

TABLE 4
Uniprot P0861 IgG4-Fc (SEQ ID NO: 13)

```
        10 20 30 40 50 60
ASTKGPSVFP LAPCSRSTSE STAALGCLVK DYFPEPVTVS WNSGALTSGV HTFPAVLQSS
                70 80 90 100 110 120
GLYSLSSVVT VPSSSLGTKT YTCNVDHKPS NTKVDKRVES KYGPPCPSCP APEFLGGPSV
              130 140 150 160 170 180
FLFPPKPKDT LMISRTPEVT CVVVDVSQED PEVQFNWYVD GVEVHNAKTK PREEQFNSTY
              190 200 210 220 230 240
RVVSVLTVLH QDWLNGKEYK CKVSNKGLPS SIEKTISKAK GQPREPQVYT LPPSQEEMTK
              250 260 270 280 290 300
NQVSLTCLVK GFYPSDIAVE WESNGQPENN YKTTPPVLDS DGSFFLYSRL TVDKSRWQEG
              310 320
NVFSCSVMHE ALHNHYTQKS LSLSLGK
1-98 CH1
99-110 Hinge
111-220 CH2
221-327 CH3
Position 108 S→P
```

TABLE 5
Uniprot P10747 CD28(SEQ ID NO: 14)

```
10 20 30 40 50 60
MLRLLLALNL FPSIQVTGNK ILVKQSPMLV AYDNAVNLSC KYSYNLFSRE FRASLHKGLD
                70 80 90 100 110 120
SAVEVCVVYG NYSQQLQVYS KTGFNCDGKL GNESVTFYLQ NLYVNQTDIY FCKIEVMYPP
              130 140 150 160 170 180
PYLDNEKSNG TIIHVKGKHL CPSPLFPGPS KPFWVLVVVG GVLACYSLLV TVAFIIFWVR
              190 200 210 220
SKRSRLLHSD YMNMTPRRPG PTRKHYQPYA PPRDFAAYRS
1-18 signal peptide
19-152 extracellular domain
153-179 transmembrane domain
180-220 intracellular domain
Position 186-187 LL→GG
```

TABLE 6
Uniprot Q07011 4-1BB(SEQ ID NO: 15)

```
        10 20 30 40 50 60
MGNSCYNIVA TLLLVLNFER TRSLQDPCSN CPAGTFCDNN RNQICSPCPP NSFSSAGGQR
                70 80 90 100 110 120
TCDICRQCKG VFRTRKECSS TSNAECDCTP GFHCLGAGCS MCEQDCKQGQ ELTKKGCKDC
              130 140 150 160 170 180
CFGTFNDQKR GICRPWTNCS LDGKSVLVNG TKERDVVCGP SPADLSPGAS SVTPPAPARE
              190 200 210 220 230 240
PGHSPQIISF FLALTSTALL FLLFFLTLRF SVVKRGRKKL LYIFKQPFMR PVQTTQEEDG
              250
CSCRFPEEEE GGCEL
1-23 signal peptide
24-186 extracellular domain
187-213 transmembrane domain
214-255 intracellular domain
```

TABLE 7
Uniprot P20963 human CD3ζ isoform 3 (SEQ ID NO: 16)

```
10 20 30 40 50 60
MKWKALFTAA ILQAQLPITE AQSFGLLDPK LCYLLDGILF IYGVILTALF LRVKFSRSAD
                70 80 90 100 110 120
APAYQQGQNQ LYNELNLGRR EEYDVLDKRR GRDPEMGGKP QRRKNPQEGL YNELQKDKMA
              130 140 150 160
EAYSEIGMKG ERRRGKGHDG LYQGLSTATK DTYDALHMQA LPPR
1-21 signal peptide
22-30 extracellular
31-51 transmembrane
52-164 intracellular domain
61-89 ITAM1
100-128 ITAM2
131-159 ITAM3
```

TABLE 8
Exemplary Hinge region Sequences

| | |
|---|---|
| Human IgG1 | EPKSCDKTHTCPPCP |
| | (SEQ ID NO: 17) |
| Human IgG2 | ERKCCVECPPCP |
| | (SEQ ID NO: 18) |
| Human IgG3 | ELKTPLGDTHTCPRCP |
| | (EPKSCDTPPPCPRCP)₃ |
| | (SEQ ID NO: 19) |
| Human IgG4 | ESKYGPPCPSCP |
| | (SEQ ID NO:20) |
| Modified Human IgG4 | ESKYGPPCPPCP |
| | (SEQ ID NO: 21) |
| Modified Human IgG4 | YGPPCPPCP |
| | (SEQ ID NO: 51) |
| Modified Human IgG4 | KYGPPCPPCP |
| | (SEQ ID NO: 52) |
| Modified Human IgG4 | EVVKYGPPCPPCP |
| | (SEQ ID NO: 53) |

TABLE 9
R12 long spacer CAR: PJ_R12-CH2—CH3-41BB-Z-T2A-tEGFR (SEQ ID NO: 37)
GTTAGACCAGATCTGAGCCTGGGAGCTCTCTGGCTAACTAGGGAACCCACTGCTTAAGC
CTCAATAAAGCTTGCCTTGAGTGCTTCAAGTAGTGTGTGCCCGTCTGTTGTGTGACTCTG
GTAACTAGAGATCCCTCAGACCCTTTTAGTCAGTGTGGAAAATCTCTAGCAGTGGCGCCC
GAACAGGGACTTGAAAGCGAAAGGGAAACCAGAGGAGCTCTCTCGACGCAGGACTCGG
CTTGCTGAAGCGCGCACGGCAAGAGGCGAGGGGCGGCGACTGGTGAGTACGCCAAAAA
TTTTGACTAGCGGAGGCTAGAAGGAGAGAGATGGGTGCGAGAGCGTCAGTATTAAGCGG
GGGAGAATTAGATCGATGGGAAAAAATTCGGTTAAGGCCAGGGGGAAAGAAAAAATAT
AAATTAAAACATATAGTATGGGCAAGCAGGGAGCTAGAACGATTCGCAGTTAATCCTGG
CCTGTTAGAAACATCAGAAGGCTGTAGACAAATACTGGGACAGCTACAACCATCCCTTC
AGACAGGATCAGAAGAACTTAGATCATTATATAATACAGTAGCAACCCTCTATTGTGTG
CATCAAAGGATAGAGATAAAAGACACCAAGGAAGCTTTAGACAAGATAGAGGAAGAGC
AAAACAAAAGTAAGAAAAAAGCACAGCAAGCAGCAGCTGACACAGGACACAGCAATCA
GGTCAGCCAAAATTACCCTATAGTGCAGAACATCCAGGGGCAAATGGTACATCAGGCCA
TATCACCTAGAACTTTAAATGCATGGGTAAAAGTAGTAGAAGAGAAGGCTTTCAGCCCA
GAAGTGATACCCATGTTTTCAGCATTATCAGAAGGAGCCACCCCACAAGATTTAAACAC
CATGCTAAACACAGTGGGGGGACATCAAGCAGCCATGCAAATGTTAAAAGAGACCATCA
ATGAGGAAGCTGCAGGCAAAGAGAAGAGTGGTGCAGAGAGAAAAAAGAGCAGTGGGA
ATAGGAGCTTTGTTCCTTGGGTTCTTGGGAGCAGCAGGAAGCACTATGGGCGCAGCGTC
AATGACGCTGACGGTACAGGCCAGACAATTATTGTCTGGTATAGTGCAGCAGCAGAACA
ATTTGCTGAGGGCTATTGAGGCGCAACAGCATCTGTTGCAACTCACAGTCTGGGGCATCA
AGCAGCTCCAGGCAAGAATCCTGGCTGTGGAAAGATACCTAAAGGATCAACAGCTCCTG
GGGATTTGGGGTTGCTCTGGAAAACTCATTTGCACCACTGCTGTGCCTTGGATCTACAAA
TGGCAGTATTCATCCACAATTTTAAAAGAAAAGGGGGGATTGGGGGGTACAGTGCAGGG
GAAAGAATAGTAGACATAATAGCAACAGACATACAAACTAAAGAATTACAAAAACAAA
TTACAAAAATTCAAAATTTTCGGGTTTATTACAGGGACAGCAGAGATCCAGTTTGGGGAT
CAATTGCATGAAGAATCTGCTTAGGGTTAGGCGTTTTGCGCTGCTTCGCGAGGATCTGCG
ATCGCTCCGGTGCCCGTCAGTGGGCAGAGCGCACATCGCCCACAGTCCCCGAGAAGTTG
GGGGGAGGGGTCGGCAATTGAACCGGTGCCTAGAGAAGGTGGCGCGGGGTAAACTGGG
AAAGTGATGTCGTGTACTGGCTCCGCCTTTTTCCCGAGGGTGGGGGAGAACCGTATATAA
GTGCAGTAGTCGCCGTGAACGTTCTTTTTCGCAACGGGTTTGCCGCCAGAACACAGCTGA
AGCTTCGAGGGGCTCGCATCTCTCCTTCACGCGCCCGCCGCCCTACCTGAGGCCGCCATC
CACGCCGGTTGAGTCGCGTTCTGCCGCCTCCCGCCTGTGGTGCCTCCTGAACTGCGTCCG
CCGTCTAGGTAAGTTTAAAGCTCAGGTCGAGACCGGGCCTTTGTCCGGCGCTCCCTTGGA
GCCTACCTAGACTCAGCCGGCTCTCCACGCTTTGCCTGACCCTGCTTGCTCAACTCTACGT
CTTTGTTTCGTTTTCTGTTCTGCGCCGTTACAGATCCAAGCTGTGACCGGCGCCTACG

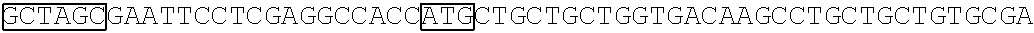

GCTGCCCCACCCCGCCTTTCTGCTGATCCCCCAGGAACAGCTCGTCGAAAGCGGCGGCA
GACTGGTGACACCTGGCGGCAGCCTGACCCTGAGCTGCAAGGCCAGCGGCTTCGACTTC
AGCGCCTACTACATGAGCTGGGTCCGCCAGGCCCCTGGCAAGGGACTGGAATGGATCGC
CACCATCTACCCCAGCAGCGGCAAGACCTACTACGCCACCTGGGTGAACGGACGGTTCA
CCATCTCCAGCGACAACGCCCAGAACACCGTGGACCTGCAGATGAACAGCCTGACAGCC
GCCGACCGGGCCACCTACTTTTGCGCCAGAGACAGCTACGCCGACGACGGCGCCCTGTT
CAACATCTGGGGCCCTGGCACCCTGGTGACAATCTCTAGCGGCGGAGGCGGATCTGGTG
GCGGAGGAAGTGGCGGCGGAGGATCTGAGCTGGTGCTGACCCAGAGCCCCTCTGTGTCT
GCTGCCCTGGGAAGCCCTGCCAAGATCACCTGTACCCTGAGCAGCGCCCACAAGACCGA
CACCATCGACTGGTATCAGCAGCTGCAGGGCGAGGCCCCCAGATACCTGATGCAGGTGC
AGAGCGACGGCAGCTACACCAAGAGGCCAGGCGTGCCCGACCGGTTCAGCGGATCTAGC
TCTGGCGCCGACCGCTACCTGATCATCCCCAGCGTGCAGGCCGATGACGAGGCCGATTA
CTACTGTGGCGCCGACTACATCGGCGGCTACGTGTTCGGCGGAGGCACCCAGCTGACCG
TGACCGGCGAGTCTAAG
IgG4 spacer

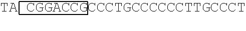

CH2
GCCCCCGAGTTCCTGGGCGGACCCAGCGTGTTCCTGTTCCCCCCCAAGCCCAAGGACACC
CTGATGATCAGCCGGACCCCCGAGGTGACCTGCGTGGTGGTGGACGTGAGCCAGGAAGA
TCCCGAGGTCCAGTTCAATTGGTACGTGGACGGCGTGGAAGTGCACAACGCCAAGACCA
AGCCCAGAGAGGAACAGTTCAACAGCACCTACCGGGTGGTGTCTGTGCTGACCGTGCTG
CACCAGGACTGGCTGAACGGCAAAGAATACAAGTGCAAGGTGTCCAACAAGGGCCTGC
CCAGCAGCATCGAAAAGACCATCAGCAAGGCCAAG
CH3
GGCCAGCCTCGCGAGCCCCAGGTGTACACCCTGCCTCCCTCCCAGGAAGAGATGACCAA
GAACCAGGTGTCCCTGACCTGCCTGGTGAAGGGCTTCTACCCCAGCGACATCGCCGTGG
AGTGGGAGAGCAACGGCCAGCCTGAGAACAACTACAAGACCACCCCTCCCGTGCTGGAC
AGCGACGGCAGCTTCTTCCTGTACAGCCGGCTGACCGTGGACAAGAGCCGGTGGCAGGA
AGGCAACGTCTTTAGCTGCAGCGTGATGCACGAGGCCCTGCACAACCACTACACCCAGA
AGAGCCTGAGCCTGTCCCTGGGCAAG
4-1BB
ATGTTCTGGGTGCTGGTGGTGGTGGGCGGGGTGCTGGCCTGCTACAGCCTGCTGGTGACA
GTGGCCTTCATCATCTTTTGGGTGAAACGGGGCAGAAAGAAACTCCTGTATATATTCAAA
CAACCATTTATGAGACCAGTACAAACTACTCAAGAGGAAGATGGCTGTAGCTGCCGATT
TCCAGAAGAAGAAGAAGGAGGATGTGAACTG
CD3 zeta
CGGGTGAAGTTCAGCAGAAGCGCCGACGCCCCTGCCTACCAGCAGGGCCAGAATCAGCT
GTACAACGAGCTGAACCTGGGCAGAAGGGAAGAGTACGACGTCCTGGATAAGCGGAGA
GGCCGGGACCCTGAGATGGGCGGCAAGCCTCGGCGGAAGAACCCCCAGGAAGGCCTGT
ATAACGAACTGCAGAAAGACAAGATGGCCGAGGCCTACAGCGAGATCGGCATGAAGGG
CGAGCGGAGGCGGGGCAAGGGCCACGACGGCCTGTATCAGGGCCTGTCCACCGCCACCA
AGGATACCTACGACGCCCTGCACATGCAGGCCCTGCCCCCAAGG
T2A

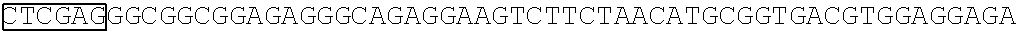

ATCCCGGCCCTAGG
tEGFR
ATGCTTCTCCTGGTGACAAGCCTTCTGCTCTGTGAGTTACCACACCCAGCATTCCTCCTGA
TCCCACGCAAAGTGTGTAACGGAATAGGTATTGGTGAATTTAAAGACTCACTCTCCATAA
ATGCTACGAATATTAAACACTTCAAAAACTGCACCTCCATCAGTGGCGATCTCCACATCC

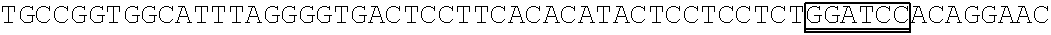

TGGATATTCTGAAAACCGTAAAGGAAATCACAGGGTTTTTGCTGATTCAGGCTTGGCCTG
AAAACAGGACGGACCTCCATGCCTTTGAGAACCTAGAAATCATACGCGGCAGGACCAAG
CAACATGGTCAGTTTTCTCTTGCAGTCGTCAGCCTGAACATAACATCCTTGGGATTACGC
TCCCTCAAGGAGATAAGTGATGGAGATGTGATAATTTCAGGAAACAAAAATTTGTGCTA
TGCAAATACAATAAACTGGAAAAAACTGTTTGGGACCTCCGGTCAGAAAACCAAAATTA
TAAGCAACAGAGGTGAAAACAGCTGCAAGGCCACAGGCCAGGTCTGCCATGCCTTGTGC
TCCCCCGAGGGCTGCTGGGGCCCGGAGCCCAGGGACTGCGTCTCTTGCCGGAATGTCAG
CCGAGGCAGGGAATGCGTGGACAAGTGCAACCTTCTGGAGGGTGAGCCAAGGGAGTTTG
TGGAGAACTCTGAGTGCATACAGTGCCACCCAGAGTGCCTGCCTCAGGCCATGAACATC
ACCTGCACAGGACGGGGACCAGACAACTGTATCCAGTGTGCCCACTACATTGACGGCCC
CCACTGCGTCAAGACCTGCCCGGCAGGAGTCATGGGAGAAAACAACACCCTGGTCTGGA
AGTACGCAGACGCCGGCCATGTGTGCCACCTGTGCCATCCAAACTGCACCTACGGATGC
ACTGGGCCAGGTCTTGAAGGCTGTCCAACGAATGGGCCTAAGATCCCGTCCATCGCCAC
TGGGATGGTGGGGGCCCTCCTCTTGCTGCTGGTGGTGGCCCTGGGGATCGGCCTCTTCAT

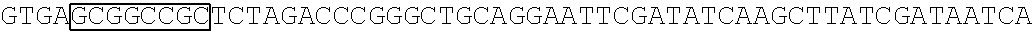

ACCTCTGGATTACAAAATTTGTGAAAGATTGACTGGTATTCTTAACTATGTTGCTCCTTTT
ACGCTATGTGGATACGCTGCTTTAATGCCTTTGTATCATGCTATTGCTTCCCGTATGGCTT
TCATTTTCTCCTCCTTGTATAAATCCTGGTTGCTGTCTCTTTATGAGGAGTTGTGGCCCGT
TGTCAGGCAACGTGGCGTGGTGTGCACTGTGTTTGCTGACGCAACCCCCACTGGTTGGGG
CATTGCCACCACCTGTCAGCTCCTTTCCGGGACTTTCGCTTTCCCCCTCCCTATTGCCACG
GCGGAACTCATCGCCGCCTGCCTTGCCCGCTGCTGGACAGGGGCTCGGCTGTTGGGCACT
GACAATTCCGTGGTGTTGTCGGGGAAATCATCGTCCTTTCCTTGGCTGCTCGCCTGTGTTG
CCACCTGGATTCTGCGCGGGACGTCCTTCTGCTACGTCCCTTCGGCCCTCAATCCAGCGG
ACCTTCCTTCCCGCGGCCTGCTGCCGGCTCTGCGGCCTCTTCCGCGTCTTCGCCTTCGCCC
TCAGACGAGTCGGATCTCCCTTTGGGCCGCCTCCCCGCATCGATACCGTCGACTAGCCGT
ACCTTTAAGACCAATGACTTACAAGGCAGCTGTAGATCTTAGCCACTTTTTAAAAGAAAA
GGGGGGACTGGAAGGGCTAATTCACTCCCAAAGAAGACAAGATCTGCTTTTTGCCTGTA
CTGGGTCTCTCTGGTTAGACCAGATCTGAGCCTGGGAGCTCTCTGGCTAACTAGGGAACC
CACTGCTTAAGCCTCAATAAAGCTTGCCTTGAGTGCTTCAAGTAGTGTGTGCCCGTCTGT
TGTGTGACTCTGGTAACTAGAGATCCCTCAGACCCTTTTAGTCAGTGTGGAAAATCTCTA

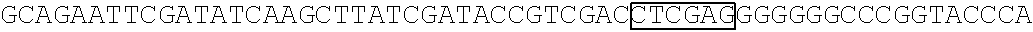

ATTCGCCCTATAGTGAGTCGTATTACAATTCACTGGCCGTCGTTTTACAACGTCGTGACT
GGGAAAACCCTGGCGTTACCCAACTTAATCGCCTTGCAGCACATCCCCCTTTCGCCAGCT
GGCGTAATAGCGAAGAGGCCCGCACCGATCGCCCTTCCCAACAGTTGCGCAGCCTGAAT
GGCGAATGGAAATTGTAAGCGTTAATATTTTGTTAAAATTCGCGTTAAATTTTTGTTAAA
TCAGCTCATTTTTTAACCAATAGGCCGAAATCGGCAAAATCCCTTATAAATCAAAAGAAT
AGACCGAGATAGGGTTGAGTGTTGTTCCAGTTTGGAACAAGAGTCCACTATTAAAGAAC
GTGGACTCCAACGTCAAAGGGCGAAAAACCGTCTATCAGGGCGATGGCCCACTACGTGA
ACCATCACCCTAATCAAGTTTTTTGGGGTCGAGGTGCCGTAAAGCACTAAATCGGAACCC
TAAAGGGAGCCCCCGATTTAGAGCTTGACGGGGAAAGCCGGCGAACGTGGCGAGAAAG
GAAGGGAAGAAAGCGAAAGGAGCGGGCGCTAGGGCGCTGGCAAGTGTAGCGGTCACGC
TGCGCGTAACCACCACACCCGCCGCGCTTAATGCGCCGCTACAGGGCGCGTCAGGTGGC
ACTTTTCGGGGAAATGTGCGCGGAACCCCTATTTGTTTATTTTTCTAAATACATTCAAATA
TGTATCCGCTCATGAGACAATAACCCTGATAAATGCTTCAATAATATTGAAAAAGGAAG
AGTATGAGTATTCAACATTTCCGTGTCGCCCTTATTCCCTTTTTTGCGGCATTTTGCCTTC
CTGTTTTTGCTCACCCAGAAACGCTGGTGAAAGTAAAAGATGCTGAAGATCAGTTGGGT
GCACGAGTGGGTTACATCGAACTGGATCTCAACAGCGGTAAGATCCTTGAGAGTTTTCG
CCCCGAAGAACGTTTTCCAATGATGAGCACTTTTAAAGTTCTGCTATGTGGCGCGGTATT
ATCCCGTATTGACGCCGGGCAAGAGCAACTCGGTCGCCGCATACACTATTCTCAGAATG
ACTTGGTTGAGTACTCACCAGTCACAGAAAAGCATCTTACGGATGGCATGACAGTAAGA
GAATTATGCAGTGCTGCCATAACCATGAGTGATAACACTGCGGCCAACTTACTTCTGACA
ACGATCGGAGGACCGAAGGAGCTAACCGCTTYTTTGCACAACATGGGGGATCATGTAAC
TCGCCTTGATCGTTGGGAACCGGAGCTGAATGAAGCCATACCAAACGACGAGCGTGACA
CCACGATGCCTGTAGCAATGGCAACAACGTTGCGCAAACTATTAACTGGCGAACTACTT
ACTCTAGCTTCCCGGCAACAATTAATAGACTGGATGGAGGCGGATAAAGTTGCAGGACC
ACTTCTGCGCTCGGCCCTTCCGGCTGGCTGGTTTATTGCTGATAAATCTGGAGCCGGTGA
GCGTGGGTCTCGCGGTATCATTGCAGCACTGGGGCCAGATGGTAAGCCCTCCCGTATCGT
AGTTATCTACACGACGGGGAGTCAGGCAACTATGGATGAACGAAATAGACAGATCGCTG
AGATAGGTGCCTCACTGATTAAGCATTGGTAACTGTCAGACCAAGTTTACTCATATATAC
TTTAGATTGATTTAAAACTTCATTTTTAATTTAAAAGGATCTAGGTGAAGATCCTTTTTGA
TAATCTCATGACCAAAATCCCTTAACGTGAGTTTTCGTTCCACTGAGCGTCAGACCCCGT
AGAAAAGATCAAAGGATCTTCTTGAGATCCTTTTTTTCTGCGCGTAATCTGCTGCTTGCA
AACAAAAAAACCACCGCTACCAGCGGTGGTTTGTTTGCCGGATCAAGAGCTACCAACTC
TTTTTCCGAAGGTAACTGGCTTCAGCAGAGCGCAGATACCAAATACTGTTCTTCTAGTGT
AGCCGTAGTTAGGCCACCACTTCAAGAACTCTGTAGCACCGCCTACATACCTCGCTCTGC
TAATCCTGTTACCAGTGGCTGCTGCCAGTGGCGATAAGTCGTGTCTTACCGGGTTGGACT
CAAGACGATAGTTACCGGATAAGGCGCAGCGGTCGGGCTGAACGGGGGGTTCGTGCACA
CAGCCCAGCTTGGAGCGAACGACCTACACCGAACTGAGATACCTACAGCGTGAGCTATG
AGAAAGCGCCACGCTTCCCGAAGGGAGAAAGGCGGACAGGTATCCGGTAAGCGGCAGG
GTCGGAACAGGAGAGCGCACGAGGGAGCTTCCAGGGGGAAACGCCTGGTATCTTTATAG
TCCTGTCGGGTTTCGCCACCTCTGACTTGAGCGTCGATTTTTGTGATGCTCGTCAGGGGG
GCGGAGCCTATGGAAAAACGCCAGCAACGCGGCCTTTTTACGGTTCCTGGCCTTTTGCTG
GCCTTTTGCTCACATGTTCTTTCCTGCGTTATCCCCTGATTCTGTGGATAACCGTATTACC
GCCTTTGAGTGAGCTGATACCGCTCGCCGCAGCCGAACGACCGAGCGCAGCGAGTCAGT
GAGCGAGGAAGCGGAAGAGCGCCCAATACGCAAACCGCCTCTCCCCGCGCGTTGGCCGA
TTCATTAATGCAGCTGGCACGACAGGTTTCCCGACTGGAAAGCGGGCAGTGAGCGCAAC
GCAATTAATGTGAGTTAGCTCACTCATTAGGCACCCCAGGCTTTACACTTTATGCTTCCG
GCTCGTATGTTGTGTGGAATTGTGAGCGGATAACAATTTCACACAGGAAACAGCTATGA
CCATGATTACGCCAAGCTCGAAATTAACCCTCACTAAAGGGAACAAAAGCTGGAGCTCC

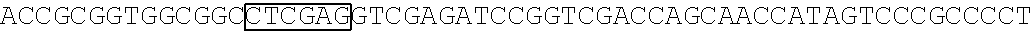

AACTCCGCCCATCCCGCCCCTAACTCCGCCCAGTTCCGCCCATTCTCCGCCCCATGGCTG
ACTAATTTTTTTTATTTATGCAGAGGCCGAGGCCGCCTCGGCCTCTGAGCTATTCCAGAA
GTAGTGAGGAGGCTTTTTTGGAGGCCTAGGCTTTTGCAAAAAGCTTCGACGGTATCGATT
GGCTCATGTCCAACATTACCGCCATGTTGACATTGATTATTGACTAGTTATTAATAGTAA
TCAATTACGGGGTCATTAGTTCATAGCCCATATATGGAGTTCCGCGTTACATAACTTACG
GTAAATGGCCCGCCTGGCTGACCGCCCAACGACCCCCGCCCATTGACGTCAATAATGAC
GTATGTTCCCATAGTAACGCCAATAGGGACTTTCCATTGACGTCAATGGGTGGAGTATTT
ACGGTAAACTGCCCACTTGGCAGTACATCAAGTGTATCATATGCCAAGTACGCCCCCTAT
TGACGTCAATGACGGTAAATGGCCCGCCTGGCATTATGCCCAGTACATGACCTTATGGG
ACTTTCCTACTTGGCAGTACATCTACGTATTAGTCATCGCTATTACCATGGTGATGCGGTT
TTGGCAGTACATCAATGGGCGTGGATAGCGGTTTGACTCACGGGGATTTCCAAGTCTCCA
CCCCATTGACGTCAATGGGAGTTTGTTTTGGCACCAAAATCAACGGGACTTTCCAAAATG
TCGTAACAACTCCGCCCCATTGACGCAAATGGGCGGTAGGCGTGTACGGAATTCGGAGT
GGCGAGCCCTCAGATCCTGCATATAAGCAGCTGCTTTTTGCCTGTACTGGGTCTCTCTG

TABLE 10
Leader _R12-Hinge-CH2—CH3-CD28tm/41BB-Z-T2A-tEGFR (SEQ ID NO: 38)
Leader: MLLLVTSLLLCELPHPAFLLIP
R12 scFv
QEQLVESGGRLVTPGGSLTLSCKASGFDFSAYYMSWVRQAPGKGLEWIATIYPSSGKTYYAT
WVNGRFTISSDNAQNTVDLQMNSLTAADRATYFCARDSYADDGALFNIWGPGTLVTISSGG
GGSGGGGSGGGGSELVLTQSPSVSAALGSPAKITCTLSSAHKTDTIDWYQQLQGEAPRYLMQ
VQSDGSYTKRPGVPDRFSGSSSGADRYLIIPSVQADDEADYYCGADYIGGYVFGGGTQLTVT
G
Hinge Spacer: ESKYGPPCPPCP
CH2

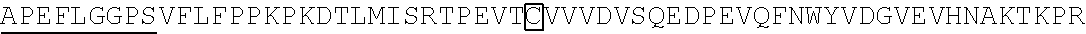

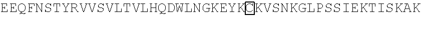

CH3

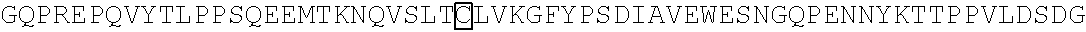

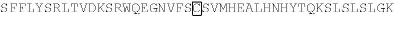

CD28: MFWVLVVVGGVLACYSLLVTVAFIIFWV
4-1BB: KRGRKKLLYIFKQPFMRPVQTTQEEDGCSCRFPEEEEGGCEL
CD3 zeta
RVKFSRSADAPAYQQGQNQLYNELNLGRREEYDVLDKRRGRDPEMGGKPRRKNPQEGLYN
ELQKDKMAEAYSEIGMKGERRRGKGHDGLYQGLSTATKDTYDALHMQALPPR
T2A
LEGGGEGRGSLLTCGDVEENPGPR
tEGFR
MLLLVTSLLLCELPHPAFLLIPRKVCNGIGIGEFKDSLSINATNIKHFKNCTSISGDLHILPVAFR
GDSFTHTPPLDPQELDILKTVKEITGFLLIQAWPENRTDLHAFENLEIIRGRTKQHGQFSLAVV
SLNITSLGLRSLKEISDGDVIISGNKNLCYANTINWKKLFGTSGQKTKIISNRGENSCKATGQV
CHALCSPEGCWGPEPRDCVSCRNVSRGRECVDKCNLLEGEPREFVENSECIQCHPECLPQAM
NITCTGRGPDNCIQCAHYIDGPHCVKTCPAGVMGENNTLVWKYADAGHVCHLCHPNCTYG
CTGPGLEGCPTNGPKIPSIATGMVGALLLLLVVALGIGLFM

TABLE 11
R12 intermediate spacer CAR: PJ_R12-CH3-41BB-Z-T2A-tEGFR (SEQ ID NO: 39)
GTTAGACCAGATCTGAGCCTGGGAGCTCTCTGGCTAACTAGGGAACCCACTGCTTAAGC
CTCAATAAAGCTTGCCTTGAGTGCTTCAAGTAGTGTGTGCCCGTCTGTTGTGTGACTCTG
GTAACTAGAGATCCCTCAGACCCTTTTAGTCAGTGTGGAAAATCTCTAGCAGTGGCGCCC
GAACAGGGACTTGAAAGCGAAAGGGAAACCAGAGGAGCTCTCTCGACGCAGGACTCGG
CTTGCTGAAGCGCGCACGGCAAGAGGCGAGGGGCGGCGACTGGTGAGTACGCCAAAAA
TTTTGACTAGCGGAGGCTAGAAGGAGAGAGATGGGTGCGAGAGCGTCAGTATTAAGCGG
GGGAGAATTAGATCGATGGGAAAAAATTCGGTTAAGGCCAGGGGGAAAGAAAAAATAT
AAATTAAAACATATAGTATGGGCAAGCAGGGAGCTAGAACGATTCGCAGTTAATCCTGG
CCTGTTAGAAACATCAGAAGGCTGTAGACAAATACTGGGACAGCTACAACCATCCCTTC
AGACAGGATCAGAAGAACTTAGATCATTATATAATACAGTAGCAACCCTCTATTGTGTG
CATCAAAGGATAGAGATAAAAGACACCAAGGAAGCTTTAGACAAGATAGAGGAAGAGC
AAAACAAAAGTAAGAAAAAAGCACAGCAAGCAGCAGCTGACACAGGACACAGCAATCA
GGTCAGCCAAAATTACCCTATAGTGCAGAACATCCAGGGGCAAATGGTACATCAGGCCA
TATCACCTAGAACTTTAAATGCATGGGTAAAAGTAGTAGAAGAGAAGGCTTTCAGCCCA
GAAGTGATACCCATGTTTTCAGCATTATCAGAAGGAGCCACCCCACAAGATTTAAACAC
CATGCTAAACACAGTGGGGGGACATCAAGCAGCCATGCAAATGTTAAAAGAGACCATCA
ATGAGGAAGCTGCAGGCAAAGAGAAGAGTGGTGCAGAGAGAAAAAAGAGCAGTGGGA
ATAGGAGCTTTGTTCCTTGGGTTCTTGGGAGCAGCAGGAAGCACTATGGGCGCAGCGTC
AATGACGCTGACGGTACAGGCCAGACAATTATTGTCTGGTATAGTGCAGCAGCAGAACA
ATTTGCTGAGGGCTATTGAGGCGCAACAGCATCTGTTGCAACTCACAGTCTGGGGCATCA
AGCAGCTCCAGGCAAGAATCCTGGCTGTGGAAAGATACCTAAAGGATCAACAGCTCCTG
GGGATTTGGGGTTGCTCTGGAAAACTCATTTGCACCACTGCTGTGCCTTGGATCTACAAA
TGGCAGTATTCATCCACAATTTTAAAAGAAAAGGGGGGATTGGGGGGTACAGTGCAGGG
GAAAGAATAGTAGACATAATAGCAACAGACATACAAACTAAAGAATTACAAAAACAAA
TTACAAAAATTCAAAATTTTCGGGTTTATTACAGGGACAGCAGAGATCCAGTTTGGGGAT
CAATTGCATGAAGAATCTGCTTAGGGTTAGGCGTTTTGCGCTGCTTCGCGAGGATCTGCG
ATCGCTCCGGTGCCCGTCAGTGGGCAGAGCGCACATCGCCCACAGTCCCCGAGAAGTTG
GGGGGAGGGGTCGGCAATTGAACCGGTGCCTAGAGAAGGTGGCGCGGGGTAAACTGGG
AAAGTGATGTCGTGTACTGGCTCCGCCTTTTTCCCGAGGGTGGGGGAGAACCGTATATAA
GTGCAGTAGTCGCCGTGAACGTTCTTTTTCGCAACGGGTTTGCCGCCAGAACACAGCTGA
AGCTTCGAGGGGCTCGCATCTCTCCTTCACGCGCCCGCCGCCCTACCTGAGGCCGCCATC
CACGCCGGTTGAGTCGCGTTCTGCCGCCTCCCGCCTGTGGTGCCTCCTGAACTGCGTCCG
CCGTCTAGGTAAGTTTAAAGCTCAGGTCGAGACCGGGCCTTTGTCCGGCGCTCCCTTGGA
GCCTACCTAGACTCAGCCGGCTCTCCACGCTTTGCCTGACCCTGCTTGCTCAACTCTACGT
CTTTGTTTCGTTTTCTGTTCTGCGCCGTTACAGATCCAAGCTGTGACCGGCGCCTACG

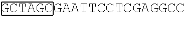

R12 ScFv

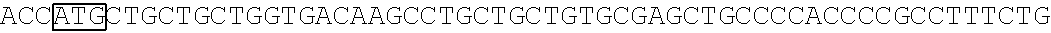

CTGATCCCCCAGGAACAGCTCGTCGAAAGCGGCGGCAGACTGGTGACACCTGGCGGCAG
CCTGACCCTGAGCTGCAAGGCCAGCGGCTTCGACTTCAGCGCCTACTACATGAGCTGGGT
CCGCCAGGCCCCTGGCAAGGGACTGGAATGGATCGCCACCATCTACCCCAGCAGCGGCA
AGACCTACTACGCCACCTGGGTGAACGGACGGTTCACCATCTCCAGCGACAACGCCCAG
AACACCGTGGACCTGCAGATGAACAGCCTGACAGCCGCCGACCGGGCCACCTACTTTTG
CGCCAGAGACAGCTACGCCGACGACGGCGCCCTGTTCAACATCTGGGGCCCTGGCACCC
TGGTGACAATCTCTAGCGGCGGAGGCGGATCTGGTGGCGGAGGAAGTGGCGGCGGAGG
ATCTGAGCTGGTGCTGACCCAGAGCCCCTCTGTGTCTGCTGCCCTGGGAAGCCCTGCCAA
GATCACCTGTACCCTGAGCAGCGCCCACAAGACCGACACCATCGACTGGTATCAGCAGC
TGCAGGGCGAGGCCCCCAGATACCTGATGCAGGTGCAGAGCGACGGCAGCTACACCAA
GAGGCCAGGCGTGCCCGACCGGTTCAGCGGATCTAGCTCTGGCGCCGACCGCTACCTGA
TCATCCCCAGCGTGCAGGCCGATGACGAGGCCGATTACTACTGTGGCGCCGACTACATC
GGCGGCTACGTGTTCGGCGGAGGCACCCAGCTGACCGTGACCGGCGAGTCTAAG
Hinge Spacer

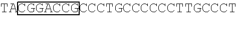

CH3
GGCCAGCCTCGCGAGCCCCAGGTGTACACCCTGCCTCCCTCCCAGGAAGAGATGACCAA
GAACCAGGTGTCCCTGACCTGCCTGGTGAAGGGCTTCTACCCCAGCGACATCGCCGTGG
AGTGGGAGAGCAACGGCCAGCCTGAGAACAACTACAAGACCACCCCTCCCGTGCTGGAC
AGCGACGGCAGCTTCTTCCTGTACAGCCGGCTGACCGTGGACAAGAGCCGGTGGCAGGA
AGGCAACGTCTTTAGCTGCAGCGTGATGCACGAGGCCCTGCACAACCACTACACCCAGA
AGAGCCTGAGCCTGTCCCTGGGCAAG
4-1BB
ATGTTCTGGGTGCTGGTGGTGGTGGGCGGGGTGCTGGCCTGCTACAGCCTGCTGGTGACA
GTGGCCTTCATCATCTTTTGGGTGAAACGGGGCAGAAAGAAACTCCTGTATATATTCAAA
CAACCATTTATGAGACCAGTACAAACTACTCAAGAGGAAGATGGCTGTAGCTGCCGATT
TCCAGAAGAAGAAGAAGGAGGATGTGAACTG
CD3zeta
CGGGTGAAGTTCAGCAGAAGCGCCGACGCCCCTGCCTACCAGCAGGGCCAGAATCAGCT
GTACAACGAGCTGAACCTGGGCAGAAGGGAAGAGTACGACGTCCTGGATAAGCGGAGA
GGCCGGGACCCTGAGATGGGCGGCAAGCCTCGGCGGAAGAACCCCCAGGAAGGCCTGT
ATAACGAACTGCAGAAAGACAAGATGGCCGAGGCCTACAGCGAGATCGGCATGAAGGG
CGAGCGGAGGCGGGGCAAGGGCCACGACGGCCTGTATCAGGGCCTGTCCACCGCCACCA
AGGATACCTACGACGCCCTGCACATGCAGGCCCTGCCCCCAAGG
T2A

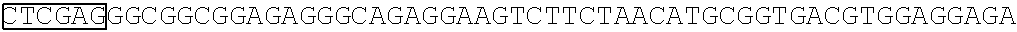

ATCCCGGCCCTAGG
tEGFR
ATGCTTCTCCTGGTGACAAGCCTTCTGCTCTGTGAGTTACCACACCCAGCATTCCTCCTGA
TCCCACGCAAAGTGTGTAACGGAATAGGTATTGGTGAATTTAAAGACTCACTCTCCATAA
ATGCTACGAATATTAAACACTTCAAAAACTGCACCTCCATCAGTGGCGATCTCCACATCC

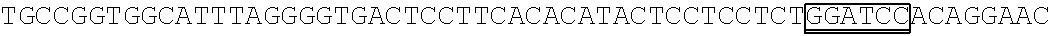

TGGATATTCTGAAAACCGTAAAGGAAATCACAGGGTTTTTGCTGATTCAGGCTTGGCCTG
AAAACAGGACGGACCTCCATGCCTTTGAGAACCTAGAAATCATACGCGGCAGGACCAAG
CAACATGGTCAGTTTTCTCTTGCAGTCGTCAGCCTGAACATAACATCCTTGGGATTACGC
TCCCTCAAGGAGATAAGTGATGGAGATGTGATAATTTCAGGAAACAAAAATTTGTGCTA
TGCAAATACAATAAACTGGAAAAAACTGTTTGGGACCTCCGGTCAGAAAACCAAAATTA
TAAGCAACAGAGGTGAAAACAGCTGCAAGGCCACAGGCCAGGTCTGCCATGCCTTGTGC
TCCCCCGAGGGCTGCTGGGGCCCGGAGCCCAGGGACTGCGTCTCTTGCCGGAATGTCAG
CCGAGGCAGGGAATGCGTGGACAAGTGCAACCTTCTGGAGGGTGAGCCAAGGGAGTTTG
TGGAGAACTCTGAGTGCATACAGTGCCACCCAGAGTGCCTGCCTCAGGCCATGAACATC
ACCTGCACAGGACGGGGACCAGACAACTGTATCCAGTGTGCCCACTACATTGACGGCCC
CCACTGCGTCAAGACCTGCCCGGCAGGAGTCATGGGAGAAAACAACACCCTGGTCTGGA
AGTACGCAGACGCCGGCCATGTGTGCCACCTGTGCCATCCAAACTGCACCTACGGATGC
ACTGGGCCAGGTCTTGAAGGCTGTCCAACGAATGGGCCTAAGATCCCGTCCATCGCCAC
TGGGATGGTGGGGGCCCTCCTCTTGCTGCTGGTGGTGGCCCTGGGGATCGGCCTCTTCAT
GTGA

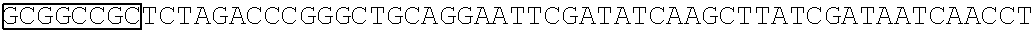

CTGGATTACAAAATTTGTGAAAGATTGACTGGTATTCTTAACTATGTTGCTCCTTTTACGC
TATGTGGATACGCTGCTTTAATGCCTTTGTATCATGCTATTGCTTCCCGTATGGCTTTCAT
TTTCTCCTCCTTGTATAAATCCTGGTTGCTGTCTCTTTATGAGGAGTTGTGGCCCGTTGTC
AGGCAACGTGGCGTGGTGTGCACTGTGTTTGCTGACGCAACCCCCACTGGTTGGGGCATT
GCCACCACCTGTCAGCTCCTTTCCGGGACTTTCGCTTTCCCCCTCCCTATTGCCACGGCGG
AACTCATCGCCGCCTGCCTTGCCCGCTGCTGGACAGGGGCTCGGCTGTTGGGCACTGACA
ATTCCGTGGTGTTGTCGGGGAAATCATCGTCCTTTCCTTGGCTGCTCGCCTGTGTTGCCAC
CTGGATTCTGCGCGGGACGTCCTTCTGCTACGTCCCTTCGGCCCTCAATCCAGCGGACCT
TCCTTCCCGCGGCCTGCTGCCGGCTCTGCGGCCTCTTCCGCGTCTTCGCCTTCGCCCTCAG
ACGAGTCGGATCTCCCTTTGGGCCGCCTCCCCGCATCGATACCGTCGACTAGCCGTACCT
TTAAGACCAATGACTTACAAGGCAGCTGTAGATCTTAGCCACTTTTTAAAAGAAAAGGG
GGGACTGGAAGGGCTAATTCACTCCCAAAGAAGACAAGATCTGCTTTTTGCCTGTACTG
GGTCTCTCTGGTTAGACCAGATCTGAGCCTGGGAGCTCTCTGGCTAACTAGGGAACCCAC
TGCTTAAGCCTCAATAAAGCTTGCCTTGAGTGCTTCAAGTAGTGTGTGCCCGTCTGTTGT
GTGACTCTGGTAACTAGAGATCCCTCAGACCCTTTTAGTCAGTGTGGAAAATCTCTAGCA

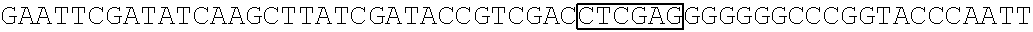

CGCCCTATAGTGAGTCGTATTACAATTCACTGGCCGTCGTTTTACAACGTCGTGACTGGG
AAAACCCTGGCGTTACCCAACTTAATCGCCTTGCAGCACATCCCCCTTTCGCCAGCTGGC
GTAATAGCGAAGAGGCCCGCACCGATCGCCCTTCCCAACAGTTGCGCAGCCTGAATGGC
GAATGGAAATTGTAAGCGTTAATATTTTGTTAAAATTCGCGTTAAATTTTTGTTAAATCA
GCTCATTTTTTAACCAATAGGCCGAAATCGGCAAAATCCCTTATAAATCAAAAGAATAG
ACCGAGATAGGGTTGAGTGTTGTTCCAGTTTGGAACAAGAGTCCACTATTAAAGAACGT
GGACTCCAACGTCAAAGGGCGAAAAACCGTCTATCAGGGCGATGGCCCACTACGTGAAC
CATCACCCTAATCAAGTTTTTTGGGGTCGAGGTGCCGTAAAGCACTAAATCGGAACCCTA
AAGGGAGCCCCCGATTTAGAGCTTGACGGGGAAAGCCGGCGAACGTGGCGAGAAAGGA
AGGGAAGAAAGCGAAAGGAGCGGGCGCTAGGGCGCTGGCAAGTGTAGCGGTCACGCTG
CGCGTAACCACCACACCCGCCGCGCTTAATGCGCCGCTACAGGGCGCGTCAGGTGGCAC
TTTTCGGGGAAATGTGCGCGGAACCCCTATTTGTTTATTTTTCTAAATACATTCAAATATG
TATCCGCTCATGAGACAATAACCCTGATAAATGCTTCAATAATATTGAAAAAGGAAGAG
TATGAGTATTCAACATTTCCGTGTCGCCCTTATTCCCTTTTTTGCGGCATTTTGCCTTCCTG
TTTTTGCTCACCCAGAAACGCTGGTGAAAGTAAAAGATGCTGAAGATCAGTTGGGTGCA
CGAGTGGGTTACATCGAACTGGATCTCAACAGCGGTAAGATCCTTGAGAGTTTTCGCCCC
GAAGAACGTTTTCCAATGATGAGCACTTTTAAAGTTCTGCTATGTGGCGCGGTATTATCC
CGTATTGACGCCGGGCAAGAGCAACTCGGTCGCCGCATACACTATTCTCAGAATGACTT
GGTTGAGTACTCACCAGTCACAGAAAAGCATCTTACGGATGGCATGACAGTAAGAGAAT
TATGCAGTGCTGCCATAACCATGAGTGATAACACTGCGGCCAACTTACTTCTGACAACGA
TCGGAGGACCGAAGGAGCTAACCGCTTTTTTGCACAACATGGGGGATCATGTAACTCGC
CTTGATCGTTGGGAACCGGAGCTGAATGAAGCCATACCAAACGACGAGCGTGACACCAC
GATGCCTGTAGCAATGGCAACAACGTTGCGCAAACTATTAACTGGCGAACTACTTACTCT
AGCTTCCCGGCAACAATTAATAGACTGGATGGAGGCGGATAAAGTTGCAGGACCACTTC
TGCGCTCGGCCCTTCCGGCTGGCTGGTTTATTGCTGATAAATCTGGAGCCGGTGAGCGTG
GGTCTCGCGGTATCATTGCAGCACTGGGGCCAGATGGTAAGCCCTCCCGTATCGTAGTTA
TCTACACGACGGGGAGTCAGGCAACTATGGATGAACGAAATAGACAGATCGCTGAGATA
GGTGCCTCACTGATTAAGCATTGGTAACTGTCAGACCAAGTTTACTCATATATACTTTAG
ATTGATTTAAAACTTCATTTTTAATTTAAAAGGATCTAGGTGAAGATCCTTTTTGATAATC
TCATGACCAAAATCCCTTAACGTGAGTTTTCGTTCCACTGAGCGTCAGACCCCGTAGAAA
AGATCAAAGGATCTTCTTGAGATCCTTTTTTTCTGCGCGTAATCTGCTGCTTGCAAACAA
AAAAACCACCGCTACCAGCGGTGGTTTGTTTGCCGGATCAAGAGCTACCAACTCTTTTTC
CGAAGGTAACTGGCTTCAGCAGAGCGCAGATACCAAATACTGTTCTTCTAGTGTAGCCGT
AGTTAGGCCACCACTTCAAGAACTCTGTAGCACCGCCTACATACCTCGCTCTGCTAATCC
TGTTACCAGTGGCTGCTGCCAGTGGCGATAAGTCGTGTCTTACCGGGTTGGACTCAAGAC
GATAGTTACCGGATAAGGCGCAGCGGTCGGGCTGAACGGGGGGTTCGTGCACACAGCCC
AGCTTGGAGCGAACGACCTACACCGAACTGAGATACCTACAGCGTGAGCTATGAGAAAG
CGCCACGCTTCCCGAAGGGAGAAAGGCGGACAGGTATCCGGTAAGCGGCAGGGTCGGA
ACAGGAGAGCGCACGAGGGAGCTTCCAGGGGGAAACGCCTGGTATCTTTATAGTCCTGT
CGGGTTTCGCCACCTCTGACTTGAGCGTCGATTTTTGTGATGCTCGTCAGGGGGGCGGAG
CCTATGGAAAAACGCCAGCAACGCGGCCTTTTTACGGTTCCTGGCCTTTTGCTGGCCTTT
TGCTCACATGTTCTTTCCTGCGTTATCCCCTGATTCTGTGGATAACCGTATTACCGCCTTT
GAGTGAGCTGATACCGCTCGCCGCAGCCGAACGACCGAGCGCAGCGAGTCAGTGAGCG
AGGAAGCGGAAGAGCGCCCAATACGCAAACCGCCTCTCCCCGCGCGTTGGCCGATTCAT
TAATGCAGCTGGCACGACAGGTTTCCCGACTGGAAAGCGGGCAGTGAGCGCAACGCAAT
TAATGTGAGTTAGCTCACTCATTAGGCACCCCAGGCTTTACACTTTATGCTTCCGGCTCGT
ATGTTGTGTGGAATTGTGAGCGGATAACAATTTCACACAGGAAACAGCTATGACCATGA
TTACGCCAAGCTCGAAATTAACCCTCACTAAAGGGAACAAAAGCTGGAGCTCCACCGCG

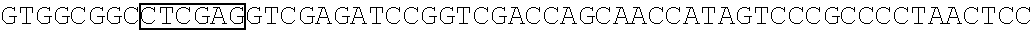

GCCCATCCCGCCCCTAACTCCGCCCAGTTCCGCCCATTCTCCGCCCCATGGCTGACTAAT
TTTTTTTATTTATGCAGAGGCCGAGGCCGCCTCGGCCTCTGAGCTATTCCAGAAGTAGTG
AGGAGGCTTTTTTGGAGGCCTAGGCTTTTGCAAAAAGCTTCGACGGTATCGATTGGCTCA
TGTCCAACATTACCGCCATGTTGACATTGATTATTGACTAGTTATTAATAGTAATCAATT
ACGGGGTCATTAGTTCATAGCCCATATATGGAGTTCCGCGTTACATAACTTACGGTAAAT
GGCCCGCCTGGCTGACCGCCCAACGACCCCCGCCCATTGACGTCAATAATGACGTATGTT
CCCATAGTAACGCCAATAGGGACTTTCCATTGACGTCAATGGGTGGAGTATTTACGGTAA
ACTGCCCACTTGGCAGTACATCAAGTGTATCATATGCCAAGTACGCCCCCTATTGACGTC
AATGACGGTAAATGGCCCGCCTGGCATTATGCCCAGTACATGACCTTATGGGACTTTCCT
ACTTGGCAGTACATCTACGTATTAGTCATCGCTATTACCATGGTGATGCGGTTTTGGCAG
TACATCAATGGGCGTGGATAGCGGTTTGACTCACGGGGATTTCCAAGTCTCCACCCCATT
GACGTCAATGGGAGTTTGTTTTGGCACCAAAATCAACGGGACTTTCCAAAATGTCGTAAC
AACTCCGCCCCATTGACGCAAATGGGCGGTAGGCGTGTACGGAATTCGGAGTGGCGAGC
CCTCAGATCCTGCATATAAGCAGCTGCTTTTTGCCTGTACTGGGTCTCTCTG

TABLE 12
Leader_R12-Hinge-CH3-CD28tm/41BB-Z-T2A-tEGFR (SEQ ID NO: 40)
Leader
MLLLVTSLLLCELPHPAFLLIP
R12 scFV
QEQLVESGGRLVTPGGSLTLSCKASGFDFSAYYMSWVRQAPGKGLEWIATIYPSSGKTYYAT
WVNGRFTISSDNAQNTVDLQMNSLTAADRATYFCARDSYADDGALFNIWGPGTLVTISSGG
GGSGGGGSGGGGSELVLTQSPSVSAALGSPAKITCTLSSAHKTDTIDWYQQLQGEAPRYLMQ
VQSDGSYTKRPGVPDRFSGSSSGADRYLIIPSVQADDEADYYCGADYIGGYVFGGGTQLTVT
G
Hinge Spacer
ESKYGPPCPPCP
CH3

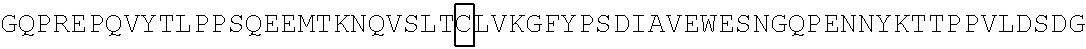

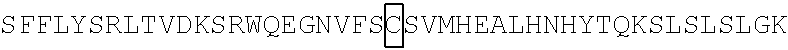

CD28tm
MFWVLVVVGGVLACYSLLVTVAFIIFWV
4-1BB
KRGRKKLLYIFKQPFMRPVQTTQEEDGCSCRFPEEEEGGCEL
CD3 zeta
RVKFSRSADAPAYQQGQNQLYNELNLGRREEYDVLDKRRGRDPEMGGKPRRKNPQEGLYN
ELQKDKMAEAYSEIGMKGERRRGKGHDGLYQGLSTATKDTYDALHMQALPPR
T2A
LEGGGEGRGSLLTCGDVEENPGPR
tEGFR
MLLLVTSLLLCELPHPAFLLIPRKVCNGIGIGEFKDSLSINATNIKHFKNCTSISGDLHILPVAFR
GDSFTHTPPLDPQELDILKTVKEITGFLLIQAWPENRTDLHAFENLEIIRGRTKQHGQFSLAVV
SLNITSLGLRSLKEISDGDVIISGNKNLCYANTINWKKLFGTSGQKTKIISNRGENSCKATGQV
CHALCSPEGCWGPEPRDCVSCRNVSRGRECVDKCNLLEGEPREFVENSECIQCHPECLPQAM
NITCTGRGPDNCIQCAHVIDGPHCVKTCPAGVMGENNTLVWKYADAGHVCHLCHPNCTYG
CTGPGLEGCPTNGPKIPSIATGMVGALLLLLVVALGIGLFM

TABLE 13
R12 short spacer CAR: PJ_R12-Hinge-41BB-Z-T2A-tEGFR (SEQ ID NO: 41)
GTTAGACCAGATCTGAGCCTGGGAGCTCTCTGGCTAACTAGGGAACCCACTGCTTAAGC
CTCAATAAAGCTTGCCTTGAGTGCTTCAAGTAGTGTGTGCCCGTCTGTTGTGTGACTCTG
GTAACTAGAGATCCCTCAGACCCTTTTAGTCAGTGTGGAAAATCTCTAGCAGTGGCGCCC
GAACAGGGACTTGAAAGCGAAAGGGAAACCAGAGGAGCTCTCTCGACGCAGGACTCGG
CTTGCTGAAGCGCGCACGGCAAGAGGCGAGGGGCGGCGACTGGTGAGTACGCCAAAAA
TTTTGACTAGCGGAGGCTAGAAGGAGAGAGATGGGTGCGAGAGCGTCAGTATTAAGCGG
GGGAGAATTAGATCGATGGGAAAAAATTCGGTTAAGGCCAGGGGGAAAGAAAAAATAT
AAATTAAAACATATAGTATGGGCAAGCAGGGAGCTAGAACGATTCGCAGTTAATCCTGG
CCTGTTAGAAACATCAGAAGGCTGTAGACAAATACTGGGACAGCTACAACCATCCCTTC
AGACAGGATCAGAAGAACTTAGATCATTATATAATACAGTAGCAACCCTCTATTGTGTG
CATCAAAGGATAGAGATAAAAGACACCAAGGAAGCTTTAGACAAGATAGAGGAAGAGC
AAAACAAAAGTAAGAAAAAAGCACAGCAAGCAGCAGCTGACACAGGACACAGCAATCA
GGTCAGCCAAAATTACCCTATAGTGCAGAACATCCAGGGGCAAATGGTACATCAGGCCA
TATCACCTAGAACTTTAAATGCATGGGTAAAAGTAGTAGAAGAGAAGGCTTTCAGCCCA
GAAGTGATACCCATGTTTTCAGCATTATCAGAAGGAGCCACCCCACAAGATTTAAACAC
CATGCTAAACACAGTGGGGGGACATCAAGCAGCCATGCAAATGTTAAAAGAGACCATCA
ATGAGGAAGCTGCAGGCAAAGAGAAGAGTGGTGCAGAGAGAAAAAAGAGCAGTGGGA
ATAGGAGCTTTGTTCCTTGGGTTCTTGGGAGCAGCAGGAAGCACTATGGGCGCAGCGTC
AATGACGCTGACGGTACAGGCCAGACAATTATTGTCTGGTATAGTGCAGCAGCAGAACA
ATTTGCTGAGGGCTATTGAGGCGCAACAGCATCTGTTGCAACTCACAGTCTGGGGCATCA
AGCAGCTCCAGGCAAGAATCCTGGCTGTGGAAAGATACCTAAAGGATCAACAGCTCCTG
GGGATTTGGGGTTGCTCTGGAAAACTCATTTGCACCACTGCTGTGCCTTGGATCTACAAA
TGGCAGTATTCATCCACAATTTTAAAAGAAAAGGGGGGATTGGGGGGTACAGTGCAGGG
GAAAGAATAGTAGACATAATAGCAACAGACATACAAACTAAAGAATTACAAAAACAAA
TTACAAAAATTCAAAATTTTCGGGTTTATTACAGGGACAGCAGAGATCCAGTTTGGGGAT
CAATTGCATGAAGAATCTGCTTAGGGTTAGGCGTTTTGCGCTGCTTCGCGAGGATCTGCG
ATCGCTCCGGTGCCCGTCAGTGGGCAGAGCGCACATCGCCCACAGTCCCCGAGAAGTTG
GGGGGAGGGGTCGGCAATTGAACCGGTGCCTAGAGAAGGTGGCGCGGGGTAAACTGGG
AAAGTGATGTCGTGTACTGGCTCCGCCTTTTTCCCGAGGGTGGGGGAGAACCGTATATAA
GTGCAGTAGTCGCCGTGAACGTTCTTTTTCGCAACGGGTTTGCCGCCAGAACACAGCTGA
AGCTTCGAGGGGCTCGCATCTCTCCTTCACGCGCCCGCCGCCCTACCTGAGGCCGCCATC
CACGCCGGTTGAGTCGCGTTCTGCCGCCTCCCGCCTGTGGTGCCTCCTGAACTGCGTCCG
CCGTCTAGGTAAGTTTAAAGCTCAGGTCGAGACCGGGCCTTTGTCCGGCGCTCCCTTGGA
GCCTACCTAGACTCAGCCGGCTCTCCACGCTTTGCCTGACCCTGCTTGCTCAACTCTACGT
CTTTGTTTCGTTTTCTGTTCTGCGCCGTTACAGATCCAAGCTGTGACCGGCGCCTACG

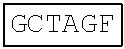

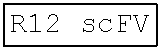

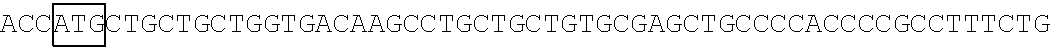

CTGATCCCCCAGGAACAGCTCGTCGAAAGCGGCGGCAGACTGGTGACACCTGGCGGCAG
CCTGACCCTGAGCTGCAAGGCCAGCGGCTTCGACTTCAGCGCCTACTACATGAGCTGGGT
CCGCCAGGCCCCTGGCAAGGGACTGGAATGGATCGCCACCATCTACCCCAGCAGCGGCA
AGACCTACTACGCCACCTGGGTGAACGGACGGTTCACCATCTCCAGCGACAACGCCCAG
AACACCGTGGACCTGCAGATGAACAGCCTGACAGCCGCCGACCGGGCCACCTACTTTTG
CGCCAGAGACAGCTACGCCGACGACGGCGCCCTGTTCAACATCTGGGGCCCTGGCACCC
TGGTGACAATCTCTAGCGGCGGAGGCGGATCTGGTGGCGGAGGAAGTGGCGGCGGAGG
ATCTGAGCTGGTGCTGACCCAGAGCCCCTCTGTGTCTGCTGCCCTGGGAAGCCCTGCCAA
GATCACCTGTACCCTGAGCAGCGCCCACAAGACCGACACCATCGACTGGTATCAGCAGC
TGCAGGGCGAGGCCCCCAGATACCTGATGCAGGTGCAGAGCGACGGCAGCTACACCAA
GAGGCCAGGCGTGCCCGACCGGTTCAGCGGATCTAGCTCTGGCGCCGACCGCTACCTGA
TCATCCCCAGCGTGCAGGCCGATGACGAGGCCGATTACTACTGTGGCGCCGACTACATC
GGCGGCTACGTGTTCGGCGGAGGCACCCAGCTGACCGTGACCGGCGAGTCTAAG
Hinge/Spacer

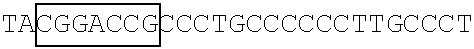

4-1BB
ATGTTCTGGGTGCTGGTGGTGGTGGGCGGGGTGCTGGCCTGCTACAGCCTGCTGGTGACA
GTGGCCTTCATCATCTTTTGGGTGAAACGGGGCAGAAAGAAACTCCTGTATATATTCAAA
CAACCATTTATGAGACCAGTACAAACTACTCAAGAGGAAGATGGCTGTAGCTGCCGATT
TCCAGAAGAAGAAGAAGGAGGATGTGAACTG
CD3 zeta
CGGGTGAAGTTCAGCAGAAGCGCCGACGCCCCTGCCTACCAGCAGGGCCAGAATCAGCT
GTACAACGAGCTGAACCTGGGCAGAAGGGAAGAGTACGACGTCCTGGATAAGCGGAGA
GGCCGGGACCCTGAGATGGGCGGCAAGCCTCGGCGGAAGAACCCCCAGGAAGGCCTGT
ATAACGAACTGCAGAAAGACAAGATGGCCGAGGCCTACAGCGAGATCGGCATGAAGGG
CGAGCGGAGGCGGGGCAAGGGCCACGACGGCCTGTATCAGGGCCTGTCCACCGCCACCA
AGGATACCTACGACGCCCTGCACATGCAGGCCCTGCCCCCAAGG
T2A

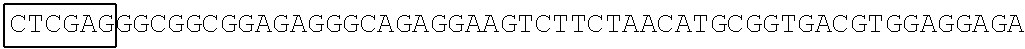

ATCCCGGCCCTAGG
tEGFR
ATGCTTCTCCTGGTGACAAGCCTTCTGCTCTGTGAGTTACCACACCCAGCATTCCTCCTGA
TCCCACGCAAAGTGTGTAACGGAATAGGTATTGGTGAATTTAAAGACTCACTCTCCATAA
ATGCTACGAATATTAAACACTTCAAAAACTGCACCTCCATCAGTGGCGATCTCCACATCC

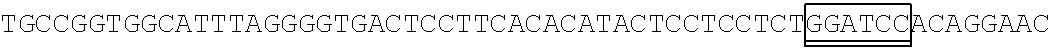

TGGATATTCTGAAAACCGTAAAGGAAATCACAGGGTTTTTGCTGATTCAGGCTTGGCCTG
AAAACAGGACGGACCTCCATGCCTTTGAGAACCTAGAAATCATACGCGGCAGGACCAAG
CAACATGGTCAGTTTTCTCTTGCAGTCGTCAGCCTGAACATAACATCCTTGGGATTACGC
TCCCTCAAGGAGATAAGTGATGGAGATGTGATAATTTCAGGAAACAAAAATTTGTGCTA
TGCAAATACAATAAACTGGAAAAAACTGTTTGGGACCTCCGGTCAGAAAACCAAAATTA
TAAGCAACAGAGGTGAAAACAGCTGCAAGGCCACAGGCCAGGTCTGCCATGCCTTGTGC
TCCCCCGAGGGCTGCTGGGGCCCGGAGCCCAGGGACTGCGTCTCTTGCCGGAATGTCAG
CCGAGGCAGGGAATGCGTGGACAAGTGCAACCTTCTGGAGGGTGAGCCAAGGGAGTTTG
TGGAGAACTCTGAGTGCATACAGTGCCACCCAGAGTGCCTGCCTCAGGCCATGAACATC
ACCTGCACAGGACGGGGACCAGACAACTGTATCCAGTGTGCCCACTACATTGACGGCCC
CCACTGCGTCAAGACCTGCCCGGCAGGAGTCATGGGAGAAAACAACACCCTGGTCTGGA
AGTACGCAGACGCCGGCCATGTGTGCCACCTGTGCCATCCAAACTGCACCTACGGATGC
ACTGGGCCAGGTCTTGAAGGCTGTCCAACGAATGGGCCTAAGATCCCGTCCATCGCCAC
TGGGATGGTGGGGGCCCTCCTCTTGCTGCTGGTGGTGGCCCTGGGGATCGGCCTCTTCAT
GTGA

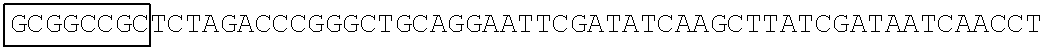

CTGGATTACAAAATTTGTGAAAGATTGACTGGTATTCTTAACTATGTTGCTCCTTTTACGC
TATGTGGATACGCTGCTTTAATGCCTTTGTATCATGCTATTGCTTCCCGTATGGCTTTCAT
TTTCTCCTCCTTGTATAAATCCTGGTTGCTGTCTCTTTATGAGGAGTTGTGGCCCGTTGTC
AGGCAACGTGGCGTGGTGTGCACTGTGTTTGCTGACGCAACCCCCACTGGTTGGGGCATT
GCCACCACCTGTCAGCTCCTTTCCGGGACTTTCGCTTTCCCCCTCCCTATTGCCACGGCGG
AACTCATCGCCGCCTGCCTTGCCCGCTGCTGGACAGGGGCTCGGCTGTTGGGCACTGACA
ATTCCGTGGTGTTGTCGGGGAAATCATCGTCCTTTCCTTGGCTGCTCGCCTGTGTTGCCAC
CTGGATTCTGCGCGGGACGTCCTTCTGCTACGTCCCTTCGGCCCTCAATCCAGCGGACCT
TCCTTCCCGCGGCCTGCTGCCGGCTCTGCGGCCTCTTCCGCGTCTTCGCCTTCGCCCTCAG
ACGAGTCGGATCTCCCTTTGGGCCGCCTCCCCGCATCGATACCGTCGACTAGCCGTACCT
TTAAGACCAATGACTTACAAGGCAGCTGTAGATCTTAGCCACTTTTTAAAAGAAAAGGG
GGGACTGGAAGGGCTAATTCACTCCCAAAGAAGACAAGATCTGCTTTTTGCCTGTACTG
GGTCTCTCTGGTTAGACCAGATCTGAGCCTGGGAGCTCTCTGGCTAACTAGGGAACCCAC
TGCTTAAGCCTCAATAAAGCTTGCCTTGAGTGCTTCAAGTAGTGTGTGCCCGTCTGTTGT
GTGACTCTGGTAACTAGAGATCCCTCAGACCCTTTTAGTCAGTGTGGAAAATCTCTAGCA

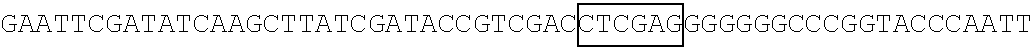

CGCCCTATAGTGAGTCGTATTACAATTCACTGGCCGTCGTTTTACAACGTCGTGACTGGG
AAAACCCTGGCGTTACCCAACTTAATCGCCTTGCAGCACATCCCCCTTTCGCCAGCTGGC
GTAATAGCGAAGAGGCCCGCACCGATCGCCCTTCCCAACAGTTGCGCAGCCTGAATGGC
GAATGGAAATTGTAAGCGTTAATATTTTGTTAAAATTCGCGTTAAATTTTTGTTAAATCA
GCTCATTTTTTAACCAATAGGCCGAAATCGGCAAAATCCCTTATAAATCAAAAGAATAG
ACCGAGATAGGGTTGAGTGTTGTTCCAGTTTGGAACAAGAGTCCACTATTAAAGAACGT
GGACTCCAACGTCAAAGGGCGAAAAACCGTCTATCAGGGCGATGGCCCACTACGTGAAC
CATCACCCTAATCAAGTTTTTTGGGGTCGAGGTGCCGTAAAGCACTAAATCGGAACCCTA
AAGGGAGCCCCCGATTTAGAGCTTGACGGGGAAAGCCGGCGAACGTGGCGAGAAAGGA
AGGGAAGAAAGCGAAAGGAGCGGGCGCTAGGGCGCTGGCAAGTGTAGCGGTCACGCTG
CGCGTAACCACCACACCCGCCGCGCTTAATGCGCCGCTACAGGGCGCGTCAGGTGGCAC
TTTTCGGGGAAATGTGCGCGGAACCCCTATTTGTTTATTTTTCTAAATACATTCAAATATG
TATCCGCTCATGAGACAATAACCCTGATAAATGCTTCAATAATATTGAAAAAGGAAGAG
TATGAGTATTCAACATTTCCGTGTCGCCCTTATTCCCTTTTTTGCGGCATTTTGCCTTCCTG
TTTTTGCTCACCCAGAAACGCTGGTGAAAGTAAAAGATGCTGAAGATCAGTTGGGTGCA
CGAGTGGGTTACATCGAACTGGATCTCAACAGCGGTAAGATCCTTGAGAGTTTTCGCCCC
GAAGAACGTTTTCCAATGATGAGCACTTTTAAAGTTCTGCTATGTGGCGCGGTATTATCC
CGTATTGACGCCGGGCAAGAGCAACTCGGTCGCCGCATACACTATTCTCAGAATGACTT
GGTTGAGTACTCACCAGTCACAGAAAAGCATCTTACGGATGGCATGACAGTAAGAGAAT
TATGCAGTGCTGCCATAACCATGAGTGATAACACTGCGGCCAACTTACTTCTGACAACGA
TCGGAGGACCGAAGGAGCTAACCGCTTTTTTGCACAACATGGGGGATCATGTAACTCGC
CTTGATCGTTGGGAACCGGAGCTGAATGAAGCCATACCAAACGACGAGCGTGACACCAC
GATGCCTGTAGCAATGGCAACAACGTTGCGCAAACTATTAACTGGCGAACTACTTACTCT
AGCTTCCCGGCAACAATTAATAGACTGGATGGAGGCGGATAAAGTTGCAGGACCACTTC
TGCGCTCGGCCCTTCCGGCTGGCTGGTTTATTGCTGATAAATCTGGAGCCGGTGAGCGTG
GGTCTCGCGGTATCATTGCAGCACTGGGGCCAGATGGTAAGCCCTCCCGTATCGTAGTTA
TCTACACGACGGGGAGTCAGGCAACTATGGATGAACGAAATAGACAGATCGCTGAGATA
GGTGCCTCACTGATTAAGCATTGGTAACTGTCAGACCAAGTTTACTCATATATACTTTAG
ATTGATTTAAAACTTCATTTTTAATTTAAAAGGATCTAGGTGAAGATCCTTTTTGATAATC
TCATGACCAAAATCCCTTAACGTGAGTTTTCGTTCCACTGAGCGTCAGACCCCGTAGAAA
AGATCAAAGGATCTTCTTGAGATCCTTTTTTTCTGCGCGTAATCTGCTGCTTGCAAACAA
AAAAACCACCGCTACCAGCGGTGGTTTGTTTGCCGGATCAAGAGCTACCAACTCTTTTTC
CGAAGGTAACTGGCTTCAGCAGAGCGCAGATACCAAATACTGTTCTTCTAGTGTAGCCGT
AGTTAGGCCACCACTTCAAGAACTCTGTAGCACCGCCTACATACCTCGCTCTGCTAATCC
TGTTACCAGTGGCTGCTGCCAGTGGCGATAAGTCGTGTCTTACCGGGTTGGACTCAAGAC
GATAGTTACCGGATAAGGCGCAGCGGTCGGGCTGAACGGGGGGTTCGTGCACACAGCCC
AGCTTGGAGCGAACGACCTACACCGAACTGAGATACCTACAGCGTGAGCTATGAGAAAG
CGCCACGCTTCCCGAAGGGAGAAAGGCGGACAGGTATCCGGTAAGCGGCAGGGTCGGA
ACAGGAGAGCGCACGAGGGAGCTTCCAGGGGGAAACGCCTGGTATCTTTATAGTCCTGT
CGGGTTTCGCCACCTCTGACTTGAGCGTCGATTTTTGTGATGCTCGTCAGGGGGGCGGAG
CCTATGGAAAAACGCCAGCAACGCGGCCTTTTTACGGTTCCTGGCCTTTTGCTGGCCTTT
TGCTCACATGTTCTTTCCTGCGTTATCCCCTGATTCTGTGGATAACCGTATTACCGCCTTT
GAGTGAGCTGATACCGCTCGCCGCAGCCGAACGACCGAGCGCAGCGAGTCAGTGAGCG
AGGAAGCGGAAGAGCGCCCAATACGCAAACCGCCTCTCCCCGCGCGTTGGCCGATTCAT
TAATGCAGCTGGCACGACAGGTTTCCCGACTGGAAAGCGGGCAGTGAGCGCAACGCAAT
TAATGTGAGTTAGCTCACTCATTAGGCACCCCAGGCTTTACACTTTATGCTTCCGGCTCGT
ATGTTGTGTGGAATTGTGAGCGGATAACAATTTCACACAGGAAACAGCTATGACCATGA
TTACGCCAAGCTCGAAATTAACCCTCACTAAAGGGAACAAAAGCTGGAGCTCCACCGCG

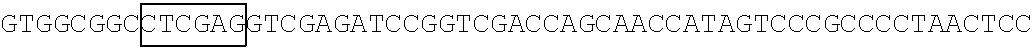

GCCCATCCCGCCCCTAACTCCGCCCAGTTCCGCCCATTCTCCGCCCCATGGCTGACTAAT
TTTTTTTATTTATGCAGAGGCCGAGGCCGCCTCGGCCTCTGAGCTATTCCAGAAGTAGTG
AGGAGGCTTTTTTGGAGGCCTAGGCTTTTGCAAAAAGCTTCGACGGTATCGATTGGCTCA
TGTCCAACATTACCGCCATGTTGACATTGATTATTGACTAGTTATTAATAGTAATCAATT
ACGGGGTCATTAGTTCATAGCCCATATATGGAGTTCCGCGTTACATAACTTACGGTAAAT
GGCCCGCCTGGCTGACCGCCCAACGACCCCCGCCCATTGACGTCAATAATGACGTATGTT
CCCATAGTAACGCCAATAGGGACTTTCCATTGACGTCAATGGGTGGAGTATTTACGGTAA
ACTGCCCACTTGGCAGTACATCAAGTGTATCATATGCCAAGTACGCCCCCTATTGACGTC
AATGACGGTAAATGGCCCGCCTGGCATTATGCCCAGTACATGACCTTATGGGACTTTCCT
ACTTGGCAGTACATCTACGTATTAGTCATCGCTATTACCATGGTGATGCGGTTTTGGCAG
TACATCAATGGGCGTGGATAGCGGTTTGACTCACGGGGATTTCCAAGTCTCCACCCCATT
GACGTCAATGGGAGTTTGTTTTGGCACCAAAATCAACGGGACTTTCCAAAATGTCGTAAC
AACTCCGCCCCATTGACGCAAATGGGCGGTAGGCGTGTACGGAATTCGGAGTGGCGAGC
CCTCAGATCCTGCATATAAGCAGCTGCTTTTTGCCTGTACTGGGTCTCTCTG

TABLE 14
Leader_R12-CD28tm/41BB-Z-T2A-tEGFR (SEQ ID NO: 42)

Leader
MLLLVTSLLLCELPHPAFLLIP
scFv R12
QEQLVESGGRLVTPGGSLTLSCKASGFDFSAYYMSWVRQAPGKGLEWIATI
YPSSGKTYYATWVNGRFTISSDNAQNTVDLQMNSLTAADRATYFCARDSYA
DDGALFNIWGPGTLVTISSGGGGSGGGGSGGGGSELVLTQSPSVSAALGSP
AKITCTLSSAHKTDTIDWYQQLQGEAPRYLMQVQSDGSYTKRPGVPDRFSG
SSSGADRYLIIPSVQADDEADYYCGADYIGGYVFGGGTQLTVTG
Hinge/spacer
ESKYGPPCPPCP
CD28tm
MFWVLVVVGGVLACYSLLVTVAFIIFWV
4-1BB
KRGRKKLLYIFKQPFMRPVQTTQEEDGCSCRFPEEEEGGCEL
CD3zeta
RVKFSRSADAPAYQQGQNQLYNELNLGRREEYDVLDKRRGRDPEMGGKPRR
KNPQEGLYNELQKDKMAEAYSEIGMKGERRRGKGHDGLYQGLSTATKDTYD
ALHMQALPPR
T2A
LEGGGEGRGSLLTCGDVEENPGPR
tEGFR
MLLLVTSLLLCELPHPAFLLIPRKVCNGIGIGEFKDSLSINATNIKHFKNC
TSISGDLHILPVAFRGDSFTHTPPLDPQELDILKTVKEITGFLLIQAWPEN
RTDLHAFENLEIIRGRTKQHGQFSLAVVSLNITSLGLRSLKEISDGDVIIS
GNKNLCYANTINWKKLFGTSGQKTKIISNRGENSCKATGQVCHALCSPEGC
WGPEPRDCVSCRNVSRGRECVDKCNLLEGEPREFVENSECIQCHPECLPQA
MNITCTGRGPDNCIQCAHYIDGPHCVKTCPAGVMGENNTLVWKYADAGHVC
HLCHPNCTYGCTGPGLEGCPTNGPKIPSIATGMVGALLLLLVVALGIGLFM

TABLE 15
R11 long spacer CAR: PJ_R11-CH2-CH3-41BB-Z-T2A-tEGFR (SEQ ID NO: 43)
GTTAGACCAGATCTGAGCCTGGGAGCTCTCTGGCTAACTAGGGAACCCACTGCTTAAGC
CTCAATAAAGCTTGCCTTGAGTGCTTCAAGTAGTGTGTGCCCGTCTGTTGTGTGACTCTG
GTAACTAGAGATCCCTCAGACCCTTTTAGTCAGTGTGGAAAATCTCTAGCAGTGGCGCCC
GAACAGGGACTTGAAAGCGAAAGGGAAACCAGAGGAGCTCTCTCGACGCAGGACTCGG
CTTGCTGAAGCGCGCACGGCAAGAGGCGAGGGGCGGCGACTGGTGAGTACGCCAAAAA
TTTTGACTAGCGGAGGCTAGAAGGAGAGAGATGGGTGCGAGAGCGTCAGTATTAAGCGG
GGGAGAATTAGATCGATGGGAAAAAATTCGGTTAAGGCCAGGGGGAAAGAAAAAATAT
AAATTAAAACATATAGTATGGGCAAGCAGGGAGCTAGAACGATTCGCAGTTAATCCTGG
CCTGTTAGAAACATCAGAAGGCTGTAGACAAATACTGGGACAGCTACAACCATCCCTTC
AGACAGGATCAGAAGAACTTAGATCATTATATAATACAGTAGCAACCCTCTATTGTGTG
CATCAAAGGATAGAGATAAAAGACACCAAGGAAGCTTTAGACAAGATAGAGGAAGAGC
AAAACAAAAGTAAGAAAAAAGCACAGCAAGCAGCAGCTGACACAGGACACAGCAATCA
GGTCAGCCAAAATTACCCTATAGTGCAGAACATCCAGGGGCAAATGGTACATCAGGCCA
TATCACCTAGAACTTTAAATGCATGGGTAAAAGTAGTAGAAGAGAAGGCTTTCAGCCCA
GAAGTGATACCCATGTTTTCAGCATTATCAGAAGGAGCCACCCCACAAGATTTAAACAC
CATGCTAAACACAGTGGGGGGACATCAAGCAGCCATGCAAATGTTAAAAGAGACCATCA
ATGAGGAAGCTGCAGGCAAAGAGAAGAGTGGTGCAGAGAGAAAAAAGAGCAGTGGGA
ATAGGAGCTTTGTTCCTTGGGTTCTTGGGAGCAGCAGGAAGCACTATGGGCGCAGCGTC
AATGACGCTGACGGTACAGGCCAGACAATTATTGTCTGGTATAGTGCAGCAGCAGAACA
ATTTGCTGAGGGCTATTGAGGCGCAACAGCATCTGTTGCAACTCACAGTCTGGGGCATCA
AGCAGCTCCAGGCAAGAATCCTGGCTGTGGAAAGATACCTAAAGGATCAACAGCTCCTG
GGGATTTGGGGTTGCTCTGGAAAACTCATTTGCACCACTGCTGTGCCTTGGATCTACAAA
TGGCAGTATTCATCCACAATTTTAAAAGAAAAGGGGGGATTGGGGGGTACAGTGCAGGG
GAAAGAATAGTAGACATAATAGCAACAGACATACAAACTAAAGAATTACAAAAACAAA
TTACAAAAATTCAAAATTTTCGGGTTTATTACAGGGACAGCAGAGATCCAGTTTGGGGAT
CAATTGCATGAAGAATCTGCTTAGGGTTAGGCGTTTTGCGCTGCTTCGCGAGGATCTGCG
ATCGCTCCGGTGCCCGTCAGTGGGCAGAGCGCACATCGCCCACAGTCCCCGAGAAGTTG
GGGGGAGGGGTCGGCAATTGAACCGGTGCCTAGAGAAGGTGGCGCGGGGTAAACTGGG
AAAGTGATGTCGTGTACTGGCTCCGCCTTTTTCCCGAGGGTGGGGGAGAACCGTATATAA
GTGCAGTAGTCGCCGTGAACGTTCTTTTTCGCAACGGGTTTGCCGCCAGAACACAGCTGA
AGCTTCGAGGGGCTCGCATCTCTCCTTCACGCGCCCGCCGCCCTACCTGAGGCCGCCATC
CACGCCGGTTGAGTCGCGTTCTGCCGCCTCCCGCCTGTGGTGCCTCCTGAACTGCGTCCG
CCGTCTAGGTAAGTTTAAAGCTCAGGTCGAGACCGGGCCTTTGTCCGGCGCTCCCTTGGA
GCCTACCTAGACTCAGCCGGCTCTCCACGCTTTGCCTGACCCTGCTTGCTCAACTCTACGT
CTTTGTTTCGTTTTCTGTTCTGCGCCGTTACAGATCCAAGCTGTGACCGGCGCCTACG

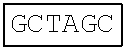

scFv R12

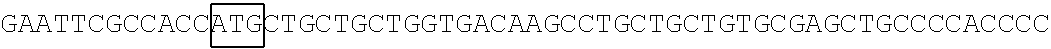

GCCTTTCTGCTGATCCCCCAGAGCGTGAAAGAGTCCGAGGGCGACCTGGTCACACCAGC
CGGCAACCTGACCCTGACCTGTACCGCCAGCGGCAGCGACATCAACGACTACCCCATCT
CTTGGGTCCGCCAGGCTCCTGGCAAGGGACTGGAATGGATCGGCTTCATCAACAGCGGC
GGCAGCACTTGGTACGCCAGCTGGGTCAAAGGCCGGTTCACCATCAGCCGGACCAGCAC
CACCGTGGACCTGAAGATGACAAGCCTGACCACCGACGACACCGCCACCTACTTTTGCG
CCAGAGGCTACAGCACCTACTACGGCGACTTCAACATCTGGGGCCCTGGCACCCTGGTC
ACAATCTCTAGCGGCGGAGGCGGCAGCGGAGGTGGAGGAAGTGGCGGCGGAGGATCCG
AGCTGGTCATGACCCAGACCCCCAGCAGCACATCTGGCGCCGTGGGCGGCACCGTGACC
ATCAATTGCCAGGCCAGCCAGAGCATCGACAGCAACCTGGCCTGGTTCCAGCAGAAGCC
CGGCCAGCCCCCCACCCTGCTGATCTACAGAGCCTCCAACCTGGCCAGCGGCGTGCCAA
GCAGATTCAGCGGCAGCAGATCTGGCACCGAGTACACCCTGACCATCTCCGGCGTGCAG
AGAGAGGACGCCGCTACCTATTACTGCCTGGGCGGCGTGGGCAACGTGTCCTACAGAAC
CAGCTTCGGCGGAGGTACTGAGGTGGTCGTCAAA
Hinge/Spacer

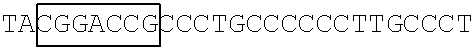

CH2
GCCCCCGAGTTCCTGGGCGGACCCAGCGTGTTCCTGTTCCCCCCCAAGCCCAAGGACACC
CTGATGATCAGCCGGACCCCCGAGGTGACCTGCGTGGTGGTGGACGTGAGCCAGGAAGA
TCCCGAGGTCCAGTTCAATTGGTACGTGGACGGCGTGGAAGTGCACAACGCCAAGACCA
AGCCCAGAGAGGAACAGTTCAACAGCACCTACCGGGTGGTGTCTGTGCTGACCGTGCTG
CACCAGGACTGGCTGAACGGCAAAGAATACAAGTGCAAGGTGTCCAACAAGGGCCTGC
CCAGCAGCATCGAAAAGACCATCAGCAAGGCCAAG
CH3
GGCCAGCCTCGCGAGCCCCAGGTGTACACCCTGCCTCCCTCCCAGGAAGAGATGACCAA
GAACCAGGTGTCCCTGACCTGCCTGGTGAAGGGCTTCTACCCCAGCGACATCGCCGTGG
AGTGGGAGAGCAACGGCCAGCCTGAGAACAACTACAAGACCACCCCTCCCGTGCTGGAC
AGCGACGGCAGCTTCTTCCTGTACAGCCGGCTGACCGTGGACAAGAGCCGGTGGCAGGA
AGGCAACGTCTTTAGCTGCAGCGTGATGCACGAGGCCCTGCACAACCACTACACCCAGA
AGAGCCTGAGCCTGTCCCTGGGCAAG
4-1BB
ATGTTCTGGGTGCTGGTGGTGGTGGGCGGGGTGCTGGCCTGCTACAGCCTGCTGGTGACA
GTGGCCTTCATCATCTTTTGGGTGAAACGGGGCAGAAAGAAACTCCTGTATATATTCAAA
CAACCATTTATGAGACCAGTACAAACTACTCAAGAGGAAGATGGCTGTAGCTGCCGATT
TCCAGAAGAAGAAGAAGGAGGATGTGAACTG
CD3zeta
CGGGTGAAGTTCAGCAGAAGCGCCGACGCCCCTGCCTACCAGCAGGGCCAGAATCAGCT
GTACAACGAGCTGAACCTGGGCAGAAGGGAAGAGTACGACGTCCTGGATAAGCGGAGA
GGCCGGGACCCTGAGATGGGCGGCAAGCCTCGGCGGAAGAACCCCCAGGAAGGCCTGT
ATAACGAACTGCAGAAAGACAAGATGGCCGAGGCCTACAGCGAGATCGGCATGAAGGG
CGAGCGGAGGCGGGGCAAGGGCCACGACGGCCTGTATCAGGGCCTGTCCACCGCCACCA
AGGATACCTACGACGCCCTGCACATGCAGGCCCTGCCCCCAAGG
T2A

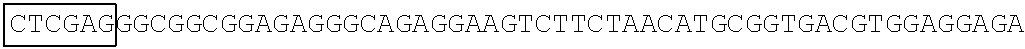

ATCCCGGCCCTAGG
tEGFR
ATGCTTCTCCTGGTGACAAGCCTTCTGCTCTGTGAGTTACCACACCCAGCATTCCTCCTGA
TCCCACGCAAAGTGTGTAACGGAATAGGTATTGGTGAATTTAAAGACTCACTCTCCATAA
ATGCTACGAATATTAAACACTTCAAAAACTGCACCTCCATCAGTGGCGATCTCCACATCC

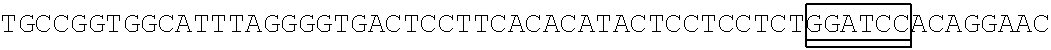

TGGATATTCTGAAAACCGTAAAGGAAATCACAGGGTTTTTGCTGATTCAGGCTTGGCCTG
AAAACAGGACGGACCTCCATGCCTTTGAGAACCTAGAAATCATACGCGGCAGGACCAAG
CAACATGGTCAGTTTTCTCTTGCAGTCGTCAGCCTGAACATAACATCCTTGGGATTACGC
TCCCTCAAGGAGATAAGTGATGGAGATGTGATAATTTCAGGAAACAAAAATTTGTGCTA
TGCAAATACAATAAACTGGAAAAAACTGTTTGGGACCTCCGGTCAGAAAACCAAAATTA
TAAGCAACAGAGGTGAAAACAGCTGCAAGGCCACAGGCCAGGTCTGCCATGCCTTGTGC
TCCCCCGAGGGCTGCTGGGGCCCGGAGCCCAGGGACTGCGTCTCTTGCCGGAATGTCAG
CCGAGGCAGGGAATGCGTGGACAAGTGCAACCTTCTGGAGGGTGAGCCAAGGGAGTTTG
TGGAGAACTCTGAGTGCATACAGTGCCACCCAGAGTGCCTGCCTCAGGCCATGAACATC
ACCTGCACAGGACGGGGACCAGACAACTGTATCCAGTGTGCCCACTACATTGACGGCCC
CCACTGCGTCAAGACCTGCCCGGCAGGAGTCATGGGAGAAAACAACACCCTGGTCTGGA
AGTACGCAGACGCCGGCCATGTGTGCCACCTGTGCCATCCAAACTGCACCTACGGATGC
ACTGGGCCAGGTCTTGAAGGCTGTCCAACGAATGGGCCTAAGATCCCGTCCATCGCCAC
TGGGATGGTGGGGGCCCTCCTCTTGCTGCTGGTGGTGGCCCTGGGGATCGGCCTCTTCAT
GTGA

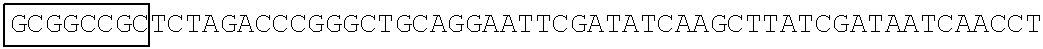

CTGGATTACAAAATTTGTGAAAGATTGACTGGTATTCTTAACTATGTTGCTCCTTTTACGC
TATGTGGATACGCTGCTTTAATGCCTTTGTATCATGCTATTGCTTCCCGTATGGCTTTCAT
TTTCTCCTCCTTGTATAAATCCTGGTTGCTGTCTCTTTATGAGGAGTTGTGGCCCGTTGTC
AGGCAACGTGGCGTGGTGTGCACTGTGTTTGCTGACGCAACCCCCACTGGTTGGGGCATT
GCCACCACCTGTCAGCTCCTTTCCGGGACTTTCGCTTTCCCCCTCCCTATTGCCACGGCGG
AACTCATCGCCGCCTGCCTTGCCCGCTGCTGGACAGGGGCTCGGCTGTTGGGCACTGACA
ATTCCGTGGTGTTGTCGGGGAAATCATCGTCCTTTCCTTGGCTGCTCGCCTGTGTTGCCAC
CTGGATTCTGCGCGGGACGTCCTTCTGCTACGTCCCTTCGGCCCTCAATCCAGCGGACCT
TCCTTCCCGCGGCCTGCTGCCGGCTCTGCGGCCTCTTCCGCGTCTTCGCCTTCGCCCTCAG
ACGAGTCGGATCTCCCTTTGGGCCGCCTCCCCGCATCGATACCGTCGACTAGCCGTACCT
TTAAGACCAATGACTTACAAGGCAGCTGTAGATCTTAGCCACTTTTTAAAAGAAAAGGG
GGGACTGGAAGGGCTAATTCACTCCCAAAGAAGACAAGATCTGCTTTTTGCCTGTACTG
GGTCTCTCTGGTTAGACCAGATCTGAGCCTGGGAGCTCTCTGGCTAACTAGGGAACCCAC
TGCTTAAGCCTCAATAAAGCTTGCCTTGAGTGCTTCAAGTAGTGTGTGCCCGTCTGTTGT
GTGACTCTGGTAACTAGAGATCCCTCAGACCCTTTTAGTCAGTGTGGAAAATCTCTAGCA

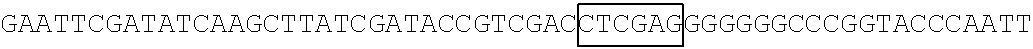

CGCCCTATAGTGAGTCGTATTACAATTCACTGGCCGTCGTTTTACAACGTCGTGACTGGG
AAAACCCTGGCGTTACCCAACTTAATCGCCTTGCAGCACATCCCCCTTTCGCCAGCTGGC
GTAATAGCGAAGAGGCCCGCACCGATCGCCCTTCCCAACAGTTGCGCAGCCTGAATGGC
GAATGGAAATTGTAAGCGTTAATATTTTGTTAAAATTCGCGTTAAATTTTTGTTAAATCA
GCTCATTTTTTAACCAATAGGCCGAAATCGGCAAAATCCCTTATAAATCAAAAGAATAG
ACCGAGATAGGGTTGAGTGTTGTTCCAGTTTGGAACAAGAGTCCACTATTAAAGAACGT
GGACTCCAACGTCAAAGGGCGAAAAACCGTCTATCAGGGCGATGGCCCACTACGTGAAC
CATCACCCTAATCAAGTTTTTTGGGGTCGAGGTGCCGTAAAGCACTAAATCGGAACCCTA
AAGGGAGCCCCCGATTTAGAGCTTGACGGGGAAAGCCGGCGAACGTGGCGAGAAAGGA
AGGGAAGAAAGCGAAAGGAGCGGGCGCTAGGGCGCTGGCAAGTGTAGCGGTCACGCTG
CGCGTAACCACCACACCCGCCGCGCTTAATGCGCCGCTACAGGGCGCGTCAGGTGGCAC
TTTTCGGGGAAATGTGCGCGGAACCCCTATTTGTTTATTTTTCTAAATACATTCAAATATG
TATCCGCTCATGAGACAATAACCCTGATAAATGCTTCAATAATATTGAAAAAGGAAGAG
TATGAGTATTCAACATTTCCGTGTCGCCCTTATTCCCTTTTTTGCGGCATTTTGCCTTCCTG
TTTTTGCTCACCCAGAAACGCTGGTGAAAGTAAAAGATGCTGAAGATCAGTTGGGTGCA
CGAGTGGGTTACATCGAACTGGATCTCAACAGCGGTAAGATCCTTGAGAGTTTTCGCCCC
GAAGAACGTTTTCCAATGATGAGCACTTTTAAAGTTCTGCTATGTGGCGCGGTATTATCC
CGTATTGACGCCGGGCAAGAGCAACTCGGTCGCCGCATACACTATTCTCAGAATGACTT
GGTTGAGTACTCACCAGTCACAGAAAAGCATCTTACGGATGGCATGACAGTAAGAGAAT
TATGCAGTGCTGCCATAACCATGAGTGATAACACTGCGGCCAACTTACTTCTGACAACGA
TCGGAGGACCGAAGGAGCTAACCGCTTTTTTGCACAACATGGGGGATCATGTAACTCGC
CTTGATCGTTGGGAACCGGAGCTGAATGAAGCCATACCAAACGACGAGCGTGACACCAC
GATGCCTGTAGCAATGGCAACAACGTTGCGCAAACTATTAACTGGCGAACTACTTACTCT
AGCTTCCCGGCAACAATTAATAGACTGGATGGAGGCGGATAAAGTTGCAGGACCACTTC
TGCGCTCGGCCCTTCCGGCTGGCTGGTTTATTGCTGATAAATCTGGAGCCGGTGAGCGTG
GGTCTCGCGGTATCATTGCAGCACTGGGGCCAGATGGTAAGCCCTCCCGTATCGTAGTTA
TCTACACGACGGGGAGTCAGGCAACTATGGATGAACGAAATAGACAGATCGCTGAGATA
GGTGCCTCACTGATTAAGCATTGGTAACTGTCAGACCAAGTTTACTCATATATACTTTAG
ATTGATTTAAAACTTCATTTTTAATTTAAAAGGATCTAGGTGAAGATCCTTTTTGATAATC
TCATGACCAAAATCCCTTAACGTGAGTTTTCGTTCCACTGAGCGTCAGACCCCGTAGAAA
AGATCAAAGGATCTTCTTGAGATCCTTTTTTTCTGCGCGTAATCTGCTGCTTGCAAACAA
AAAAACCACCGCTACCAGCGGTGGTTTGTTTGCCGGATCAAGAGCTACCAACTCTTTTTC
CGAAGGTAACTGGCTTCAGCAGAGCGCAGATACCAAATACTGTTCTTCTAGTGTAGCCGT
AGTTAGGCCACCACTTCAAGAACTCTGTAGCACCGCCTACATACCTCGCTCTGCTAATCC
TGTTACCAGTGGCTGCTGCCAGTGGCGATAAGTCGTGTCTTACCGGGTTGGACTCAAGAC
GATAGTTACCGGATAAGGCGCAGCGGTCGGGCTGAACGGGGGGTTCGTGCACACAGCCC
AGCTTGGAGCGAACGACCTACACCGAACTGAGATACCTACAGCGTGAGCTATGAGAAAG
CGCCACGCTTCCCGAAGGGAGAAAGGCGGACAGGTATCCGGTAAGCGGCAGGGTCGGA
ACAGGAGAGCGCACGAGGGAGCTTCCAGGGGGAAACGCCTGGTATCTTTATAGTCCTGT
CGGGTTTCGCCACCTCTGACTTGAGCGTCGATTTTTGTGATGCTCGTCAGGGGGGCGGAG
CCTATGGAAAAACGCCAGCAACGCGGCCTTTTTACGGTTCCTGGCCTTTTGCTGGCCTTT
TGCTCACATGTTCTTTCCTGCGTTATCCCCTGATTCTGTGGATAACCGTATTACCGCCTTT
GAGTGAGCTGATACCGCTCGCCGCAGCCGAACGACCGAGCGCAGCGAGTCAGTGAGCG
AGGAAGCGGAAGAGCGCCCAATACGCAAACCGCCTCTCCCCGCGCGTTGGCCGATTCAT
TAATGCAGCTGGCACGACAGGTTTCCCGACTGGAAAGCGGGCAGTGAGCGCAACGCAAT
TAATGTGAGTTAGCTCACTCATTAGGCACCCCAGGCTTTACACTTTATGCTTCCGGCTCGT
ATGTTGTGTGGAATTGTGAGCGGATAACAATTTCACACAGGAAACAGCTATGACCATGA
TTACGCCAAGCTCGAAATTAACCCTCACTAAAGGGAACAAAAGCTGGAGCTCCACCGCG

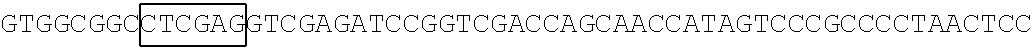

GCCCATCCCGCCCCTAACTCCGCCCAGTTCCGCCCATTCTCCGCCCCATGGCTGACTAAT
TTTTTTTATTTATGCAGAGGCCGAGGCCGCCTCGGCCTCTGAGCTATTCCAGAAGTAGTG
AGGAGGCTTTTTTGGAGGCCTAGGCTTTTGCAAAAAGCTTCGACGGTATCGATTGGCTCA
TGTCCAACATTACCGCCATGTTGACATTGATTATTGACTAGTTATTAATAGTAATCAATT
ACGGGGTCATTAGTTCATAGCCCATATATGGAGTTCCGCGTTACATAACTTACGGTAAAT
GGCCCGCCTGGCTGACCGCCCAACGACCCCCGCCCATTGACGTCAATAATGACGTATGTT
CCCATAGTAACGCCAATAGGGACTTTCCATTGACGTCAATGGGTGGAGTATTTACGGTAA
ACTGCCCACTTGGCAGTACATCAAGTGTATCATATGCCAAGTACGCCCCCTATTGACGTC
AATGACGGTAAATGGCCCGCCTGGCATTATGCCCAGTACATGACCTTATGGGACTTTCCT
ACTTGGCAGTACATCTACGTATTAGTCATCGCTATTACCATGGTGATGCGGTTTTGGCAG
TACATCAATGGGCGTGGATAGCGGTTTGACTCACGGGGATTTCCAAGTCTCCACCCCATT
GACGTCAATGGGAGTTTGTTTTGGCACCAAAATCAACGGGACTTTCCAAAATGTCGTAAC
AACTCCGCCCCATTGACGCAAATGGGCGGTAGGCGTGTACGGAATTCGGAGTGGCGAGC
CCTCAGATCCTGCATATAAGCAGCTGCTTTTTGCCTGTACTGGGTCTCTCTG

TABLE 16
Leader_R11-Hinge-CH2-CH3-CD28tm/41BB-Z-T2A-tEGFR (SEQ ID NO: 44)
Leader: MLLLVTSLLLCELPHPAFLLIP
R11 scFv
QSVKESEGDLVTPAGNLTLTCTASGSDINDYPISWVRQAPGKGLEWIGFINSGGSTWYASWV
KGRFTISRTSTTVDLKMTSLTTDDTATYFCARGYSTYYGDFNIWGPGTLVTISSGGGGSGGG
GSGGGGSELVMTQTPSSTSGAVGGTVTINCQASQSIDSNLAWFQQKPGQPPTLLIYRASNLAS
GVPSRFSGSRSGTEYTLTISGVQREDAATYYCLGGVGNVSYRTSFGGGTEVVVK
Hinge/Spacer: ESKYGPPCPPCP
CH2

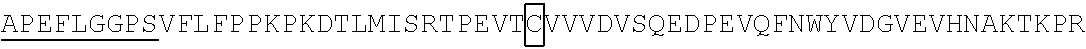

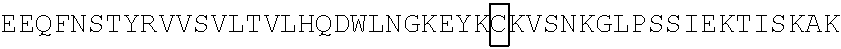

CH3

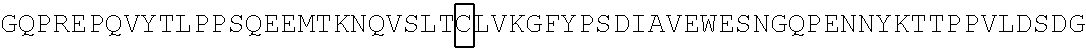

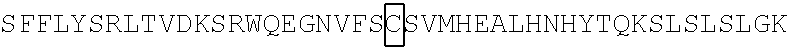

CD28tm: MFWVLVVVGGVLACYSLLVTVAFIIFWV
4-1BB: KRGRKKLLYIFKQPFMRPVQTTQEEDGCSCRFPEEEEGGCEL
CD3zeta
RVKFSRSADAPAYQQGQNQLYNELNLGRREEYDVLDKRRGRDPEMGGKPRRKNPQEGLYN
ELQKDKMAEAYSEIGMKGERRRGKGHDGLYQGLSTATKDTYDALHMQALPPR
T2A: LEGGGEGRGSLLTCGDVEENPGPR
tEGFR
MLLLVTSLLLCELPHPAFLLIPRKVCNGIGIGEFKDSLSINATNIKHFKNCTSISGDLHILPVAFR
GDSFTHTPPLDPQELDILKTVKEITGFLLIQAWPENRTDLHAFENLEIIRGRTKQHGQFSLAVV
SLNITSLGLRSLKEISDGDVIISGNKNLCYANTINWKKLFGTSGQKTKIISNRGENSCKATGQV
CHALCSPEGCWGPEPRDCVSCRNVSRGRECVDKCNLLEGEPREFVENSECIQCHPECLPQAM
NITCTGRGPDNCIQCAHYIDGPHCVKTCPAGVMGENNTLVWKYADAGHVCHLCHPNCTYG
CTGPGLEGCPTNGPKIPSIATGMVGALLLLLVVALGIGLFM

TABLE 17
R11 intermediate spacer CAR: PJ_R11-CH3-41BB-Z-T2A-tEGFR (SEQ ID NO: 45)
GTTAGACCAGATCTGAGCCTGGGAGCTCTCTGGCTAACTAGGGAACCCACTGCTTAAGC
CTCAATAAAGCTTGCCTTGAGTGCTTCAAGTAGTGTGTGCCCGTCTGTTGTGTGACTCTG
GTAACTAGAGATCCCTCAGACCCTTTTAGTCAGTGTGGAAAATCTCTAGCAGTGGCGCCC
GAACAGGGACTTGAAAGCGAAAGGGAAACCAGAGGAGCTCTCTCGACGCAGGACTCGG
CTTGCTGAAGCGCGCACGGCAAGAGGCGAGGGGCGGCGACTGGTGAGTACGCCAAAAA
TTTTGACTAGCGGAGGCTAGAAGGAGAGAGATGGGTGCGAGAGCGTCAGTATTAAGCGG
GGGAGAATTAGATCGATGGGAAAAAATTCGGTTAAGGCCAGGGGGAAAGAAAAAATAT
AAATTAAAACATATAGTATGGGCAAGCAGGGAGCTAGAACGATTCGCAGTTAATCCTGG
CCTGTTAGAAACATCAGAAGGCTGTAGACAAATACTGGGACAGCTACAACCATCCCTTC
AGACAGGATCAGAAGAACTTAGATCATTATATAATACAGTAGCAACCCTCTATTGTGTG
CATCAAAGGATAGAGATAAAAGACACCAAGGAAGCTTTAGACAAGATAGAGGAAGAGC
AAAACAAAAGTAAGAAAAAAGCACAGCAAGCAGCAGCTGACACAGGACACAGCAATCA
GGTCAGCCAAAATTACCCTATAGTGCAGAACATCCAGGGGCAAATGGTACATCAGGCCA
TATCACCTAGAACTTTAAATGCATGGGTAAAAGTAGTAGAAGAGAAGGCTTTCAGCCCA
GAAGTGATACCCATGTTTTCAGCATTATCAGAAGGAGCCACCCCACAAGATTTAAACAC
CATGCTAAACACAGTGGGGGGACATCAAGCAGCCATGCAAATGTTAAAAGAGACCATCA
ATGAGGAAGCTGCAGGCAAAGAGAAGAGTGGTGCAGAGAGAAAAAAGAGCAGTGGGA
ATAGGAGCTTTGTTCCTTGGGTTCTTGGGAGCAGCAGGAAGCACTATGGGCGCAGCGTC
AATGACGCTGACGGTACAGGCCAGACAATTATTGTCTGGTATAGTGCAGCAGCAGAACA
ATTTGCTGAGGGCTATTGAGGCGCAACAGCATCTGTTGCAACTCACAGTCTGGGGCATCA
AGCAGCTCCAGGCAAGAATCCTGGCTGTGGAAAGATACCTAAAGGATCAACAGCTCCTG
GGGATTTGGGGTTGCTCTGGAAAACTCATTTGCACCACTGCTGTGCCTTGGATCTACAAA
TGGCAGTATTCATCCACAATTTTAAAAGAAAAGGGGGGATTGGGGGGTACAGTGCAGGG
GAAAGAATAGTAGACATAATAGCAACAGACATACAAACTAAAGAATTACAAAAACAAA
TTACAAAAATTCAAAATTTTCGGGTTTATTACAGGGACAGCAGAGATCCAGTTTGGGGAT
CAATTGCATGAAGAATCTGCTTAGGGTTAGGCGTTTTGCGCTGCTTCGCGAGGATCTGCG
ATCGCTCCGGTGCCCGTCAGTGGGCAGAGCGCACATCGCCCACAGTCCCCGAGAAGTTG
GGGGGAGGGGTCGGCAATTGAACCGGTGCCTAGAGAAGGTGGCGCGGGGTAAACTGGG
AAAGTGATGTCGTGTACTGGCTCCGCCTTTTTCCCGAGGGTGGGGGAGAACCGTATATAA
GTGCAGTAGTCGCCGTGAACGTTCTTTTTCGCAACGGGTTTGCCGCCAGAACACAGCTGA
AGCTTCGAGGGGCTCGCATCTCTCCTTCACGCGCCCGCCGCCCTACCTGAGGCCGCCATC
CACGCCGGTTGAGTCGCGTTCTGCCGCCTCCCGCCTGTGGTGCCTCCTGAACTGCGTCCG
CCGTCTAGGTAAGTTTAAAGCTCAGGTCGAGACCGGGCCTTTGTCCGGCGCTCCCTTGGA
GCCTACCTAGACTCAGCCGGCTCTCCACGCTTTGCCTGACCCTGCTTGCTCAACTCTACGT
CTTTGTTTCGTTTTCTGTTCTGCGCCGTTACAGATCCAAGCTGTGACCGGCGCCTACG

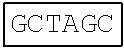

R11 scFV

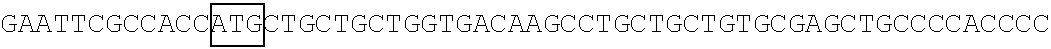

GCCTTTCTGCTGATCCCCCAGAGCGTGAAAGAGTCCGAGGGCGACCTGGTCACACCAGC
CGGCAACCTGACCCTGACCTGTACCGCCAGCGGCAGCGACATCAACGACTACCCCATCT
CTTGGGTCCGCCAGGCTCCTGGCAAGGGACTGGAATGGATCGGCTTCATCAACAGCGGC
GGCAGCACTTGGTACGCCAGCTGGGTCAAAGGCCGGTTCACCATCAGCCGGACCAGCAC
CACCGTGGACCTGAAGATGACAAGCCTGACCACCGACGACACCGCCACCTACTTTTGCG
CCAGAGGCTACAGCACCTACTACGGCGACTTCAACATCTGGGGCCCTGGCACCCTGGTC
ACAATCTCTAGCGGCGGAGGCGGCAGCGGAGGTGGAGGAAGTGGCGGCGGAGGATCCG
AGCTGGTCATGACCCAGACCCCCAGCAGCACATCTGGCGCCGTGGGCGGCACCGTGACC
ATCAATTGCCAGGCCAGCCAGAGCATCGACAGCAACCTGGCCTGGTTCCAGCAGAAGCC
CGGCCAGCCCCCCACCCTGCTGATCTACAGAGCCTCCAACCTGGCCAGCGGCGTGCCAA
GCAGATTCAGCGGCAGCAGATCTGGCACCGAGTACACCCTGACCATCTCCGGCGTGCAG
AGAGAGGACGCCGCTACCTATTACTGCCTGGGCGGCGTGGGCAACGTGTCCTACAGAAC
CAGCTTCGGCGGAGGTACTGAGGTGGTCGTCAAA
Hinge/Spacer

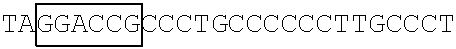

CH2
GCCCCCGAGTTCCTGGGCGGACCCAGCGTGTTCCTGTTCCCCCCCAAGCCCAAGGACACC
CTGATGATCAGCCGGACCCCCGAGGTGACCTGCGTGGTGGTGGACGTGAGCCAGGAAGA
TCCCGAGGTCCAGTTCAATTGGTACGTGGACGGCGTGGAAGTGCACAACGCCAAGACCA
AGCCCAGAGAGGAACAGTTCAACAGCACCTACCGGGTGGTGTCTGTGCTGACCGTGCTG
CACCAGGACTGGCTGAACGGCAAAGAATACAAGTGCAAGGTGTCCAACAAGGGCCTGC
CCAGCAGCATCGAAAAGACCATCAGCAAGGCCAAG
CH3
GGCCAGCCTCGCGAGCCCCAGGTGTACACCCTGCCTCCCTCCCAGGAAGAGATGACCAA
GAACCAGGTGTCCCTGACCTGCCTGGTGAAGGGCTTCTACCCCAGCGACATCGCCGTGG
AGTGGGAGAGCAACGGCCAGCCTGAGAACAACTACAAGACCACCCCTCCCGTGCTGGAC
AGCGACGGCAGCTTCTTCCTGTACAGCCGGCTGACCGTGGACAAGAGCCGGTGGCAGGA
AGGCAACGTCTTTAGCTGCAGCGTGATGCACGAGGCCCTGCACAACCACTACACCCAGA
AGAGCCTGAGCCTGTCCCTGGGCAAG
4-1BB
ATGTTCTGGGTGCTGGTGGTGGTGGGCGGGGTGCTGGCCTGCTACAGCCTGCTGGTGACA
GTGGCCTTCATCATCTTTTGGGTGAAACGGGGCAGAAAGAAACTCCTGTATATATTCAAA
CAACCATTTATGAGACCAGTACAAACTACTCAAGAGGAAGATGGCTGTAGCTGCCGATT
TCCAGAAGAAGAAGAAGGAGGATGTGAACTG
CD3zeta
CGGGTGAAGTTCAGCAGAAGCGCCGACGCCCCTGCCTACCAGCAGGGCCAGAATCAGCT
GTACAACGAGCTGAACCTGGGCAGAAGGGAAGAGTACGACGTCCTGGATAAGCGGAGA
GGCCGGGACCCTGAGATGGGCGGCAAGCCTCGGCGGAAGAACCCCCAGGAAGGCCTGT
ATAACGAACTGCAGAAAGACAAGATGGCCGAGGCCTACAGCGAGATCGGCATGAAGGG
CGAGCGGAGGCGGGGCAAGGGCCACGACGGCCTGTATCAGGGCCTGTCCACCGCCACCA
AGGATACCTACGACGCCCTGCACATGCAGGCCCTGCCCCCAAGG
T2A

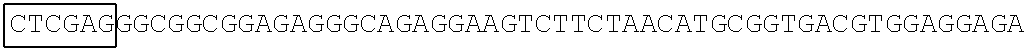

ATCCCGGCCCTAGG
tEGFR
ATGCTTCTCCTGGTGACAAGCCTTCTGCTCTGTGAGTTACCACACCCAGCATTCCTCCTGA
TCCCACGCAAAGTGTGTAACGGAATAGGTATTGGTGAATTTAAAGACTCACTCTCCATAA
ATGCTACGAATATTAAACACTTCAAAAACTGCACCTCCATCAGTGGCGATCTCCACATCC

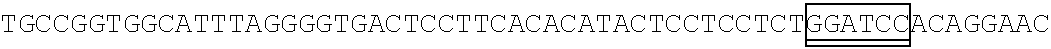

TGGATATTCTGAAAACCGTAAAGGAAATCACAGGGTTTTTGCTGATTCAGGCTTGGCCTG
AAAACAGGACGGACCTCCATGCCTTTGAGAACCTAGAAATCATACGCGGCAGGACCAAG
CAACATGGTCAGTTTTCTCTTGCAGTCGTCAGCCTGAACATAACATCCTTGGGATTACGC
TCCCTCAAGGAGATAAGTGATGGAGATGTGATAATTTCAGGAAACAAAAATTTGTGCTA
TGCAAATACAATAAACTGGAAAAAACTGTTTGGGACCTCCGGTCAGAAAACCAAAATTA
TAAGCAACAGAGGTGAAAACAGCTGCAAGGCCACAGGCCAGGTCTGCCATGCCTTGTGC
TCCCCCGAGGGCTGCTGGGGCCCGGAGCCCAGGGACTGCGTCTCTTGCCGGAATGTCAG
CCGAGGCAGGGAATGCGTGGACAAGTGCAACCTTCTGGAGGGTGAGCCAAGGGAGTTTG
TGGAGAACTCTGAGTGCATACAGTGCCACCCAGAGTGCCTGCCTCAGGCCATGAACATC
ACCTGCACAGGACGGGGACCAGACAACTGTATCCAGTGTGCCCACTACATTGACGGCCC
CCACTGCGTCAAGACCTGCCCGGCAGGAGTCATGGGAGAAAACAACACCCTGGTCTGGA
AGTACGCAGACGCCGGCCATGTGTGCCACCTGTGCCATCCAAACTGCACCTACGGATGC
ACTGGGCCAGGTCTTGAAGGCTGTCCAACGAATGGGCCTAAGATCCCGTCCATCGCCAC
TGGGATGGTGGGGGCCCTCCTCTTGCTGCTGGTGGTGGCCCTGGGGATCGGCCTCTTCAT

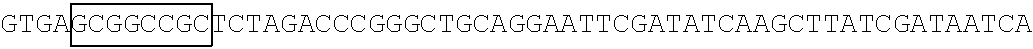

ACCTCTGGATTACAAAATTTGTGAAAGATTGACTGGTATTCTTAACTATGTTGCTCCTTTT
ACGCTATGTGGATACGCTGCTTTAATGCCTTTGTATCATGCTATTGCTTCCCGTATGGCTT
TCATTTTCTCCTCCTTGTATAAATCCTGGTTGCTGTCTCTTTATGAGGAGTTGTGGCCCGT
TGTCAGGCAACGTGGCGTGGTGTGCACTGTGTTTGCTGACGCAACCCCCACTGGTTGGGG
CATTGCCACCACCTGTCAGCTCCTTTCCGGGACTTTCGCTTTCCCCCTCCCTATTGCCACG
GCGGAACTCATCGCCGCCTGCCTTGCCCGCTGCTGGACAGGGGCTCGGCTGTTGGGCACT
GACAATTCCGTGGTGTTGTCGGGGAAATCATCGTCCTTTCCTTGGCTGCTCGCCTGTGTTG
CCACCTGGATTCTGCGCGGGACGTCCTTCTGCTACGTCCCTTCGGCCCTCAATCCAGCGG
ACCTTCCTTCCCGCGGCCTGCTGCCGGCTCTGCGGCCTCTTCCGCGTCTTCGCCTTCGCCC
TCAGACGAGTCGGATCTCCCTTTGGGCCGCCTCCCCGCATCGATACCGTCGACTAGCCGT
ACCTTTAAGACCAATGACTTACAAGGCAGCTGTAGATCTTAGCCACTTTTTAAAAGAAAA
GGGGGGACTGGAAGGGCTAATTCACTCCCAAAGAAGACAAGATCTGCTTTTTGCCTGTA
CTGGGTCTCTCTGGTTAGACCAGATCTGAGCCTGGGAGCTCTCTGGCTAACTAGGGAACC
CACTGCTTAAGCCTCAATAAAGCTTGCCTTGAGTGCTTCAAGTAGTGTGTGCCCGTCTGT
TGTGTGACTCTGGTAACTAGAGATCCCTCAGACCCTTTTAGTCAGTGTGGAAAATCTCTA

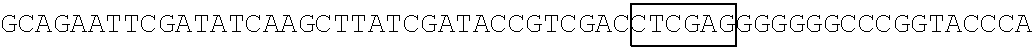

ATTCGCCCTATAGTGAGTCGTATTACAATTCACTGGCCGTCGTTTTACAACGTCGTGACT
GGGAAAACCCTGGCGTTACCCAACTTAATCGCCTTGCAGCACATCCCCCTTTCGCCAGCT
GGCGTAATAGCGAAGAGGCCCGCACCGATCGCCCTTCCCAACAGTTGCGCAGCCTGAAT
GGCGAATGGAAATTGTAAGCGTTAATATTTTGTTAAAATTCGCGTTAAATTTTTGTTAAA
TCAGCTCATTTTTTAACCAATAGGCCGAAATCGGCAAAATCCCTTATAAATCAAAAGAAT
AGACCGAGATAGGGTTGAGTGTTGTTCCAGTTTGGAACAAGAGTCCACTATTAAAGAAC
GTGGACTCCAACGTCAAAGGGCGAAAAACCGTCTATCAGGGCGATGGCCCACTACGTGA
ACCATCACCCTAATCAAGTTTTTTGGGGTCGAGGTGCCGTAAAGCACTAAATCGGAACCC
TAAAGGGAGCCCCCGATTTAGAGCTTGACGGGGAAAGCCGGCGAACGTGGCGAGAAAG
GAAGGGAAGAAAGCGAAAGGAGCGGGCGCTAGGGCGCTGGCAAGTGTAGCGGTCACGC
TGCGCGTAACCACCACACCCGCCGCGCTTAATGCGCCGCTACAGGGCGCGTCAGGTGGC
ACTTTTCGGGGAAATGTGCGCGGAACCCCTATTTGTTTATTTTTCTAAATACATTCAAATA
TGTATCCGCTCATGAGACAATAACCCTGATAAATGCTTCAATAATATTGAAAAAGGAAG
AGTATGAGTATTCAACATTTCCGTGTCGCCCTTATTCCCTTTTTTGCGGCATTTTGCCTTC
CTGTTTTTGCTCACCCAGAAACGCTGGTGAAAGTAAAAGATGCTGAAGATCAGTTGGGT
GCACGAGTGGGTTACATCGAACTGGATCTCAACAGCGGTAAGATCCTTGAGAGTTTTCG
CCCCGAAGAACGTTTTCCAATGATGAGCACTTTTAAAGTTCTGCTATGTGGCGCGGTATT
ATCCCGTATTGACGCCGGGCAAGAGCAACTCGGTCGCCGCATACACTATTCTCAGAATG
ACTTGGTTGAGTACTCACCAGTCACAGAAAAGCATCTTACGGATGGCATGACAGTAAGA
GAATTATGCAGTGCTGCCATAACCATGAGTGATAACACTGCGGCCAACTTACTTCTGACA
ACGATCGGAGGACCGAAGGAGCTAACCGCTTTTTTGCACAACATGGGGGATCATGTAAC
TCGCCTTGATCGTTGGGAACCGGAGCTGAATGAAGCCATACCAAACGACGAGCGTGACA
CCACGATGCCTGTAGCAATGGCAACAACGTTGCGCAAACTATTAACTGGCGAACTACTT
ACTCTAGCTTCCCGGCAACAATTAATAGACTGGATGGAGGCGGATAAAGTTGCAGGACC
ACTTCTGCGCTCGGCCCTTCCGGCTGGCTGGTTTATTGCTGATAAATCTGGAGCCGGTGA
GCGTGGGTCTCGCGGTATCATTGCAGCACTGGGGCCAGATGGTAAGCCCTCCCGTATCGT
AGTTATCTACACGACGGGGAGTCAGGCAACTATGGATGAACGAAATAGACAGATCGCTG
AGATAGGTGCCTCACTGATTAAGCATTGGTAACTGTCAGACCAAGTTTACTCATATATAC
TTTAGATTGATTTAAAACTTCATTTTTAATTTAAAAGGATCTAGGTGAAGATCCTTTTTGA
TAATCTCATGACCAAAATCCCTTAACGTGAGTTTTCGTTCCACTGAGCGTCAGACCCCGT
AGAAAAGATCAAAGGATCTTCTTGAGATCCTTTTTTTCTGCGCGTAATCTGCTGCTTGCA
AACAAAAAAACCACCGCTACCAGCGGTGGTTTGTTTGCCGGATCAAGAGCTACCAACTC
TTTTTCCGAAGGTAACTGGCTTCAGCAGAGCGCAGATACCAAATACTGTTCTTCTAGTGT
AGCCGTAGTTAGGCCACCACTTCAAGAACTCTGTAGCACCGCCTACATACCTCGCTCTGC
TAATCCTGTTACCAGTGGCTGCTGCCAGTGGCGATAAGTCGTGTCTTACCGGGTTGGACT
CAAGACGATAGTTACCGGATAAGGCGCAGCGGTCGGGCTGAACGGGGGGTTCGTGCACA
CAGCCCAGCTTGGAGCGAACGACCTACACCGAACTGAGATACCTACAGCGTGAGCTATG
AGAAAGCGCCACGCTTCCCGAAGGGAGAAAGGCGGACAGGTATCCGGTAAGCGGCAGG
GTCGGAACAGGAGAGCGCACGAGGGAGCTTCCAGGGGGAAACGCCTGGTATCTTTATAG
TCCTGTCGGGTTTCGCCACCTCTGACTTGAGCGTCGATTTTTGTGATGCTCGTCAGGGGG
GCGGAGCCTATGGAAAAACGCCAGCAACGCGGCCTTTTTACGGTTCCTGGCCTTTTGCTG
GCCTTTTGCTCACATGTTCTTTCCTGCGTTATCCCCTGATTCTGTGGATAACCGTATTACC
GCCTTTGAGTGAGCTGATACCGCTCGCCGCAGCCGAACGACCGAGCGCAGCGAGTCAGT
GAGCGAGGAAGCGGAAGAGCGCCCAATACGCAAACCGCCTCTCCCCGCGCGTTGGCCGA
TTCATTAATGCAGCTGGCACGACAGGTTTCCCGACTGGAAAGCGGGCAGTGAGCGCAAC
GCAATTAATGTGAGTTAGCTCACTCATTAGGCACCCCAGGCTTTACACTTTATGCTTCCG
GCTCGTATGTTGTGTGGAATTGTGAGCGGATAACAATTTCACACAGGAAACAGCTATGA
CCATGATTACGCCAAGCTCGAAATTAACCCTCACTAAAGGGAACAAAAGCTGGAGCTCC

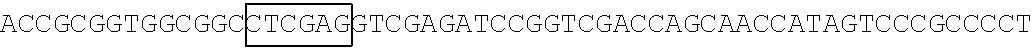

AACTCCGCCCATCCCGCCCCTAACTCCGCCCAGTTCCGCCCATTCTCCGCCCCATGGCTG
ACTAATTTTTTTTATTTATGCAGAGGCCGAGGCCGCCTCGGCCTCTGAGCTATTCCAGAA
GTAGTGAGGAGGCTTTTTTGGAGGCCTAGGCTTTTGCAAAAAGCTTCGACGGTATCGATT
GGCTCATGTCCAACATTACCGCCATGTTGACATTGATTATTGACTAGTTATTAATAGTAA
TCAATTACGGGGTCATTAGTTCATAGCCCATATATGGAGTTCCGCGTTACATAACTTACG
GTAAATGGCCCGCCTGGCTGACCGCCCAACGACCCCCGCCCATTGACGTCAATAATGAC
GTATGTTCCCATAGTAACGCCAATAGGGACTTTCCATTGACGTCAATGGGTGGAGTATTT
ACGGTAAACTGCCCACTTGGCAGTACATCAAGTGTATCATATGCCAAGTACGCCCCCTAT
TGACGTCAATGACGGTAAATGGCCCGCCTGGCATTATGCCCAGTACATGACCTTATGGG
ACTTTCCTACTTGGCAGTACATCTACGTATTAGTCATCGCTATTACCATGGTGATGCGGTT
TTGGCAGTACATCAATGGGCGTGGATAGCGGTTTGACTCACGGGGATTTCCAAGTCTCCA
CCCCATTGACGTCAATGGGAGTTTGTTTTGGCACCAAAATCAACGGGACTTTCCAAAATG
TCGTAACAACTCCGCCCCATTGACGCAAATGGGCGGTAGGCGTGTACGGAATTCGGAGT
GGCGAGCCCTCAGATCCTGCATATAAGCAGCTGCTTTTTGCCTGTACTGGGTCTCTCTG

TABLE 18
Leader_R11-Hinge-CH3-CD28tm/41BB-Z-T2A-tEGFR (SEQ ID NO:46)
Leader: MLLLVTSLLLCELPHPAFLLIP
scFV R11
QSVKESEGDLVTPAGNLTLTCTASGSDINDYPISWVRQAPGKGLEWIGFINSGGSTWYASWV
KGRFTISRTSTTVDLKMTSLTTDDTATYFCARGYSTYYGDFNIWGPGTLVTISSGGGGSGGG
GSGGGGSELVMTQTPSSTSGAVGGTVTINCQASQSIDSNLAWFQQKPGQPPTLLIYRASNLAS
GVPSRFSGSRSGTEYTLTISGVQREDAATYYCLGGVGNVSYRTSFGGGTEVVVK
Hinge/Spacer: ESKYGPPCPPCP
CH3

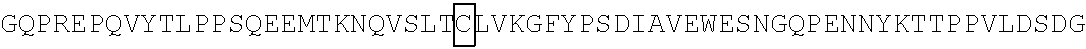

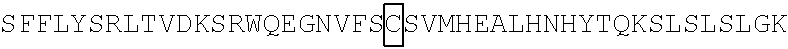

CD28tm: MFWVLVVVGGVLACYSLLVTVAFIIFWV
4-1BB: KRGRKKLLYIFKQPFMRPVQTTQEEDGCSCRFPEEEEGGCEL
CD3zeta
RVKFSRSADAPAYQQGQNQLYNELNLGRREEYDVLDKRRGRDPEMGGKPRRKNPQEGLYN
ELQKDKMAEAYSEIGMKGERRRGKGHDGLYQGLSTATKDTYDALHMQALPPR
T2A: LEGGGEGRGSLLTCGDVEENPGPRM
tEGFR
LLLVTSLLLCELPHPAFLLIPRKVCNGIGIGEFKDSLSINATNIKHFKNCTSISGDLHILPVAFRG
DSFTHTPPLDPQELDILKTVKEITGFLLIQAWPENRTDLHAFENLEIIRGRTKQHGQFSLAVVS
LNITSLGLRSLKEISDGDVIISGNKNLCYANTINWKKLFGTSGQKTKIISNRGENSCKATGQVC
HALCSPEGCWGPEPRDCVSCRNVSRGRECVDKCNLLEGEPREFVENSECIQCHPECLPQAMN
ITCTGRGPDNCIQCAHYIDGPHCVKTCPAGVMGENNTLVWKYADAGHVCHLCHPNCTYGC
TGPGLEGCPTNGPKIPSIATGMVGALLLLLVVALGIGLFM

TABLE 19
R11 short spacer CAR: PJ_R11-41BB-Z-T2A-tEGFR(SEQ ID NO: 47)
GTTAGACCAGATCTGAGCCTGGGAGCTCTCTGGCTAACTAGGGAACCCACTGCTTAAGC
CTCAATAAAGCTTGCCTTGAGTGCTTCAAGTAGTGTGTGCCCGTCTGTTGTGTGACTCTG
GTAACTAGAGATCCCTCAGACCCTTTTAGTCAGTGTGGAAAATCTCTAGCAGTGGCGCCC
GAACAGGGACTTGAAAGCGAAAGGGAAACCAGAGGAGCTCTCTCGACGCAGGACTCGG
CTTGCTGAAGCGCGCACGGCAAGAGGCGAGGGGCGGCGACTGGTGAGTACGCCAAAAA
TTTTGACTAGCGGAGGCTAGAAGGAGAGAGATGGGTGCGAGAGCGTCAGTATTAAGCGG
GGGAGAATTAGATCGATGGGAAAAAATTCGGTTAAGGCCAGGGGGAAAGAAAAAATAT
AAATTAAAACATATAGTATGGGCAAGCAGGGAGCTAGAACGATTCGCAGTTAATCCTGG
CCTGTTAGAAACATCAGAAGGCTGTAGACAAATACTGGGACAGCTACAACCATCCCTTC
AGACAGGATCAGAAGAACTTAGATCATTATATAATACAGTAGCAACCCTCTATTGTGTG
CATCAAAGGATAGAGATAAAAGACACCAAGGAAGCTTTAGACAAGATAGAGGAAGAGC
AAAACAAAAGTAAGAAAAAAGCACAGCAAGCAGCAGCTGACACAGGACACAGCAATCA
GGTCAGCCAAAATTACCCTATAGTGCAGAACATCCAGGGGCAAATGGTACATCAGGCCA
TATCACCTAGAACTTTAAATGCATGGGTAAAAGTAGTAGAAGAGAAGGCTTTCAGCCCA
GAAGTGATACCCATGTTTTCAGCATTATCAGAAGGAGCCACCCCACAAGATTTAAACAC
CATGCTAAACACAGTGGGGGGACATCAAGCAGCCATGCAAATGTTAAAAGAGACCATCA
ATGAGGAAGCTGCAGGCAAAGAGAAGAGTGGTGCAGAGAGAAAAAAGAGCAGTGGGA
ATAGGAGCTTTGTTCCTTGGGTTCTTGGGAGCAGCAGGAAGCACTATGGGCGCAGCGTC
AATGACGCTGACGGTACAGGCCAGACAATTATTGTCTGGTATAGTGCAGCAGCAGAACA
ATTTGCTGAGGGCTATTGAGGCGCAACAGCATCTGTTGCAACTCACAGTCTGGGGCATCA
AGCAGCTCCAGGCAAGAATCCTGGCTGTGGAAAGATACCTAAAGGATCAACAGCTCCTG
GGGATTTGGGGTTGCTCTGGAAAACTCATTTGCACCACTGCTGTGCCTTGGATCTACAAA
TGGCAGTATTCATCCACAATTTTAAAAGAAAAGGGGGGATTGGGGGGTACAGTGCAGGG
GAAAGAATAGTAGACATAATAGCAACAGACATACAAACTAAAGAATTACAAAAACAAA
TTACAAAAATTCAAAATTTTCGGGTTTATTACAGGGACAGCAGAGATCCAGTTTGGGGAT
CAATTGCATGAAGAATCTGCTTAGGGTTAGGCGTTTTGCGCTGCTTCGCGAGGATCTGCG
ATCGCTCCGGTGCCCGTCAGTGGGCAGAGCGCACATCGCCCACAGTCCCCGAGAAGTTG
GGGGGAGGGGTCGGCAATTGAACCGGTGCCTAGAGAAGGTGGCGCGGGGTAAACTGGG
AAAGTGATGTCGTGTACTGGCTCCGCCTTTTTCCCGAGGGTGGGGGAGAACCGTATATAA
GTGCAGTAGTCGCCGTGAACGTTCTTTTTCGCAACGGGTTTGCCGCCAGAACACAGCTGA
AGCTTCGAGGGGCTCGCATCTCTCCTTCACGCGCCCGCCGCCCTACCTGAGGCCGCCATC
CACGCCGGTTGAGTCGCGTTCTGCCGCCTCCCGCCTGTGGTGCCTCCTGAACTGCGTCCG
CCGTCTAGGTAAGTTTAAAGCTCAGGTCGAGACCGGGCCTTTGTCCGGCGCTCCCTTGGA
GCCTACCTAGACTCAGCCGGCTCTCCACGCTTTGCCTGACCCTGCTTGCTCAACTCTACGT
CTTTGTTTCGTTTTCTGTTCTGCGCCGTTACAGATCCAAGCTGTGACCGGCGCCTACG

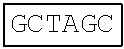

scFV R11

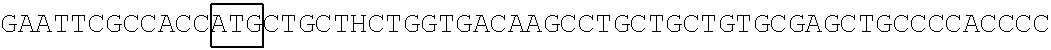

GCCTTTCTGCTGATCCCCCAGAGCGTGAAAGAGTCCGAGGGCGACCTGGTCACACCAGC
CGGCAACCTGACCCTGACCTGTACCGCCAGCGGCAGCGACATCAACGACTACCCCATCT
CTTGGGTCCGCCAGGCTCCTGGCAAGGGACTGGAATGGATCGGCTTCATCAACAGCGGC
GGCAGCACTTGGTACGCCAGCTGGGTCAAAGGCCGGTTCACCATCAGCCGGACCAGCAC
CACCGTGGACCTGAAGATGACAAGCCTGACCACCGACGACACCGCCACCTACTTTTGCG
CCAGAGGCTACAGCACCTACTACGGCGACTTCAACATCTGGGGCCCTGGCACCCTGGTC
ACAATCTCTAGCGGCGGAGGCGGCAGCGGAGGTGGAGGAAGTGGCGGCGGAGGATCCG
AGCTGGTCATGACCCAGACCCCCAGCAGCACATCTGGCGCCGTGGGCGGCACCGTGACC
ATCAATTGCCAGGCCAGCCAGAGCATCGACAGCAACCTGGCCTGGTTCCAGCAGAAGCC
CGGCCAGCCCCCCACCCTGCTGATCTACAGAGCCTCCAACCTGGCCAGCGGCGTGCCAA
GCAGATTCAGCGGCAGCAGATCTGGCACCGAGTACACCCTGACCATCTCCGGCGTGCAG
AGAGAGGACGCCGCTACCTATTACTGCCTGGGCGGCGTGGGCAACGTGTCCTACAGAAC
CAGCTTCGGCGGAGGTACTGAGGTGGTCGTCAAA
Hinge/spacer

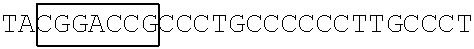

CH3
GGCCAGCCTCGCGAGCCCCAGGTGTACACCCTGCCTCCCTCCCAGGAAGAGATGACCAA
GAACCAGGTGTCCCTGACCTGCCTGGTGAAGGGCTTCTACCCCAGCGACATCGCCGTGG
AGTGGGAGAGCAACGGCCAGCCTGAGAACAACTACAAGACCACCCCTCCCGTGCTGGAC
AGCGACGGCAGCTTCTTCCTGTACAGCCGGCTGACCGTGGACAAGAGCCGGTGGCAGGA
AGGCAACGTCTTTAGCTGCAGCGTGATGCACGAGGCCCTGCACAACCACTACACCCAGA
AGAGCCTGAGCCTGTCCCTGGGCAAG
4-1BB
ATGTTCTGGGTGCTGGTGGTGGTGGGCGGGGTGCTGGCCTGCTACAGCCTGCTGGTGACA
GTGGCCTTCATCATCTTTTGGGTGAAACGGGGCAGAAAGAAACTCCTGTATATATTCAAA
CAACCATTTATGAGACCAGTACAAACTACTCAAGAGGAAGATGGCTGTAGCTGCCGATT
TCCAGAAGAAGAAGAAGGAGGATGTGAACTG
CD3zeta
CGGGTGAAGTTCAGCAGAAGCGCCGACGCCCCTGCCTACCAGCAGGGCCAGAATCAGCT
GTACAACGAGCTGAACCTGGGCAGAAGGGAAGAGTACGACGTCCTGGATAAGCGGAGA
GGCCGGGACCCTGAGATGGGCGGCAAGCCTCGGCGGAAGAACCCCCAGGAAGGCCTGT
ATAACGAACTGCAGAAAGACAAGATGGCCGAGGCCTACAGCGAGATCGGCATGAAGGG
CGAGCGGAGGCGGGGCAAGGGCCACGACGGCCTGTATCAGGGCCTGTCCACCGCCACCA
AGGATACCTACGACGCCCTGCACATGCAGGCCCTGCCCCCAAGG
T2A

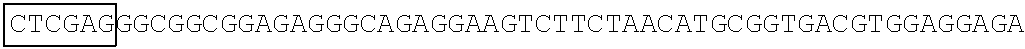

ATCCCGGCCCTAGG
tEGFR
ATGCTTCTCCTGGTGACAAGCCTTCTGCTCTGTGAGTTACCACACCCAGCATTCCTCCTGA
TCCCACGCAAAGTGTGTAACGGAATAGGTATTGGTGAATTTAAAGACTCACTCTCCATAA
ATGCTACGAATATTAAACACTTCAAAAACTGCACCTCCATCAGTGGCGATCTCCACATCC

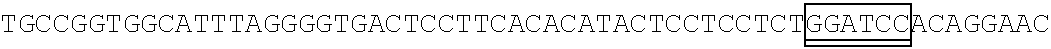

TGGATATTCTGAAAACCGTAAAGGAAATCACAGGGTTTTTGCTGATTCAGGCTTGGCCTG
AAAACAGGACGGACCTCCATGCCTTTGAGAACCTAGAAATCATACGCGGCAGGACCAAG
CAACATGGTCAGTTTTCTCTTGCAGTCGTCAGCCTGAACATAACATCCTTGGGATTACGC
TCCCTCAAGGAGATAAGTGATGGAGATGTGATAATTTCAGGAAACAAAAATTTGTGCTA
TGCAAATACAATAAACTGGAAAAAACTGTTTGGGACCTCCGGTCAGAAAACCAAAATTA
TAAGCAACAGAGGTGAAAACAGCTGCAAGGCCACAGGCCAGGTCTGCCATGCCTTGTGC
TCCCCCGAGGGCTGCTGGGGCCCGGAGCCCAGGGACTGCGTCTCTTGCCGGAATGTCAG
CCGAGGCAGGGAATGCGTGGACAAGTGCAACCTTCTGGAGGGTGAGCCAAGGGAGTTTG
TGGAGAACTCTGAGTGCATACAGTGCCACCCAGAGTGCCTGCCTCAGGCCATGAACATC
ACCTGCACAGGACGGGGACCAGACAACTGTATCCAGTGTGCCCACTACATTGACGGCCC
CCACTGCGTCAAGACCTGCCCGGCAGGAGTCATGGGAGAAAACAACACCCTGGTCTGGA
AGTACGCAGACGCCGGCCATGTGTGCCACCTGTGCCATCCAAACTGCACCTACGGATGC
ACTGGGCCAGGTCTTGAAGGCTGTCCAACGAATGGGCCTAAGATCCCGTCCATCGCCAC
TGGGATGGTGGGGGCCCTCCTCTTGCTGCTGGTGGTGGCCCTGGGGATCGGCCTCTTCAT

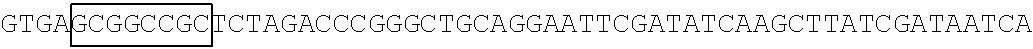

ACCTCTGGATTACAAAATTTGTGAAAGATTGACTGGTATTCTTAACTATGTTGCTCCTTTT
ACGCTATGTGGATACGCTGCTTTAATGCCTTTGTATCATGCTATTGCTTCCCGTATGGCTT
TCATTTTCTCCTCCTTGTATAAATCCTGGTTGCTGTCTCTTTATGAGGAGTTGTGGCCCGT
TGTCAGGCAACGTGGCGTGGTGTGCACTGTGTTTGCTGACGCAACCCCCACTGGTTGGGG
CATTGCCACCACCTGTCAGCTCCTTTCCGGGACTTTCGCTTTCCCCCTCCCTATTGCCACG
GCGGAACTCATCGCCGCCTGCCTTGCCCGCTGCTGGACAGGGGCTCGGCTGTTGGGCACT
GACAATTCCGTGGTGTTGTCGGGGAAATCATCGTCCTTTCCTTGGCTGCTCGCCTGTGTTG
CCACCTGGATTCTGCGCGGGACGTCCTTCTGCTACGTCCCTTCGGCCCTCAATCCAGCGG
ACCTTCCTTCCCGCGGCCTGCTGCCGGCTCTGCGGCCTCTTCCGCGTCTTCGCCTTCGCCC
TCAGACGAGTCGGATCTCCCTTTGGGCCGCCTCCCCGCATCGATACCGTCGACTAGCCGT
ACCTTTAAGACCAATGACTTACAAGGCAGCTGTAGATCTTAGCCACTTTTTAAAAGAAAA
GGGGGGACTGGAAGGGCTAATTCACTCCCAAAGAAGACAAGATCTGCTTTTTGCCTGTA
CTGGGTCTCTCTGGTTAGACCAGATCTGAGCCTGGGAGCTCTCTGGCTAACTAGGGAACC
CACTGCTTAAGCCTCAATAAAGCTTGCCTTGAGTGCTTCAAGTAGTGTGTGCCCGTCTGT
TGTGTGACTCTGGTAACTAGAGATCCCTCAGACCCTTTTAGTCAGTGTGGAAAATCTCTA

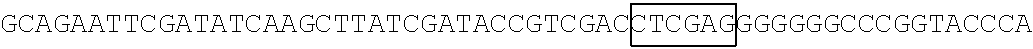

ATTCGCCCTATAGTGAGTCGTATTACAATTCACTGGCCGTCGTTTTACAACGTCGTGACT
GGGAAAACCCTGGCGTTACCCAACTTAATCGCCTTGCAGCACATCCCCCTTTCGCCAGCT
GGCGTAATAGCGAAGAGGCCCGCACCGATCGCCCTTCCCAACAGTTGCGCAGCCTGAAT
GGCGAATGGAAATTGTAAGCGTTAATATTTTGTTAAAATTCGCGTTAAATTTTTGTTAAA
TCAGCTCATTTTTTAACCAATAGGCCGAAATCGGCAAAATCCCTTATAAATCAAAAGAAT
AGACCGAGATAGGGTTGAGTGTTGTTCCAGTTTGGAACAAGAGTCCACTATTAAAGAAC
GTGGACTCCAACGTCAAAGGGCGAAAAACCGTCTATCAGGGCGATGGCCCACTACGTGA
ACCATCACCCTAATCAAGTTTTTTGGGGTCGAGGTGCCGTAAAGCACTAAATCGGAACCC
TAAAGGGAGCCCCCGATTTAGAGCTTGACGGGGAAAGCCGGCGAACGTGGCGAGAAAG
GAAGGGAAGAAAGCGAAAGGAGCGGGCGCTAGGGCGCTGGCAAGTGTAGCGGTCACGC
TGCGCGTAACCACCACACCCGCCGCGCTTAATGCGCCGCTACAGGGCGCGTCAGGTGGC
ACTTTTCGGGGAAATGTGCGCGGAACCCCTATTTGTTTATTTTTCTAAATACATTCAAATA
TGTATCCGCTCATGAGACAATAACCCTGATAAATGCTTCAATAATATTGAAAAAGGAAG
AGTATGAGTATTCAACATTTCCGTGTCGCCCTTATTCCCTTTTTTGCGGCATTTTGCCTTC
CTGTTTTTGCTCACCCAGAAACGCTGGTGAAAGTAAAAGATGCTGAAGATCAGTTGGGT
GCACGAGTGGGTTACATCGAACTGGATCTCAACAGCGGTAAGATCCTTGAGAGTTTTCG
CCCCGAAGAACGTTTTCCAATGATGAGCACTTTTAAAGTTCTGCTATGTGGCGCGGTATT
ATCCCGTATTGACGCCGGGCAAGAGCAACTCGGTCGCCGCATACACTATTCTCAGAATG
ACTTGGTTGAGTACTCACCAGTCACAGAAAAGCATCTTACGGATGGCATGACAGTAAGA
GAATTATGCAGTGCTGCCATAACCATGAGTGATAACACTGCGGCCAACTTACTTCTGACA
ACGATCGGAGGACCGAAGGAGCTAACCGCTTYTTTGCACAACATGGGGGATCATGTAAC
TCGCCTTGATCGTTGGGAACCGGAGCTGAATGAAGCCATACCAAACGACGAGCGTGACA
CCACGATGCCTGTAGCAATGGCAACAACGTTGCGCAAACTATTAACTGGCGAACTACTT
ACTCTAGCTTCCCGGCAACAATTAATAGACTGGATGGAGGCGGATAAAGTTGCAGGACC
ACTTCTGCGCTCGGCCCTTCCGGCTGGCTGGTTTATTGCTGATAAATCTGGAGCCGGTGA
GCGTGGGTCTCGCGGTATCATTGCAGCACTGGGGCCAGATGGTAAGCCCTCCCGTATCGT
AGTTATCTACACGACGGGGAGTCAGGCAACTATGGATGAACGAAATAGACAGATCGCTG
AGATAGGTGCCTCACTGATTAAGCATTGGTAACTGTCAGACCAAGTTTACTCATATATAC
TTTAGATTGATTTAAAACTTCATTTTTAATTTAAAAGGATCTAGGTGAAGATCCTTTTTGA
TAATCTCATGACCAAAATCCCTTAACGTGAGTTTTCGTTCCACTGAGCGTCAGACCCCGT
AGAAAAGATCAAAGGATCTTCTTGAGATCCTTTTTTTCTGCGCGTAATCTGCTGCTTGCA
AACAAAAAAACCACCGCTACCAGCGGTGGTTTGTTTGCCGGATCAAGAGCTACCAACTC
TTTTTCCGAAGGTAACTGGCTTCAGCAGAGCGCAGATACCAAATACTGTTCTTCTAGTGT
AGCCGTAGTTAGGCCACCACTTCAAGAACTCTGTAGCACCGCCTACATACCTCGCTCTGC
TAATCCTGTTACCAGTGGCTGCTGCCAGTGGCGATAAGTCGTGTCTTACCGGGTTGGACT
CAAGACGATAGTTACCGGATAAGGCGCAGCGGTCGGGCTGAACGGGGGGTTCGTGCACA
CAGCCCAGCTTGGAGCGAACGACCTACACCGAACTGAGATACCTACAGCGTGAGCTATG
AGAAAGCGCCACGCTTCCCGAAGGGAGAAAGGCGGACAGGTATCCGGTAAGCGGCAGG
GTCGGAACAGGAGAGCGCACGAGGGAGCTTCCAGGGGGAAACGCCTGGTATCTTTATAG
TCCTGTCGGGTTTCGCCACCTCTGACTTGAGCGTCGATTTTTGTGATGCTCGTCAGGGGG
GCGGAGCCTATGGAAAAACGCCAGCAACGCGGCCTTTTTACGGTTCCTGGCCTTTTGCTG
GCCTTTTGCTCACATGTTCTTTCCTGCGTTATCCCCTGATTCTGTGGATAACCGTATTACC
GCCTTTGAGTGAGCTGATACCGCTCGCCGCAGCCGAACGACCGAGCGCAGCGAGTCAGT
GAGCGAGGAAGCGGAAGAGCGCCCAATACGCAAACCGCCTCTCCCCGCGCGTTGGCCGA
TTCATTAATGCAGCTGGCACGACAGGTTTCCCGACTGGAAAGCGGGCAGTGAGCGCAAC
GCAATTAATGTGAGTTAGCTCACTCATTAGGCACCCCAGGCTTTACACTTTATGCTTCCG
GCTCGTATGTTGTGTGGAATTGTGAGCGGATAACAATTTCACACAGGAAACAGCTATGA
CCATGATTACGCCAAGCTCGAAATTAACCCTCACTAAAGGGAACAAAAGCTGGAGCTCC

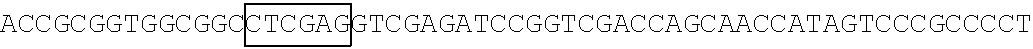

AACTCCGCCCATCCCGCCCCTAACTCCGCCCAGTTCCGCCCATTCTCCGCCCCATGGCTG
ACTAATTTTTTTTATTTATGCAGAGGCCGAGGCCGCCTCGGCCTCTGAGCTATTCCAGAA
GTAGTGAGGAGGCTTTTTTGGAGGCCTAGGCTTTTGCAAAAAGCTTCGACGGTATCGATT
GGCTCATGTCCAACATTACCGCCATGTTGACATTGATTATTGACTAGTTATTAATAGTAA
TCAATTACGGGGTCATTAGTTCATAGCCCATATATGGAGTTCCGCGTTACATAACTTACG
GTAAATGGCCCGCCTGGCTGACCGCCCAACGACCCCCGCCCATTGACGTCAATAATGAC
GTATGTTCCCATAGTAACGCCAATAGGGACTTTCCATTGACGTCAATGGGTGGAGTATTT
ACGGTAAACTGCCCACTTGGCAGTACATCAAGTGTATCATATGCCAAGTACGCCCCCTAT
TGACGTCAATGACGGTAAATGGCCCGCCTGGCATTATGCCCAGTACATGACCTTATGGG
ACTTTCCTACTTGGCAGTACATCTACGTATTAGTCATCGCTATTACCATGGTGATGCGGTT
TTGGCAGTACATCAATGGGCGTGGATAGCGGTTTGACTCACGGGGATTTCCAAGTCTCCA
CCCCATTGACGTCAATGGGAGTTTGTTTTGGCACCAAAATCAACGGGACTTTCCAAAATG
TCGTAACAACTCCGCCCCATTGACGCAAATGGGCGGTAGGCGTGTACGGAATTCGGAGT
GGCGAGCCCTCAGATCCTGCATATAAGCAGCTGCTTTTTGCCTGTACTGGGTCTCTCTG

TABLE 20
Leader_R11-Hinge- CD28tm/41BB-Z-T2A-tEGFR
(SEQ ID NO: 48)

Leader: MLLLVTSLLLCELPHPAFLLIP
ScFv R11
QSVKESEGDLVTPAGNLTLTCTASGSDINDYPISWVRQAPGKGLEWIGFIN
SGGSTWYASWVKGRFTISRTSTTVDLKMTSLTTDDTATYFCARGYSTYYGD
FNIWGPGTLVTISSGGGGSGGGGSGGGGSELVMTQTPSSTSGAVGGTVTIN
CQASQSIDSNLAWFQQKPGQPPTLLIYRASNLASGVPSRFSGSRSGTEYTL
TISGVQREDAATYYCLGGVGNVSYRTSFGGGTEVVVK
Spacer/Hinge: ESKYGPPCPPCP
CD28tm: MFWVLVVVGGVLACYSLLVTVAFIIFWV
4-1BB: KRGRKKLLYIFKQPFMRPVQTTQEEDGCSCRFPEEEEGGCEL
CD3zeta
RVKFSRSADAPAYQQGQNQLYNELNLGRREEYDVLDKRRGRDPEMGGKPRR
KNPQEGLYNELQKDKMAEAYSEIGMKGERRRGKGHDGLYQGLSTATKDTYD
ALHMQALPPR
T2A: LEGGGEGRGSLLTCGDVEENPGPR
tEGFR
MLLLVTSLLLCELPHPAFLLIPRKVCNGIGIGEFKDSLSINATNIKHFKNC
TSISGDLHILPVAFRGDSFTHTPPLDPQELDILKTVKEITGFLLIQAWPEN
RTDLHAFENLEIIRGRTKQHGQFSLAVVSLNITSLGLRSLKEISDGDVIIS
GNKNLCYANTINWKKLFGTSGQKTKIISNRGENSCKATGQVCHALCSPEGC
WGPEPRDCVSCRNVSRGRECVDKCNLLEGEPREFVENSECIQCHPECLPQA
MNITCTGRGPDNCIQCAHYIDGPHCVKTCPAGVMGENNTLVWKYADAGHVC
HLCHPNCTYGCTGPGLEGCPTNGPKIPSIATGMVGALLLLLVVALGIGLFM

TABLE 21
Intermediate Spacer (SEQ ID NO: 49)
Hinge/spacer: ESKYGPPCPPCP
CH3

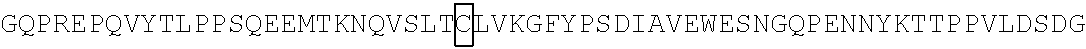

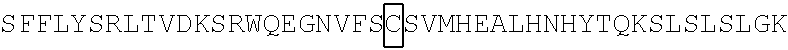

Long spacer (SEQ ID NO: 50)
Hinge: ESKYGPPCPPCP
CH2

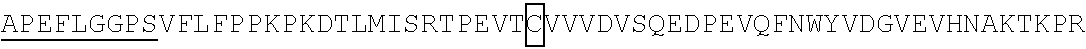

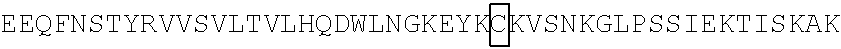

CH3

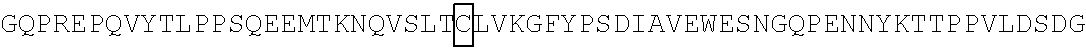

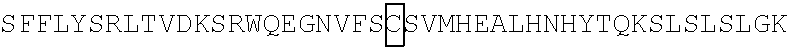

TABLE 22
Her 2 Construct-Short Spacer (SEQ ID NO: 54)
GMCSFss-Her2scFv-IgG4hinge-CD28tm-41BB-Zeta-
T2A-EGFRt

Leader
Atgcttctcctggtgacaagccttctgctctgtgagttaccacacccagca
ttcctcctgatccca
Her2scFV
gatatccagatgacccagtccccgagctccctgtccgcctctgtgggcgat
agggtcaccatcacctgccgtgccagtcaggatgtgaatactgctgtagcc
tggtatcaacagaaaccaggaaaagctccgaaactactgatttactcggca
tccttcctctactctggagtcccttctcgcttctctggttccagatctggg
acggatttcactctgaccatcagcagtctgcagccggaagacttcgcaact
tattactgtcagcaacattatactactcctcccacgttcggacagggtacc
aaggtggagatcaaaggcagtactagoggcggtggctccgggggcggatcc
ggtgggggcggcagcagcgaggttcagctggtggagtctggcggtggcctg
gtgcagccagggggctcactccgtngtcctgtgcagcnctggcttcaacat
taaagacacctatatacactgggtgcgtcaggccccgggtaagggcctgga
atgggttgcaaggatttatcctacgaatggttatactagatatgccgatag
cgtcaagggccgtncactataagcgcagacacatccaaaaacacagcctac
ctgcagatgaacagcctgcgtgctgaggacactgccgtctattattgttct
agatggggaggggacggottctatgctatggactactggggtcaaggaacc
ctggtcaccgtctcgagt
Hinge spacer
Gagagcaagtacggaccgccctgccccccttgccct
CD28tm
atgttctgggtgctggtggtggtcggaggcgtgctggcctgctacagcctg
ctggtcaccgtggccttcatcatcttttgggtg
4-1BB
Aaacggggcagaaagaaactcctgtatatattcaaacaaccatttatgaga
ccagtacaaactactcaagaggaagatggctgtagctgccgatttccagaa
gaagaagaaggaggatgtgaactg
CD3 zeta
Cgggtgaagttcagcagaagcgccgacgcccctgcctaccagcagggccag
aatcagctgtacaacgagctgaacctgggcagaagggaagagtacgacgtc
ctggataagoggagaggccgggaccctgagatgggcggcaagcctcggcgg
aagaacccccaggaaggcctgtataacgaactgcagaaagacaagatggcc
gaggcctacagcgagatcggcatgaagggcgagoggaggoggggcaagggc
cacgacggcctgtatcagggcctgtccaccgccaccaaggatacctacgac
gccctgcacatgcaggccctgcccccaagg
T2A
Ctcgagggcggcggagagggcagaggaagtcttctaacatgcggtgacgtg
gaggagaatcccggccctagg
tEGFR
atgcttctcctggtgacaagccttctgctctgtgagttaccacacccagca
ttcctcctgatcccacgcaaagtgtgtaacggaataggtattggtgaattt
aaagactcactctccataaatgctacgaatattaaacacttcaaaaactgc
acctccatcagtggcgatctccacatcctgccggtggcatttaggggtgac
tccttcacacatactcctcctctggatccacaggaactggatattctgaaa
accgtaaaggaaatcacagggtttttgctgattcaggcttggcctgaaaac
aggacggacctccatgcctttgagaacctagaaatcatacgcggcaggacc
aagcaacatggtcagttttctcttgcagtcgtcagcctgaacataacatcc
ttgggattacgctccctcaaggagataagtgatggagatgtgataatttca
ggaaacaaaaatttgtgctatgcaaatacaataaactggaaaaaactgttt
gggacctccggtcagaaaaccaaaattataagcaacagaggtgaaaacagc
tgcaaggccacaggccaggtctgccatgccttgtgctcccccgagggctgc
tggggcccggagcccagggactgcgtctcttgccggaatgtcagccgaggc
agggaatgcgtggacaagtgcaaccttctggagggtgagccaagggagttt
gtggagaactctgagtgcatacagtgccacccagagtgcctgcctcaggcc
atgaacatcacctgcacaggacggggaccagacaactgtatccagtgtgcc
cactacattgacggcccccactgcgtcaagacctgcccggcaggagtcatg
ggagaaaacaacaccctggtctggaagtacgcagacgccggccatgtgtgc
cacctgtgccatccaaactgcacctacggatgcactgggccaggtcttgaa
ggctgtccaacgaatgggcctaagatcccgtccatcgccactgggatggtg
ggggccctcctcttgctgctggtggtggccctggggatcggcctcttcatg
tga

TABLE 23
Her 2 construct-intermediate spacer (SEQ ID NO: 55)

Leader
Atgcttctcctggtgacaagccttctgctctgtgagttaccacaccca
Her2scFv
Gcattcctcctgatcccagatatccagatgacccagtccccgagctccctg
tccgcctctgtgggcgatagggtcaccatcacctgccgtgccagtcaggat
gtgaatactgctgtagcctggtatcaacagaaaccaggaaaagctccgaaa
ctactgatttactcggcatccttcctctactctggagtcccttctcgcttc
tctggttccagatctgggacggatttcactctgaccatcagcagtctgcag
ccggaagacttcgcaacttattactgtcagcaacattatactactcctccc
acgttcggacagggtaccaaggtggagatcaaaggcagtactagcggcggt
ggctccgggggcggatccggtgggggcggcagcagcgaggttcagctggtg
gagtctggcggtggcctggtgcagccagggggctcactccgtagtcctgtg
cagcttctggcttcaacattaaagacacctatatacactgggtgcgtcagg
ccccgggtaagggcctggaatgggttgcaaggatttatcctacgaatggtt
atactagatatgccgatagcgtcaagggccgtacactataagcgcagacac
atccaaaaacacagcctacctgcagatgaacagcctgcgtgctgaggacac
tgccgtctattattgttctagatggggaggggacggcttctatgctatgga
ctactggggtcaaggaaccctggtcaccgtctcgagt
Hinge spacer
GagagcaagtacggaccgccctgccccccttgccctGgccagcctagagaa
ccccaggtgtacaccctgcctcccagccaggaagagatgaccaagaaccag
gtgtccctgacctgcctggtcaaaggcttctaccccagcgatatcgccgtg
gaatgggagagcaacggccagcccgagaacaactacaagaccaccccccct
gtgctggacagcgacggcagcttcttcctgtactcccggctgaccgtggac
aagagccggtggcaggaaggcaacgtcttcagctgcagcgtgatgcacgag
gccctgcacaaccactacacccagaagtccctgagcctgagcctgggcaag
CD28tm
Atgttctgggtgctggtggtggtcggaggcgtgctggcctgctacagcctg
ctggtcaccgtggccttcatcatcttttgggtg
4-1BB
Aaacggggcagaaagaaactcctgtatatattcaaacaaccatttatgaga
ccagtacaaactactcaagaggaagatggctgtagctgccgatttccagaa
gaagaagaaggaggatgtgaactg
CD3 zeta
Cgggtgaagttcagcagaagcgccgacgcccctgcctaccagcagggccag
aatcagctgtacaacgagctgaacctgggcagaagggaagagtacgacgtc
ctggataagcggagaggccgggaccctgagatgggcggcaagcctcggcgg
aagaacccccaggaaggcctgtataacgaactgcagaaagacaagatggcc
gaggcctacagcgagatcggcatgaagggcgagcggaggcggggcaagggc
cacgacggcctgtatcagggcctgtccaccgccaccaaggatacctacgac
gccctgcacatgcaggccctgcccccaagg
T2A
Ctcgagggcggcggagagggcagaggaagtcttctaacatgcggtgacgtg
gaggagaatcccggccctagg
tEGFR
atgcttctcctggtgacaagccttctgctctgtgagttaccacacccagca
ttcctcctgatcccacgcaaagtgtgtaacggaataggtattggtgaattt
aaagactcactctccataaatgctacgaatattaaacacttcaaaaactgc
acctccatcagtggcgatctccacatcctgccggtggcatttaggggtgac
tccttcacacatactcctcctctggatccacaggaactggatattctgaaa
accgtaaaggaaatcacagggtttttgctgattcaggcttggcctgaaaac
aggacggacctccatgcctttgagaacctagaaatcatacgcggcaggacc
aagcaacatggtcagttttctcttgcagtcgtcagcctgaacataacatcc
ttgggattacgctccctcaaggagataagtgatggagatgtgataatttca
ggaaacaaaaatttgtgctatgcaaatacaataaactggaaaaaactgttt
gggacctccggtcagaaaaccaaaattataagcaacagaggtgaaaacagc
tgcaaggccacaggccaggtctgccatgccttgtgctcccccgagggctgc
tggggcccggagcccagggactgcgtctcttgccggaatgtcagccgaggc
agggaatgcgtggacaagtgcaaccttctggagggtgagccaagggagttt
gtggagaactctgagtgcatacagtgccacccagagtgcctgcctcaggcc
atgaacatcacctgcacaggacggggaccagacaactgtatccagtgtgcc
cactacattgacggcccccactgcgtcaagacctgcccggcaggagtcatg
ggagaaaacaacaccctggtctggaagtacgcagacgccggccatgtgtgc
cacctgtgccatccaaactgcacctacggatgcactgggccaggtcttgaa
ggctgtccaacgaatgggcctaagatcccgtccatcgccactgggatggtg
ggggccctcctcttgctgctggtggtggccctggggatcggcctcttcatg
tga

TABLE 24
Her 2 construct-long spacer (SEQ ID NO: 56)

Leader: atgcttctcctggtgacaagccttctgctctgtgagttacca
caccca
Her2 scFv
gcattcctcctgatcccagatatccagatgacccagtccccgagctccct
gtccgcctctgtgggcgatagggtcaccatcacctgccgtgccagtcagg
atgtgaatactgctgtagcctggtatcaacagaaaccaggaaaagctccg
aaactactgatttactcggcatccttcctctactctggagtcccttctcg
cttctctggttccagatctgggacggatttcactctgaccatcagcagtc
tgcagccggaagacttcgcaacttattactgtcagcaacattatactact
cctcccacgttcggacagggtaccaaggtggagatcaaaggcagtactag
cggcggtggctccgggggcggatccggtgggggcggcagcagcgaggttc
agctggtggagtctggcggtggcctggtgcagccagggggctcactccgt
ttgtcctgtgcagcttctggcttcaacattaaagacacctatatacactg
ggtgcgtcaggccccgggtaagggcctggaatgggttgcaaggatttatc
ctacgaatggttatactagatatgccgatagcgtcaagggccgtttcact
ataagcgcagacacatccaaaaacacagcctacctgcagatgaacagcct
gcgtgctgaggacactgccgtctattattgttctagatggggaggggacg
gcttctatgctatggactactggggtcaaggaaccctggtcaccgtctcg
agt
long spacer
gagagcaagtacggaccgccctgccccccttgccctgcccccgagttcct
gggcggacccagcgtgttcctgttcccccccaagcccaaggacaccctga
tgatcagccggacccccgaggtgacctgcgtggtggtggacgtgagccag
gaagatcccgaggtccagttcaattggtacgtggacggcgtggaagtgca
caacgccaagaccaagcccagagaggaacagttcaacagcacctaccggg
tggtgtctgtgctgaccgtgctgcaccaggactggctgaacggcaaagaa
tacaagtgcaaggtgtccaacaagggcctgcccagcagcatcgaaaagac
catcagcaaggccaagggccagcctcgcgagccccaggtgtacaccctgc
ctccctcccaggaagagatgaccaagaaccaggtgtccctgacctgcctg
gtgaagggcttctaccccagcgacatcgccgtggagtgggagagcaacgg
ccagcctgagaacaactacaagaccacccctcccgtgctggacagcgacg
gcagcttcttcctgtacagccggctgaccgtggacaagagccggtggcag
gaaggcaacgtctttagctgcagcgtgatgcacgaggccctgcacaacca
ctacacccagaagagcctgagcctgtccctgggcaag
CD28tm: atgttctgggtgctggtggtggtgggcggggtgctggcctgc
tacagcctgctggtgacagtggccttcatcatcttttgggtg
4-1BB
aaacggggcagaaagaaactcctgtatatattcaaacaaccatttatgag
accagtacaaactactcaagaggaagatggctgtagctgccgatttccag
aagaagaagaaggaggatgtgaactg
CD3zeta
Cgggtgaagttcagcagaagcgccgacgcccctgcctaccagcagggcca
gaatcagctgtacaacgagctgaacctgggcagaagggaagagtacgacg
tcctggataagcggagaggccgggaccctgagatgggcggcaagcctcgg
cggaagaacccccaggaaggcctgtataacgaactgcagaaagacaagat
ggccgaggcctacagcgagatcggcatgaagggcgagcggaggcggggca
agggccacgacggcctgtatcagggcctgtccaccgccaccaaggatacc
tacgacgccctgcacatgcaggccctgcccccaagg
T2A
Ctcgagggcggcggagagggcagaggaagtcttctaacatgcggtgacgt
ggaggagaatcccggccctagg
tEGFR
atgcttctcctggtgacaagccttctgctctgtgagttaccacacccagc
attcctcctgatcccacgcaaagtgtgtaacggaataggtattggtgaat
ttaaagactcactctccataaatgctacgaatattaaacacttcaaaaac
tgcacctccatcagtggcgatctccacatcctgccggtggcatttagggg
tgactccttcacacatactcctcctctggatccacaggaactggatattc
tgaaaaccgtaaaggaaatcacagggtttttgctgattcaggcttggcct
gaaaacaggacggacctccatgcctttgagaacctagaaatcatacgcgg
caggaccaagcaacatggtcagttttctcttgcagtcgtcagcctgaaca
taacatccttgggattacgctccctcaaggagataagtgatggagatgtg
ataatttcaggaaacaaaaatttgtgctatgcaaatacaataaactggaa
aaaactgtttgggacctccggtcagaaaaccaaaattataagcaacagag
gtgaaaacagctgcaaggccacaggccaggtctgccatgccttgtgctcc
cccgagggctgctggggcccggagcccagggactgcgtctcttgccggaa
tgtcagccgaggcagggaatgcgtggacaagtgcaaccttctggagggtg
agccaagggagtttgtggagaactctgagtgcatacagtgccacccagag
tgcctgcctcaggccatgaacatcacctgcacaggacggggaccagacaa
ctgtatccagtgtgcccactacattgacggcccccactgcgtcaagacct
gcccggcaggagtcatgggagaaaacaacaccctggtctggaagtacgca
gacgccggccatgtgtgccacctgtgccatccaaactgcacctacggatg
cactgggccaggtcttgaaggctgtccaacgaatgggcctaagatcccgt
ccatcgccactgggatggtgggggccctcctcttgctgctggtggtggcc
ctggggatcggcctcttcatgtga

1-35. (canceled)

36. A chimeric receptor comprising:
a) a ligand binding domain comprising a variable light chain (VL) domain comprising a VL domain present in the amino acid sequence encoded by SEQ ID NO:3, and a variable heavy chain (VH) domain comprising a VH domain present in the amino acid sequence encoded by SEQ ID NO:3;
b) a transmembrane domain;
c) a polypeptide spacer located between the ligand binding domain and the transmembrane domain, wherein the polypeptide spacer is 15 amino acids or less in length and comprises the amino acid sequence of SEQ ID NO:51; and
d) an intracellular signaling domain that comprises the signaling domain of CD3ζ and a costimulatory domain.

37. The chimeric receptor of claim 36, wherein the ligand binding domain is a single chain variable fragment (scFv) comprising the VH domain and the VL domain separated by a peptide linker.

38. The chimeric receptor of claim 37, wherein the scFv has a VL-linker-VH orientation.

39. The chimeric receptor of claim 36, wherein the polypeptide spacer is 12 amino acids in length.

40. The chimeric receptor of claim 36, wherein the polypeptide spacer comprises the amino acid sequence of SEQ ID NO:21.

41. The chimeric receptor of claim 36, wherein the polypeptide spacer consists of the amino acid sequence of SEQ ID NO:21.

42. The chimeric receptor of claim 40, wherein the ligand binding domain is a single chain variable fragment (scFv) comprising the VH domain and the VL domain separated by a peptide linker.

43. The chimeric receptor of claim 42, wherein the scFv has a VL-linker-VH orientation.

44. The chimeric receptor of claim 39, wherein the ligand binding domain is a single chain variable fragment (scFv) comprising the VH domain and the VL domain separated by a peptide linker.

45. The chimeric receptor of claim 44, wherein the scFv has a VL-linker-VH orientation.

46. The chimeric receptor of claim 36, wherein the transmembrane domain comprises a transmembrane domain of CD8 or CD28.

47. The chimeric receptor of claim 36, wherein the transmembrane domain comprises a transmembrane domain of CD28 comprising the amino acid sequence encoded by SEQ ID NO:5.

48. The chimeric receptor of claim 36, wherein the costimulatory domain comprises the signaling domain of 4-1BB or the signaling domain of CD28.

49. The chimeric receptor of claim 36, wherein the costimulatory domain is a 4-1BB signaling domain that comprises amino acids 214-255 of SEQ ID NO:15.

50. The chimeric receptor of claim 36, wherein the costimulatory domain is a CD28 signaling domain that comprises amino acids 180-220 of SEQ ID NO:14.

51. The chimeric receptor of claim 36, wherein the CD3ζ signaling domain comprises the amino acid sequence encoded by SEQ ID NO: 7.

52. The chimeric receptor of claim 36, wherein:
the transmembrane domain comprises the amino acid sequence encoded by the polynucleotide sequence set forth in SEQ ID NO:5; and
the intracellular signaling domain comprises the amino acid sequence encoded by the polynucleotide sequence set forth in SEQ ID NO:6 and the amino acid sequence encoded by the polynucleotide sequence set forth in SEQ ID NO: 7.

53. The chimeric receptor of claim 40, wherein:
the transmembrane domain comprises the amino acid sequence encoded by the polynucleotide sequence set forth in SEQ ID NO:5; and
the intracellular signaling domain comprises the amino acid sequence encoded by the polynucleotide sequence set forth in SEQ ID NO:6 and the amino acid sequence encoded by the polynucleotide sequence set forth in SEQ ID NO: 7.

54. The chimeric receptor of claim 41, wherein:
the transmembrane domain comprises the amino acid sequence encoded by the polynucleotide sequence set forth in SEQ ID NO:5; and
the intracellular signaling domain comprises the amino acid sequence encoded by the polynucleotide sequence set forth in SEQ ID NO:6 and the amino acid sequence encoded by the polynucleotide sequence set forth in SEQ ID NO: 7.

55. A nucleic acid encoding the chimeric receptor of claim 36.

56. An expression vector comprising the nucleic acid of claim 55.

57. A lymphocyte comprising the chimeric receptor of claim 36.

58. The lymphocyte of claim 57, wherein the lymphocyte is a T cell.

59. The lymphocyte of claim 57, wherein the lymphocyte is from a subject with a disease or disorder to be treated by a cellular immunotherapy comprising the chimeric receptor.

60. The lymphocyte of claim 57, wherein the lymphocyte is from a healthy donor.

61. A composition comprising the lymphocyte of claim 57 in a pharmaceutically acceptable excipient.

62. A method of performing cellular immunotherapy in a subject having a disease or disorder comprising administering the composition of claim 61 to the subject.

63. The method of claim 62, wherein the disease or disorder is a cancer.


SEQUENCE LISTING (79 sequences)

SEQ ID NO: 1 - skipped

SEQ ID NO: 2 - DNA, 66 bp, synthetic construct
  misc_feature 1..66: note = Synthetic sequence
  source 1..66: mol_type = other DNA
atgctgctgctggtgaccagcctgctgctgtgcgagctgccccaccccgcctttctgctgatcccc

SEQ ID NO: 3 - DNA, 735 bp, synthetic construct
  misc_feature 1..735: note = Synthetic sequence
  source 1..735: mol_type = other DNA
gacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagc

SEQ ID NO: 4 - DNA, 36 bp, synthetic construct
  misc_feature 1..36: note = Synthetic sequence
  source 1..36: mol_type = other DNA
gaatctaagtacggaccgccctgccccccttgccct

SEQ ID NO: 5 - DNA, 84 bp, synthetic construct
  misc_feature 1..84: note = Synthetic sequence
  source 1..84: mol_type = other DNA
atgttctgggtgctggtggtggtcggaggcgtgctggcctgctacagcctgctggtcaccgtggccttcatcatcttttgggtg

SEQ ID NO: 6 - DNA, 126 bp, synthetic construct
  misc_feature 1..126: note = Synthetic sequence
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 7 - DNA, 336 bp, synthetic construct
  misc_feature 1..336: note = Synthetic sequence
  source 1..336: mol_type = other DNA
cgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 8 - DNA, 72 bp, synthetic construct
  misc_feature 1..72: note = Synthetic sequence
  source 1..72: mol_type = other DNA
ctcgagggcggcggagagggcagaggaagtcttctaacatgcggtgacgtggaggagaatcccggccctagg

SEQ ID NO: 9 - DNA, 1074 bp, synthetic construct
  misc_feature 1..1074: note = Synthetic sequence
  source 1..1074: mol_type = other DNA
atgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtga

SEQ ID NO: 10 - DNA, 2529 bp, synthetic construct
  misc_feature 1..2529: note = Synthetic sequence
  source 1..2529: mol_type = other DNA
atgctgctgctggtgaccagcctgctgctgtgcgagctgccccaccccgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcgagagcaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggccctaggatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtga

SEQ ID NO: 11 - protein, 841 aa, synthetic construct
  REGION 1..841: note = Synthetic sequence
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 12 - protein, 15 aa, synthetic construct
  REGION 1..15: note = Synthetic sequence
GGGGSGGGGSGGGGS

SEQ ID NO: 13 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 15 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 16 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 17 - protein, 15 aa, Homo sapiens
EPKSCDKTHTCPPCP

SEQ ID NO: 18 - protein, 12 aa, synthetic construct
  REGION 1..12: note = Synthetic sequence
ERKCCVECPPCP

SEQ ID NO: 19 - protein, 61 aa, Homo sapiens
ELKTPLGDTHTCPRCPEPKSCDTPPPCPRCPEPKSCDTPPPCPRCPEPKSCDTPPPCPRCP

SEQ ID NO: 20 - protein, 12 aa, Homo sapiens
ESKYGPPCPSCP

SEQ ID NO: 21 - protein, 12 aa, synthetic construct
  REGION 1..12: note = Synthetic sequence
ESKYGPPCPPCP

SEQ ID NO: 22 - DNA, 26 bp, synthetic construct
  misc_feature 1..26: note = Primer sequence
  source 1..26: mol_type = other DNA
atcaaaagaatagaccgagatagggt

SEQ ID NO: 23 - DNA, 26 bp, synthetic construct
  misc_feature 1..26: note = Primer sequence
  source 1..26: mol_type = other DNA
ccgtacctttaagaccaatgacttac

SEQ ID NO: 24 - DNA, 18 bp, synthetic construct
  misc_feature 1..18: note = Primer sequence
  source 1..18: mol_type = other DNA
ttgagagttttcgccccg

SEQ ID NO: 25 - DNA, 24 bp, synthetic construct
  misc_feature 1..24: note = Primer sequence
  source 1..24: mol_type = other DNA
aatagacagatcgctgagataggt

SEQ ID NO: 26 - DNA, 18 bp, synthetic construct
  misc_feature 1..18: note = Primer sequence
  source 1..18: mol_type = other DNA
caggtatccggtaagcgg

SEQ ID NO: 27 - DNA, 19 bp, synthetic construct
  misc_feature 1..19: note = Primer sequence
  source 1..19: mol_type = other DNA
cgaccagcaaccatagtcc

SEQ ID NO: 28 - DNA, 18 bp, synthetic construct
  misc_feature 1..18: note = Primer sequence
  source 1..18: mol_type = other DNA
tagcggtttgactcacgg

SEQ ID NO: 29 - DNA, 20 bp, synthetic construct
  misc_feature 1..20: note = Primer sequence
  source 1..20: mol_type = other DNA
gcagggagctagaacgattc

SEQ ID NO: 30 - DNA, 22 bp, synthetic construct
  misc_feature 1..22: note = Primer sequence
  source 1..22: mol_type = other DNA
attgtctggtatagtgcagcag

SEQ ID NO: 31 - DNA, 16 bp, synthetic construct
  misc_feature 1..16: note = Primer sequence
  source 1..16: mol_type = other DNA
tcgcaacgggtttgcc

SEQ ID NO: 32 - DNA, 21 bp, synthetic construct
  misc_feature 1..21: note = Primer sequence
  source 1..21: mol_type = other DNA
aggaagatatcgccacctact

SEQ ID NO: 33 - DNA, 20 bp, synthetic construct
  misc_feature 1..20: note = Primer sequence
  source 1..20: mol_type = other DNA
cgggtgaagttcagcagaag

SEQ ID NO: 34 - DNA, 20 bp, synthetic construct
  misc_feature 1..20: note = Primer sequence
  source 1..20: mol_type = other DNA
actgtgtttgctgacgcaac

SEQ ID NO: 35 - DNA, 20 bp, synthetic construct
  misc_feature 1..20: note = Primer sequence
  source 1..20: mol_type = other DNA
atgcttctcctggtgacaag

SEQ ID NO: 36 - protein, 18 aa, synthetic construct
  REGION 1..18: note = Synthetic sequence
GSTSGSGKPGSGEGSTKG

SEQ ID NO: 37 - DNA, 10051 bp, synthetic construct
  misc_feature 1..10051: note = Synthetic sequence
  source 1..10051: mol_type = other DNA
gttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggatctacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggggatcaattgcatgaagaatctgcttagggttaggcgttttgcgctgcttcgcgaggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacggctagcgaattcctcgaggccaccatgctgctgctggtgacaagcctgctgctgtgcgagctgccccaccccgcctttctgctgatcccccaggaacagctcgtcgaaagcggcggcagactggtgacacctggcggcagcctgaccctgagctgcaaggccagcggcttcgacttcagcgcctactacatgagctgggtccgccaggcccctggcaagggactggaatggatcgccaccatctaccccagcagcggcaagacctactacgccacctgggtgaacggacggttcaccatctccagcgacaacgcccagaacaccgtggacctgcagatgaacagcctgacagccgccgaccgggccacctacttttgcgccagagacagctacgccgacgacggcgccctgttcaacatctggggccctggcaccctggtgacaatctctagcggcggaggcggatctggtggcggaggaagtggcggcggaggatctgagctggtgctgacccagagcccctctgtgtctgctgccctgggaagccctgccaagatcacctgtaccctgagcagcgcccacaagaccgacaccatcgactggtatcagcagctgcagggcgaggcccccagatacctgatgcaggtgcagagcgacggcagctacaccaagaggccaggcgtgcccgaccggttcagcggatctagctctggcgccgaccgctacctgatcatccccagcgtgcaggccgatgacgaggccgattactactgtggcgccgactacatcggcggctacgtgttcggcggaggcacccagctgaccgtgaccggcgagtctaagtacggaccgccctgccccccttgccctgcccccgagttcctgggcggacccagcgtgttcctgttcccccccaagcccaaggacaccctgatgatcagccggacccccgaggtgacctgcgtggtggtggacgtgagccaggaagatcccgaggtccagttcaattggtacgtggacggcgtggaagtgcacaacgccaagaccaagcccagagaggaacagttcaacagcacctaccgggtggtgtctgtgctgaccgtgctgcaccaggactggctgaacggcaaagaatacaagtgcaaggtgtccaacaagggcctgcccagcagcatcgaaaagaccatcagcaaggccaagggccagcctcgcgagccccaggtgtacaccctgcctccctcccaggaagagatgaccaagaaccaggtgtccctgacctgcctggtgaagggcttctaccccagcgacatcgccgtggagtgggagagcaacggccagcctgagaacaactacaagaccacccctcccgtgctggacagcgacggcagcttcttcctgtacagccggctgaccgtggacaagagccggtggcaggaaggcaacgtctttagctgcagcgtgatgcacgaggccctgcacaaccactacacccagaagagcctgagcctgtccctgggcaagatgttctgggtgctggtggtggtgggcggggtgctggcctgctacagcctgctggtgacagtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggccctaggatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtgagcggccgctctagacccgggctgcaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagccgtacctttaagaccaatgacttacaaggcagctgtagatcttagccactttttaaaagaaaaggggggactggaagggctaattcactcccaaagaagacaagatctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagaattcgatatcaagcttatcgataccgtcgacctcgagggggggcccggtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggagctccaccgcggtggcggcctcgaggtcgagatccggtcgaccagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttcgacggtatcgattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggaattcggagtggcgagccctcagatcctgcatataagcagctgctttttgcctgtactgggtctctctg

SEQ ID NO: 38 - protein, 1062 aa, synthetic construct
  REGION 1..1062: note = Synthetic sequence
MLLLVTSLLLCELPHPAFLLIPQEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSGGGGSGGGGSGGGGSELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVTGESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 39 - DNA, 9721 bp, synthetic construct
  misc_feature 1..9721: note = Synthetic sequence
  source 1..9721: mol_type = other DNA
gttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggatctacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggggatcaattgcatgaagaatctgcttagggttaggcgttttgcgctgcttcgcgaggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacggctagcgaattcctcgaggccaccatgctgctgctggtgacaagcctgctgctgtgcgagctgccccaccccgcctttctgctgatcccccaggaacagctcgtcgaaagcggcggcagactggtgacacctggcggcagcctgaccctgagctgcaaggccagcggcttcgacttcagcgcctactacatgagctgggtccgccaggcccctggcaagggactggaatggatcgccaccatctaccccagcagcggcaagacctactacgccacctgggtgaacggacggttcaccatctccagcgacaacgcccagaacaccgtggacctgcagatgaacagcctgacagccgccgaccgggccacctacttttgcgccagagacagctacgccgacgacggcgccctgttcaacatctggggccctggcaccctggtgacaatctctagcggcggaggcggatctggtggcggaggaagtggcggcggaggatctgagctggtgctgacccagagcccctctgtgtctgctgccctgggaagccctgccaagatcacctgtaccctgagcagcgcccacaagaccgacaccatcgactggtatcagcagctgcagggcgaggcccccagatacctgatgcaggtgcagagcgacggcagctacaccaagaggccaggcgtgcccgaccggttcagcggatctagctctggcgccgaccgctacctgatcatccccagcgtgcaggccgatgacgaggccgattactactgtggcgccgactacatcggcggctacgtgttcggcggaggcacccagctgaccgtgaccggcgagtctaagtacggaccgccctgccccccttgccctggccagcctcgcgagccccaggtgtacaccctgcctccctcccaggaagagatgaccaagaaccaggtgtccctgacctgcctggtgaagggcttctaccccagcgacatcgccgtggagtgggagagcaacggccagcctgagaacaactacaagaccacccctcccgtgctggacagcgacggcagcttcttcctgtacagccggctgaccgtggacaagagccggtggcaggaaggcaacgtctttagctgcagcgtgatgcacgaggccctgcacaaccactacacccagaagagcctgagcctgtccctgggcaagatgttctgggtgctggtggtggtgggcggggtgctggcctgctacagcctgctggtgacagtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggccctaggatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtgagcggccgctctagacccgggctgcaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagccgtacctttaagaccaatgacttacaaggcagctgtagatcttagccactttttaaaagaaaaggggggactggaagggctaattcactcccaaagaagacaagatctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagaattcgatatcaagcttatcgataccgtcgacctcgagggggggcccggtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggagctccaccgcggtggcggcctcgaggtcgagatccggtcgaccagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttcgacggtatcgattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggaattcggagtggcgagccctcagatcctgcatataagcagctgctttttgcctgtactgggtctctctg

SEQ ID NO: 40 - protein, 952 aa, synthetic construct
  REGION 1..952: note = Synthetic sequence
MLLLVTSLLLCELPHPAFLLIPQEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSGGGGSGGGGSGGGGSELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVTGESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 41 - DNA, 9400 bp, synthetic construct
  misc_feature 1..9400: note = Synthetic sequence
  source 1..9400: mol_type = other DNA
gttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggatctacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggggatcaattgcatgaagaatctgcttagggttaggcgttttgcgctgcttcgcgaggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacggctagcgaattcctcgaggccaccatgctgctgctggtgacaagcctgctgctgtgcgagctgccccaccccgcctttctgctgatcccccaggaacagctcgtcgaaagcggcggcagactggtgacacctggcggcagcctgaccctgagctgcaaggccagcggcttcgacttcagcgcctactacatgagctgggtccgccaggcccctggcaagggactggaatggatcgccaccatctaccccagcagcggcaagacctactacgccacctgggtgaacggacggttcaccatctccagcgacaacgcccagaacaccgtggacctgcagatgaacagcctgacagccgccgaccgggccacctacttttgcgccagagacagctacgccgacgacggcgccctgttcaacatctggggccctggcaccctggtgacaatctctagcggcggaggcggatctggtggcggaggaagtggcggcggaggatctgagctggtgctgacccagagcccctctgtgtctgctgccctgggaagccctgccaagatcacctgtaccctgagcagcgcccacaagaccgacaccatcgactggtatcagcagctgcagggcgaggcccccagatacctgatgcaggtgcagagcgacggcagctacaccaagaggccaggcgtgcccgaccggttcagcggatctagctctggcgccgaccgctacctgatcatccccagcgtgcaggccgatgacgaggccgattactactgtggcgccgactacatcggcggctacgtgttcggcggaggcacccagctgaccgtgaccggcgagtctaagtacggaccgccctgccccccttgccctatgttctgggtgctggtggtggtgggcggggtgctggcctgctacagcctgctggtgacagtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggccctaggatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtgagcggccgctctagacccgggctgcaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagccgtacctttaagaccaatgacttacaaggcagctgtagatcttagccactttttaaaagaaaaggggggactggaagggctaattcactcccaaagaagacaagatctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagaattcgatatcaagcttatcgataccgtcgacctcgagggggggcccggtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggagctccaccgcggtggcggcctcgaggtcgagatccggtcgaccagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttcgacggtatcgattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggaattcggagtggcgagccctcagatcctgcatataagcagctgctttttgcctgtactgggtctctctg

SEQ ID NO: 42 - protein, 845 aa, synthetic construct
  REGION 1..845: note = Synthetic sequence
MLLLVTSLLLCELPHPAFLLIPQEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSGGGGSGGGGSGGGGSELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVTGESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 43 - DNA, 10015 bp, synthetic construct
  misc_feature 1..10015: note = Synthetic sequence
  source 1..10015: mol_type = other DNA
gttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggatctacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggggatcaattgcatgaagaatctgcttagggttaggcgttttgcgctgcttcgcgaggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacggctagcgaattcgccaccatgctgctgctggtgacaagcctgctgctgtgcgagctgccccaccccgcctttctgctgatcccccagagcgtgaaagagtccgagggcgacctggtcacaccagccggcaacctgaccctgacctgtaccgccagcggcagcgacatcaacgactaccccatctcttgggtccgccaggctcctggcaagggactggaatggatcggcttcatcaacagcggcggcagcacttggtacgccagctgggtcaaaggccggttcaccatcagccggaccagcaccaccgtggacctgaagatgacaagcctgaccaccgacgacaccgccacctacttttgcgccagaggctacagcacctactacggcgacttcaacatctggggccctggcaccctggtcacaatctctagcggcggaggcggcagcggaggtggaggaagtggcggcggaggatccgagctggtcatgacccagacccccagcagcacatctggcgccgtgggcggcaccgtgaccatcaattgccaggccagccagagcatcgacagcaacctggcctggttccagcagaagcccggccagccccccaccctgctgatctacagagcctccaacctggccagcggcgtgccaagcagattcagcggcagcagatctggcaccgagtacaccctgaccatctccggcgtgcagagagaggacgccgctacctattactgcctgggcggcgtgggcaacgtgtcctacagaaccagcttcggcggaggtactgaggtggtcgtcaaatacggaccgccctgccccccttgccctgcccccgagttcctgggcggacccagcgtgttcctgttcccccccaagcccaaggacaccctgatgatcagccggacccccgaggtgacctgcgtggtggtggacgtgagccaggaagatcccgaggtccagttcaattggtacgtggacggcgtggaagtgcacaacgccaagaccaagcccagagaggaacagttcaacagcacctaccgggtggtgtctgtgctgaccgtgctgcaccaggactggctgaacggcaaagaatacaagtgcaaggtgtccaacaagggcctgcccagcagcatcgaaaagaccatcagcaaggccaagggccagcctcgcgagccccaggtgtacaccctgcctccctcccaggaagagatgaccaagaaccaggtgtccctgacctgcctggtgaagggcttctaccccagcgacatcgccgtggagtgggagagcaacggccagcctgagaacaactacaagaccacccctcccgtgctggacagcgacggcagcttcttcctgtacagccggctgaccgtggacaagagccggtggcaggaaggcaacgtctttagctgcagcgtgatgcacgaggccctgcacaaccactacacccagaagagcctgagcctgtccctgggcaagatgttctgggtgctggtggtggtgggcggggtgctggcctgctacagcctgctggtgacagtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggccctaggatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtgagcggccgctctagacccgggctgcaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagccgtacctttaagaccaatgacttacaaggcagctgtagatcttagccactttttaaaagaaaaggggggactggaagggctaattcactcccaaagaagacaagatctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagaattcgatatcaagcttatcgataccgtcgacctcgagggggggcccggtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggagctccaccgcggtggcggcctcgaggtcgagatccggtcgaccagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttcgacggtatcgattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggaattcggagtggcgagccctcagatcctgcatataagcagctgctttttgcctgtactgggtctctctg

SEQ ID NO: 44 - protein, 1055 aa, synthetic construct [sequence omitted]

SEQ ID NO: 45 - DNA, 10015 bp, synthetic construct; identical to SEQ ID NO: 43
  misc_feature 1..10015: note = Synthetic sequence
  source 1..10015: mol_type = other DNA

SEQ ID NO: 46 - protein, 945 aa, synthetic construct
  REGION 1..945: note = Synthetic sequence
MLLLVTSLLLCELPHPAFLLIPQSVKESEGDLVTPAGNLTLTCTASGSDINDYPISWVRQAPGKGLEWIGFINSGGSTWYASWVKGRFTISRTSTTVDLKMTSLTTDDTATYFCARGYSTYYGDFNIWGPGTLVTISSGGGGSGGGGSGGGGSELVMTQTPSSTSGAVGGTVTINCQASQSIDSNLAWFQQKPGQPPTLLIYRASNLASGVPSRFSGSRSGTEYTLTISGVQREDAATYYCLGGVGNVSYRTSFGGGTEVVVKESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 47 - DNA, 9685 bp, synthetic construct
  misc_feature 1..9685: note = Synthetic sequence
  source 1..9685: mol_type = other DNA
gttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggatctacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggggatcaattgcatgaagaatctgcttagggttaggcgttttgcgctgcttcgcgaggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacggctagcgaattcgccaccatgctgctgctggtgacaagcctgctgctgtgcgagctgccccaccccgcctttctgctgatcccccagagcgtgaaagagtccgagggcgacctggtcacaccagccggcaacctgaccctgacctgtaccgccagcggcagcgacatcaacgactaccccatctcttgggtccgccaggctcctggcaagggactggaatggatcggcttcatcaacagcggcggcagcacttggtacgccagctgggtcaaaggccggttcaccatcagccggaccagcaccaccgtggacctgaagatgacaagcctgaccaccgacgacaccgccacctacttttgcgccagaggctacagcacctactacggcgacttcaacatctggggccctggcaccctggtcacaatctctagcggcggaggcggcagcggaggtggaggaagtggcggcggaggatccgagctggtcatgacccagacccccagcagcacatctggcgccgtgggcggcaccgtgaccatcaattgccaggccagccagagcatcgacagcaacctggcctggttccagcagaagcccggccagccccccaccctgctgatctacagagcctccaacctggccagcggcgtgccaagcagattcagcggcagcagatctggcaccgagtacaccctgaccatctccggcgtgcagagagaggacgccgctacctattactgcctgggcggcgtgggcaacgtgtcctacagaaccagcttcggcggaggtactgaggtggtcgtcaaatacggaccgccctgccccccttgccctggccagcctcgcgagccccaggtgtacaccctgcctccctcccaggaagagatgaccaagaaccaggtgtccctgacctgcctggtgaagggcttctaccccagcgacatcgccgtggagtgggagagcaacggccagcctgagaacaactacaagaccacccctcccgtgctggacagcgacggcagcttcttcctgtacagccggctgaccgtggacaagagccggtggcaggaaggcaacgtctttagctgcagcgtgatgcacgaggccctgcacaaccactacacccagaagagcctgagcctgtccctgggcaagatgttctgggtgctggtggtggtgggcggggtgctggcctgctacagcctgctggtgacagtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggccctaggatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtgagcggccgctctagacccgggctgcaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagccgtacctttaagaccaatgacttacaaggcagctgtagatcttagccactttttaaaagaaaaggggggactggaagggctaattcactcccaaagaagacaagatctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagaattcgatatcaagcttatcgataccgtcgacctcgagggggggcccggtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggagctccaccgcggtggcggcctcgaggtcgagatccggtcgaccagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttcgacggtatcgattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggaattcggagtggcgagccctcagatcctgcatataagcagctgctttttgcctgtactgggtctctctg

SEQ ID NO: 48 - protein, 838 aa, synthetic construct
  REGION 1..838: note = Synthetic sequence
MLLLVTSLLLCELPHPAFLLIPQSVKESEGDLVTPAGNLTLTCTASGSDINDYPISWVRQAPGKGLEWIGFINSGGSTWYASWVKGRFTISRTSTTVDLKMTSLTTDDTATYFCARGYSTYYGDFNIWGPGTLVTISSGGGGSGGGGSGGGGSELVMTQTPSSTSGAVGGTVTINCQASQSIDSNLAWFQQKPGQPPTLLIYRASNLASGVPSRFSGSRSGTEYTLTISGVQREDAATYYCLGGVGNVSYRTSFGGGTEVVVKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 49 - protein, 119 aa, synthetic construct
  REGION 1..119: note = Synthetic sequence
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 50 - protein, 229 aa, synthetic construct
  REGION 1..229: note = Synthetic sequence
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 51 - protein, 9 aa, synthetic construct
  REGION 1..9: note = Synthetic sequence
YGPPCPPCP

SEQ ID NO: 52 - protein, 10 aa, synthetic construct
  REGION 1..10: note = Synthetic sequence
KYGPPCPPCP

SEQ ID NO: 53 - protein, 14 aa, synthetic construct
  REGION 1..14: note = Synthetic sequence
EVVVKYGPPCPPCP

SEQ ID NO: 54 - DNA, 2529 bp, synthetic construct
  misc_feature 1..2529: note = Synthetic sequence
  source 1..2529: mol_type = other DNA
atgcttctcctggtgacaagccttctgctctgtgagttaccacacccagcattcctcctgatcccagatatccagatgacccagtccccgagctccctgtccgcctctgtgggcgatagggtcaccatcacctgccgtgccagtcaggatgtgaatactgctgtagcctggtatcaacagaaaccaggaaaagctccgaaactactgatttactcggcatccttcctctactctggagtcccttctcgcttctctggttccagatctgggacggatttcactctgaccatcagcagtctgcagccggaagacttcgcaacttattactgtcagcaacattatactactcctcccacgttcggacagggtaccaaggtggagatcaaaggcagtactagcggcggtggctccgggggcggatccggtgggggcggcagcagcgaggttcagctggtggagtctggcggtggcctggtgcagccagggggctcactccgtttgtcctgtgcagcttctggcttcaacattaaagacacctatatacactgggtgcgtcaggccccgggtaagggcctggaatgggttgcaaggatttatcctacgaatggttatactagatatgccgatagcgtcaagggccgtttcactataagcgcagacacatccaaaaacacagcctacctgcagatgaacagcctgcgtgctgaggacactgccgtctattattgttctagatggggaggggacggcttctatgctatggactactggggtcaaggaaccctggtcaccgtctcgagtgagagcaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggccctaggatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtga

SEQ ID NO: 55 - DNA, 2850 bp, synthetic construct
  misc_feature 1..2850: note = Synthetic sequence
  source 1..2850: mol_type = other DNA
atgcttctcctggtgacaagccttctgctctgtgagttaccacacccagcattcctcctgatcccagatatccagatgacccagtccccgagctccctgtccgcctctgtgggcgatagggtcaccatcacctgccgtgccagtcaggatgtgaatactgctgtagcctggtatcaacagaaaccaggaaaagctccgaaactactgatttactcggcatccttcctctactctggagtcccttctcgcttctctggttccagatctgggacggatttcactctgaccatcagcagtctgcagccggaagacttcgcaacttattactgtcagcaacattatactactcctcccacgttcggacagggtaccaaggtggagatcaaaggcagtactagcggcggtggctccgggggcggatccggtgggggcggcagcagcgaggttcagctggtggagtctggcggtggcctggtgcagccagggggctcactccgtttgtcctgtgcagcttctggcttcaacattaaagacacctatatacactgggtgcgtcaggccccgggtaagggcctggaatgggttgcaaggatttatcctacgaatggttatactagatatgccgatagcgtcaagggccgtttcactataagcgcagacacatccaaaaacacagcctacctgcagatgaacagcctgcgtgctgaggacactgccgtctattattgttctagatggggaggggacggcttctatgctatggactactggggtcaaggaaccctggtcaccgtctcgagtgagagcaagtacggaccgccctgccccccttgccctggccagcctagagaaccccaggtgtacaccctgcctcccagccaggaagagatgaccaagaaccaggtgtccctgacctgcctggtcaaaggcttctaccccagcgatatcgccgtggaatgggagagcaacggccagcccgagaacaactacaagaccaccccccctgtgctggacagcgacggcagcttcttcctgtactcccggctgaccgtggacaagagccggtggcaggaaggcaacgtcttcagctgcagcgtgatgcacgaggccctgcacaaccactacacccagaagtccctgagcctgagcctgggcaagatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggccctaggatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtga

SEQ ID NO: 56 - DNA, 3180 bp, synthetic construct
  misc_feature 1..3180: note = Synthetic sequence
  source 1..3180: mol_type = other DNA
atgcttctcctggtgacaagccttctgctctgtgagttaccacacccagcattcctcctgatcccagatatccagatgacccagtccccgagctccctgtccgcctctgtgggcgatagggtcaccatcacctgccgtgccagtcaggatgtgaatactgctgtagcctggtatcaacagaaaccaggaaaagctccgaaactactgatttactcggcatccttcctctactctggagtcccttctcgcttctctggttccagatctgggacggatttcactctgaccatcagcagtctgcagccggaagacttcgcaacttattactgtcagcaacattatactactcctcccacgttcggacagggtaccaaggtggagatcaaaggcagtactagcggcggtggctccgggggcggatccggtgggggcggcagcagcgaggttcagctggtggagtctggcggtggcctggtgcagccagggggctcactccgtttgtcctgtgcagcttctggcttcaacattaaagacacctatatacactgggtgcgtcaggccccgggtaagggcctggaatgggttgcaaggatttatcctacgaatggttatactagatatgccgatagcgtcaagggccgtttcactataagcgcagacacatccaaaaacacagcctacctgcagatgaacagcctgcgtgctgaggacactgccgtctattattgttctagatggggaggggacggcttctatgctatggactactggggtcaaggaaccctggtcaccgtctcgagtgagagcaagtacggaccgccctgccccccttgccctgcccccgagttcctgggcggacccagcgtgttcctgttcccccccaagcccaaggacaccctgatgatcagccggacccccgaggtgacctgcgtggtggtggacgtgagccaggaagatcccgaggtccagttcaattggtacgtggacggcgtggaagtgcacaacgccaagaccaagcccagagaggaacagttcaacagcacctaccgggtggtgtctgtgctgaccgtgctgcaccaggactggctgaacggcaaagaatacaagtgcaaggtgtccaacaagggcctgcccagcagcatcgaaaagaccatcagcaaggccaagggccagcctcgcgagccccaggtgtacaccctgcctccctcccaggaagagatgaccaagaaccaggtgtccctgacctgcctggtgaagggcttctaccccagcgacatcgccgtggagtgggagagcaacggccagcctgagaacaactacaagaccacccctcccgtgctggacagcgacggcagcttcttcctgtacagccggctgaccgtggacaagagccggtggcaggaaggcaacgtctttagctgcagcgtgatgcacgaggccctgcacaaccactacacccagaagagcctgagcctgtccctgggcaagatgttctgggtgctggtggtggtgggcggggtgctggcctgctacagcctgctggtgacagtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggccctaggatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtga

SEQ ID NO: 57 - protein, 11 aa, synthetic construct
  REGION 1..11: note = Synthetic sequence
RASQDISKYLN

SEQ ID NO: 58 - protein, 7 aa, synthetic construct
  REGION 1..7: note = Synthetic sequence
SRLHSGV

SEQ ID NO: 59 - protein, 9 aa, synthetic construct
  REGION 1..9: note = Synthetic sequence
GNTLPYTFG

SEQ ID NO: 60 - protein, 16 aa, synthetic construct
  REGION 1..16: note = Synthetic sequence
VIWGSETTYYNSALKS

SEQ ID NO: 61 - protein, 7 aa, synthetic construct
  REGION 1..7: note = Synthetic sequence
YAMDYWG

SEQ ID NO: 62 - protein, 11 aa, synthetic construct
  REGION 1..11: note = Synthetic sequence
ASGFDFSAYYM

SEQ ID NO: 63 - protein, 7 aa, synthetic construct
  REGION 1..7: note = Synthetic sequence
TIYPSSG

SEQ ID NO: 64 - protein, 9 aa, synthetic construct
  REGION 1..9: note = Synthetic sequence
ADRATYFCA

SEQ ID NO: 65 - protein, 6 aa, synthetic construct
  REGION 1..6: note = Synthetic sequence
DTIDWY

SEQ ID NO: 66 - protein, 16 aa, synthetic construct
  REGION 1..16: note = Synthetic sequence
VQSDGSYTKRPGVPDR

SEQ ID NO: 67 - protein, 8 aa, synthetic construct
  REGION 1..8: note = Synthetic sequence
YIGGYVFG

SEQ ID NO: 68 - protein, 11 aa, synthetic construct
  REGION 1..11: note = Synthetic sequence
SGSDINDYPIS

SEQ ID NO: 69 - protein, 7 aa, synthetic construct
  REGION 1..7: note = Synthetic sequence
INSGGST

SEQ ID NO: 70 - protein, 8 aa, synthetic construct
  REGION 1..8: note = Synthetic sequence
YFCARGYS

SEQ ID NO: 71 - protein, 5 aa, synthetic construct
  REGION 1..5: note = Synthetic sequence
SNLAW

SEQ ID NO: 72 - protein, 16 aa, synthetic construct
  REGION 1..16: note = Synthetic sequence
RASNLASGVPSRFSGS

SEQ ID NO: 73 - protein, 8 aa, synthetic construct
  REGION 1..8: note = Synthetic sequence
NVSYRTSF

SEQ ID NO: 74 - protein, 5 aa, synthetic construct
  REGION 1..5: note = Synthetic sequence
DYGVS

SEQ ID NO: 75 - protein, 28 aa, synthetic construct
  REGION 1..28: note = Synthetic sequence
MFWVLVVVGGVLACYSLLVTVAFIIFWV

SEQ ID NO: 76 - protein, 248 aa, synthetic construct
  REGION 1..248: note = Synthetic sequence
QEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSGGGGSGGGGSGGGGSELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVTG

SEQ ID NO: 77 - protein, 241 aa, synthetic construct
  REGION 1..241: note = Synthetic sequence
QSVKESEGDLVTPAGNLTLTCTASGSDINDYPISWVRQAPGKGLEWIGFINSGGSTWYASWVKGRFTISRTSTTVDLKMTSLTTDDTATYFCARGYSTYYGDFNIWGPGTLVTISSGGGGSGGGGSGGGGSELVMTQTPSSTSGAVGGTVTINCQASQSIDSNLAWFQQKPGQPPTLLIYRASNLASGVPSRFSGSRSGTEYTLTISGVQREDAATYYCLGGVGNVSYRTSFGGGTEVVVK

SEQ ID NO: 78 - DNA, 753 bp, synthetic construct
  misc_feature 1..753: note = Synthetic sequence
  source 1..753: mol_type = other DNA
gcattcctcctgatcccagatatccagatgacccagtccccgagctccctgtccgcctctgtgggcgatagggtcaccatcacctgccgtgccagtcaggatgtgaatactgctgtagcctggtatcaacagaaaccaggaaaagctccgaaactactgatttactcggcatccttcctctactctggagtcccttctcgcttctctggttccagatctgggacggatttcactctgaccatcagcagtctgcagccggaagacttcgcaacttattactgtcagcaacattatactactcctcccacgttcggacagggtaccaaggtggagatcaaaggcagtactagcggcggtggctccgggggcggatccggtgggggcggcagcagcgaggttcagctggtggagtctggcggtggcctggtgcagccagggggctcactccgtttgtcctgtgcagcttctggcttcaacattaaagacacctatatacactgggtgcgtcaggccccgggtaagggcctggaatgggttgcaaggatttatcctacgaatggttatactagatatgccgatagcgtcaagggccgtttcactataagcgcagacacatccaaaaacacagcctacctgcagatgaacagcctgcgtgctgaggacactgccgtctattattgttctagatggggaggggacggcttctatgctatggactactggggtcaaggaaccctggtcaccgtctcgagt

SEQ ID NO: 79 - DNA, 84 bp, synthetic construct
  misc_feature 1..84: note = Synthetic sequence
  source 1..84: mol_type = other DNA
atgttctgggtgctggtggtggtgggcggggtgctggcctgctacagcctgctggtgacagtggccttcatcatcttttgggtg